(19) United States
(12) Patent Application Publication
(10) Pub. No.: US 2007/0237764 A1
(43) Pub. Date: Oct. 11, 2007

(54) BINDING POLYPEPTIDES WITH RESTRICTED DIVERSITY SEQUENCES

(75) Inventors: Sara C. Birtalan, Menlo Park, CA (US); Frederic Fellouse, Toronto (CA); Sachdev S. Sidhu, San Francisco, CA (US)

Correspondence Address: MERCHANT & GOULD PC, P.O. BOX 2903, MINNEAPOLIS, MN, 55402-0903, US

(73) Assignee: Genentech, Inc., South San Francisco, CA (US)

(21) Appl. No.: 11/565,880

(22) Filed: Dec. 1, 2006

Related U.S. Application Data
(60) Provisional application No. 60/805,553, filed on Jun. 22, 2006.
(60) Provisional application No. 60/742,184, filed on Dec. 2, 2005.

(51) Int. Cl. C07K 16/18; A61K 39/395; C07H 21/04; C07K 16/46; C12N 5/16; G01N 33/563; C12N 15/63; C07K 1/00; C07K 14/00

(52) U.S. Cl. 424/133.1; 435/326; 436/512; 530/350; 530/387.1; 530/387.3; 536/23.53

(57) ABSTRACT

The invention provides variant CDRs comprising highly restricted amino acid sequence diversity. These polypeptides provide a flexible and simple source of sequence diversity that can be used as a source for identifying novel antigen binding polypeptides. The invention also provides these polypeptides as fusion polypeptides to heterologous polypeptides such as at least a portion of phage or viral coat proteins, tags and linkers. Libraries comprising a plurality of these polypeptides are also provided. In addition, methods of and compositions for generating and using these polypeptides and libraries are provided.

### CROSS REFERENCE TO RELATED APPLICATIONS

This application is a nonprovisional application which claims priority to U.S. Ser. No. 60/742,184 filed Dec. 2, 2005 and U.S. Ser. No. 60/805,553 filed Jun. 22, 2006, all of which applications are incorporated by reference herein.

### FIELD OF THE INVENTION

The invention generally relates to variant CDRs diversified using highly limited amino acid repertoires, and libraries comprising a plurality of such sequences. The invention also relates to fusion polypeptides comprising these variant CDRs. The invention also relates to methods and compositions useful for identifying novel binding polypeptides that can be used therapeutically or as reagents.

### BACKGROUND

Phage display technology has provided a powerful tool for generating and selecting novel proteins that bind to a ligand, such as an antigen. Using the techniques of phage display allows the generation of large libraries of protein variants that can be rapidly sorted for those sequences that bind to a target antigen with high affinity. Nucleic acids encoding variant polypeptides are fused to a nucleic acid sequence encoding a viral coat protein, such as the gene III protein or the gene VIII protein. Monovalent phage display systems where the nucleic acid sequence encoding the protein or polypeptide is fused to a nucleic acid sequence encoding a portion of the gene III protein have been developed. (Bass, S., Proteins, 8:309 (1990); Lowman and Wells, Methods: A Companion to Methods in Enzymology, 3:205 (1991)). In a monovalent phage display system, the gene fusion is expressed at low levels and wild type gene III proteins are also expressed so that infectivity of the particles is retained. Methods of generating peptide libraries and screening those libraries have been disclosed in many patents (e.g. U.S. Pat. No. 5,723,286, U.S. Pat. No. 5,432, 018, U.S. Pat. No. 5,580,717, U.S. Pat. No. 5,427,908 and U.S. Pat. No. 5,498,530).

The demonstration of expression of peptides on the surface of filamentous phage and the expression of functional antibody fragments in the periplasm of E. coli was important in the development of antibody phage display libraries. (Smith et al., Science (1985), 228:1315; Skerra and Pluckthun, Science (1988), 240:1038). Libraries of antibodies or antigen binding polypeptides have been prepared in a number of ways including by altering a single gene by inserting random DNA sequences or by cloning a family of related genes. Methods for displaying antibodies or antigen binding fragments using phage display have been described in U.S. Pat. Nos. 5,750,373, 5,733,743, 5,837,242, 5,969,108, 6,172,197, 5,580,717, and 5,658,727. The library is then screened for expression of antibodies or antigen binding proteins with desired characteristics.

Phage display technology has several advantages over conventional hybridoma and recombinant methods for preparing antibodies with the desired characteristics. This technology allows the development of large libraries of antibodies with diverse sequences in less time and without the use of animals. Preparation of hybridomas or preparation of humanized antibodies can easily require several months of preparation. In addition, since no immunization is required, phage antibody libraries can be generated for antigens which are toxic or have low antigenicity (Hogenboom, Immunotechniques (1988), 4:1-20). Phage antibody libraries can also be used to generate and identify novel human antibodies.

Antibodies have become very useful as therapeutic agents for a wide variety of conditions. For example, humanized antibodies to HER-2, a tumor antigen, are useful in the diagnosis and treatment of cancer. Other antibodies, such as anti-INF-γ antibody, are useful in treating inflammatory conditions such as Crohn's disease. Phage display libraries have been used to generate human antibodies from immunized and non-immunized humans, germ line sequences, or naïve B cell Ig repertories (Barbas & Burton, Trends Biotech (1996), 14:230; Griffiths et al., EMBO J. (1994), 13:3245; Vaughan et al., Nat. Biotech. (1996), 14:309; Winter EP 0368 684 B1). Naïve, or nonimmune, antigen binding libraries have been generated using a variety of lymphoidal tissues. Some of these libraries are commercially available, such as those developed by Cambridge Antibody Technology and Morphosys (Vaughan et al., Nature Biotech 14:309 (1996); Knappik et al., J. Mol. Biol. 296:57 (1999)). However, many of these libraries have limited diversity.

The ability to identify and isolate high affinity antibodies from a phage display library is important in isolating novel human antibodies for therapeutic use. Isolation of high affinity antibodies from a library is traditionally thought to be dependent, at least in part, on the size of the library, the efficiency of production in bacterial cells and the diversity of the library. See, e.g., Knappik et al., J. Mol. Biol. (1999), 296:57. The size of the library is decreased by inefficiency of production due to improper folding of the antibody or antigen binding protein and the presence of stop codons. Expression in bacterial cells can be inhibited if the antibody or antigen binding domain is not properly folded. Expression can be improved by mutating residues in turns at the surface of the variable/constant interface, or at selected CDR residues. (Deng et al., J. Biol. Chem. (1994), 269:9533, Ulrich et al., PNAS (1995), 92:11907-11911; Forsberg et al., J. Biol. Chem. (1997), 272 :12430). The sequence of the framework region is a factor in providing for proper folding when antibody phage libraries are produced in bacterial cells.

Generating a diverse library of antibodies or antigen binding proteins is also important to isolation of high affinity antibodies. Libraries with diversification in limited CDRs have been generated using a variety of approaches. See, e.g., Tomlinson, Nature Biotech. (2000), 18:989-994. CDR3 regions are of interest in part because they often are found to participate in antigen binding. CDR3 regions on the heavy chain vary greatly in size, sequence and structural conformation.

Others have also generated diversity by randomizing CDR regions of the variable heavy and light chains using all 20 amino acids at each position. It was thought that using all 20 amino acids would result in a large diversity of sequences of variant antibodies and increase the chance of identifying novel antibodies. (Barbas, PNAS 91:3809 (1994); Yelton, D E, J. Immunology, 155:1994 (1995); Jackson, J. R., J. Immunology, 154:3310 (1995) and Hawkins, R E, J. Mol. Biology, 226:889 (1992)).

There have also been attempts to create diversity by restricting the group of amino acid substitutions in some CDRs to reflect the amino acid distribution in naturally occurring antibodies. See, Garrard & Henner, Gene (1993), 128:103; Knappik et al., J. Mol. Biol. (1999), 296:57. However, these attempts have had varying success and have not been applied in a systematic and quantitative manner. Creating diversity in the CDR regions while minimizing the number of amino acid changes has been a challenge. Furthermore, in some instances, once a first library has been generated according to one set of criteria, it may be desirable to further enhance the diversity of the first library. However, this requires that the first library has sufficient diversity and yet remain sufficiently small in size such that further diversity can be introduced without substantially exceeding practical limitations such as yield, etc.

Some groups have reported theoretical and experimental analyses of the minimum number of amino acid repertoire that is needed for generating proteins. However, these analyses have generally been limited in scope and nature, and substantial skepticism and questions remain regarding the feasibility of generating polypeptides having complex functions using a restricted set of amino acid types. See, e.g., Riddle et al., Nat. Struct. Biol. (1997), 4(10):805-809; Shang et al., Proc. Natl. Acad. Sci. USA (1994), 91:8373-8377; Heinz et al., Proc. Natl. Acad. Sci. USA (1992), 89:3751-3755; Regan & Degrado, Science (1988), 241:976-978; Kamteker et al., Science (1993), 262:1680-1685; Wang & Wang, Nat. Struct. Biol. (1999), 6(11): 1033-1038; Xiong et al., Proc. Natl. Acad. Sci. USA (1995), 92:6349-6353; Heinz et al., Proc. Natl. Acad. Sci. USA (1992), 89:3751-3755; Cannata et al., Bioinformatics (2002), 18(8):1102-1108; Davidson et al., Nat. Struct. Biol. (1995), 2(10):856-863; Murphy et al., Prot. Eng. (2000), 13(3):149-152; Brown & Sauer, Proc. Natl. Acad. Sci. USA (1999), 96:1983-1988; Akanuma et al., Proc. Natl. Acad. Sci. (2002), 99(21):13549-13553; Chan, Nat. Struct. Biol. (1999), 6(11):994-996.

Thus, there remains a need to improve methods of generating libraries that comprise functional polypeptides having a sufficient degree of sequence diversity, yet are sufficiently amenable for further manipulations directed at further diversification, high yield expression, etc. The invention described herein meets this need and provides other benefits.

### DISCLOSURE OF THE INVENTION

The present invention provides simplified and flexible methods of generating polypeptides comprising variant CDRs that comprise sequences with restricted diversity yet retain target antigen binding capability. Unlike conventional methods that are based on the proposition that adequate diversity of target binders can be generated only if a particular CDR(s), or all CDRs are diversified, and unlike conventional notions that adequate diversity is dependent upon the broadest range of amino acid substitutions (generally by substitution using all or most of the 20 amino acids), the invention provides methods capable of generating high quality target binders that are not necessarily dependent upon diversifying a particular CDR(s) or a particular number of CDRs of a reference polypeptide or source antibody. The invention is based, at least in part, on the surprising and unexpected finding that highly diverse libraries of high quality comprising functional polypeptides capable of binding target antigens can be generated by diversifying a minimal number of amino acid positions with a highly restricted number of amino acid residues. Methods of the invention are rapid, convenient and flexible, based on using restricted codon sets that encode a low number of amino acids. The restricted sequence diversity, and thus generally smaller size of the populations (e.g., libraries) of polypeptides generated by methods of the invention allows for further diversification of these populations, where necessary or desired. This is an advantage generally not provided by conventional methods. Candidate binder polypeptides generated by the invention possess high-quality target binding characteristics and have structural characteristics that provide for high yield of production in cell culture. The invention provides methods for generating these binder polypeptides, methods for using these polypeptides, and compositions comprising the same.

In one aspect, the invention provides fusion polypeptides comprising diversified CDR(s) and a heterologous polypeptide sequence (in certain embodiments, that of at least a portion of a viral polypeptide), as single polypeptides and as a member of a plurality of unique individual polypeptides that are candidate binders to targets of interest. Compositions (such as libraries) comprising such polypeptides find use in a variety of applications, for example, as pools of candidate immunoglobulin polypeptides (for example, antibodies and antibody fragments) that bind to targets of interest. Such polypeptides may also be generated using non-immunoglobulin scaffolds (for example, proteins, such as human growth hormone, etc.). The invention encompasses various aspects, including polynucleotides and polypeptides generated according to methods of the invention, and systems, kits and articles of manufacture for practicing methods of the invention, and/or using polypeptides/polynucleotides and/or compositions of the invention.

In one aspect, the invention provides a method of generating a polypeptide comprising at least one, two, three, four, five or all variant CDRs selected from the group consisting of H1, H2, H3, L1, L2 and L3, wherein said polypeptide is capable of binding a target antigen of interest, said method comprising identifying at least one (or any number up to all) solvent accessible and highly diverse amino acid position in a reference CDR corresponding to the variant CDR; and (ii) varying the amino acid at the solvent accessible and high diverse position by generating variant copies of the CDR using a restricted codon set (the definition of “restricted codon set” as provided below).

Various aspects and embodiments of methods of the invention are useful for generating and/or using a pool comprising a plurality of polypeptides of the invention, in particular for selecting and identifying candidate binders to target antigens of interest. For example, the invention provides a method of generating a composition comprising a plurality of polypeptides, each polypeptide comprising at least one, two, three, four, five or all variant CDRs selected from the group consisting of H1, H2, H3, L1, L2 and L3, wherein said polypeptide is capable of binding a target antigen of interest, said method comprising identifying at least one (or any number up to all) solvent accessible and highly diverse amino acid position in a reference CDR corresponding to the variant CDR; and (ii) varying the amino acid at the solvent accessible and high diverse position by generating variant copies of the CDR using a restricted codon set; wherein a plurality of polypeptides are generated by amplifying a template polynucleotide with a set of oligonucleotides comprising highly restricted degeneracy in the sequence encoding a variant amino acid, wherein said restricted degeneracy reflects the limited number of codon sequences of the restricted codon set.

In another example, the invention provides a method comprising: constructing an expression vector comprising a polynucleotide sequence which encodes a light chain, a heavy chain, or both the light chain and the heavy chain variable domains of a source antibody comprising at least one, two, three, four, five or all CDRs selected from the group consisting of CDR L1, L2, L3, H1, H2 and H3; and mutating at least one, two, three, four, five or all CDRs of the source antibody at at least one (or any number up to all) solvent accessible and highly diverse amino acid position using a restricted codon set.

In another example, the invention provides a method comprising: constructing a library of phage or phagemid particles displaying a plurality of polypeptides of the invention; contacting the library of particles with a target antigen under conditions suitable for binding of the particles to the target antigen; and separating the particles that bind from those that do not bind to the target antigen.

In any of the methods of the invention described herein, a solvent accessible and/or highly diverse amino acid position can be any that meet the criteria as described herein, in particular any combination of the positions as described herein, for example any combination of the positions described for the polypeptides of the invention (as described in greater detail herein). Suitable variant amino acids can be any that meet the criteria as described herein, for example variant amino acids in polypeptides of the invention as described in greater detail below.

Designing diversity in CDRs may involve designing diversity in the length and/or in sequence of the CDR. For example, CDRH3 may be diversified in length to be, e.g., 7 to 21 amino acids in length, and/or in its sequence, for example by varying highly diverse and/or solvent accessible positions with amino acids encoded by a restricted codon set. In some embodiments, a portion of CDRH3 has a length ranging from 5 to 21, 7 to 20, 9 to 15, or 11 to 13 amino acids, and has a variant amino acid at one or more positions encoded by a restricted codon set that encodes a limited number of amino acids such as codon sets encoding no more than 19, 15, 10, 8, 6, 4 or 2 amino acids. In some embodiments, the C terminal end has an amino acid sequence AM or AMDY.

In some embodiments, polypeptides of the invention can be in a variety of forms as long as the target binding function of the polypeptides is retained. In some embodiments, a polypeptide of the invention is a fusion polypeptide (i.e. a fusion of two or more sequences from heterologous polypeptides). Polypeptides with diversified CDRs according to the invention can be prepared as fusion polypeptides to at least a portion of a viral coat protein, for example, for use in phage display. Viral coat proteins that can be used for display of the polypeptides of the invention comprise protein p III, major coat protein pVIII, Soc (T4 phage), Hoc (T4 phage), gpD (lambda phage), pVI, or variants or fragments thereof. In some embodiments, the fusion polypeptide is fused to at least a portion of a viral coat protein, such as a viral coat protein selected from the group consisting of pIII, pVIII, Soc, Hoc, gpD, pVI, and variants or fragments thereof.

In some embodiments, in which the polypeptide with diversified CDRs is one or more antibody variable domains, the antibody variable domains can be displayed on the surface of the virus in a variety of formats including ScFv, Fab, ScFv₂, F(ab′)₂ and F(ab)₂. For display of the polypeptides in bivalent manner, the fusion protein in certain embodiments includes a dimerization domain. The dimerization domain can comprise a dimerization sequence and/or a sequence comprising one or more cysteine residues. The dimerization domain can be linked, directly or indirectly, to the C-terminal end of a heavy chain variable or constant domain (e.g., CH1). The structure of the dimerization domain can be varied depending on whether the antibody variable domain is produced as a fusion protein component with the viral coat protein component (e.g., without an amber stop codon after dimerization domain) or whether the antibody variable domain is produced predominantly without the viral coat protein component (e.g., with an amber stop codon after dimerization domain). When the antibody variable domain is produced predominantly as a fusion protein with the viral coat protein component, one or more disulfide bonds and/or a single dimerization sequence provides for bivalent display. For antibody variable domains predominantly produced without being fused to a viral coat protein component (e.g. with an amber stop codon), the dimerization domain can comprise both a cysteine residue and a dimerization sequence.

In addition, optionally, a fusion polypeptide can comprise a tag that may be useful in purification, detection and/or screening such as FLAG, poly-his, gD tag, c-myc, fluorescence protein or B-galactosidase. In one embodiment, a fusion polypeptide comprises a light chain variable or constant domain fused to a polypeptide tag.

In another aspect of the invention, a polypeptide such as an antibody variable domain is obtained from a single source or template molecule. The source or template molecule can be selected or designed for characteristics such as good yield and stability when produced in prokaryotic or eukaryotic cell culture, and/or to accommodate CDRH3 regions of varying lengths. The sequence of the template molecule can be altered to improve folding and/or display of the variable domain when presented as a fusion protein with a phage coat protein component. For example, a source antibody may comprise the amino acid sequence of the variable domains of humanized antibody 4D5 (light chain variable domain (FIG. 1; SEQ ID NO: 1)); (heavy chain variable domain (FIG. 1; SEQ ID NO: 2)). For example, in an antibody variable domain of a heavy or light chain, framework region residues can be modified or altered from the source or template molecule to improve folding, yield, display or affinity of the antibody variable domain. In some embodiments, framework residues are selected to be modified from the source or template molecule when the amino acid in the framework position of the source molecule is different from the amino acid or amino acids commonly found at that position in naturally occurring antibodies or in a subgroup consensus sequence. The amino acids at those positions can be changed to the amino acids most commonly found in the naturally occurring antibodies or in a subgroup consensus sequence at that position. In one embodiment, framework residue 71 of the heavy chain may be R, V or A. In another example, framework residue 93 of the heavy chain may be S or A. In yet another example, framework residue 94 may be R, K or T or encoded by MRT. In another example, framework residue 93 is A and framework residue 94 is R. In yet another example, framework residue 49 in the heavy chain may be alanine or glycine. Framework residues in the light chain may also be changed. For example, the amino acid at position 66 may be arginine or glycine. Framework regions for the wild-type humanized antibody 4D5-8 light chain and heavy chain sequences are shown in FIG. 16 (SEQ ID NOS: 1099-1102 and 1103-1106, respectively). Framework regions for variant versions of the humanized antibody 4D5-8 light chain and heavy chain sequences wherein the light chain is modified at position 66 and the heavy chain is modified at positions 71, 73, and 78 are shown in FIG. 17 (SEQ ID NOS: 1107-1110 and 1111-1114).

Methods of the invention are capable of generating a large variety of polypeptides comprising a diverse set of CDR sequences.

Immunoglobulin heavy chain variable domains randomized to provide diversity are provided. In one embodiment, a polypeptide comprising an immunoglobulin heavy chain variable domain is provided, wherein:
(i) CDRH1 comprises an amino acid sequence G-F-N-X1-X2-X3-X4-X5-X6-H (SEQ ID NO: 2629), wherein G is position 26 and X1 is position 29 according to the Kabat numbering system; wherein X1 is selected from F, L, I, and V; wherein X2 is selected from Y and S; wherein X3 is selected from Y and S; wherein X4 is selected from Y and S; wherein X5 is selected from Y and S, and wherein X6 is selected from M and I;
(ii) CDRH2 comprises an amino acid sequence: X1-I-X2-X3-X4-X5-X6-X7-T-X8-Y-A-D-S-V-K-G (SEQ ID NO: 2630), wherein X1 is position 50 according to the Kabat numbering system; wherein X1 is selected from Y and S; wherein X2 is selected from Y and S; wherein X3 is selected from P and S; wherein X4 is selected from Y and S; wherein X5 is selected from Y and S; wherein X6 is selected from G and S; wherein X7 is selected from Y and S; and wherein X8 is selected from Y and S; and
(iii) CDRH3 comprises an amino acid sequence: X1-X2-X3-X4-X5-X6-X7-X8-X9-X10-X11-X12-X13-X14-X15-X16-X17-X18-X19-D-Y (SEQ ID NO: 2631), wherein X1 is position 95 according to the Kabat numbering system, and wherein X1 is selected from Y and S; X2 is selected from Y and S; X3 is selected from Y and S, X4 is selected from Y and S; X5 is selected from Y and S; X6 is selected from Y and S; X7 is selected from Y and S or is not present; X8 is selected from Y and S or is not present; X9 is selected from Y and S or is not present; X10 is selected from Y and S or is not present; X11 is selected from Y and S or is not present; X12 is selected from Y and S or is not present; X13 is selected from Y and S or is not present; X14 is selected from Y and S or is not present; X15 is selected from Y and S or is not present; X16 is selected from Y and S or is not present; X17 is selected from Y and S or is not present; X18 is selected from G and A; and X19 is selected from F, L, I, and M.
   In one aspect, CDRH1 comprises an amino acid sequence selected from SEQ ID NOs: 52-66. In one aspect, CDRH2 comprises an amino acid sequence selected from SEQ ID NOs: 67-81. In one aspect, CDRH3 comprises an amino acid sequence selected from SEQ ID NOs: 82-96.

In another embodiment, a polypeptide comprising an immunoglobulin heavy chain variable domain is provided, wherein:
(i) CDRH1 comprises an amino acid sequence G-F-N-X1-X2-X3-X4-X5-X6-H (SEQ ID NO: 2629), wherein G is position 26 and X1 is position 29 according to the Kabat numbering system; wherein X1 is selected from F, L, I, and V; wherein X2 is selected from Y and S; wherein X3 is selected from Y and S; wherein X4 is selected from Y and S; wherein X5 is selected from Y and S, and wherein X6 is selected from M and I;
(ii) CDRH2 comprises an amino acid sequence: X1-I-X2-X3-X4-X5-X6-X7-T-X8-Y-A-D-S-V-K-G (SEQ ID NO: 2630), wherein X1 is position 50 according to the Kabat numbering system; wherein X1 is selected from Y and S; wherein X2 is selected from Y and S; wherein X3 is selected from P and S; wherein X4 is selected from Y and S; wherein X5 is selected from Y and S; wherein X6 is selected from G and S; wherein X7 is selected from Y and S; and wherein X8 is selected from Y and S; and
(iii) CDRH3 comprises an amino acid sequence: X1-X2-X3-X4-X5-X6-X7-X8-X9-X10-X11-X12-X13-X14-X15-X16-X17-X18-X19-D-Y (SEQ ID NO: 2632), wherein X1 is position 95 according to the Kabat numbering system, and wherein the amino acids at each of positions X1-X6 are selected from a pool of amino acids in a molar ratio of 20% Y, 15% S, 15% G, 3.125% A, 3.125% D, 3.125% E, 3.125% F, 3.125% H, 3.125% I, 3.125% K, 3.125% L, 3.125% M, 3.125% N, 3.125% P, 3.125% Q, 3.125% R, 3.125% T, 3.125% V, and 3.125% W; wherein the amino acids at each of positions X7-X17 are selected from a pool of amino acids in a molar ratio of 20% Y, 15% S, 15% G, 3.125% A, 3.125% D, 3.125% E, 3.125% F, 3.125% H, 3.125% I, 3.125% K, 3.125% L, 3.125% M, 3.125% N, 3.125% P, 3.125% Q, 3.125% R, 3.125% T, 3.125% V, and 3.125% W, or are not present;

wherein X18 is selected from G and A; and wherein X19 is selected from F, L, I, and M.

In one aspect, CDRH1 comprises an amino acid sequence selected from SEQ ID NOs: 111-125. In one aspect, CDRH2 comprises an amino acid sequence selected from SEQ ID NOs: 126-141. In one aspect, CDRH3 comprises an amino acid sequence selected from SEQ ID NOs: 142 and 144-157.

In another embodiment, a polypeptide comprising an immunoglobulin heavy chain variable domain is provided, wherein:
(i) CDRH1 comprises an amino acid sequence G-F-N-X1-X2-X3-X4-X5-X6-H (SEQ ID NO: 2629), wherein G is position 26 and X1 is position 29 according to the Kabat numbering system; wherein X1 is selected from F, L, I, and V; wherein X2 is selected from Y and S; wherein X3 is selected from Y and S; wherein X4 is selected from Y and S; wherein X5 is selected from Y and S; and wherein X6 is selected from M and I;
(ii) CDRH2 comprises an amino acid sequence: X1-I-X2-X3-X4-X5-X6-X7-T-X8-Y-A-D-S-V-K-G (SEQ ID NO: 2930), wherein X1 is position 50 according to the Kabat numbering system; wherein X1 is selected from Y and S; wherein X2 is selected from Y and S; wherein X3 is selected from P and S; wherein X4 is selected from Y and S; wherein X5 is selected from Y and S; wherein X6 is selected from G and S; wherein X7 is selected from Y and S; and wherein X8 is selected from Y and S; and
(iii) CDRH3 comprises an amino acid sequence: X1-X2-X3-X4-X5-X6-X7-D-Y (SEQ ID NO: 2633), wherein X1 is position 95 according to the Kabat numbering system, and wherein the amino acids at each of positions X1-X5 are selected from a pool of amino acids in a molar ratio of 20% Y, 15% S, 15% G, 3.125% A, 3.125% D, 3.125% E, 3.125% F, 3.125% H, 3.125% I, 3.125% K, 3.125% L, 3.125% M, 3.125% N, 3.125% P, 3.125% Q, 3.125% R, 3.125% T, 3.125% V, and 3.125% W; wherein X6 is selected from G and A; and
wherein X7 is selected from F, L, I and M.
   In one aspect, CDRH3 comprises the amino acid sequence of SEQ ID NO: 143.

In another embodiment, a polypeptide comprising an immunoglobulin heavy chain variable domain is provided, wherein:
(i) CDRH1 comprises an amino acid sequence G-F-X1-I-X2-X3-X4-X5-I-H (SEQ ID NO: 2634), wherein G is position 26 and X1 is position 28 according to the Kabat numbering system; wherein X1 is selected from Y and S; wherein X2 is selected from Y and S; wherein X3 is selected from Y and S; wherein X4 is selected from Y and S; and wherein X5 is selected from Y and S;
(ii) CDRH2 comprises an amino acid sequence: X1-I-X2-P-X3-X4-G-X5-T-X6-Y-A-D-S-V-K-G (SEQ ID NO: 2635), wherein X1 is position 50 according to the Kabat numbering system; wherein X1 is selected from Y and S; wherein X2 is selected from Y and S; wherein X3 is selected from Y and S; wherein X4 is selected from Y and S; wherein X5 is selected from Y and S; and wherein X6 is selected from Y and S; and
(iii) CDRH3 comprises an amino acid sequence: X1-X2-X3-X4-X5-X6-X7-X8-X9-X10-X11-X12-X13-X14-X15-X16-X17-X18-X19-D-Y (SEQ ID NO: 2636), wherein X1 is position 95 according to the Kabat numbering system, and wherein the amino acids at each of positions X1-X6 are selected from a pool of amino acids in a molar ratio of 50% Y, 25% S, 25% G; wherein the amino acids at each of positions X7-X17 are selected from a pool of amino acids in a molar ratio of 50% Y, 25% S, and 25% G, or are not present; wherein X18 is selected from G and A; and wherein X19 is selected from I, M, L, and F.

In another embodiment, a polypeptide comprising an immunoglobulin heavy chain variable domain is provided, wherein:
(i) CDRH1 comprises an amino acid sequence G-F-X1-I-X2-X3-X4-X5-I-H (SEQ ID NO: 2634), wherein G is position 26 and X1 is position 28 according to the Kabat numbering system; wherein X1 is selected from Y and S; wherein X2 is selected from Y and S; wherein X3 is selected from Y and S; wherein X4 is selected from Y and S; and wherein X5 is selected from Y and S;
(ii) CDRH2 comprises an amino acid sequence: X1-I-X2-P-X3-X4-G-X5-T-X6-Y-A-D-S-V-K-G (SEQ ID NO: 2635), wherein X1 is position 50 according to the Kabat numbering system; wherein X1 is selected from Y and S; wherein X2 is selected from Y and S; wherein X3 is selected from Y and S; wherein X4 is selected from Y and S; wherein X5 is selected from Y and S; and wherein X6 is selected from Y and S; and
(iii) CDRH3 comprises an amino acid sequence: X1-X2-X3-X4-X5-X6-X7-X8-X9-X10-X11-X12-X13-X14-X15-X16-X17-X18-X19-D-Y (SEQ ID NO: 2637), wherein X1 is position 95 according to the Kabat numbering system, and wherein the amino acids at each of positions X1-X6 are selected from a pool of amino acids in a molar ratio of 25% Y, 50% S, and 25% R; wherein the amino acids at each of positions X7-X17 are selected from a pool of amino acids in a molar ratio of 25% Y, 50% S, and 25% R, or are not present; wherein X18 is selected from G and A; and wherein X19 is selected from I, M, L, and F.

In another embodiment, a polypeptide comprising an immunoglobulin heavy chain variable domain is provided, wherein:
(i) CDRH1 comprises an amino acid sequence G-F-X1-I-X2-X3-X4-X5-I-H (SEQ ID NO: 2634), wherein G is position 26 and X1 is position 28 according to the Kabat numbering system; wherein X1 is selected from Y and S; wherein X2 is selected from Y and S; wherein X3 is selected from Y and S; wherein X4 is selected from Y and S; and wherein X5 is selected from Y and S;
(ii) CDRH2 comprises an amino acid sequence: X1-I-X2-P-X3-X4-G-X5-T-X6-Y-A-D-S-V-K-G (SEQ ID NO: 2635), wherein X1 is position 50 according to the Kabat numbering system; wherein X1 is selected from Y and S; wherein X2 is selected from Y and S; wherein X3 is selected from Y and S; wherein X4 is selected from Y and S; wherein X5 is selected from Y and S; and wherein X6 is selected from Y and S; and
(iii) CDRH3 comprises an amino acid sequence: X1-X2-X3-X4-X5-X6-X7-X8-X9-X10-X11-X12-X13-X14-X15-X16-X17-X18-X19-D-Y (SEQ ID NO: 2638), wherein X1 is position 95 according to the Kabat numbering system, and wherein the amino acids at each of positions X1-X6 are selected from a pool of amino acids in a molar ratio of 38% Y, 25% S, 25% G, and 12% R; wherein the amino acids at each of positions X7-X17 are selected from a pool of amino acids in a molar ratio of 38% Y, 25% S, 25% G, and 12% R, or are not present; wherein X18 is selected from G and A; and wherein X19 is selected from I, M, L, and F.

In another embodiment, a polypeptide comprising an immunoglobulin heavy chain variable domain, wherein:
(i) CDRH1 comprises an amino acid sequence G-F-X1-I-X2-X3-X4-X5-I-H (SEQ ID NO: 2634), wherein G is position 26 and X1 is position 28 according to the Kabat numbering system; wherein X1 is selected from Y and S; wherein X2 is selected from Y and S; wherein X3 is selected from Y and S; wherein X4 is selected from Y and S; and wherein X5 is selected from Y and S;
(ii) CDRH2 comprises an amino acid sequence: X1-I-X2-P-X3-X4-G-X5-T-X6-Y-A-D-S-V-K-G (SEQ ID NO: 2635), wherein X1 is position 50 according to the Kabat numbering system; wherein X1 is selected from Y and S; wherein X2 is selected from Y and S; wherein X3 is selected from Y and S; wherein X4 is selected from Y and S; wherein X5 is selected from Y and S; and wherein X6 is selected from Y and S; and
(iii) CDRH3 comprises an amino acid sequence: X1-X2-X3-X4-X5-X6-X7-X8-X9-X10-X11-X12-X13-X14-X15-X16-X17-X18-X19-D-Y (SEQ ID NO: 2639), wherein X1 is position 95 according to the Kabat numbering system, and wherein the amino acids at each of positions X1-X6 are selected from a pool of amino acids in a molar ratio of 20% Y, 26% S, 26% G, 13% R, 1% A, 1% D, 1% E, 1% F, 1% H, 1% I, 1% K, 1% L, 1% M, 1% N, 1% P, 1% Q, 1% T, 1% V, and 1% W; wherein the amino acids at each of positions X7-X17 are selected from a pool of amino acids in a molar ratio of 20% Y, 26% S, 26% G, 13% R, 1% A, 1% D, 1% E, 1% F, 1% H, 1% I, 1% K, 1% L, 1% M, 1% N, 1% P, 1% Q, 1% T, 1% V, 1% W, or are not present; wherein X18 is selected from G and A; and wherein X19 is selected from I, M, L, and F.

In one aspect, CDRH1 comprises an amino acid sequence selected from SEQ ID NOS: 318-439 or any of CDRH1 sequences in FIGS. 14 and 15. In one aspect, CDRH2 comprises an amino acid sequence selected from SEQ ID NOS: 440-561 or any of CDRH2 sequences in FIGS. 14 and 15. In one aspect, CDRH3 comprises an amino acid sequence selected from SEQ ID NOS: 562-683 or any of CDRH3 sequences in FIGS. 14 and 15. In one aspect, CDRH1 comprises an amino acid sequence selected from SEQ ID NOS:784-888 or any of CDRH1 sequences in FIGS. 14 and 15. In one aspect, CDRH2 comprises an amino acid sequence selected from SEQ ID NOS:889-993 or any of CDRH2 sequences in FIGS. 14 and 15. In one aspect, CDRH3 comprises an amino acid sequence selected from SEQ ID NOS:994-1098 or any of CDRH3 sequences in FIGS. 14 and 15.

In another embodiment, a polypeptide comprising an immunoglobulin heavy chain variable domain is provided, wherein:
(i) CDRH1 comprises an amino acid sequence G-F-X1-I-X2-X3-X4-X5-I-H (SEQ ID NO: 2634), wherein G is position 26 and X1 is position 28 according to the Kabat numbering system; wherein X1 is selected from Y and S; wherein X2 is selected from Y and S; wherein X3 is selected from Y and S; wherein X4 is selected from Y and S; and wherein X5 is selected from Y and S;
(ii) CDRH2 comprises an amino acid sequence: X1-I-X2-P-X3-X4-S-X5-T-X6-Y-A-D-S-V-K-G (SEQ ID NO: 2635), wherein X1 is position 50 according to the Kabat numbering system; wherein X1 is selected from Y and S; wherein X2 is selected from Y and S; wherein X3 is selected from Y and S; wherein X4 is selected from Y and S; wherein X5 is selected from Y and S; and wherein X6 is selected from Y and S; and
(iii) CDRH3 comprises an amino acid sequence: X1-X2-X3-X4-X5-X6-X7-X8-X9-X10-X11-X12-X13-X14-X15-X16-X17-X18-X19 (SEQ ID NO: 2640), wherein X1 is position 95 according to the Kabat numbering system, and wherein the amino acids at each of positions X1-X19 are selected from S and one of A, C, F, G, I, L, N, P, R, T, W, or Y, or are not present; wherein X18 is selected from G and A; and wherein X19 is selected from F, L, I, and M.

In one aspect, CDRH1 comprises an amino acid sequence selected from SEQ ID NOS: 1340-1396, 1538-1564, 1653-1686, 1805-1854, and 1963-1970 or any of the CDRH1 sequences in FIGS. 21, 22, 23, 24 and 25. In another aspect, CDRH2 comprises an amino acid sequence selected from SEQ ID NOS: 1397-1453, 1565-1591, 1687-1720, 1855-1904, and 1971-1978 or any of the CDRH2 sequences in FIGS. 21, 22, 23, 24 and 25. In another aspect, CDRH3 comprises an amino acid sequence selected from SEQ ID NOS: 1454-1510, 1592-1618, 1721-1754, 1905-1954, and 1979-1986 or any of the CDRH3 sequences in FIGS. 21, 22, 23, 24 and 25.

In another embodiment, a polypeptide comprising an immunoglobulin heavy chain variable domain is provided, wherein:
(i) CDRH1 comprises an amino acid sequence G-F-X1-I-X2-X3-X4-X5-I-H (SEQ ID NO: 2641), wherein G is position 26 and X1 is position 28 according to the Kabat numbering system; wherein the amino acid at each of positions X1-X5 is selected from S and one of Y, W, R, or F;
(ii) CDRH2 comprises an amino acid sequence: X1-I-X2-P-X3-X4-S-X5-T-X6-Y-A-D-S-V-K-G (SEQ ID NO: 2642), wherein X1 is position 50 according to the Kabat numbering system; wherein the amino acid at each of positions X1-X6 is selected from S and one of Y, W, R, or F; and
(iii) CDRH3 comprises an amino acid sequence: X1-X2-X3-X4-X5-X6-X7-X8-X9-X10-X11-X12-X13-X14-X15-X16-X17-X18-X19 (SEQ ID NO: 2643), wherein X1 is position 95 according to the Kabat numbering system, and wherein the amino acids at each of positions X1-X19 are selected from S and one of Y, W, R, or F, or are not present; wherein X18 is selected from G and A; and wherein X19 is selected from F, L, I, and M.
   In one aspect, CDRH1 comprises an amino acid sequence selected from SEQ ID NOS: 2027-2057, 2147-2173, 2239-2249, 2300-2327, and 2395-2405 or any of the CDRH1 sequences in FIGS. 28, 29, 30, 31 and 32. In another aspect, CDRH2 comprises an amino acid sequence selected from SEQ ID NOS: 2058-2088, 2174-2200, 2250-2260, 2328-2355, and 2406-2416 or any of the CDRH2 sequences in FIGS. 28, 29, 30, 31 and 32. In another aspect, CDRH3 comprises an amino acid sequence selected from SEQ ID NOS: 2089-2119, 2201-2227, 2261-2271, 2356-2383, and 2417-2427 or any of the CDRH3 sequences in FIGS. 28, 29, 30, 31 and 32.

In another embodiment, a polypeptide comprising an immunoglobulin heavy chain variable domain is provided, wherein:
(i) CDRH1 comprises an amino acid sequence G-F-N-X1-X2-X3-X4-X5-X6-H (SEQ ID NO: 2644), wherein G is position 26 and X1 is position 29 according to the Kabat numbering system; and wherein X1-X6 are naturally occurring amino acids other than cysteine;
(ii) CDRH2 comprises an amino acid sequence: X6-I-X7-X8-X9-X10-X11-X12-T-X13-Y-A-D-S-V-K-G (SEQ ID NO: 2645), wherein X6 is position 50 according to the Kabat numbering system, and wherein X6-X13 are naturally occurring amino acids other than cysteine; and
(iii) CDRH3 comprises an amino acid sequence: X14-X15-X16-X17-X18-(X19)ₙ-X20-X21-D-Y (SEQ ID NO: 2646), wherein X14 is position 95 according to the Kabat numbering system, and wherein n is a suitable number that would retain the functional activity of the heavy chain variable domain, and wherein X14-X21 are naturally occurring amino acids other than cysteine.
   In one aspect, n is 1 to 12. In one aspect, X1 is selected from F, L, I, and V; X2 is selected from Y and S; X3 is selected from Y and S; X4 is selected from Y and S; X5 is selected from Y and S, and X6 is selected from M and I. In one aspect, X6 is selected from Y and S; X7 is selected from Y and S; X8 is selected from P and S; X9 is selected from Y and S; X10 is selected from Y and S; X11 is selected from G and S; X12 is selected from Y and S; and X13 is selected from Y and S. In one aspect, X14 is selected from Y and S; X15 is selected from Y and S; X16 is selected from Y and S, X17 is selected from Y and S; X18 is selected from Y and S; X19 is selected from Y and S; X20 is selected from G and A; and X21 is selected from F, L, I, and M. In an alternative aspect, the amino acids at each of positions X14-X19 are selected from a pool of amino acids in a molar ratio of 20% Y, 15% S, 15% G, 3.125% A, 3.125% D, 3.125% E, 3.125% F, 3.125% H, 3.125% I, 3.125% K, 3.125% L, 3.125% M, 3.125% N, 3.125% P, 3.125% Q, 3.125% R, 3.125% T, 3.125% V, and 3.125% W; X20 is selected from G and A; and X21 is selected from F, L, I, and M. In one aspect, CDRH1 comprises an amino acid sequence selected from SEQ ID NOs: 52-66 and 111-125. In one aspect, CDRH2 comprises an amino acid sequence selected from SEQ ID NOs: 67-81 and 126-141. In one aspect, CDRH3 comprises an amino acid sequence selected from SEQ ID NOs: 82-96 and 142-157.

In another embodiment, a polypeptide comprising an immunoglobulin heavy chain variable domain is provided, wherein:
(i) CDRH1 comprises an amino acid sequence G-F-X1-I-X2-X3-X4-X5-I-H (SEQ ID NO: 2647), wherein G is position 26 and X1 is position 28 according to the Kabat numbering system; and wherein X1-X5 are naturally occurring amino acids other than cysteine;
(ii) CDRH2 comprises an amino acid sequence: X6-I-X7-P-X8-X9-G-X10-T-X11-Y-A-D-S-V-K-G (SEQ ID NO: 2648), wherein X6 is position 50 according to the Kabat numbering system, and wherein X6-X11 are naturally occurring amino acids other than cysteine; and
(iii) CDRH3 comprises an amino acid sequence: X12-X13-X14-X15-X16-(X17)ₙ-X18-X19-D-Y (SEQ ID NO: 2649), wherein X12 is position 95 according to the Kabat numbering system, and wherein n is a suitable number that would retain the functional activity of the heavy chain variable domain, and wherein X12-X19 are naturally occurring amino acids other than cysteine.

In one aspect, n is 1 to 12. In one aspect, X1 is selected from Y and S; X2 is selected from Y and S; X3 is selected from Y and S; X4 is selected from Y and S; X5 is selected from Y and S, and X6 is selected from Y and S. In one aspect, X6 is selected from Y and S; X7 is selected from Y and S; X8 is selected from Y and S; X9 is selected from Y and S; X10 is selected from Y and S; and X11 is selected from Y and S. In one aspect, the amino acids at each of positions X12-X17 are selected from a pool of amino acids in a molar ratio of 50% Y, 25% S, and 25% G, X18 is selected from G and A, and X19 is selected from I, M, L, and F. In an alternative aspect, the amino acids at each of positions X12-X17 are selected from a pool of amino acids in a molar ratio of 25% Y, 50% S, and 25% R, X18 is selected from G and A, and X19 is selected from I, M, L, and F. In another alternative aspect, the amino acids at each of positions X12-X17 are selected from a pool of amino acids in a molar ratio of 38% Y, 25% S, 25% G, and 12% R, X18 is selected from G and A, and X19 is selected from I, M, L, and F. In another alternative aspect, the amino acids at each of positions X12-X17 are selected from a pool of amino acids in a molar ratio of 20% Y, 26% S, 26% G, 13% R, 1% A, 1% D, 1% E, 1% F, 1% H, 1% I, 1% K, 1% L, 1% M, 1% N, 1% P, 1% Q, 1% T, 1% V, and 1% W, X18 is selected from G and A, and X19 is selected from I, M, L, and F. In one aspect, CDRH1 comprises an amino acid sequence selected from SEQ ID NOS: 318-439 or 734-888 or any of the CDRH1 sequences in FIGS. 14 or 15. In one aspect, CDRH2 comprises an amino acid sequence selected from SEQ ID NOS: 440-561 or 989-993 or any of the CDRH2 sequences in FIGS. 14 or 15. In one aspect, CDRH3 comprises an amino acid sequence selected from SEQ ID NOS: 562-683 or 994-1098 or any of the CDRH3 sequences in FIGS. 14 or 15.

In another embodiment, a polypeptide comprising an immunoglobulin heavy chain variable domain is provided, wherein:
(i) CDRH1 comprises an amino acid sequence G-F-X1-I-X2-X3-X4-X5-I-H (SEQ ID NO: 2647), wherein G is position 26 and X1 is position 28 according to the Kabat numbering system; and wherein X1-X5 are naturally occurring amino acids other than cysteine;
(ii) CDRH2 comprises an amino acid sequence: X6-I-X7-P-X8-X9-S-X10-T-X11-Y-A-D-S-V-K-G (SEQ ID NO: 2650), wherein X6 is position 50 according to the Kabat numbering system, and wherein X6-X11 are naturally occurring amino acids other than cysteine; and
(iii) CDRH3 comprises an amino acid sequence: X12-X13-X14-(X15)ₙ-X16-X17 (SEQ ID NO: 2651), wherein X14 is position 95 according to the Kabat numbering system, and wherein n is a suitable number that would retain the functional activity of the heavy chain variable domain, and wherein X12-X17 are naturally occurring amino acids other than cysteine.

In one aspect, n is 1 to 14. In another aspect, X1 is selected from Y and S; X2 is selected from Y and S; X3 is selected from Y and S; X4 is selected from Y and S; and X5 is selected from Y and S. In another aspect, X1 is selected from W and S; X2 is selected from W and S; X3 is selected from W and S; X4 is selected from W and S; and X5 is selected from W and S. In another aspect, X1 is selected from R and S; X2 is selected from R and S; X3 is selected from R and S; X4 is selected from R and S; and X5 is selected from R and S. In another aspect, X1 is selected from F and S; X2 is selected from F and S; X3 is selected from F and S; X4 is selected from F and S; and X5 is selected from F and S. In another aspect, X6 is selected from Y and S; X7 is selected from Y and S; X8 is selected from Y and S; X9 is selected from Y and S; X10 is selected from Y and S; and X11 is selected from Y and S. In another aspect, X6 is selected from W and S; X7 is selected from W and S; X8 is selected from W and S; X9 is selected from W and S; X10 is selected from W and S; and X11 is selected from W and S. In another aspect, X6 is selected from R and S; X7 is selected from R and S; X8 is selected from R and S; X9 is selected from R and S; X10 is selected from R and S; and X11 is selected from R and S. In another aspect, X6 is selected from F and S; X7 is selected from F and S; X8 is selected from F and S; X9 is selected from F and S; X10 is selected from F and S; and X11 is selected from F and S. In another aspect, X12 is selected from Y and S; X13 is selected from Y and S; X14 is selected from Y and S; X15 is selected from Y and S; X16 is selected from G and A; and X17 is selected from F, L, I, and M. In another aspect, X12 is selected from W and S; X13 is selected from W and S; X14 is selected from W and S; X15 is selected from W and S; X16 is selected from G and A; and X17 is selected from F, L, I, and M. In another aspect, X12 is selected from R and S; X13 is selected from R and S; X14 is selected from R and S; X15 is selected from R and S; X16 is selected from G and A; and X17 is selected from F, L, I, and M. In another aspect, X12 is selected from F and S; wherein X13 is selected from F and S; X14 is selected from F and S; X15 is selected from F and S; X16 is selected from G and A; and X17 is selected from F, L, I, and M.

In another aspect, the amino acids at each of positions X12-X15 are selected from S and one of A, C, F, G, I, L, N, P, R, T, W, and Y; X16 is selected from G and A; and X17 is selected from F, L, I, and M.

In another aspect, CDRH1 comprises an amino acid sequence selected from SEQ ID NOs: 1340-1396, 1538-1564, 1653-1686, 1805-1854, 1963-1970, 2027-2057, 2147-2173, 2239-2249, 2300-2327, and 2395-2405 or any of the CDRH1 sequences in any of FIGS. 21-25. In another aspect, CDRH2 comprises an amino acid sequence selected from SEQ ID NOs: 1397-1453, 1565-1591, 1687-1720, 1855-1904, 1971-1978, 2058-2088, 2174-2200, 2250-2260, 2328-2355, and 2406-2416 or any of the CDRH2 sequences in any of FIGS. 21-25. In another aspect, CDRH3 comprises an amino acid sequence selected from SEQ ID NOs: 1454-1510, 1592-1618, 1721-1754, 1905-1954, 1979-1986, 2089-2119, 2201-2227, 2261-2271, 2356-2383, and 2417-2427 or any of the CDRH3 sequences in FIGS. 21-25.

Immunoglobulin light chain variable domains randomized to provide diversity are also provided. In one embodiment, a polypeptide comprising an immunoglobulin light chain variable domain is provided, wherein CDRL3 comprises an amino acid sequence: Q-Q-X1-X2-X3-X4-X5-X6-X7-X8-T (SEQ ID NO: 2652), wherein X1 is position 91 according to the Kabat numbering system, wherein X1 is selected from Y and S; wherein X2 is selected from Y and S; wherein X3 is selected from Y and S; wherein X4 is selected from Y and S; wherein X5 is selected from Y and S, or is not present; wherein X6 is selected from Y and S, or is not present; wherein X7 is selected from P and L; and wherein X8 is selected from F, L, I, and V. In one aspect, CDRL3 comprises an amino acid sequence selected from SEQ ID NOs: 37-51 and 97-110.

In another embodiment, a polypeptide comprising an immunoglobulin light chain variable domain is provided, wherein CDRL3 comprises an amino acid sequence: Q-Q-X1-X2-X3-X4-P-X5-T (SEQ ID NO: 2653), wherein X1 is position 91 according to the Kabat numbering system, wherein X1 is selected from Y and S, wherein X2 is selected from Y and S; wherein X3 is selected from Y and S; wherein X4 is selected from Y and S; and wherein X5 is selected from Y and S. In one aspect, CDRL3 comprises an amino acid sequence selected from SEQ ID NOs: 209-317, 684-783, 1283-1339, 1511-1537, 1619-1652, 1755-1804, 1955-1962 or any of the CDRL3 sequences in FIGS. 14, 15 or 21-25.

In another embodiment, a polypeptide comprising an immunoglobulin light chain variable domain is provided, wherein CDRL3 comprises an amino acid sequence: Q-Q-X1-X2-X3-X4-P-X5-T (SEQ ID NO: 2654), wherein X1 is position 91 according to the Kabat numbering system, and wherein the amino acids at each of positions X1-X5 are selected from S and one of Y, W, R, or F. In one aspect, CDRL3 comprises an amino acid sequence selected from SEQ ID NOs: 1996-2026, 2120-2146, 2228-2238, 2272-2299, and 2384-2394 or any of the CDRL3 sequences in FIGS. 28-32.

In another embodiment, a polypeptide comprising an immunoglobulin light chain variable domain is provided, wherein:
(i) CDRL1 comprises a first consensus hypervariable sequence or variant thereof comprising substitution at one or more positions compared to a corresponding consensus hypervariable sequence;
(ii) CDRL2 comprises a second consensus hypervariable sequence or variant thereof comprising substitution at one or more positions compared to a corresponding consensus hypervariable sequence; and
(iii) CDRL3 comprises an amino acid sequence: Q-Q-X1-X2-X3-(X4)ₙ-X5-X6-T (SEQ ID NO: 2655), wherein X1-X6 are any naturally occurring amino acids other than cysteine, and wherein X1 is position 91 according to the Kabat numbering system.
   In one aspect, X1 is position 91 according to the Kabat numbering system, X1 is selected from Y and S; X2 is selected from Y and S; X3 is selected from Y and S; X4 is selected from Y and S; X5 is selected from P and L; and X6 is selected from F, L, I, and V. In one aspect, n is 1 to 3. In one aspect, CDRL3 comprises an amino acid sequence selected from SEQ ID NOs: 37-51 and 97-110. In one aspect, the first consensus hypervariable sequence is R-A-S-Q-D-V-N-T-A-V-A (SEQ ID NO: 6). In one aspect, the second consensus hypervariable sequence is S-A-S-S-L-Y-S (SEQ ID NO: 7).

In another embodiment, a polypeptide comprising an immunoglobulin light chain variable domain is provided, wherein:
(i) CDRL1 comprises a first consensus hypervariable sequence or variant thereof comprising substitution at one or more positions compared to a corresponding consensus hypervariable sequence;
(ii) CDRL2 comprises a second consensus hypervariable sequence or variant thereof comprising substitution at one or more positions compared to a corresponding consensus hypervariable sequence; and
(iii) CDRL3 comprises an amino acid sequence: Q-Q-X1-X2-X3-X4-P-X5-T (SEQ ID NO: 2656), wherein X1-X5 are any naturally occurring amino acids other than cysteine, and X1 is position 91 according to the Kabat numbering system.
   In one aspect, X1 is position 91 according to the Kabat numbering system, X1 is selected from Y and S, X2 is selected from Y and S; X3 is selected from Y and S; X4 is selected from Y and S; and X5 is selected from Y and S. In another aspect, X1 is position 91 according to the Kabat numbering system, and the amino acids at each of positions X1-X5 are selected from S and one of Y, W, R, and F. In one aspect, CDRL3 comprises an amino acid sequence selected from SEQ ID NOs: 209-317, 684-783, 1283-1339, 1511-1537, 1619-1652, 1755-1804, 1955-1962, 1996-2026, 2120-2146, 2228-2238, 2272-2299, and 2384-2394 or any of the CDRL3 sequences in any of FIGS. 14, 15 or 21-25. In another aspect, the first consensus hypervariable sequence is R-A-S-Q-D-V-N-T-A-V-A (SEQ ID NO: 6). In another aspect, the second consensus hypervariable sequence is S-A-S-S-L-Y-S (SEQ ID NO: 7).

In certain embodiments, a polypeptide comprising at least two antibody variable domains comprising: (a) a heavy chain antibody variable domain comprising any of the above-recited heavy chain polypeptides, and (b) a light chain antibody variable domain comprising any of the above-recited light chain polypeptides is provided.

In certain embodiments, an antibody comprising a polypeptide comprising an immunoglobulin heavy chain variable domain according to any of the above-recited heavy chain polypeptides, and a polypeptide comprising an immunoglobulin light chain variable domain according to any of the above-recited light chain polypeptides is provided.

In certain aspects, the above-recited polypeptides and antibodies further comprise a dimerization domain linked to the C-terminal region of a heavy chain antibody variable domain. In certain such aspects, the dimerization domain comprises a leucine zipper domain or a sequence comprising at least one cysteine residue. In certain such aspects, the dimerization domain comprises a hinge region from an antibody and a leucine zipper. In certain other aspects, the dimerization domain is a single cysteine.

In one embodiment, a fusion polypeptide comprising any of the above-recited polypeptides is provided, wherein an antibody variable domain comprising the above-recited polypeptide is fused to at least a portion of a viral coat protein. In one aspect, the viral coat protein is selected from the group consisting of protein pIII, major coat protein pVIII, Soc, Hoc, gpD, pv1, and variants thereof. In one aspect, the fusion polypeptide further comprises a dimerization domain between the variable domain and the viral coat protein. In one such aspect, the variable domain is a heavy chain variable domain. In another aspect, the fusion polypeptide further comprises a variable domain fused to a peptide tag. In one such aspect, the variable domain is a light chain variable domain. In another such aspect, the peptide tag is selected from the group consisting of gD, c-myc, poly-his, a fluorescence protein, and β-galactosidase.

In one embodiment, one or more of the above-described polypeptides further comprise framework regions FR1, FR2, FR3, and/or FR4 for an antibody variable domain corresponding to the variant CDRH1, CDRH2, CDRH3, and/or CDRL3, wherein the framework regions are obtained from a single antibody template. In certain such embodiments, each of the framework regions comprises an amino acid sequence corresponding to the framework region amino acid sequences of antibody 4D5 (SEQ ID NOS: 1099-1102 and 1103-1106) or a variant of antibody 4D5 (SEQ ID NOS: 1107-1110 and 1111-1114).

In one embodiment, a library is provided that comprises a plurality of one or more of the above-described polypeptides, wherein the library has at least 1×10⁴ distinct antibody variable domain sequences.

In one embodiment, a method of generating a composition comprising a plurality of polypeptides is provided, comprising:
(a) generating a plurality of polypeptides comprising:
   (i) CDRH1 comprising an amino acid sequence G-F-N-X1-X2-X3-X4-X5-X6-H (SEQ ID NO: 2629), wherein G is position 26 and X1 is position 29 according to the Kabat numbering system; wherein X1 is selected from F, L, I, and V; wherein X2 is selected from Y and S; wherein X3 is selected from Y and S; wherein X4 is selected from Y and S; wherein X5 is selected from Y and S, and wherein X6 is selected from M and I;
   (ii) CDRH2 comprising an amino acid sequence: X1-I-X2-X3-X4-X5-X6-X7-T-X8-Y-A-D-S-V-K-G (SEQ ID NO: 2630), wherein X1 is position 50 according to the Kabat numbering system; wherein X1 is selected from Y and S; wherein X2 is selected from Y and S; wherein X3 is selected from P and S; wherein X4 is selected from Y and S; wherein X5 is selected from Y and S; wherein X6 is selected from G and S; wherein X7 is selected from Y and S; and wherein X8 is selected from Y and S; and
   (iii) CDRH3 comprising an amino acid sequence: X1-X2-X3-X4-X5-X6-X7-X8-X9-X10-X11-X12-X13-X14-X15-X16-X17-X18-X19-D-Y (SEQ ID NO: 2631), wherein X1 is position 95 according to the Kabat numbering system, and wherein X1 is selected from Y and S; X2 is selected from Y and S; X3 is selected from Y and S, X4 is selected from Y and S; X5 is selected from Y and S; X6 is selected from Y and S; X7 is selected from Y and S or is not present; X8 is selected from Y and S or is not present; X9 is selected from Y and S or is not present; X10 is selected from Y and S or is not present; X11 is selected from Y and S or is not present; X12 is selected from Y and S or is not present; X13 is selected from Y and S or is not present; X14 is selected from Y and S or is not present; X15 is selected from Y and S or is not present; X16 is selected from Y and S or is not present; X17 is selected from Y and S or is not present; X18 is selected from G and A; and X19 is selected from F, L, I, and M.
      In one aspect, the method further comprises:
(b) generating a plurality of polypeptides comprising:
   (i) CDRL1 comprising a first consensus hypervariable sequence or variant thereof comprising substitution at one or more positions compared to a corresponding consensus hypervariable sequence;
   (ii) CDRL2 comprising a second consensus hypervariable sequence or variant thereof comprising substitution at one or more positions compared to a corresponding consensus hypervariable sequence; and
   (iii) CDRL3 comprising an amino acid sequence: Q-Q-X1-X2-X3-X4-X5-X6-X7-X8-T (SEQ ID NO: 2652), wherein X1 is position 91 according to the Kabat numbering system; an wherein X1 is position 91 according to the Kabat numbering system, wherein X1 is selected from Y and S; wherein X2 is selected from Y and S; wherein X3 is selected from Y and S; wherein X4 is selected from Y and S; wherein X5 is selected from Y and S, or is not present; wherein X6 is selected from Y and S, or is not present; wherein X7 is selected from P and L; and wherein X8 is selected from F, L, I, and V.
      In one aspect, the first consensus hypervariable sequence comprises a Kabat consensus CDRL1 sequence. In one such aspect, the first consensus hypervariable sequence is R-A-S-Q-D-V-N-T-A-V-A (SEQ ID NO: 6). In one aspect, the second consensus hypervariable sequence comprises a Kabat consensus CDRL2 sequence. In one such aspect, the second consensus hypervariable sequence is S-A-S-S-L-Y-S (SEQ ID NO: 7). In one aspect, the plurality of polypeptides are encoded by a plurality of polynucleotides.

In another embodiment, a method of generating a composition comprising a plurality of polypeptides is provided, comprising:
(a) generating a plurality of polypeptides comprising:
   (i) CDRH1 comprising an amino acid sequence G-F-N-X1-X2-X3-X4-X5-X6-H (SEQ ID NO: 2629), wherein G is position 26 and X1 is position 29 according to the Kabat numbering system; wherein X1 is selected from F, L, I, and V; wherein X2 is selected from Y and S; wherein X3 is selected from Y and S; wherein X4 is selected from Y and S; wherein X5 is selected from Y and S, and wherein X6 is selected from M and I;
   (ii) CDRH2 comprising an amino acid sequence: X1-I-X2-X3-X4-X5-X6-X7-T-X8-Y-A-D-S-V-K-G (SEQ ID NO: 2630), wherein X1 is position 50 according to the Kabat numbering system; wherein X1 is selected from Y and S; wherein X2 is selected from Y and S; wherein X3 is selected from P and S; wherein X4 is selected from Y and S; wherein X5 is selected from Y and S; wherein X6 is selected from G and S; wherein X7 is selected from Y and S; and wherein X8 is selected from Y and S; and
   (iii) CDRH3 comprising an amino acid sequence: X1-X2-X3-X4-X5-X6-X7-X8-X9-X10-X11-X12-X13-X14-X15-X16-X17-X18-X19-D-Y (SEQ ID NO: 2632), wherein X1 is position 95 according to the Kabat numbering system, and wherein the amino acids at each of positions X1-X6 are selected from a pool of amino acids in a molar ratio of 20% Y, 15% S, 15% G, 3.125% A, 3.125% D, 3.125% E, 3.125% F, 3.125% H, 3.125% I, 3.125% K, 3.125% L, 3.125% M, 3.125% N, 3.125% P, 3.125% Q, 3.125% R, 3.125% T, 3.125% V, and 3.125% W; wherein the amino acids at each of positions X7-X17 are selected from a pool of amino acids in a molar ratio of 20% Y, 15% S, 15% G, 3.125% A, 3.125% D, 3.125% E, 3.125% F, 3.125% H, 3.125% I, 3.125% K, 3.125% L, 3.125% M, 3.125% N, 3.125% P, 3.125% Q, 3.125% R, 3.125% T, 3.125% V, and 3.125% W, or are not present, wherein X18 is selected from G and A; and wherein X19 is selected from F, L, I and M.
      In one aspect, the method further comprises:
(b) generating a plurality of polypeptides comprising:
   (i) CDRL1 comprising a first consensus hypervariable sequence or variant thereof comprising substitution at one or more positions compared to a corresponding consensus hypervariable sequence;
   (ii) CDRL2 comprising a second consensus hypervariable sequence or variant thereof comprising substitution at one or more positions compared to a corresponding consensus hypervariable sequence; and
   (iii) CDRL3 comprising an amino acid sequence: Q-Q-X1-X2-X3-X4-X5-X6-X7-X8-T (SEQ ID NO: 2652), wherein X1 is position 91 according to the Kabat numbering system; an wherein X1 is position 91 according to the Kabat numbering system, wherein X1 is selected from Y and S; wherein X2 is selected from Y and S; wherein X3 is selected from Y and S; wherein X4 is selected from Y and S; wherein X5 is selected from Y and S, or is not present; wherein X6 is selected from Y and S, or is not present; wherein X7 is selected from P and L; and wherein X8 is selected from F, L, I, and V.
      In one aspect, the plurality of polypeptides are encoded by a plurality of polynucleotides.

In another embodiment, a method of generating a composition comprising a plurality of polypeptides is provided, comprising:
(a) generating a plurality of polypeptides comprising:
   (i) CDRH1 comprises an amino acid sequence G-F-X1-I-X2-X3-X4-X5-I-H (SEQ ID NO: 2634), wherein G is position 26 and X1 is position 28 according to the Kabat numbering system; wherein X1 is selected from Y and S; wherein X2 is selected from Y and S; wherein X3 is selected from Y and S; wherein X4 is selected from Y and S; and wherein X5 is selected from Y and S;
   (ii) CDRH2 comprises an amino acid sequence: X1-I-X2-P-X3-X4-G-X5-T-X6-Y-A-D-S-V-K-G (SEQ ID NO: 2635), wherein X1 is position 50 according to the Kabat numbering system; wherein X1 is selected from Y and S; wherein X2 is selected from Y and S; wherein X3 is selected from Y and S; wherein X4 is selected from Y and S; wherein X5 is selected from Y and S; and wherein X6 is selected from Y and S; and
   (iii) CDRH3 comprises an amino acid sequence: X1-X2-X3-X4-X5-X6-X7-X8-X9-X10-X11-X12-X13-X14-X15-X16-X17-X18-X19-D-Y (SEQ ID NO: 2636), wherein X1 is position 95 according to the Kabat numbering system, and wherein the amino acids at each of positions X1-X6 are selected from a pool of amino acids in a molar ratio of 50% Y, 25% S, and 25% G; wherein the amino acids at each of positions X7-X17 are selected from a pool of amino acids in a molar ratio of 50% Y, 25% S, and 25% G, or are not present; wherein X18 is selected from G and A; and

wherein X19 is selected from I, M, L, and F.

In one aspect, the method further comprises:

(b) generating a plurality of polypeptides comprising:
   (i) CDRL1 comprising a first consensus hypervariable sequence or variant thereof comprising substitution at one or more positions compared to a corresponding consensus hypervariable sequence;
   (ii) CDRL2 comprising a second consensus hypervariable sequence or variant thereof comprising substitution at one or more positions compared to a corresponding consensus hypervariable sequence; and
   (iii) CDRL3 comprising an amino acid sequence: Q-Q-X1-X2-X3-X4-P-X5-T (SEQ ID NO: 2653), wherein X1 is position 91 according to the Kabat numbering system, and wherein X1 is selected from Y and S, wherein X2 is selected from Y and S; wherein X3 is selected from Y and S; wherein X4 is selected from Y and S; and
   wherein X5 is selected from Y and S.
      In one aspect, the plurality of polypeptides are encoded by a plurality of polynucleotides.

In another embodiment, a method of generating a composition comprising a plurality of polypeptides is provided, comprising:
(a) generating a plurality of polypeptides comprising:
   (i) CDRH1 comprising an amino acid sequence G-F-X1-I-X2-X3-X4-X5-I-H (SEQ ID NO: 2634), wherein G is position 26 and X1 is position 28 according to the Kabat numbering system; wherein X1 is selected from Y and S; wherein X2 is selected from Y and S; wherein X3 is selected from Y and S; wherein X4 is selected from Y and S; and wherein X5 is selected from Y and S;
   (ii) CDRH2 comprising an amino acid sequence: X1-I-X2-P-X3-X4-G-X5-T-X6-Y-A-D-S-V-K-G (SEQ ID NO: 2635), wherein X1 is position 50 according to the Kabat numbering system; wherein X1 is selected from Y and S; wherein X2 is selected from Y and S; wherein X3 is selected from Y and S; wherein X4 is selected from Y and S; wherein X5 is selected from Y and S; and wherein X6 is selected from Y and S; and
   (iii) CDRH3 comprising an amino acid sequence: X1-X2-X3-X4-X5-X6-X7-X8-X9-X10-X11-X12-X13-X14-X15-X16-X17-X18-X19-D-Y (SEQ ID NO: 2637), wherein X1 is position 95 according to the Kabat numbering system, and wherein the amino acids at each of positions X1-X6 are selected from a pool of amino acids in a molar ratio of 25% Y, 50% S, and 25% R; wherein the amino acids at each of positions X7-X17 are selected from a pool of amino acids in a molar ratio of 25% Y, 50% S, and 25% R; wherein X18 is selected from G and A; and wherein X19 is selected from I, M, L, and F.
      In one aspect, the method further comprises:
(b) generating a plurality of polypeptides comprising:
   (i) CDRL1 comprising a first consensus hypervariable sequence or variant thereof comprising substitution at one or more positions compared to a corresponding consensus hypervariable sequence;
   (ii) CDRL2 comprising a second consensus hypervariable sequence or variant thereof comprising substitution at one or more positions compared to a corresponding consensus hypervariable sequence; and
   (iii) CDRL3 comprising an amino acid sequence: Q-Q-X1-X2-X3-X4-P-X5-T (SEQ ID NO: 2653), wherein X1 is position 91 according to the Kabat numbering system, and wherein X1 is selected from Y and S, wherein X2 is selected from Y and S; wherein X3 is selected from Y and S; wherein X4 is selected from Y and S; and
   wherein X5 is selected from Y and S.
      In one aspect, the plurality of polypeptides are encoded by a plurality of polynucleotides.

In another embodiment, a method of generating a composition comprising a plurality of polypeptides is provided, comprising:
(a) generating a plurality of polypeptides comprising:
   (i) CDRH1 comprising an amino acid sequence G-F-X1-I-X2-X3-X4-X5-I-H (SEQ ID NO: 2634), wherein G is position 26 and X1 is position 28 according to the Kabat numbering system; wherein X1 is selected from Y and S; wherein X2 is selected from Y and S; wherein X3 is selected from Y and S; wherein X4 is selected from Y and S; and wherein X5 is selected from Y and S;
   (ii) CDRH2 comprising an amino acid sequence: X1-I-X2-P-X3-X4-G-X5-T-X6-Y-A-D-S-V-K-G (SEQ ID NO: 2635), wherein X1 is position 50 according to the Kabat numbering system; wherein X1 is selected from Y and S; wherein X2 is selected from Y and S; wherein X3 is selected from Y and S; wherein X4 is selected from Y and S; wherein X5 is selected from Y and S; and wherein X6 is selected from Y and S; and
   (iii) CDRH3 comprising an amino acid sequence: X1-X2-X3-X4-X5-X6-X7-X8-X9-X10-X11-X12-X13-X14-X15-X16-X17-X18-X19-D-Y (SEQ ID NO: 2638), wherein X1 is position 95 according to the Kabat numbering system, and wherein the amino acids at each of positions X1-X6 are selected from a pool of amino acids in a molar ratio of 38% Y, 25% S, 25% G, and 12% R; wherein the amino acids at each of positions X7-X17 are selected from a pool of amino acids in a molar ratio of 38% Y, 25% S, 25% G, and 12% R, or are not present; wherein X18 is selected from G and A; and wherein X19 is selected from I, M, L, and F.
      In one aspect, the method further comprises:
(b) generating a plurality of polypeptides comprising:
   (i) CDRL1 comprising a first consensus hypervariable sequence or variant thereof comprising substitution at one or more positions compared to a corresponding consensus hypervariable sequence;
   (ii) CDRL2 comprising a second consensus hypervariable sequence or variant thereof comprising substitution at one or more positions compared to a corresponding consensus hypervariable sequence; and
   (iii) CDRL3 comprising an amino acid sequence: Q-Q-X1-X2-X3-X4-P-X5-T (SEQ ID NO: 2653), wherein X1 is position 91 according to the Kabat numbering system, and wherein X1 is selected from Y and S, wherein X2 is selected from Y and S; wherein X3 is selected from Y and S; wherein X4 is selected from Y and S; and
   wherein X5 is selected from Y and S.
      In one aspect, the plurality of polypeptides are encoded by a plurality of polynucleotides.

In another embodiment, a method of generating a composition comprising a plurality of polypeptides is provided, comprising:
(a) generating a plurality of polypeptides comprising:
   (i) CDRH1 comprising an amino acid sequence G-F-X1-I-X2-X3-X4-X5-I-H (SEQ ID NO: 2634), wherein G is position 26 and X1 is position 28 according to the Kabat numbering system; wherein X1 is selected from Y and S; wherein X2 is selected from Y and S; wherein X3 is selected from Y and S; wherein X4 is selected from Y and S; and wherein X5 is selected from Y and S;
   (ii) CDRH2 comprising an amino acid sequence: X1-I-X2-P-X3-X4-G-X5-T-X6-Y-A-D-S-V-K-G (SEQ ID NO: 2635), wherein Xl is position 50 according to the Kabat numbering system; wherein X1 is selected from Y and S; wherein X2 is selected from Y and S; wherein X3 is selected from Y and S; wherein X4 is selected from Y and S; wherein X5 is selected from Y and S; and wherein X6 is selected from Y and S; and
   (iii) CDRH3 comprising an amino acid sequence: X1-X2-X3-X4-X5-X6-X7-X8-X9-X10-X11-X12-X13-X14-X15-X16-X17-X18-X19-D-Y (SEQ ID NO: 2639), wherein X1 is position 95 according to the Kabat numbering system, and wherein the amino acids at each of positions X1-X6 are selected from a pool of amino acids in a molar ratio of 20% Y, 26% S, 26% G, 13% R, 1% A, 1% D, 1% E, 1% F, 1% H, 1% I, 1% K, 1% L, 1% M, 1% N, 1% P, 1% Q, 1% T, 1% V, and 1% W; wherein the amino acids at each of positions X7-X17 are selected from a pool of amino acids in a molar ratio of 20% Y, 26% S, 26% G, 13% R, 1% A, 1% D, 1% E, 1% F, 1% H, 1% I, 1% K, 1% L, 1% M, 1% N, 1% P, 1% Q, 1% T, 1% V, and 1% W, or are not present; wherein X18 is selected from G and A; and wherein X19 is selected from I, M, L, and F.
      In one aspect, the method further comprises:
(b) generating a plurality of polypeptides comprising:
   (i) CDRL1 comprising a first consensus hypervariable sequence or variant thereof comprising substitution at one or more positions compared to a corresponding consensus hypervariable sequence;
   (ii) CDRL2 comprising a second consensus hypervariable sequence or variant thereof comprising substitution at one or more positions compared to a corresponding consensus hypervariable sequence; and
   (iii) CDRL3 comprising an amino acid sequence: Q-Q-X1-X2-X3-X4-P-X5-T (SEQ ID NO: 2653), wherein X1 is position 91 according to the Kabat numbering system, and wherein X1 is selected from Y and S, wherein X2 is selected from Y and S; wherein X3 is selected from Y and S; wherein X4 is selected from Y and S; and
   wherein X5 is selected from Y and S.
      In one aspect, the first consensus hypervariable sequence comprises a Kabat consensus CDRLl sequence. In one such aspect, the first consensus hypervariable sequence is R-A-S-Q-D-V-N-T-A-V-A (SEQ ID NO: 6). In one aspect, the second consensus hypervariable sequence comprises a Kabat consensus CDRL2 sequence. In one such aspect, the second consensus hypervariable sequence is S-A-S-S-L-Y-S (SEQ ID NO: 7). In one aspect, the plurality of polypeptides are encoded by a plurality of polynucleotides.

In one embodiment, a method of generating a composition comprising a plurality of polypeptides is provided, comprising:
(a) generating a plurality of polypeptides comprising:
   (i) CDRH1 comprising an amino acid sequence G-F-X1-I-X2-X3-X4-X5-I-H (SEQ ID NO: 2634), wherein G is position 26 and X1 is position 28 according to the Kabat numbering system; wherein X1 is selected from Y and S; wherein X2 is selected from Y and S; wherein X3 is selected from Y and S; wherein X4 is selected from Y and S; and wherein X5 is selected from Y and S;
   (ii) CDRH2 comprising an amino acid sequence: X1-I-X2-P-X3-X4-S-X5-T-X6-Y-A-D-S-V-K-G (SEQ ID NO: 2657), wherein X1 is position 50 according to the Kabat numbering system; wherein X1 is selected from Y and S; wherein X2 is selected from Y and S; wherein X3 is selected from Y and S; wherein X4 is selected from Y and S; wherein X5 is selected from Y and S; and wherein X6 is selected from Y and S; and
   (iii) CDRH3 comprising an amino acid sequence: X1-X2-X3-X4-X5-X6-X7-X8-X9-X10-X11-X12-X13-X14-X15-X16-X17-X18-X19 (SEQ ID NO: 2640), where X1 is position 95 according to the Kabat numbering system, and wherein the amino acids at each of positions X1-X19 are selected from S and one of A, C, F, G, I, L, N, P, R, T, W, or Y, or are not present; wherein X18 is selected from G and A; and

wherein X19 is selected from F, L, I, and M.

In one aspect, the method further comprises:

(b) generating a plurality of polypeptides comprising:
   (i) CDRL1 comprising a first consensus hypervariable sequence or variant thereof comprising substitution at one or more positions compared to a corresponding consensus hypervariable sequence;
   (ii) CDRL2 comprising a second consensus hypervariable sequence or variant thereof comprising substitution at one or more positions compared to a corresponding consensus hypervariable sequence; and
   (iii) CDRL3 comprising an amino acid sequence: Q-Q-X1-X2-X3-X4-P-X5-T (SEQ ID NO: 2653), wherein X1 is position 91 according to the Kabat numbering system; wherein X1 is selected from Y and S; wherein X2 is selected from Y and S;
   wherein X3 is selected from Y and S; wherein X4 is selected from Y and S; and
   wherein X5 is selected from Y and S.

In one embodiment, a method of generating a composition comprising a plurality of polypeptides is provided, comprising:
(a) generating a plurality of polypeptides comprising:
   (i) CDRH1 comprises an amino acid sequence G-F-X1-I-X2-X3-X4-X5-I-H (SEQ ID NO: 2641), wherein G is position 26 and X1 is position 28 according to the Kabat numbering system; wherein the amino acid at each of positions X1-X5 is selected from S and one of Y, W, R, or F;
   (ii) CDRH2 comprises an amino acid sequence: X1-I-X2-P-X3-X4-S-X5-T-X6-Y-A-D-S-V-K-G (SEQ ID NO: 2642), wherein X1 is position 50 according to the Kabat numbering system; wherein the amino acid at each of positions X1-X6 is selected from S and one of Y, W, R, or F; and
   (iii) CDRH3 comprises an amino acid sequence: X1-X2-X3-X4-X5-X6-X7-X8-X9-X10-X11-X12-X13-X14-X15-X16-X17-X18-X19 (SEQ ID NO: 2643), wherein X1 is position 95 according to the Kabat numbering system, and wherein the amino acids at each of positions X1-X19 are selected from S and one of Y, W, R, or F, or are not present; wherein X18 is selected from G and A; and wherein X19 is selected from F, L, I, and M.
      In another aspect, the method further comprises:
(b) generating a plurality of polypeptides comprising:
   (i) CDRL1 comprising a first consensus hypervariable sequence or variant thereof comprising substitution at one or more positions compared to a corresponding consensus hypervariable sequence;
   (ii) CDRL2 comprising a second consensus hypervariable sequence or variant thereof comprising substitution at one or more positions compared to a corresponding consensus hypervariable sequence; and
   (iii) CDRL3 comprising an amino acid sequence: Q-Q-X1-X2-X3-X4-P-X5-T (SEQ ID NO: 2654), wherein X1 is position 91 according to the Kabat numbering system; and wherein the amino acids at each of positions X1-X5 are selected from S and one of Y, W, R, and F.

In one embodiment, a method of generating one or more of the above-described CDRH1, CDRH2, CDRH3, CDRL1, CDRL2, and CDRL3 sequences is provided, comprising:
(a) constructing an expression vector comprising a polynucleotide sequence which encodes a light chain variable domain, a heavy chain variable domain, or both of a source antibody comprising at least one, two, three, four, five or all CDRs of the source antibody selected from the group consisting of CDRL1, CDRL2, CDRL3, CDRH1, CDRH2, and CDRH3; and
(b) mutating at least one, two three, four, five or all CDRs of the source antibody to generate one or more of the above-described hypervariable regions.

In one embodiment, a method of selecting for a polypeptide that binds to a target antigen is provided, comprising:
(a) generating a composition with a plurality of one or more of the above-described polypeptides;
(b) selecting one or more polypeptides from the composition that binds to a target antigen;
(c) isolating the one or more polypeptides that bind to the target antigen from polypeptides that do not bind to the target antigen; and
(d) identifying the one or more polypeptides that bind to the target antigen that have a desired affinity for the target antigen.

In one embodiment, a method of selecting for an antigen binding variable domain that binds to a target antigen from a library of antibody variable domains is provided, comprising:
(a) contacting one or more of the above-described libraries with a target antigen;
(b) separating one or more polypeptides that specifically bind to the target antigen from polypeptides that do not specifically bind to the target antigen, recovering the one or more polypeptides that specifically bind to the target antigen, and incubating the one or more polypeptides that specifically bind to the target antigen in a series of solutions comprising decreasing amounts of the target antigen in a concentration from about 0.1 nM to about 1000 nM; and
(c) selecting the one or more polypeptides that specifically bind to the target antigen and that can bind to the lowest concentration of the target antigen or that have an affinity of about 0.1 nM to about 200 nM.
   In one aspect, the target antigen is VEGF, insulin, HER2, IGF-1, or HGH. In one aspect, the concentration of the target antigen is about 100 to about 250 nM. In one aspect, the concentration of target antigen is about 25 to about 100 nM. In some embodiments, one or more of the libraries, clones or polypeptides are screened against a panel of antigens including the target antigen. In some embodiments, those clones or polypeptides that specifically bind to the target antigen and do not substantially crossreact with any of the other antigen on the panel are selected. The panel of antigens can include at least three and up to 100 different antigens. In some cases, the panel of antigens includes 3 to 100, 3 to 50, 3 to 25, or 3 to 10 different antigens.

In one embodiment, a method of selecting for a polypeptide that binds to a target antigen from a library of polypeptides is provided, comprising:
(a) isolating one or more polypeptides that specifically bind to the target antigen by contacting a library comprising a plurality of any of the above-described polypeptides with an immobilized target antigen under conditions suitable for binding;
(b) separating the one or more polypeptides that specifically bind to the target antigen from polypeptides that do not specifically bind to the target antigen, and recovering the one or more polypeptides that specifically bind to the target antigen to obtain a subpopulation enriched for the one or more polypeptides that specifically bind to the target antigen; and
(c) optionally, repeating steps (a)-(b) at least twice, each repetition using the subpopulation enriched for the one or more polypeptides that specifically bind to the target antigen obtained from the previous round of selection.
   In one aspect, the method further comprises:
(d) incubating the subpopulation with a concentration of labeled target antigen in the range of about 0.1 nM to about 1000 nM to form a mixture, under conditions suitable for binding;
(e) contacting the mixture with an immobilized agent that binds to the label on the target antigen;
(f) detecting the one or more polypeptides that specifically bind to the labeled target antigen, and recovering the one or more polypeptides that specifically bind to the labeled target antigen from the labeled target antigen; and
(g) optionally, repeating steps (d) to (f) at least twice, each repetition using the subpopulation enriched for the one or more polypeptides that specifically bind to the labeled target antigen obtained from the previous round of selection, and using a lower concentration of labeled target antigen than the previous round of selection.
   In one aspect, the method further comprises adding an excess of unlabeled target antigen to the mixture and incubating the mixture for a period of time sufficient to recover one or more polypeptides that specifically bind to the target antigen with low affinity. In some embodiments, in any of the methods described herein, one or more of the libraries, clones or polypeptides are screened against a panel of antigens including the target antigen. In some embodiments, those clones or polypeptides that specifically bind to the target antigen and do not substantially crossreact with any of the other antigen on the panel are selected. The panel of antigens can include at least three and up to 100 different antigens. In some cases, the panel of antigens includes 3 to 100, 3 to 50, 3 to 25, or 3 to 10 different antigens.

In one embodiment, a method of isolating one or more polypeptides that specifically bind to a target antigen with high affinity is provided, comprising:
(a) contacting a library comprising a plurality of any of the above-described polypeptides with a target antigen at a concentration of at least about 0.1 nM to about 1000 nM to isolate one or more polypeptides that specifically bind to the target antigen;
(b) recovering the one or more polypeptides that specifically bind to the target antigen from the target antigen to obtain a subpopulation enriched for the one or more polypeptides that specifically bind to the target antigen; and
(c) optionally repeating steps (a) and (b) at least twice, each repetition using the subpopulation obtained from the previous round of selection and using a decreased concentration of target antigen from that used in the previous round to isolate one or more polypeptides that bind specifically to the target antigen at the lowest concentration of target antigen.

In one embodiment, an assay for selecting one or more polypeptides that bind to a target antigen from a library comprising a plurality of any of the above-described polypeptides is provided, comprising:
(a) contacting the library with a concentration of labeled target antigen at a concentration range of about 0.1 nM to about 1000 nM, under conditions suitable for formation of one or more complexes between the labeled target antigen and one or more polypeptides that specifically bind the target antigen;
(b) isolating the one or more complexes and separating the one or more polypeptides that specifically bind the target antigen from the labeled target antigen to obtain a subpopulation enriched for the one or more polypeptides that specifically bind the target antigen; and
(c) optionally, repeating steps (a) and (b) at least twice, each time using the subpopulation obtained from the previous round of selection and using a lower concentration of target antigen than was used in the previous round.
   In one aspect, the assay further comprises adding an excess of unlabeled target antigen to the one or more complexes. In one aspect, steps (a) and (b) are repeated twice, wherein the concentration of target antigen in the first round of selection is about 100 nM to about 250 nM, wherein the concentration of target antigen in the second round of selection is about 25 nM to about 100 nM, and wherein the concentration of target antigen in the third round of selection is about 0.1 nM to about 25 nM.

In one embodiment, a method of screening a library comprising a plurality of any of the above-described polypeptides is provided, comprising:
(a) incubating a first sample of the library with a target antigen under conditions suitable for binding of the polypeptides to the target antigen;
(b) incubating a second sample of the library in the absence of a target antigen;
(c) contacting each of the first sample and the second sample with immobilized target antigen under conditions suitable for binding of the polypeptide to the immobilized target antigen;
(d) detecting the polypeptide bound to immobilized target antigen for each sample; and
(e) determining the affinity of the polypeptide for the target antigen by calculating the ratio of the amounts of bound polypeptide from the first sample over the amount of bound polypeptide from the second sample.

In one embodiment, one or more of the above-described polypeptides specifically binds human VEGF. In one aspect, the polypeptide is an antibody that specifically binds human VEGF. In one such aspect, the antibody comprises the framework regions of the 4D5 antibody. In one such aspect, the antibody comprises the framework regions of a variant 4D5 antibody. In one such aspect, the antibody is a monoclonal antibody. In one such aspect, the antibody is a bispecific antibody. In one such aspect, the antibody is a synthetic antibody.

In one aspect, an antibody that specifically binds human VEGF comprises a CDRH1 amino acid sequence comprising at least one sequence selected from SEQ ID NOS: 52-66, 111-125, 318-439, 1340-1396, and 2027-2057 or at least one sequence in any of FIGS. 10, 14, 21 or 28. In one aspect, an antibody that specifically binds human VEGF comprises a CDRH2 amino acid sequence comprising at least one sequence selected from SEQ ID NOS: 67-81, 126-141, 440-561, 1397-1453, and 2058-2088 or at least one sequence in any of FIGS. 10, 14, 21 or 28. In one aspect, an antibody that specifically binds human VEGF comprises a CDRH3 amino acid sequence comprising at least one sequence selected from SEQ ID NOS: 82-96, 142-157, 562-683, 1454-1510, and 2089-2119 or at least one sequence in any of FIGS. 10, 14, 21 or 28. In one aspect, an antibody that specifically binds human VEGF comprises a CDRL3 amino acid sequence comprising at least one sequence selected from SEQ ID NOS: 37-51, 97-110, 209-317, 1283-1339, and 1996-2026 or at least one sequence in any of FIGS. 10, 14, 21 or 28. In one aspect, an antibody that specifically binds human VEGF comprises CDRH1, CDRH2, CDRH3, and CDRL3 sequences corresponding to the CDRH1, CDRH2, CDRH3, and CDRL3 sequences set forth in FIG. 10 for any one of Fabs 1-31. In one aspect, an antibody that specifically binds human VEGF comprises CDRH1, CDRH2, CDRH3, and CDRL3 sequences corresponding to the CDRH1, CDRH2, CDRH3, and CDRL3 sequences set forth in FIGS. 14A-C for any one of clones 1-122. In another aspect, an antibody that specifically binds human VEGF comprises CDRH1, CDRH2, CDRH3, and CDRL3 sequences corresponding to the CDRH1, CDRH2, CDRH3, and CDRL3 sequences set forth in FIGS. 21A-21B for any one of clones A1-A60. In another aspect, an antibody that specifically binds human VEGF comprises CDRH1, CDRH2, CDRH3, and CDRL3 sequences corresponding to the CDRH1, CDRH2, CDRH3, and CDRL3 sequences set forth in FIG. 28A for any one of clones F1-F148.

In one embodiment, an isolated polynucleotide encoding any of the above-described antibodies that specifically binds human VEGF is provided. In one embodiment, a vector comprising an isolated polynucleotide encoding any of the above-described antibodies that specifically binds human VEGF is provided. In one embodiment, a host cell transformed with a vector comprising an isolated polynucleotide encoding any of the above-described antibodies that specifically bind human VEGF is provided. In one embodiment, a process of producing an antibody is provided, comprising culturing a host cell transformed with a vector comprising an isolated polynucleotide encoding any of the above-described antibodies that specifically bind human VEGF such that the polynucleotide is expressed. In one aspect, the process further comprises recovering the antibody from the host cell culture. In one aspect, the process further comprises recovering the antibody from the host cell culture medium.

In one embodiment, a method of using one or more of the above-described antibodies that specifically bind human VEGF for treating a disorder associated with abnormal angiogenesis in a mammal in need of treatment thereof is provided, comprising the step of administering the one or more antibodies to the mammal. In one aspect, the disorder is cancer. In one such aspect, the cancer is selected from breast cancer, colorectal cancer, non-small cell lung cancer, non-Hodgkins lymphoma (NHL), renal cancer, prostate cancer, liver cancer, head and neck cancer, melanoma, ovarian cancer, mesothelioma, and multiple myeloma. In another aspect, the treatment further comprises the step of administering a second therapeutic agent simultaneously or sequentially with the antibody. In one such aspect, the second therapeutic agent is selected from an anti-angiogenic agent, an anti-neoplastic agent, a chemotherapeutic agent, and a cytotoxic agent. In one such aspect, the anti-angiogenic agent is an anti-hVEGF antibody capable of binding to the same VEGF epitope as the antibody A4.6.1.

In one embodiment, a method of treating a mammal suffering from or at risk of developing an inflammatory or immune disorder is provided, comprising the step of treating the mammal with one or more Fabs of one or more of the above-described antibodies that specifically bind human VEGF. In one aspect, the inflammatory or immune disorder is rheumatoid arthritis.

In one aspect, an antibody that specifically binds HER2 comprises a CDRH1 amino acid sequence comprising at least one sequence selected from SEQ ID NOS: 1538-1564 and 2147-2173. In one aspect, an antibody that specifically binds HER2 comprises a CDRH2 amino acid sequence comprising at least one sequence selected from SEQ ID NOS: 1565-1591 and 2174-2200 or at least one sequence selected from any of the sequences in FIG. 22 or FIG. 29. In one aspect, an antibody that specifically binds HER2 comprises a CDRH3 amino acid sequence comprising at least one sequence selected from SEQ ID NOS: 1592-1618 and 2201-2227 or at least one sequence selected from any of the sequences in FIG. 22 or FIG. 29. In one aspect, an antibody that specifically binds HER2 comprises a CDRL3 amino acid sequence comprising at least one sequence selected from SEQ ID NOS: 1511-1537 and 2120-2146 or at least one sequence selected from any of the sequences in FIG. 22 or FIG. 29. In one aspect, an antibody that specifically binds HER2 comprises CDRH1, CDRH2, CDRH3, and CDRL3 sequences corresponding to the CDRH1, CDRH2, CDRH3, and CDRL3 sequences set forth in FIG. 22A for any one of clones B1-B28. In one aspect, an antibody that specifically binds HER2 comprises CDRH1, CDRH2, CDRH3, and CDRL3 sequences corresponding to the CDRH1, CDRH2, CDRH3, and CDRL3 sequences set forth in FIG. 29A for any one of clones G29-G61.

In one embodiment, an isolated polynucleotide encoding any of the above-described antibodies that specifically binds HER2 is provided. In one embodiment, a vector comprising an isolated polynucleotide encoding any of the above-described antibodies that specifically binds HER2 is provided. In one embodiment, a host cell transformed with a vector comprising an isolated polynucleotide encoding any of the above-described antibodies that specifically bind HER2 is provided. In one embodiment, a process of producing an antibody is provided, comprising culturing a host cell transformed with a vector comprising an isolated polynucleotide encoding any of the above-described antibodies that specifically bind HER2 such that the polynucleotide is expressed. In one aspect, the process further comprises recovering the antibody from the host cell culture. In one aspect, the process further comprises recovering the antibody from the host cell culture medium.

In one embodiment, a method of using one or more of the above-described antibodies that specifically bind HER2 for treating a HER2-related disorder, comprising the step of administering the one or more antibodies to the mammal. In another aspect, the treatment further comprises the step of administering a second therapeutic agent simultaneously or sequentially with the antibody. In one such aspect, the second therapeutic agent is selected from an anti-angiogenic agent, an anti-neoplastic agent, a chemotherapeutic agent, and a cytotoxic agent.

In one embodiment, a method of treating a mammal suffering from or at risk of developing a HER2-related disorder, comprising the step of treating the mammal with one or more Fabs of one or more of the above-described antibodies that specifically bind HER2. In one embodiment, one or more of the above-described polypeptides specifically binds insulin. In one aspect, the polypeptide is an antibody that specifically binds insulin. In one such aspect, the antibody comprises the framework regions of the 4D5 antibody. In one such aspect, the antibody comprises the framework regions of a variant 4D5 antibody. In one such aspect, the antibody is a monoclonal antibody. In one such aspect, the antibody is a bispecific antibody. In one such aspect, the antibody is a synthetic antibody.

In one aspect, an antibody that specifically binds insulin comprises a CDRH1 amino acid sequence comprising at least one sequence selected from SEQ ID NOS: 784-888, 1653-1686, and 2239-2249 or at least one sequence selected from any of the CDRH1 sequences in FIGS. 15, 23 or 30. In one aspect, an antibody that specifically binds insulin comprises a CDRH2 amino acid sequence comprising at least one sequence selected from SEQ ID NOS: 889-993, 1687-1720, and 2250-2260 or at least one sequence selected from any of the CDRH2 sequences in FIGS. 15, 23 or 30. In one aspect, an antibody that specifically binds insulin comprises a CDRH3 amino acid sequence comprising at least one sequence selected from SEQ ID NOS: 994-1098, 1721-1754, and 2261-2271 or at least one sequence selected from any of the CDRH3 sequences in FIGS. 15, 23 or 30. In one aspect, an antibody that specifically binds insulin comprises a CDRL3 amino acid sequence comprising at least one sequence selected from SEQ ID NOS: 684-783, 1619-1652, and 2228-2238 or at least one sequence selected from any of the CDRL3 sequences in FIGS. 15, 23 or 30. In one aspect, an antibody that specifically binds insulin comprises CDRH1, CDRH2, CDRH3, and CDRL3 sequences corresponding to the CDRH1, CDRH2, CDRH3, and CDRL3 sequences set forth in FIGS. 15A-15B for any one of clones 1-105. In another aspect, an antibody that specifically binds insulin comprises CDRH1, CDRH2, CDRH3, and CDRL3 sequences corresponding to the CDRH1, CDRH2, CDRH3, and CDRL3 sequences set forth in FIG. 23A for any one of clones C1-C47. In another aspect, an antibody that specifically binds insulin comprises CDRH1, CDRH2, CDRH3, and CDRL3 sequences corresponding to the CDRH1, CDRH2, CDRH3, and CDRL3 sequences set forth in FIG. 30A for any one of clones H43-H65.

In one embodiment, an isolated polynucleotide encoding any of the above-described antibodies that specifically binds insulin is provided. In one embodiment, a vector comprising an isolated polynucleotide encoding any of the above-described antibodies that specifically binds insulin is provided. In one embodiment, a host cell transformed with a vector comprising an isolated polynucleotide encoding any of the above-described antibodies that specifically bind insulin is provided. In one embodiment, a process of producing an antibody is provided, comprising culturing a host cell transformed with a vector comprising an isolated polynucleotide encoding any of the above-described antibodies that specifically bind insulin such that the polynucleotide is expressed. In one aspect, the process further comprises recovering the antibody from the host cell culture. In one aspect, the process further comprises recovering the antibody from the host cell culture medium.

In one embodiment, a method of using one or more of the above-described antibodies that specifically bind insulin for treating an insulin-related disorder in a mammal in need of treatment thereof is provided, comprising the step of administering the one or more antibodies to the mammal. In one embodiment, a method of treating a mammal suffering from or at risk of developing an insulin-related disorder is provided, comprising the step of treating the mammal with one or more Fabs of one or more of the above-described antibodies that specifically bind insulin.

In one aspect, an antibody that specifically binds human IGF-1 comprises a CDRH1 amino acid sequence comprising at least one sequence selected from SEQ ID NOS: 1805-1854 and 2300-2327 or at least one CDRH1 sequence selected from sequences in FIGS. 24 or 31. In one aspect, an antibody that specifically binds human IGF-1 comprises a CDRH2 amino acid sequence comprising at least one sequence selected from SEQ ID NOS: 1855-1904 and 2328-2355 or at least one CDRH2 sequence selected from sequences in FIGS. 24 or 31. In one aspect, an antibody that specifically binds human IGF-1 comprises a CDRH3 amino acid sequence comprising at least one sequence selected from SEQ ID NOS: 1905-1954 and 2356-2383 or at least one CDRH3 sequence selected from sequences in FIGS. 24 or 31. In one aspect, an antibody that specifically binds human IGF-1 comprises a CDRL3 amino acid sequence comprising at least one sequence selected from SEQ ID NOS: 1755-1804 and 2272-2299 or at least one CDRL3 sequence selected from sequences in FIGS. 24 or 31. In one aspect, an antibody that specifically binds human IGF-1 comprises CDRH1, CDRH2, CDRH3, and CDRL3 sequences corresponding to the CDRH1, CDRH2, CDRH3, and CDRL3 sequences set forth in FIG. 24A for any one of clones D44-D159. In one aspect, an antibody that specifically binds human IGF-1 comprises CDRH1, CDRH2, CDRH3, and CDRL3 sequences corresponding to the CDRH1, CDRH2, CDRH3, and CDRL3 sequences set forth in FIG. 31 A for any one of clones I67-I161.

In one embodiment, an isolated polynucleotide encoding any of the above-described antibodies that specifically binds human IGF-1 is provided. In one embodiment, a vector comprising an isolated polynucleotide encoding any of the above-described antibodies that specifically binds human IGF-1 is provided. In one embodiment, a host cell transformed with a vector comprising an isolated polynucleotide encoding any of the above-described antibodies that specifically bind human IGF-1 is provided. In one embodiment, a process of producing an antibody is provided, comprising culturing a host cell transformed with a vector comprising an isolated polynucleotide encoding any of the above-described antibodies that specifically bind human IGF-1 such that the polynucleotide is expressed. In one aspect, the process further comprises recovering the antibody from the host cell culture. In one aspect, the process further comprises recovering the antibody from the host cell culture medium.

In one embodiment, a method of using one or more of the above-described antibodies that specifically bind human IGF-1 for treating an IGF-1-related disorder, comprising the step of administering the one or more antibodies to the mammal. In another aspect, the treatment further comprises the step of administering a second therapeutic agent simultaneously or sequentially with the antibody. In one such aspect, the second therapeutic agent is selected from an anti-angiogenic agent, an anti-neoplastic agent, a chemotherapeutic agent, and a cytotoxic agent.

In one embodiment, a method of treating a mammal suffering from or at risk of developing an IGF-1-related disorder is provided, comprising the step of treating the mammal with one or more Fabs of one or more of the above-described antibodies that specifically bind human IGF-1.

In one aspect, an antibody that specifically binds human growth hormone (HGH) comprises a CDRH1 amino acid sequence comprising at least one sequence selected from SEQ ID NOS: 1963-1970 and 2395-2405 or at least one sequence selected from any of the sequences in FIGS. 25 or 32. In one aspect, an antibody that specifically binds HGH comprises a CDRH2 amino acid sequence comprising at least one sequence selected from SEQ ID NOS: 1971-1978 and 2406-2416 or at least one sequence selected from any of the sequences in FIGS. 25 or 32. In one aspect, an antibody that specifically binds HGH comprises a CDRH3 amino acid sequence comprising at least one sequence selected from SEQ ID NOS: 1979-1986 and 2417-2427 or at least one sequence selected from any of the sequences in FIGS. 25 or 32. In one aspect, an antibody that specifically binds HGH comprises a CDRL3 amino acid sequence comprising at least one sequence selected from SEQ ID NOS: 1955-1962 and 2384-2394 or at least one sequence selected from any of the sequences in FIGS. 25 or 32. In one aspect, an antibody that specifically binds HGH comprises CDRH1, CDRH2, CDRH3, and CDRL3 sequences corresponding to the CDRH1, CDRH2, CDRH3, and CDRL3 sequences set forth in FIG. 25A for any one of clones E35-E43. In one aspect, an antibody that specifically binds HGH comprises CDRH1, CDRH2, CDRH3, and CDRL3 sequences corresponding to the CDRH1, CDRH2, CDRH3, and CDRL3 sequences set forth in FIG. 32A for any one of clones J56-J66.

In one embodiment, an isolated polynucleotide encoding any of the above-described antibodies that specifically binds HGH is provided. In one embodiment, a vector comprising an isolated polynucleotide encoding any of the above-described antibodies that specifically binds HGH is provided. In one embodiment, a host cell transformed with a vector comprising an isolated polynucleotide encoding any of the above-described antibodies that specifically bind HGH is provided. In one embodiment, a process of producing an antibody is provided, comprising culturing a host cell transformed with a vector comprising an isolated polynucleotide encoding any of the above-described antibodies that specifically bind HGH such that the polynucleotide is expressed. In one aspect, the process further comprises recovering the antibody from the host cell culture. In one aspect, the process further comprises recovering the antibody from the host cell culture medium.

In one embodiment, a method of using one or more of the above-described antibodies that specifically bind HGH for treating a GH-related disorder, comprising the step of administering the one or more antibodies to the mammal. In one aspect, the disorder is a growth disorder. In another aspect, the disorder is cancer. In another aspect, the treatment further comprises the step of administering a second therapeutic agent simultaneously or sequentially with the antibody. In one such aspect, the second therapeutic agent is selected from an anti-angiogenic agent, an anti-neoplastic agent, a chemotherapeutic agent, and a cytotoxic agent.

In one embodiment, a method of treating a mammal suffering from or at risk of developing a growth disorder is provided, comprising the step of treating the mammal with one or more Fabs of one or more of the above-described antibodies that specifically bind human growth hormone.

In one aspect, a polypeptide of the invention comprises at least one, or both, of heavy chain and light chain antibody variable domains, wherein the antibody variable domain comprises one, two or three variant CDRs as described herein (e.g., as described in the foregoing).

In some embodiments, a polypeptide of the invention (in particular those comprising an antibody variable domain) further comprises an antibody framework sequence, e.g., FR1, FR2, FR3 and/or FR4 for an antibody variable domain corresponding to the variant CDR, the FR sequences obtained from a single antibody template. In one embodiment, the FR sequences are obtained from a human antibody. In one embodiment, the FR sequences are obtained from a human consensus sequence (e.g., subgroup III consensus sequence). In one embodiment, the framework sequences comprise a modified consensus sequence as described herein (e.g., comprising modifications at position 49, 71, 93 and/or 94 in the heavy chain, and/or position 66 in the light chain). In one embodiment, framework regions have the sequences of the framework regions from wild-type humanized antibody 4D5-8 light chain and heavy chain (shown in FIG. 16 (SEQ ID NOS: 1099-1102 and 1103-1106, respectively)). In one embodiment, framework regions have the sequences of the framework regions from a variant version of the humanized antibody 4D5-8 light chain and heavy chain, wherein the light chain is modified at position 66 and the heavy chain is modified at positions 71, 73, and 78 (shown in FIG. 17 (SEQ ID NOS: 1107-1110 and 1111-1114)).

In some embodiments, a polypeptide of the invention comprises a light chain and a heavy chain antibody variable domain, wherein the light chain variable domain comprises at least 1, 2 or 3 variant CDRs selected from the group consisting of CDR L1, L2 and L3, and the heavy chain variable domain comprises at least 1, 2 or 3 variant CDRs selected from the group consisting of CDR H1, H2 and H3.

In some embodiments, a polypeptide of the invention is an ScFv. In some embodiments, it is a Fab fragment. In some embodiments, it is a F(ab)₂ or F(ab′)₂. Accordingly, in some embodiments, a polypeptide of the invention further comprises a dimerization domain. In some embodiments, the dimerization domain is located between an antibody heavy chain or light chain variable domain and at least a portion of a viral coat protein. The dimerization domain can comprise a dimerization sequence, and/or sequence comprising one or more cysteine residues. The dimerization domain can be linked, directly or indirectly, to the C-terminal end of a heavy chain variable or constant domain. The structure of the dimerization domain can be varied depending on whether the antibody variable domain is produced as a fusion protein component with the viral coat protein component (without an amber stop codon after dimerization domain) or whether the antibody variable domain is produced predominantly without viral coat protein component (e.g., with an amber stop codon after dimerization domain). When the antibody variable domain is produced predominantly as a fusion protein with viral coat protein component, one or more disulfide bond and/or a single dimerization sequence provides for bivalent display. For antibody variable domains predominantly produced without being fused to a viral coat protein component (e.g. with amber stop), it is preferable, though not required, to have a dimerization domain comprising both a cysteine residue and a dimerization sequence. In some embodiments, heavy chains of the F(ab)₂ dimerize at a dimerization domain not including a hinge region. The dimerization domain may comprise a leucine zipper sequence (for example, a GCN4 sequence such as GRMKQLEDKVEELLSKNYHLFNEVARLKKLVGERG (SEQ ID NO: 3)).

In some embodiments, a polypeptide of the invention further comprises a light chain constant domain fused to a light chain variable domain, which in some embodiments comprises at least one, two or three variant CDRs. In some embodiments of polypeptides of the invention, the polypeptide comprises a heavy chain constant domain fused to a heavy chain variable domain, which in some embodiments comprises at least one, two or three variant CDRs.

In some instances, it may be preferable to mutate a framework residue such that it is variant with respect to a reference polypeptide or source antibody. For example, framework residue 71 of the heavy chain may be amino acid R, V or A. In another example, framework residue 93 of the heavy chain may be amino acid S or A. In yet another example, framework residue 94 of the heavy chain may be amino acid R, K or T or encoded by MRT. In yet another example, framework residue 49 of the heavy chain may be amino acid A or G. Framework residues in the light chain may also be mutated. For example, framework residue 66 in the light chain may be amino acid R or G.

As described herein, a variant CDR refers to a CDR with a sequence variance as compared to the corresponding CDR of a single reference polypeptide/source antibody. Accordingly, the CDRs of a single polypeptide of the invention can in certain embodiments correspond to the set of CDRs of a single reference polypeptide or source antibody. Polypeptides of the invention may comprise any one or combinations of variant CDRs. For example, a polypeptide of the invention may comprise a variant CDRH1 and variant CDRH2. A polypeptide of the invention may comprise a variant CDRH1, variant CDRH2 and a variant CDRH3. In another example, a polypeptide of the invention may comprise a variant CDRH1, variant CDRH2, variant CDRH3 and variant CDRL3. In another example, a polypeptide of the invention comprises a variant CDRL1, variant CDRL2 and variant CDRL3. Any polypeptide of the invention may further comprise a variant CDRL3. Any polypeptide of the invention may further comprise a variant CDRH3.

In one embodiment, a polypeptide of the invention comprises one or more variant CDR sequences as depicted in FIG. 10. In one embodiment, a polypeptide of the invention comprises one or more variant CDR sequences as depicted in FIGS. 14A-C. In one embodiment, a polypeptide of the invention comprises one or more variant CDR sequences as depicted in FIGS. 15A-15B. In another embodiment, a polypeptide of the invention comprises one or more variant CDR sequences as depicted in FIGS. 21A-21B. In another embodiment, a polypeptide of the invention comprises one or more variant CDR sequences as depicted in FIG. 22A. In another embodiment, a polypeptide of the invention comprises one or more variant CDR sequences as depicted in FIG. 23A. In another embodiment, a polypeptide of the invention comprises one or more variant CDR sequences as depicted in FIGS. 24A-B. In another embodiment, a polypeptide of the invention comprises one or more variant CDR sequences as depicted in FIG. 25A. In another embodiment, a polypeptide of the invention comprises one or more variant CDR sequences as depicted in FIGS. 28A-C. In another embodiment, a polypeptide of the invention comprises one or more variant CDR sequences as depicted in FIG. 29A. In another embodiment, a polypeptide of the invention comprises one or more variant CDR sequences as depicted in FIG. 30A. In another embodiment, a polypeptide of the invention comprises one or more variant CDR sequences as depicted in FIGS. 31A-B. In another embodiment, a polypeptide of the invention comprises one or more variant CDR sequences as depicted in FIG. 32A.

Polypeptides of the invention may be in a complex with one another. For example, the invention provides a polypeptide complex comprising two polypeptides, wherein each polypeptide is a polypeptide of the invention, and wherein one of said polypeptides comprises at least one, two or all of variant CDRs H1, H2 and H3, and the other polypeptide comprises a variant light chain CDR (e.g., CDR L3). A polypeptide complex may comprise a first and a second polypeptide (wherein the first and second polypeptides are polypeptides of the invention), wherein the first polypeptide comprises at least one, two or three variant light chain CDRs, and the second polypeptide comprises at least one, two or three variant heavy chain CDRs. The invention also provides complexes of polypeptides that comprise the same variant CDR sequences. Complexing can be mediated by any suitable technique, including by dimerization/multimerization at a dimerization/multimerization domain such as those described herein or covalent interactions (such as through a disulfide linkage) (which in some contexts is part of a dimerization domain, for example a dimerization domain may contain a leucine zipper sequence and a cysteine).

In another aspect, the invention provides compositions comprising polypeptides and/or polynucleotides of the invention. For example, the invention provides a composition comprising a plurality of any of the polypeptides of the invention described herein. Said plurality may comprise polypeptides encoded by a plurality of polynucleotides generated using a set of oligonucleotides comprising degeneracy in the sequence encoding a variant amino acid, wherein said degeneracy is that of the multiple codon sequences of the restricted codon set encoding the variant amino acid. A composition comprising a polynucleotide or polypeptide or library of the invention may be in the form of a kit or an article of manufacture (optionally packaged with instructions, buffers, etc.).

In one aspect, the invention provides a polynucleotide encoding a polypeptide of the invention as described herein. In another aspect, the invention provides a vector comprising a sequence encoding a polypeptide of the invention. The vector can be, for example, a replicable expression vector (for example, the replicable expression vector can be M13, f1, fd, Pf3 phage or a derivative thereof, or a lambdoid phage, such as lambda, 21, phi80, phi81, 82, 424, 434, etc., or a derivative thereof). The vector can comprise a promoter region linked to the sequence encoding a polypeptide of the invention. The promoter can be any suitable for expression of the polypeptide, for example, the lac Z promoter system, the alkaline phosphatase pho A promoter (Ap), the bacteriophage l_{PL} promoter (a temperature sensitive promoter), the tac promoter, the tryptophan promoter, and the bacteriophage T7 promoter. Thus, the invention also provides a vector comprising a promoter selected from the group consisting of the foregoing promoter systems.

Polypeptides of the invention can be displayed in any suitable form in accordance with the need and desire of the practitioner. For example, a polypeptide of the invention can be displayed on a viral surface, for example, a phage or phagemid viral particle. Accordingly, the invention provides viral particles comprising a polypeptide of the invention and/or polynucleotide encoding a polypeptide of the invention.

In one aspect, the invention provides a population comprising a plurality of polypeptide or polynucleotide of the invention, wherein each type of polypeptide or polynucleotide is a polypeptide or polynucleotide of the invention as described herein.

In some embodiments, polypeptides and/or polynucleotides are provided as a library, for example, a library comprising a plurality of at least about 1×10⁴, 1×10⁵, 1×10⁶, 1×10⁷, 1×10⁸ distinct polypeptide and/or polynucleotide sequences of the invention. In another aspect, the invention also provides a library comprising a plurality of the viruses or viral particles of the invention, each virus or virus particle displaying a polypeptide of the invention. A library of the invention may comprise viruses or viral particles displaying any number of distinct polypeptides (sequences), for example, at least about 1×10⁴, 1×10⁵, 1×10⁶, 1×10⁷, 1×10⁸ distinct polypeptides.

In another aspect, the invention provides host cells comprising a polynucleotide or vector comprising a sequence encoding a polypeptide of the invention.

In another aspect, the invention provides methods for selecting for high affinity binders to specific target antigens. In certain such embodiments, the specific target antigen includes, but is not limited to, vascular endothelial growth factor (VEGF), HER2, insulin, IGF-1, or HGH.

The methods of the invention provide populations of polypeptides (for example, libraries of polypeptides (e.g., antibody variable domains)) with one or more diversified CDR regions. These libraries are sorted (selected) and/or screened to identify high affinity binders to a target antigen. In one aspect, polypeptide binders from the library are selected for binding to target antigens, and for affinity. The polypeptide binders selected using one or more of these selection strategies, may then be screened for affinity and/or for specificity (binding only to target antigen and not to non-target antigens).

In one aspect, a method of the invention comprises generating a plurality of polypeptides with one or more diversified CDR regions, sorting the plurality of polypeptides for binders to a target antigen by contacting the plurality of polypeptides with a target antigen under conditions suitable for binding; separating the binders to the target antigen from those that do not bind; isolating the binders; and identifying the high affinity binders (or any binders having a desired binding affinity). The affinity of the binders that bind to the target antigen can be determined using a variety of techniques known in the art, for example, competition ELISA such as described herein. Optionally, the polypeptides can be fused to a polypeptide tag, such as gD, poly his or FLAG, which can be used to sort binders in combination with sorting for the target antigen.

Another embodiment provides a method of isolating or selecting for an antibody variable domain that binds to a target antigen from a library of antibody variable domains, said method comprising: a) contacting a population comprising a plurality of polypeptides of the invention with an immobilized target antigen under conditions suitable for binding to isolate target antigen polypeptide binders; b) separating the polypeptide binders from nonbinders, and eluting the binders from the target antigen; c) optionally, repeating steps a-b at least once (in some embodiments, at least twice).

In some embodiments, a method may further comprise: d) incubating the polypeptide binders with a concentration of labeled target antigen in the range of 0.1 nM to 1000 nM under conditions suitable for binding to form a mixture; e) contacting the mixture with an immobilized agent that binds to the label on the target antigen; f) eluting the polypeptide binders from the labeled target antigen; g) optionally, repeating steps d) to f) at least once (in some embodiments, at least twice), using a successively lower concentration of labeled target antigen each time. Optionally, the method may comprise adding an excess of unlabelled target antigen to the mixture and incubating for a period of time sufficient to elute low affinity binders from the labeled target antigen.

Another aspect of the invention provides a method of isolating or selecting for high affinity binders (or binders having a desired binding affinity) to a target antigen. In one embodiment, said method comprises: a) contacting a population comprising a plurality of polypeptides of the invention with a target antigen, wherein the antigen is provided at a concentration in the range of about 0.1 nM to 1000 nM to isolate polypeptide binders to the target antigen; b) separating the polypeptide binders from the target antigen; c) optionally, repeating steps a-b at least once (in some embodiments, at least twice), each time with a successively lower concentration of target antigen to isolate polypeptide binders that bind to lowest concentration of target antigen; d) selecting the polypeptide binder that binds to the lowest concentration of the target antigen for high affinity (or any desired affinity) by incubating the polypeptide binders with several different dilutions of the target antigen and determining the IC50 of the polypeptide binder; and e) identifying a polypeptide binder that has a desired affinity for the target antigen. Said affinity can be, for example, about 0.1 nM to 200 nM, 0.5 nM to 150 nM, 1 nM to 100 nM, and/or 25 nM to 75 nM.

Another embodiment provides an assay for isolating or selecting polypeptide binders comprising (a) contacting a population comprising a plurality of polypeptides of the invention with a labeled target antigen, wherein the labeled target antigen is provided at a concentration in a range of 0.1 nM to 1000 nM, under conditions suitable for binding to form a complex of a polypeptide binder and the labeled target antigen; b) isolating the complexes and separating the polypeptide binder from the labeled target antigen; c) optionally, repeating steps a-b at least once, each time using a lower concentration of target antigen. Optionally, the method may further comprise contacting the complex of polypeptide binder and target antigen with an excess of unlabelled target antigen. In one embodiment, the steps of the method are repeated twice and the concentration of target in a first round of selection is in the range of about 100 nM to 250 nM, and, in a second round of selection (if performed) is in the range of about 25 nM to 100 nM, and in the third round of selection (if performed) is in the range of about 0.1 nM to 25 nM.

The invention also includes a method of screening a population comprising a plurality of polypeptides of the invention, said method comprising: a) incubating a first sample of the population of polypeptides with a target antigen under conditions suitable for binding of the polypeptides to the target antigen; b) subjecting a second sample of the population of polypeptides to a similar incubation but in the absence of the target antigen; (c) contacting each of the first and second sample with immobilized target antigen under conditions suitable for binding of the polypeptides to the immobilized target antigen; d) detecting amount of polypeptides bound to immobilized target antigen for each sample; e) determining affinity of a particular polypeptide for the target antigen by calculating the ratio of the amount of the particular polypeptide that is bound in the first sample over the amount of the particular polypeptide that is bound in the second sample.

The libraries generated as described herein may also be screened for binding to a specific target and for lack of binding to nontarget antigens. In one aspect, the invention provides a method of screening for a polypeptide, such as an antibody variable domain of the invention, that binds to a specific target antigen from a library of antibody variable domains, said method comprising: a) generating a population comprising a plurality of polypeptides of the invention; b) contacting the population of polypeptides with a target antigen under conditions suitable for binding; c) separating a binder polypeptide in the library from nonbinder polypeptides; d) identifying a target antigen-specific binder polypeptide by determining whether the binder polypeptide binds to a non-target antigen; and e) isolating a target antigen-specific binder polypeptide. In some embodiments, step (e) comprises eluting the binder polypeptide from the target antigen, and amplifying a replicable expression vector encoding said binder polypeptide. In some embodiments, one or more of the libraries, clones or polypeptides are screened against a panel of antigens including the target antigen. In some embodiments, those clones or polypeptides that specifically bind to the target antigen and do not substantially crossreact with any of the other antigen on the panel are selected. The panel of antigens can include at least three and up to 100 different antigens. In some cases, the panel of antigens includes 3 to 100, 3 to 50, 3 to 25, or 3 to 10 different antigens.

Combinations of any of the sorting/selection methods described above may be combined with the screening methods. For example, in one embodiment, polypeptide binders are first selected for binding to an immobilized target antigen. Polypeptide binders that bind to the immobilized target antigen can then be screened for binding to the target antigen and for lack of binding to nontarget antigens. Polypeptide binders that bind specifically to the target antigen can be amplified as necessary. These polypeptide binders can be selected for higher affinity by contact with a concentration of a labeled target antigen to form a complex, wherein the concentration range of labeled target antigen is from about 0.1 nM to about 1000 nM, and the complexes are isolated by contact with an agent that binds to the label on the target antigen. A polypeptide binder can then be eluted from the labeled target antigen and optionally, the rounds of selection are repeated, and each time a lower concentration of labeled target antigen is used. The binder polypeptides that can be isolated using this selection method can then be screened for high affinity using for example, the solution phase ELISA assay as described, e.g., in Examples 2 and 4 or other conventional methods known in the art. Populations of polypeptides of the invention used in methods of the invention can be provided in any form suitable for the selection/screening steps. For example, the polypeptides can be in free soluble form, attached to a matrix, or present at the surface of a viral particle such as phage or phagemid particle. In some embodiments of methods of the invention, the plurality of polypeptides are encoded by a plurality of replicable vectors provided in the form of a library. In selection/screening methods described herein, vectors encoding a binder polypeptide may be further amplified to provide sufficient quantities of the polypeptide for use in repetitions of the selection/screening steps (which, as indicated above, are optional in methods of the invention).

In one embodiment, the invention provides a method of selecting for a polypeptide that binds to a target antigen comprising:
a) generating a composition comprising a plurality of polypeptides of the invention as described herein;
b) selecting a polypeptide binder that binds to a target antigen from the composition;
c) isolating the polypeptide binder from the nonbinders;
d) identifying binders of the desired affinity from the isolated polypeptide binders.

In another embodiment, the invention provides a method of selecting for an antigen binding variable domain that binds to a target antigen from a library of antibody variable domains comprising:
a) contacting the library of antibody variable domains of the invention (as described herein) with a target antigen;
b) separating binders from nonbinders, and eluting the binders from the target antigen and incubating the binders in a solution with decreasing amounts of the target antigen in a concentration from about 0.1 nM to 1000 nM;
c) selecting the binders that can bind to the lowest concentration of the target antigen and that have an affinity of about 0.1 nM to 200 nM.

In some embodiments, the concentration of target antigen is about 100 to 250 nM, or about 25 to 100 nM.

In one embodiment, the invention provides a method of selecting for a polypeptide that binds to a target antigen from a library of polypeptides comprising:
a) isolating polypeptide binders to a target antigen by contacting a library comprising a plurality of polypeptides of the invention (as described herein) with an immobilized target antigen under conditions suitable for binding;
b) separating the polypeptide binders in the library from nonbinders and eluting the binders from the target antigen to obtain a subpopulation enriched for the binders; and
c) optionally, repeating steps a-b at least once (in some embodiments at least twice), each repetition using the subpopulation of binders obtained from the previous round of selection.

In some embodiments, methods of the invention further comprise the steps of:
d) incubating the subpopulation of polypeptide binders with a concentration of labeled target antigen in the range of 0.1nM to 1000 nM under conditions suitable for binding to form a mixture;
e) contacting the mixture with an immobilized agent that binds to the label on the target antigen;
f) detecting the polypeptide binders bound to labeled target antigens and eluting the polypeptide binders from the labeled target antigen;
g) optionally, repeating steps d) to f) at least once (in some embodiments, at least twice), each repetition using the subpopulation of binders obtained from the previous round of selection and using a lower concentration of labeled target antigen than the previous round.

In some embodiments, these methods further comprise adding an excess of unlabelled target antigen to the mixture and incubating for a period of time sufficient to elute low affinity binders from the labeled target antigen.

In another embodiment, the invention provides a method of isolating high affinity binders to a target antigen comprising:
a) contacting a library comprising a plurality of polypeptides of the invention (as described herein) with a target antigen in a concentration of at least about 0.1 nM to 1000 nM to isolate polypeptide binders to the target antigen;
b) separating the polypeptide binders from the target antigen to obtain a subpopulation enriched for the polypeptide binders; and
c) optionally, repeating steps a) and b) at least once (in some embodiments, at least twice), each repetition using the subpopulation of binders obtained from the previous round of selection and using a decreased concentration of target antigen than the previous round to isolate polypeptide binders that bind to the lowest concentration of target antigen.

In one aspect, the invention provides an assay for selecting polypeptide binders from a library comprising a plurality of polypeptides of the invention (as described herein) comprising:
a) contacting the library with a concentration of labeled target antigen in a concentration range of 0.1 nM to 1000 nM, under conditions suitable for binding to form a complex of a polypeptide binder and the labeled target antigen;
b) isolating the complexes and separating the polypeptide binders from the labeled target antigen to obtain a subpopulation enriched for the binders;
c) optionally, repeating steps a-b at least once (in some embodiments, at least twice), each time using the subpopulation of binders obtained from the previous round of selection and using a lower concentration of target antigen than the previous round.

In some embodiments, the method further comprises adding an excess of unlabelled target antigen to the complex of the polypeptide binder and target antigen. In some embodiments, the steps set forth above are repeated at least once (in some embodiments, at least twice) and the concentration of target in the first round of selection is about 100 nM to 250 nM, and in the second round of selection is about 25 nM to 100 nM, and in the third round of selection is about 0.1 nM to 25 nM.

In another aspect, the invention provides a method of screening a library comprising a plurality of polypeptides of the invention, said method comprising:
a) incubating a first sample of the library with a concentration of a target antigen under conditions suitable for binding of the polypeptides to the target antigen;
b) incubating a second sample of the library without a target antigen;
c) contacting each of the first and second sample with immobilized target antigen under conditions suitable for binding of the polypeptide to the immobilized target antigen;
d) detecting the polypeptide bound to immobilized target antigen for each sample;
e) determining affinity of the polypeptide for the target antigen by calculating the ratio of the amounts of bound polypeptide from the first sample over the amount of bound polypeptide from the second sample.

Diagnostic and therapeutic uses for binder polypeptides of the invention are contemplated. In one diagnostic application, the invention provides a method for determining the presence of a protein of interest comprising exposing a sample suspected of containing the protein to a binder polypeptide of the invention and determining binding of the binder polypeptide to the sample. For this use, the invention provides a kit comprising the binder polypeptide and instructions for using the binder polypeptide to detect the protein.

The invention further provides: isolated nucleic acid encoding the binder polypeptide; a vector comprising the nucleic acid, optionally, operably linked to control sequences recognized by a host cell transformed with the vector; a host cell transformed with the vector; a process for producing the binder polypeptide comprising culturing this host cell so that the nucleic acid is expressed and, optionally, recovering the binder polypeptide from the host cell culture (e.g. from the host cell culture medium).

The invention also provides a composition comprising a binder polypeptide of the invention and a carrier (e.g., a pharmaceutically acceptable carrier) or diluent. This composition for therapeutic use is sterile and may be lyophilized. Also contemplated is the use of a binder polypeptide of this invention in the manufacture of a medicament for treating an indication described herein. The composition can further comprise a second therapeutic agent such as a chemotherapeutic agent, a cytotoxic agent or an anti-angiogenic agent.

The invention further provides a method for treating a mammal, comprising administering an effective amount of a binder polypeptide of the invention to the mammal. The mammal to be treated in the method may be a nonhuman mammal, e.g. a primate suitable for gathering preclinical data or a rodent (e.g., mouse or rat or rabbit). The nonhuman mammal may be healthy (e.g. in toxicology studies) or may be suffering from a disorder to be treated with the binder polypeptide of interest. In one embodiment, the mammal is suffering from a VEGF-related disorder. In another embodiment, the mammal is suffering from an insulin-related disorder. In another embodiment, the mammal is suffering from a GH-related disorder. In another embodiment, the mammal is suffering from a HER2-related disorder. In another embodiment, the mammal is suffering from an IGF-1-related disorder.

In one embodiment, the mammal is suffering from or is at risk of developing abnormal angiogenesis (e.g., pathological angiogenesis). In one specific embodiment, the disorder is a cancer selected from the group consisting of colorectal cancer, renal cell carcinoma, ovarian cancer, lung cancer, non-small-cell lung cancer (NSCLC), bronchoalveolar carcinoma and pancreatic cancer. In another embodiment, the disorder is a disease caused by ocular neovascularisation, e.g., diabetic blindness, retinopathies, primarily diabetic retinopathy, age-induced macular degeneration and rubeosis. In another embodiment, the mammal to be treated is suffering from or is at risk of developing an edema (e.g., an edema associated with brain tumors, an edema associated with stroke, or a cerebral edema). In another embodiment, the mammal is suffering from or at risk of developing a disorder or illness selected from the group consisting of rheumatoid arthritis, inflammatory bowel disease, refractory ascites, psoriasis, sarcoidosis, arterial arteriosclerosis, sepsis, burns and pancreatitis. According to another embodiment, the mammal is suffering from or is at risk of developing a genitourinary illness selected from the group consisting of polycystic ovarian disease (POD), endometriosis and uterine fibroids. In one embodiment, the disorder is a disease caused by dysregulation of cell survival (e.g., abnormal amount of cell death), including but not limited to cancer, disorders of the immune system, disorders of the nervous system and disorders of the vascular system. The amount of binder polypeptide of the invention that is administered will be a therapeutically effective amount to treat the disorder. In dose escalation studies, a variety of doses of the binder polypeptide may be administered to the mammal. In another embodiment, a therapeutically effective amount of the binder polypeptide is administered to a human patient to treat a disorder in that patient. In one embodiment, binder polypeptides of this invention useful for treating tumors, malignancies, and other disorders related to abnormal angiogenesis, including inflammatory or immunologic disorders and/or diabetes or other insulin-related disorders described herein are Fab or scFv antibodies. Accordingly, such binder polypeptides can be used in the manufacture of a medicament for treating an inflammatory or immune disease. A mammal that is suffering from or is at risk for developing a disorder or illness described herein can be treated by administering, a second therapeutic agent, simultaneously, sequentially or in combination with, a polypeptide (e.g., an antibody) of this invention. It should be understood that other therapeutic agents, in addition to the second therapeutic agent, can be administered to the mammal or used in the manufacture of a medicament for the desired indications.

These polypeptides can be used to understand the role of host stromal cell collaboration in the growth of implanted non-host tumors, such as in mouse models wherein human tumors have been implanted. These polypeptides can be used in methods of identifying human tumors that can escape therapeutic treatment by observing or monitoring the growth of the tumor implanted into a rodent or rabbit after treatment with a polypeptide of this invention. The polypeptides of this invention can also be used to study and evaluate combination therapies with a polypeptide of this invention and other therapeutic agents. The polypeptides of this invention can be used to study the role of a target molecule of interest in other diseases by administering the polypeptides to an animal suffering from the disease or a similar disease and determining whether one or more symptoms of the disease are alleviated.

For the sake of clarity, in the description herein, unless specifically or contextually indicated otherwise, all amino acid numberings are according to Kabat et al. (see further elaboration in “Definitions” below).

### BRIEF DESCRIPTION OF THE FIGURES

FIG. 1 depicts the sequences of 4D5 light chain and heavy chain variable domain (SEQ ID NOS: 1 & 2, respectively).

FIG. 2 shows a 3-D modeled structure of humanized 4D5 showing CDR residues that form contiguous patches. Contiguous patches are formed by amino acid residues. 28, 29, 30, 31 and 32 in CDRL1; amino acids residues 50 and 53 of CDRL2; amino acid residues 91, 92, 93, 94 and 96 of CDRL3; amino acid residues 28, 30, 31, 32, 33 in CDRH1; and amino acid residues 50, 52, 53, 54, 56, and 58 in CDRH2.

FIG. 3 shows the frequency of amino acids (identified by single letter code) in human antibody light chain CDR sequences from the Kabat database. The frequency of each amino acid at a particular amino acid position is shown starting with the most frequent amino acid at that position at the left and continuing on to the right to the least frequent amino acid. The number below the amino acid represents the number of naturally occurring sequences in the Kabat database that have that amino acid in that position.

FIG. 4 shows the frequency of amino acids (identified by single letter code) in human antibody heavy chain CDR sequences from the Kabat database. The frequency of each amino acid at a particular amino acid position is shown starting with the most frequent amino acid at that position at the left and continuing on to the right to the least frequent amino acid. The number below the amino acid represents the number of naturally occurring sequences in the Kabat database that have that amino acid in that position. Framework amino acid positions 71, 93 and 94 are also shown.

FIG. 5 schematically illustrates a bicistronic vector allowing expression of separate transcripts for display of F(ab)₂. A suitable promoter drives expression of the first and second cistron. The first cistron encodes a secretion signal sequence (malE or stII), a light chain variable and constant domain and a gD tag. The second cistron encodes a secretion signal, a sequence encoding heavy chain variable domain and constant domain 1 (CH1) and cysteine dimerization domain and at least a portion of the viral coat protein.

FIG. 6 illustrates CDR positions diversified to create the YS-C and YS-D libraries, as described in Example 1. CDR positions shown are numbered according to the Kabat nomenclature.

FIG. 7 illustrates the randomization scheme for each diversified CDR position in the YS-C and YS-D libraries, as described in Example 1.

FIGS. 8A and 8B show mutagenic oligonucleotides used in the construction of the YS-C and YS-D libraries, as described in Example 1 (SEQ ID NOS:8-36). Equimolar DNA degeneracies are represented in the codon sets (W=A/T, K=G/T, M=A/C, N=A/C/G/T, R=A/G, S=G/C, Y=T/C). Codon sets are represented in the IUB code. The notation “XXX” in the H3-D6 to H3-D17 oligonucleotides represents Tyr/Ser/Gly/Ala/Asp/Glu/Phe/His/Ile/Lys/Leu/Met/Asn/Pro/Gln/Arg/Thr/Val/Trp-encoding codons at a molar ratio of 20/15/15/3.125/3.125/3.125/3.125/3.125/3.125/3.125/3.125/3.125/3.125/3.125/3.125/3.125/3.125/3.125/3.125, respectively.

FIG. 9 shows enrichment ratios for libraries YS-C and YS-D following 5 rounds of selection against human VEGF, as described in Example 2. Numbers are shown as X/Y, with X representing the number of specific or non-specific clones and Y representing the number of clones screened for a given library. Specific clones are identified as those exhibiting binding to human VEGF that was at least 15 times greater (based on ELISA signal read at 450 nm) than binding to bovine serum albumin (BSA).

FIG. 10 shows the amino acid sequences and affinity data for specific binders to human VEGF from the YS-C and YS-D libraries, as described in Example 2 (CDR sequences shown in SEQ ID NOS:37-157). The fraction of each Fab-expressing phage remaining uncomplexed in the presence of 1000 nM or 100 nM human VEGF is also provided. The on-rates (kₐ), off-rates (k_{d}), and dissociation constants (K_{D}) for certain of the Fabs as determined by BIACORE analysis are provided under the heading “kinetic parameters.” The language “N.D.B.” means that there was no detectable binding for the indicated Fab.

FIG. 11 illustrates the randomization scheme for each diversified CDR position in the YSGR-A, YSGR-B, YSGR-C, and YSGR-D libraries, as described in Example 3.

FIGS. 12A-12D show mutagenic oligonucleotides used in the construction of the YSGR-A, YSGR-B, YSGR-C, and YSGR-D libraries, as described in Example 3 (SEQ ID NOS:158-208). Equimolar DNA degeneracies are represented in the codon sets (W=A/T, K=G/T, M=A/C, N=A/C/G/T, R=A/G, S=G/C, Y=T/C). Codon sets are represented in the IUB code. The notation “XXX” in the H3-A6-H3-A17 oligonucleotides represents Tyr/Ser/Gly-encoding codons at a molar ratio of 50/25/25, respectively. The notation “XXX” in the H3-B6-H3-B17 oligonucleotides represents Tyr/Ser/Arg-encoding codons at a molar ratio of 25/50/25, respectively. The notation “XXX” in the H3-C6-H3-C17 oligonucleotides represents Tyr/Ser/Gly/Arg-encoding codons at a molar ratio of 38/25/25/12, respectively. The notation “XXX” in the H3-D6 to H3-D17 oligonucleotides represents Tyr/Ser/Gly/Arg/Asp/Glu/Phe/His/Ile/Lys/Leu/Met/Asn/Gln/Thr/Val/Trp/Pro/Ala-encoding codons at a molar ratio of 20/26/26/13/1/1/1/1/1/1/1/1/1/1/1/1/1, respectively.

FIG. 13 shows enrichment ratios for library YSGR-A-D following 5 rounds of selection against human VEGF or human insulin, as described in Example 4. Numbers are shown as X/Y, with X representing the number of unique clones and Y representing the number of clones specifically binding to human VEGF or human insulin. Specific clones are identified as those exhibiting binding to human VEGF or to human insulin that was at least ten times greater (based on ELISA signal read at 450 nm) than binding to bovine serum albumin (BSA).

FIGS. 14A-14C show amino acid sequences for CDRH1, CDRH2, CDRH3, and CDRL3 from the specific binders to human VEGF isolated from the YSGR-A-D library, as described in Example 4 (SEQ ID NOS:209-683, 1318 and 2428-2431). FIGS. 14D-14F show the results of ELISA assays for each of the clones set forth in FIGS. 14A-14C. Dark shading indicates strong binding (signal of 2 to 10) and light shading indicates weak binding (signal of 0.25 to 2).

FIGS. 15A and 15B show amino acid sequences for CDRH1, CDRH2, CDRH3, and CDRL3 from the specific binders to human insulin isolated from the YSGR-A-D library, as described in Example 4 (SEQ ID NOS:684-1098 and 1098). FIGS. 15C and 15D show the results of ELISA assays for each of the clones set forth in FIGS. 15A and 15B. Dark shading indicates strong binding (signal of 2 to 10), and light shading indicates weak binding (signal of 0.25 to 2).

FIG. 16 depicts framework region sequences of huMAb4D5-8 light and heavy chains. Numbers in superscript/bold indicate amino acid positions according to Kabat. (SEQ ID NOS:1099-1106)

FIG. 17 depicts modified/variant framework region sequences of huMAb4D5-8 light and heavy chains. Numbers in superscript/bold indicate amino acid positions according to Kabat. (SEQ ID NOS:1107-1114)

FIGS. 18A and 18B illustrate the randomization scheme for each diversified CDR position in the Binary H3 libraries (SAH3, SCH3, SFH3, SGH3, SIH3, SLH3, SNH3, SPH3, SRH3, STH3, SWH3, and SYH3), as described in Example 5. The indicated amino acid positions are numbered according to Kabat. Positions 100x refer to the two amino acid positions right before position 101. The actual numeric designation may change depending on length of CDRH3 region.

FIGS. 19A-19L show mutagenic oligonucleotides used in the construction of the Binary H3 libraries (SAH3 (FIG. 19A), SCH3 (FIG. 19B), SFH3 (FIG. 19C), SGH3 (FIG. 19D), SIH3 (FIG. 19E), SLH3 (FIG. 19F), SNH3 (FIG. 19G), SPH3 (FIG. 19H), SRH3 (FIG. 19I), STH3 (FIG. 19J), SWH3 (FIG. 19K), and SYH3 (FIG. 19L)), as described in Example 5 (SEQ ID NOS:158-160 and SEQ ID NOS:1115-1282). Equimolar DNA degeneracies are represented in the codon sets (W=A/T, K=G/T, M=A/C, N=A/C/G/T, R=A/G, S=G/C, Y=T/C). Codon sets are represented in the IUB code.

FIG. 20 shows enrichment ratios for the Binary H3 libraries (pooled SAH3, SCH3, SFH3, SGH3, SIH3, SLH3, SNH3, SPH3, SRH3, STH3, SWH3, and SYH3) and the Surface Binary libraries (pooled SY, SF, SR, and SW) following 5 rounds of selection against human VEGF, as described in Examples 6 and 8. Numbers are shown as X/Y, with X representing the number of specific or non-specific clones and Y representing the number of clones screened for a given library. Specific clones are identified as those exhibiting binding to human VEGF that was at least 10-fold greater on target-coated plates (based on ELISA signal read at 450 nm) in comparison with BSA-coated plates.

FIGS. 21A and 21B show amino acid sequences for CDRL3, CDRH1, CDRH2, and CDRH3, from the specific binders to human VEGF isolated from the pooled Binary H3 libraries (SXH3), as described in Example 6 (SEQ ID NOS:1283-1510). FIGS. 21C and 21D show the results of ELISA assays for each of the clones set forth in FIGS. 21 A and 21B. Dark shading indicates strong binding (signal of 2 to 10), and light shading indicates weak binding (signal of 0.25 to 2).

FIG. 22A shows amino acid sequences for CDRL3, CDRH1, CDRH2, and CDRH3 from the specific binders to HER2 isolated from the pooled Binary H3 libraries (SXH3), as described in Example 6 (SEQ ID NOS:1511-1618). FIG. 22B shows the results of ELISA assays for each of the clones set forth in FIG. 22A. Dark shading indicates strong binding (signal of 2 to 10).

FIG. 23A shows amino acid sequences for CDRL3, CDRH1, CDRH2, and CDRH3 from the specific binders to human insulin isolated from the pooled Binary H3 libraries (SXH3), as described in Example 6 (SEQ ID NOS:1619-1754, 224, 257, 261, 694, 1318, 724, 331, 369, 794, 379, 378, 326, 948, 967, 2422, 542, 2433, 444, 1722, 1721, 1725, 2434, 2435, and 2438). FIG. 23B shows the results of ELISA assays for some of the clones set forth in FIG. 23A. Dark shading indicates strong binding (signal of 2 to 10), and light shading indicates weak binding (signal of 0.25 to 2).

FIG. 24A and 24B shows amino acid sequences for CDRL3, CDRH1, CDRH2, and CDRH3 from the specific binders to human IGF-1 isolated from the pooled Binary H3 libraries (SXH3), as described in Example 6 (SEQ ID NOS:1755-1954, 1318, 1334, 238, 215, 303, 239, 1554, 2163, 383, 358, 320, 369, 80, 126, 444, 133, 510, 69, 1397, 2442-2447, 249, 773, 233, 690, 258, 257, 213, 216, 262, 694, 773, 210, 756, 694, 214, 223, 272, 262, 309, 259, 222, 773, 690,1535, 279, 756, 379, 320, 795, 341, 880, 1559, 1853, 418, 2439, 847, 861, 802, 793, and 2448-2473). FIG. 24C shows the results of ELISA assays for some of the clones set forth in FIGS. 24A and B. Dark shading indicates strong binding (signal of 2 to 10), and light shading indicates weak binding (signal of 0.25 to 2).

FIG. 25A shows amino acid sequence for CDRL3, CDRH1, CDRH2, and CDRH3 from the specific binders to human growth hormone (HGH) isolated from the pooled Binary H3 libraries (SXH3), as described in Example 6 (SEQ ID NOS:1955-1986). FIG. 25B shows the results of ELISA assays for each of the clones set forth in FIG. 25A. Dark shading indicates strong binding (signal of 2 to 10), and light shading indicates weak binding (signal of 0.25 to 2).

FIG. 26 illustrates the randomization scheme for each diversified CDR position in the Binary Surface libraries (SY, SW, SR, and SF), as described in Example 7. The indicated amino acid positions are numbered according to Kabat. Positions 100x refer to the two amino acid positions right before position 101. The actual numeric designation may change depending on length of CDRH3 region.

FIG. 27 shows mutagenic oligonucleotides used in the construction of certain of the Binary Surface libraries (SW, SR, and SF), as described in Example 7 (SEQ ID NOS:1987-1995). Equimolar DNA degeneracies are represented in the codon sets (W=A/T, K=G/T, M=A/C, N=A/C/G/T, R=A/G, S=G/C, Y=T/C). Codon sets are represented in the IUB code.

FIGS. 28A-C shows amino acid sequences for CDRL3, CDRH1, CDRH2, and CDRH3 from the specific binders to human VEGF isolated from the pooled Surface Binary libraries (SX-surface), as described in Example 8 (SEQ ID NOS:1996-2119, 69, 71-72, 74, 76, 78, 80, 215-216, 257, 279-280, 318-320, 326, 330, 338-339, 376, 444-445, 461, 690, 694, 701, 740, 743, 751, 773-774, 779, 849, 1287-1288, 1291, 1300-1301, 1312, 1318, 1330, 1369, 1373, 1375, 1459, 1474-1476, 1478-1481, 1485, 1490, 1649, 1766, 1772, 1956, 1962, 2010, 2015, 2094, 2104, and 2474-2562). FIG. 28D shows the results of ELISA assays for some of the clones set forth in FIG. 28A. Dark shading indicates strong binding (signal of 2 to 10), and light shading indicates weak binding (signal of 0.25 to 2).

FIG. 29A shows amino acid sequences for CDRL3, CDRH1, CDRH2, and CDRH3 from the specific binders to HER2 isolated from the pooled Surface Binary libraries (SX-surface), as described in Example 8 (SEQ ID NOS:2120-2227). FIG. 29B shows the results of ELISA assays for each of the clones set forth in FIG. 29A. Dark shading indicates strong binding (signal of 2 to 10), and light shading indicates weak binding (signal of 0.25 to 2).

FIG. 30A shows amino acid sequences for CDRL3, CDRH1, CDRH2, and CDRH3 from the specific binders to human insulin isolated from the pooled Surface Binary libraries (SX-surface), as described in Example 8 (SEQ ID NOS:2228-2271, 2563-2565, 2568-2572, 2581-2588, and 2595-2602). FIG. 30B shows the results of ELISA assays for some of the clones set forth in FIG. 30A. Dark shading indicates strong binding (signal of 2 to 10), and light shading indicates weak binding (signal of 0.25 to 2).

FIGS. 31A-B shows amino acid sequences for CDRL3, CDRH1, CDRH2, and CDRH3 from the specific binders to human IGF-1 isolated from the pooled Surface Binary libraries (SX-surface), as described in Example 8 (SEQ ID NOS:2272-2383, 67-68, 71, 78, 133, 211, 230, 233, 238, 279, 262, 303, 309, 320, 338, 418, 483, 491, 502, 510, 689, 694, 690, 733, 756, 724, 847, 861, 880, 910, 983, 1318, 1397, 1535, 1559, 1853, 1912, 2404, 2410, 2566-2567, 2573-2576, 2578-2580, 2589, 2590-2594, 2603-2609, and 2611-2625). FIG. 31C shows the results of ELISA assays for some of the clones set forth in FIGS. 31A-B. Dark shading indicates strong binding (signal of 2 to 10), and light shading indicates weak binding (signal of 0.25 to 2).

FIG. 32A shows amino acid sequences for CDRL3, CDRH1, CDRH2, and CDRH3 from the specific binders to HGH isolated from the pooled Surface Binary libraries (SX-surface), as described in Example 8 (SEQ ID NOS:2384-2427). FIG. 32B shows the results of ELISA assays for each of the clines set forth in FIG. 32A. Dark shading indicates strong binding (signal of 2 to 10), and light shading indicates weak binding (signal of 0.25 to 2).

FIGS. 33A and B depict surface plasmon resonance binding analyses of soluble Fab proteins from three HER2-binding clones (clone B11, clone G54 and clone YSGR-A-42) to immobilized HER2. Clone B11 had a kₐ of 1.9×10⁶ M⁻¹s⁻¹, a k_{d} of 1.7×10⁻³ s⁻¹, and a K_{D} of 890 pM. Clone G54 had a kₐ of 2.0×10⁵ M⁻¹s⁻¹, a k_{d} of 2.2×10⁻³ s⁻¹, and a K_{D} of 11 nM.

FIG. 34 graphically depicts the binary composition of isolated unique clones that specifically bind to VEGF, HER2, IGF-1, or insulin from each of the SXH3 and SX-surface libraries. Of the specific binders isolated from the SXH3 library, the greatest number of unique clones binding VEGF included S:Y, the greatest number of unique clones binding HER2 included S:W, the greatest number of unique clones binding IGF-1 included S:R, and the greatest number of unique clones binding insulin included S:R. Of the specific binders isolated from the SX-surface library, the greatest number of unique clones binding to VEGF or to IGF-1 included S:Y, the greatest number of unique clones binding to HER2 included S:W, and the greatest number of unique clones binding to insulin included S:R.

FIG. 35 graphically depicts the specificity of Fabs containing different binary amino acid combinations (Ser:Tyr, Ser:Trp, Ser:Arg, or Ser:Phe) obtained herein from the binary SXH3 library or the binary SX-surface library.

FIG. 36 shows the results of flow cytometric analyses of binding of anti-HER2 fabs isolated from each of the YSGR (clone A-42), SX-surface (clones G37 and G54), and SXH3 libraries (clone B11) to NR6 or H2NR6-4D5 cells, as described in Example 8.

FIG. 37 shows the sequences for CDRH1, CDRH2, CDRH3, and CDRL3 for each of HER2-binding IgGs B11, G37, G54, YSGR-A-42, YSGR-A-27, B27, G43, and YSGR-D-104 (SEQ ID NOS: 213, 216, 219, 724, 727, 331, 358, 793, 794, 802, 518, 942, 967, 1397, 1596-1598, 1617, 2124, 2147, 2159, 2186, 2194, 2626-2678, 1617, and 2213). FIG. 37 also shows the IC50 values for the Fab version of each clone.

FIG. 38 shows the results of competitive binding assays described in Example 8 to determine the ability of each of the indicated HER2-specific IgGs to compete for binding to HER2 with Omnitarg, Herceptin, and each of the other IgGs. Shaded numbers represent positive controls. Numbers in bold indicate binding competition.

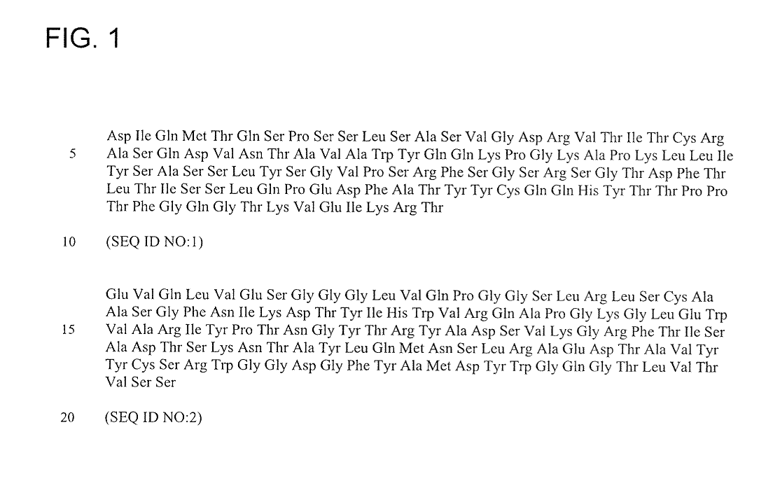

### MODES FOR CARRYING OUT THE INVENTION

The invention provides novel, unconventional, greatly simplified and flexible methods for diversifying CDR sequences (including antibody variable domain sequences), and libraries comprising a multiplicity, generally a great multiplicity of diversified CDRs (including antibody variable domain sequences). Such libraries provide combinatorial libraries useful for, for example, selecting and/or screening for synthetic antibody clones with desirable activities such as binding affinities and avidities. These libraries are useful for identifying immunoglobulin polypeptide sequences that are capable of interacting with any of a wide variety of target antigens. For example, libraries comprising diversified immunoglobulin polypeptides of the invention expressed as phage displays are particularly useful for, and provide a high throughput, efficient and automatable systems of, selecting and/or screening for antigen binding molecules of interest. The methods of the invention are designed to provide high affinity binders to target antigens with minimal changes to a source or template molecule and provide for good production yields when the antibody or antigens binding fragments are produced in cell culture.

Methods and compositions of the invention provide numerous additional advantages. For example, relatively simple variant CDR sequences can be generated, using codon sets encoding a restricted number of amino acids (as opposed to the conventional approach of using codon sets encoding the maximal number of amino acids), while retaining sufficient diversity of unique target binding sequences. The simplified nature (and generally relatively smaller size) of sequence populations generated according to the invention permits further diversification once a population, or sub-population thereof, has been identified to possess the desired characteristics.

The simplified nature of sequences of target antigen binders obtained by methods of the invention leaves significantly greater room for individualized further sequence modifications to achieve the desired results. For example, such sequence modifications are routinely performed in affinity maturation, humanization, etc. By basing diversification on restricted codon sets that encode only a limited number of amino acids, it would be possible to target different epitopes using different restricted codon sets, thus providing the practitioner greater control of the diversification approach as compared with randomization based on a maximal number of amino acids. An added advantage of using restricted codon sets is that undesirable amino acids can be eliminated from the process, for example, methionine or stop codons, thus improving the overall quality and productivity of a library. Furthermore, in some instances, it may be desirable to limit the conformational diversity of potential binders. Methods and compositions of the invention provide the flexibility for achieving this objective. For example, the presence of certain amino acids, such as tyrosine, in a sequence results in fewer rotational conformations.

Definitions

Amino acids are represented herein as either a single letter code or as the three letter code or both.

The term “affinity purification” means the purification of a molecule based on a specific attraction or binding of the molecule to a chemical or binding partner to form a combination or complex which allows the molecule to be separated from impurities while remaining bound or attracted to the partner moiety.

The term “antibody” is used in the broadest sense and specifically covers single monoclonal antibodies (including agonist and antagonist antibodies), antibody compositions with polyepitopic specificity, affinity matured antibodies, humanized antibodies, chimeric antibodies, as well as antigen binding fragments (e.g., Fab, F(ab′)₂, scFv and Fv), so long as they exhibit the desired biological activity. In one embodiment, the term “antibody” also includes human antibodies. As used herein, “antibody variable domain” refers to the portions of the light and heavy chains of antibody molecules that include amino acid sequences of Complementarity Determining Regions (CDRs; i.e., CDR1, CDR2, and CDR3), and Framework Regions (FRs). V_{H} refers to the variable domain of the heavy chain. V_{L} refers to the variable domain of the light chain. According to the compositions and methods used in this invention, the amino acid positions assigned to CDRs and FRs may be defined according to Kabat (Sequences of Proteins of Immunological Interest (National Institutes of Health, Bethesda, Md., 1987 and 1991)). Amino acid numbering of antibodies or antigen binding fragments is also according to that of Kabat.

As used herein, the term “Complementarity Determining Regions (CDRs; i.e., CDR1, CDR2, and CDR3) refers to the amino acid residues of an antibody variable domain the presence of which are necessary for antigen binding. Each variable domain typically has three CDR regions identified as CDR1, CDR2 and CDR3. Each complementarity determining region may comprise amino acid residues from a “complementarity determining region” as defined by Kabat (i.e. about residues 24-34 (L1), 50-56 (L2) and 89-97 (L3) in the light chain variable domain and 31-35 (H1), 50-65 (H2) and 95-102 (H3) in the heavy chain variable domain; Kabat et al., Sequences of Proteins of Immunological Interest, 5th Ed. Public Health Service, National Institutes of Health, Bethesda, Md. (1991)) and/or those residues from a “hypervariable loop” (i.e. about residues 26-32 (L1), 50-52 (L2) and 91-96 (L3) in the light chain variable domain and 26-32 (H1), 53-55 (H2) and 96-101 (H3) in the heavy chain variable domain; Chothia and Lesk J. Mol. Biol. 196:901-917 (1987)). In some instances, a complementarity determining region can include amino acids from both a CDR region defined according to Kabat and a hypervariable loop. For example, the CDRH1 of the heavy chain of antibody 4D5 includes amino acids 26 to 35. The consensus sequence for CDRL1 (according to the Kabat definition) in the 4D5 antibody is R-A-S-Q-D-V-N-T-A-V-A (SEQ ID NO: 6). The consensus sequence for CDRL2 (according to the Kabat definition) in the 4D5 antibody is S-A-S-S-L-Y-S (SEQ ID NO: 7).

“Framework regions” (hereinafter “FR”) are those variable domain residues other than the CDR residues. Each variable domain typically has four FRs identified as FR1, FR2, FR3 and FR4. If the CDRs are defined according to Kabat, the light chain FR residues are positioned at about residues 1-23 (LCFR1), 35-49 (LCFR2), 57-88 (LCFR3), and 98-107 (LCFR4) and the heavy chain FR residues are positioned about at residues 1-30 (HCFR1), 36-49 (HCFR2), 66-94 (HCFR3), and 103-113 (HCFR4) in the heavy chain residues. If the CDRs comprise amino acid residues from hypervariable loops, the light chain FR residues are positioned about at residues 1-25 (LCFR1), 33-49 (LCFR2), 53-90 (LCFR3), and 97-107 (LCFR4) in the light chain and the heavy chain FR residues are positioned about at residues 1-25 (HCFR1), 33-52 (HCFR2), 56-95 (HCFR3), and 102-113 (HCFR4) in the heavy chain residues. In some instances, when the CDR comprises amino acids from both a CDR as defined by Kabat and those of a hypervariable loop, the FR residues can be adjusted accordingly. For example, when CDRH1 includes amino acids H26-H35, the heavy chain FR1 residues are at positions 1-25 and the FR2 residues are at positions 36-49.

As used herein, “codon set” refers to a set of different nucleotide triplet sequences used to encode desired variant amino acids. A set of oligonucleotides can be synthesized, for example, by solid phase synthesis, including sequences that represent all possible combinations of nucleotide triplets provided by the codon set and that will encode the desired group of amino acids. A standard form of codon designation is that of the IUB code, which is known in the art and described herein. A codon set typically is represented by 3 capital letters in italics, e.g. NNK, NNS, XYZ, DVK and the like. Synthesis of oligonucleotides with selected nucleotide “degeneracy” at certain positions is well known in that art, for example the TRIM approach (Knappek et al.; J. Mol. Biol. (1999), 296:57-86); Garrard & Henner, Gene (1993), 128:103). Such sets of oligonucleotides having certain codon sets can be synthesized using commercial nucleic acid synthesizers (available from, for example, Applied Biosystems, Foster City, Calif.), or can be obtained commercially (for example, from Life Technologies, Rockville, Md.). Therefore, a set of oligonucleotides synthesized having a particular codon set will typically include a plurality of oligonucleotides with different sequences, the differences established by the codon set within the overall sequence. Oligonucleotides, as used according to the invention, have sequences that allow for hybridization to a variable domain nucleic acid template and also can, but does not necessarily, include restriction enzyme sites useful for, for example, cloning purposes.

The term “restricted codon set”, and variations thereof, as used herein refers to a codon set that encodes a much more limited number of amino acids than the codon sets typically utilized in art methods of generating sequence diversity. In one aspect of the invention, restricted codon sets used for sequence diversification encode from 2 to 10, from 2 to 8, from 2 to 6, from 2 to 4, or only 2 amino acids. In some embodiments, a restricted codon set used for sequence diversification encodes at least 2 but 10 or fewer, 8 or fewer, 6 or fewer, 4 or fewer amino acids. In a typical example, a tetranomial codon set is used. Examples of tetranomial codon sets include RMC, RMG, RRC, RSA, MKC, YMT, RST, KMT, SRC, MRT and WMT, as known in the art. In another typical example, a binomial codon set is used. Examples of binomial codon sets include TMT, KAT, YAC, WAC, TWC, TYT, YTC, WTC, KTT, YCT, MCG, SCG, MGC, SGT, GRT, GKT and GYT. Determination of suitable restricted codons, and the identification of specific amino acids encoded by a particular restricted codon, is well known and would be evident to one skilled in the art. Determination of suitable amino acid sets to be used for diversification of a CDR sequence can be empirical and/or guided by criteria known in the art (e.g., inclusion of a combination of hydrophobic and hydrophilic amino acid types, etc.).

An “Fv” fragment is an antibody fragment which contains a complete antigen recognition and binding site. This region consists of a dimer of one heavy and one light chain variable domain in tight association, which can be covalent in nature, for example in scFv. It is in this configuration that the three CDRs of each variable domain interact to define an antigen binding site on the surface of the V_{H}-V_{L} dimer. Collectively, the six CDRs or a subset thereof confer antigen binding specificity to the antibody. However, even a single variable domain (or half of an Fv comprising only three CDRs specific for an antigen) has the ability to recognize and bind antigen, although usually at a lower affinity than the entire binding site.

The “Fab” fragment contains a variable and constant domain of the light chain and a variable domain and the first constant domain (CH1) of the heavy chain. F(ab′)₂ antibody fragments comprise a pair of Fab fragments which are generally covalently linked near their carboxy termini by hinge cysteines between them. Other chemical couplings of antibody fragments are also known in the art.

“Single-chain Fv” or “scFv” antibody fragments comprise the V_{H} and V_{L} domains of antibody, wherein these domains are present in a single polypeptide chain. Generally the Fv polypeptide further comprises a polypeptide linker between the V_{H} and V_{L} domains, which enables the scFv to form the desired structure for antigen binding. For a review of scFv, see Pluckthun in The Pharmacology of Monoclonal Antibodies, Vol 113, Rosenburg and Moore eds. Springer-Verlag, N.Y., pp. 269-315 (1994).

The term “diabodies” refers to small antibody fragments with two antigen-binding sites, which fragments comprise a heavy chain variable domain (V_{H}) connected to a light chain variable domain (V_{L}) in the same polypeptide chain (V_{H} and V_{L}). By using a linker that is too short to allow pairing between the two domains on the same chain, the domains are forced to pair with the complementary domains of another chain and create two antigen-binding sites. Diabodies are described more fully in, for example, EP 404,097; WO 93/11161; and Hollinger et al., Proc. Natl. Acad. Sci. USA, 90:6444-6448 (1993).

The expression “linear antibodies” refers to the antibodies described in Zapata et al., Protein Eng., 8(10):1057-1062 (1995). Briefly, these antibodies comprise a pair of tandem Fd segments (V_{H}-C_{H}1-V_{H}-C_{H}1) which, together with complementary light chain polypeptides, form a pair of antigen binding regions. Linear antibodies can be bispecific or monospecific.

The term “monoclonal antibody” as used herein refers to an antibody obtained from a population of substantially homogeneous antibodies, i.e., the individual antibodies comprising the population are identical except for possible naturally occurring mutations that may be present in minor amounts. Monoclonal antibodies are highly specific, being directed against a single antigenic site. Furthermore, in contrast to conventional (polyclonal) antibody preparations which typically include different antibodies directed against different determinants (epitopes), each monoclonal antibody is directed against a single determinant on the antigen. The modifier “monoclonal” indicates the character of the antibody as being obtained from a substantially homogeneous population of antibodies, and is not to be construed as requiring production of the antibody by any particular method. For example, the monoclonal antibodies to be used in accordance with the present invention may be made by the hybridoma method first described by Kohler et al., Nature 256:495 (1975), or may be made by recombinant DNA methods (see, e.g., U.S. Pat. No. 4,816,567). The “monoclonal antibodies” may also be isolated from phage antibody libraries using the techniques described in Clackson et al., Nature 352:624-628 (1991) and Marks et al., J. Mol. Biol. 222:581-597 (1991), for example.

The monoclonal antibodies herein specifically include “chimeric” antibodies (immunoglobulins) in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity (U.S. Pat. No. 4,816,567; and Morrison et al., Proc. Natl. Acad. Sci. USA 81:6851-6855 (1984)).

“Humanized” forms of non-human (e.g., murine) antibodies are chimeric antibodies which contain minimal sequence derived from non-human immunoglobulin. For the most part, humanized antibodies are human immunoglobulins (recipient antibody) in which residues from a hypervariable region of the recipient are replaced by residues from a hypervariable region of a non-human species (donor antibody) such as mouse, rat, rabbit or nonhuman primate having the desired specificity, affinity, and capacity. In some instances, Fv framework region (FR) residues of the human immunoglobulin are replaced by corresponding non-human residues. Furthermore, humanized antibodies may comprise residues which are not found in the recipient antibody or in the donor antibody. These modifications are made to further refine antibody performance. In general, the humanized antibody will comprise substantially all of at least one, and typically two, variable domains, in which all or substantially all of the hypervariable loops correspond to those of a non-human immunoglobulin and all or substantially all of the FR regions are those of a human immunoglobulin sequence. The humanized antibody optionally also will comprise at least a portion of an immunoglobulin constant region (Fc), typically that of a human immunoglobulin. For further details, see Jones et al., Nature 321:522-525 (1986); Riechmann et al., Nature 332:323-329 (1988); and Presta, Curr. Op. Struct. Biol. 2:593-596 (1992).

A “species-dependent antibody” is one which has a stronger binding affinity for an antigen from a first mammalian species than it has for a homologue of that antigen from a second mammalian species. Normally, the species-dependent antibody “binds specifically” to a human antigen (i.e. has a binding affinity (K_{d}) value of no more than about 1×10⁻⁷ M, for example no more than about 1×10⁻⁸ M and as a further example no more than about 1×10⁻⁹ M) but has a binding affinity for a homologue of the antigen from a second nonhuman mammalian species which is at least about 50 fold, or at least about 500 fold, or at least about 1000 fold, weaker than its binding affinity for the human antigen. The species-dependent antibody can be any of the various types of antibodies as defined above, but preferably is a humanized or human antibody.

As used herein, “antibody mutant” or “antibody variant” refers to an amino acid sequence variant of the species-dependent antibody wherein one or more of the amino acid residues of the species-dependent antibody have been modified. Such mutants necessarily have less than 100% sequence identity or similarity with the species-dependent antibody. In one embodiment, the antibody mutant will have an amino acid sequence having at least 75% amino acid sequence identity or similarity with the amino acid sequence of either the heavy or light chain variable domain of the species-dependent antibody, for example at least 80%, for example at least 85%, for example at least 90%, and for example at least 95%. Identity or similarity with respect to this sequence is defined herein as the percentage of amino acid residues in the candidate sequence that are identical (i.e same residue) or similar (i.e. amino acid residue from the same group based on common side-chain properties, see below) with the species-dependent antibody residues, after aligning the sequences and introducing gaps, if necessary, to achieve the maximum percent sequence identity. None of N-terminal, C-terminal, or internal extensions, deletions, or insertions into the antibody sequence outside of the variable domain shall be construed as affecting sequence identity or similarity.

An “isolated” antibody is one which has been identified and separated and/or recovered from a component of its natural environment. Contaminant components of its natural environment are materials which would interfere with diagnostic or therapeutic uses for the antibody, and may include enzymes, hormones, and other proteinaceous or nonproteinaceous solutes. In certain embodiments, the antibody will be purified (1) to greater than 95% by weight of antibody as determined by the Lowry method, e.g., to more than 99% by weight, (2) to a degree sufficient to obtain at least 15 residues of N-terminal or internal amino acid sequence by use of a spinning cup sequenator, or (3) to homogeneity by SDS-PAGE under reducing or nonreducing conditions using Coomassie blue or, preferably, silver stain. Isolated antibody includes the antibody in situ within recombinant cells since at least one component of the antibody's natural environment will not be present. Ordinarily, however, isolated antibody will be prepared by at least one purification step.

“Cell”, “cell line”, and “cell culture” are used interchangeably herein and such designations include all progeny of a cell or cell line. Thus, for example, terms like “transformants” and “transformed cells” include the primary subject cell and cultures derived therefrom without regard for the number of transfers. It is also understood that all progeny may not be precisely identical in DNA content, due to deliberate or inadvertent mutations. Mutant progeny that have the same function or biological activity as screened for in the originally transformed cell are included. Where distinct designations are intended, it will be clear from the context.

“Control sequences” when referring to expression means DNA sequences necessary for the expression of an operably linked coding sequence in a particular host organism. The control sequences that are suitable for prokaryotes, for example, include a promoter, optionally an operator sequence, a ribosome binding site, and possibly, other as yet poorly understood sequences. Eukaryotic cells are known to utilize promoters, polyadenylation signals, and enhancers.

The term “coat protein” means a protein, at least a portion of which is present on the surface of the virus particle. From a functional perspective, a coat protein is any protein which associates with a virus particle during the viral assembly process in a host cell, and remains associated with the assembled virus until it infects another host cell. The coat protein may be the major coat protein or may be a minor coat protein. A “major” coat protein is generally a coat protein which is present in the viral coat at at least about 5, at least about 7, at least about 10 copies of the protein or more. A major coat protein may be present in tens, hundreds or even thousands of copies per virion. An example of a major coat protein is the p8 protein of filamentous phage.

The “detection limit” for a chemical entity in a particular assay is the minimum concentration of that entity which can be detected above the background level for that assay. For example, in the phage ELISA, the “detection limit” for a particular phage displaying a particular antigen binding fragment is the phage concentration at which the particular phage produces an ELISA signal above that produced by a control phage not displaying the antigen binding fragment.

A “fusion protein” and a “fusion polypeptide” refer to a polypeptide having two portions covalently linked together, where each of the portions is a polypeptide having a different property. The property may be a biological property, such as activity in vitro or in vivo. The property may also be a simple chemical or physical property, such as binding to a target antigen, catalysis of a reaction, etc. The two portions may be linked directly by a single peptide bond or through a peptide linker containing one or more amino acid residues. Generally, the two portions and the linker will be in reading frame with each other. In certain embodiments, the two portions of the polypeptide are obtained from heterologous or different polypeptides.

“Heterologous DNA” is any DNA that is introduced into a host cell. The DNA may be derived from a variety of sources including genomic DNA, cDNA, synthetic DNA and fusions or combinations of these. The DNA may include DNA from the same cell or cell type as the host or recipient cell or DNA from a different cell type, for example, from a mammal or plant. The DNA may, optionally, include marker or selection genes, for example, antibiotic resistance genes, temperature resistance genes, etc.

As used herein, “highly diverse position” refers to a position of an amino acid located in the variable regions of the light and heavy chains that have a number of different amino acids represented at the position when the amino acid sequences of known and/or naturally occurring antibodies or antigen binding fragments are compared. The highly diverse positions are typically in the CDR regions. In one aspect, the ability to determine highly diverse positions in known and/or naturally occurring antibodies is facilitated by the data provided by Kabat, Sequences of Proteins of Immunological Interest (National Institutes of Health, Bethesda, Md., 1987 and 1991). An internet-based database located at http://www.bioinf.org.uk/abs/structures.html provides an extensive collection and alignment of light (http://www.bioinf.org.uk/abs/lc.align) and heavy chain (http://www.bioinf.org.uk/abs/hc.align) sequences and facilitates determination of highly diverse positions in these sequences. According to the invention, an amino acid position is highly diverse if it has from about 2 to about 11, from about 4 to about 9, and/or from about 5 to about 7 different possible amino acid residue variations at that position. In some embodiments, an amino acid position is highly diverse if it has at least about 2, at least about 4, at least about 6, and/or at least about 8 different possible amino acid residue variations at that position.

As used herein, “library” refers to a plurality of antibody or antibody fragment sequences (for example, polypeptides of the invention), or the nucleic acids that encode these sequences, the sequences being different in the combination of variant amino acids that are introduced into these sequences according to the methods of the invention.

“Ligation” is the process of forming phosphodiester bonds between two nucleic acid fragments. For ligation of the two fragments, the ends of the fragments must be compatible with each other. In some cases, the ends will be directly compatible after endonuclease digestion. However, it may be necessary first to convert the staggered ends commonly produced after endonuclease digestion to blunt ends to make them compatible for ligation. For blunting the ends, the DNA is treated in a suitable buffer for at least 15 minutes at 15° C. with about 10 units of the Klenow fragment of DNA polymerase I or T4 DNA polymerase in the presence of the four deoxyribonucleotide triphosphates. The DNA is then purified by phenol-chloroform extraction and ethanol precipitation or by silica purification. The DNA fragments that are to be ligated together are put in solution in about equimolar amounts. The solution will also contain ATP, ligase buffer, and a ligase such as T4 DNA ligase at about 10 units per 0.5 μg of DNA. If the DNA is to be ligated into a vector, the vector is first linearized by digestion with the appropriate restriction endonuclease(s). The linearized fragment is then treated with bacterial alkaline phosphatase or calf intestinal phosphatase to prevent self-ligation during the ligation step. Other ligation methods are well known in the art.

A “mutation” is a deletion, insertion, or substitution of a nucleotide(s) relative to a reference nucleotide sequence, such as a wild type sequence.

As used herein, “natural” or “naturally occurring” antibodies, refers to antibodies identified from a nonsynthetic source, for example, from a differentiated antigen-specific B cell obtained ex vivo, or its corresponding hybridoma cell line, or from antibodies obtained from the serum of an animal. These antibodies can include antibodies generated in any type of immune response, either natural or otherwise induced. Natural antibodies include the amino acid sequences, and the nucleotide sequences that constitute or encode these antibodies, for example, as identified in the Kabat database. As used herein, natural antibodies are different than “synthetic antibodies”, synthetic antibodies referring to antibody sequences that have been changed from a source or template sequence, for example, by the replacement, deletion, or addition, of an amino acid, or more than one amino acid, at a certain position with a different amino acid, the different amino acid providing an antibody sequence different from the source antibody sequence.

“Operably linked” when referring to nucleic acids means that the nucleic acids are placed in a functional relationship with another nucleic acid sequence. For example, DNA for a presequence or secretory leader is operably linked to DNA for a polypeptide if it is expressed as a preprotein that participates in the secretion of the polypeptide; a promoter or enhancer is operably linked to a coding sequence if it affects the transcription of the sequence; or a ribosome binding site is operably linked to a coding sequence if it is positioned so as to facilitate translation. Generally, “operably linked” means that the DNA sequences being linked are contiguous and, in the case of a secretory leader, contingent and in reading frame. However, enhancers do not have to be contiguous. Linking is accomplished by ligation at convenient restriction sites. If such sites do not exist, the synthetic oligonucleotide adapters or linkers are used in accord with conventional practice.

“Phage display” is a technique by which variant polypeptides are displayed as fusion proteins to at least a portion of coat protein on the surface of phage, e.g., filamentous phage, particles. A utility of phage display lies in the fact that large libraries of randomized protein variants can be rapidly and efficiently sorted for those sequences that bind to a target antigen with high affinity. Display of peptide and protein libraries on phage has been used for screening millions of polypeptides for ones with specific binding properties. Polyvalent phage display methods have been used for displaying small random peptides and small proteins through fusions to either gene III or gene VIII of filamentous phage. Wells and Lowman, Curr. Opin. Struct. Biol., 3:355-362 (1992), and references cited therein. In monovalent phage display, a protein or peptide library is fused to a gene III or a portion thereof, and expressed at low levels in the presence of wild type gene III protein so that phage particles display one copy or none of the fusion proteins. Avidity effects are reduced relative to polyvalent phage so that sorting is on the basis of intrinsic ligand affinity, and phagemid vectors are used, which simplify DNA manipulations. Lowman and Wells, Methods: A companion to Methods in Enzymology, 3:205-0216 (1991).

A “phagemid” is a plasmid vector having a bacterial origin of replication, e.g., Co1E1, and a copy of an intergenic region of a bacteriophage. The phagemid may be used on any known bacteriophage, including filamentous bacteriophage and lambdoid bacteriophage. The plasmid will also generally contain a selectable marker for antibiotic resistance. Segments of DNA cloned into these vectors can be propagated as plasmids. When cells harboring these vectors are provided with all genes necessary for the production of phage particles, the mode of replication of the plasmid changes to rolling circle replication to generate copies of one strand of the plasmid DNA and package phage particles. The phagemid may form infectious or non-infectious phage particles. This term includes phagemids which contain a phage coat protein gene or fragment thereof linked to a heterologous polypeptide gene as a gene fusion such that the heterologous polypeptide is displayed on the surface of the phage particle.

The term “phage vector” means a double stranded replicative form of a bacteriophage containing a heterologous gene and capable of replication. The phage vector has a phage origin of replication allowing phage replication and phage particle formation. In certain embodiments, the phage is a filamentous bacteriophage, such as an M13, f1, fd, Pf3 phage or a derivative thereof, or a lambdoid phage, such as lambda, 21, phi80, phi81, 82, 424, 434, etc., or a derivative thereof.

“Oligonucleotides” are short-length, single- or double-stranded polydeoxynucleotides that are chemically synthesized by known methods (such as phosphotriester, phosphite, or phosphoramidite chemistry, using solid-phase techniques such as described in EP 266,032 published 4 May 1988, or via deoxynucleoside H-phosphonate intermediates as described by Froeshler et al., Nucl. Acids, Res., 14:5399-5407 (1986)). Further methods include the polymerase chain reaction defined below and other autoprimer methods and oligonucleotide syntheses on solid supports. All of these methods are described in Engels et al., Agnew. Chem. Int. Ed. Engl., 28:716-734 (1989). These methods are used if the entire nucleic acid sequence of the gene is known, or the sequence of the nucleic acid complementary to the coding strand is available. Alternatively, if the target amino acid sequence is known, one may infer potential nucleic acid sequences using known and preferred coding residues for each amino acid residue. The oligonucleotides can be purified on polyacrylamide gels or molecular sizing columns or by precipitation.

DNA is “purified” when the DNA is separated from non-nucleic acid impurities. The impurities may be polar, non-polar, ionic, etc.

A “source antibody”, as used herein, refers to an antibody or antigen binding fragment whose antigen binding sequence serves as the template sequence upon which diversification according to the criteria described herein is performed. In certain embodiments, an antigen binding sequence generally includes an antibody variable region, and at least one CDR including framework regions.

As used herein, “solvent accessible position” refers to a position of an amino acid residue in the variable regions of the heavy and light chains of a source antibody or antigen binding fragment that is determined, based on structure, ensemble of structures and/or modeled structure of the antibody or antigen binding fragment, as potentially available for solvent access and/or contact with a molecule, such as an antibody-specific antigen. These positions are typically found in the CDRs and on the exterior of the protein. The solvent accessible positions of an antibody or antigen binding fragment, as defined herein, can be determined using any of a number of algorithms known in the art. In certain embodiments, solvent accessible positions are determined using coordinates from a 3-dimensional model of an antibody (or portion thereof, e.g., an antibody variable domain, or CDR segment(s)), using a computer program such as the InsightII program (Accelrys, San Diego, Calif.). Solvent accessible positions can also be determined using algorithms known in the art (e.g., Lee and Richards, J. Mol. Biol. 55, 379 (1971) and Connolly, J. Appl. Cryst. 16, 548 (1983)). Determination of solvent accessible positions can be performed using software suitable for protein modeling and 3-dimensional structural information obtained from an antibody (or portion thereof). Software that can be utilized for these purposes includes SYBYL Biopolymer Module software (Tripos Associates). Generally, in certain embodiments, where an algorithm (program) requires a user input size parameter, the “size” of a probe which is used in the calculation is set at about 1.4 Angstrom or smaller in radius. In addition, determination of solvent accessible regions and area methods using software for personal computers has been described by Pacios ((1994) “ARVOMOL/CONTOUR: molecular surface areas and volumes on Personal Computers.” Comput. Chem. 18(4): 377-386; and (1995). “Variations of Surface Areas and Volumes in Distinct Molecular Surfaces of Biomolecules.” J. Mol. Model. 1: 46-53.)

A “transcription regulatory element” will contain one or more of the following components: an enhancer element, a promoter, an operator sequence, a repressor gene, and a transcription termination sequence. These components are well known in the art. U.S. Pat. No. 5,667,780.

A “transformant” is a cell which has taken up and maintained DNA as evidenced by the expression of a phenotype associated with the DNA (e.g., antibiotic resistance conferred by a protein encoded by the DNA).

“Transformation” means a process whereby a cell takes up DNA and becomes a “transformant”. The DNA uptake may be permanent or transient.

A “human antibody” is one which possesses an amino acid sequence which corresponds to that of an antibody produced by a human and/or has been made using any of the techniques for making human antibodies as disclosed herein. This definition of a human antibody specifically excludes a humanized antibody comprising non-human antigen-binding residues.

An “affinity matured” antibody is one with one or more alterations in one or more CDRs thereof which result in an improvement in the affinity of the antibody for antigen, compared to a parent antibody which does not possess those alteration(s). In certain embodiments, affinity matured antibodies will have nanomolar or even picomolar affinities for the target antigen. Affinity matured antibodies are produced by procedures known in the art. Marks et al. Bio/Technology 10:779-783 (1992) describes affinity maturation by VH and VL domain shuffling. Random mutagenesis of CDR and/or framework residues is described by: Barbas et al. Proc Nat. Acad. Sci, USA 91:3809-3813 (1994); Schier et al. Gene 169:147-155 (1995); Yelton et al. J. Immunol. 155:1994-2004 (1995); Jackson et al., J. Immunol. 154(7):3310-9 (1995); and Hawkins et al, J. Mol. Biol. 226:889-896 (1992).

A “blocking” antibody or an “antagonist” antibody is one which inhibits or reduces biological activity of the antigen it binds. In certain embodiments, blocking antibodies or antagonist antibodies substantially or completely inhibit the biological activity of the antigen.

An “agonist antibody”, as used herein, is an antibody which mimics at least one of the functional activities of a polypeptide of interest.

To increase the half-life of the antibodies or polypeptide containing the amino acid sequences of this invention, one can attach a salvage receptor binding epitope to the antibody (especially an antibody fragment), as described, e.g., in U.S. Pat. No. 5,739,277. For example, a nucleic acid molecule encoding the salvage receptor binding epitope can be linked in frame to a nucleic acid encoding a polypeptide sequence of this invention so that the fusion protein expressed by the engineered nucleic acid molecule comprises the salvage receptor binding epitope and a polypeptide sequence of this invention. As used herein, the term “salvage receptor binding epitope” refers to an epitope of the Fc region of an IgG molecule (e.g., IgG₁, IgG₂, IgG₃, or IgG₄) that is responsible for increasing the in vivo serum half-life of the IgG molecule (e.g., Ghetie, V et al., (2000) Ann. Rev. Immunol. 18:739-766, Table 1). Antibodies with substitutions in an Fc region thereof and increased serum half-lives are also described in WO00/42072 (Presta, L.), WO 02/060919; Shields, R. L., et al., (2001) JBC 276(9):6591-6604; Hinton, P. R., (2004) JBC 279(8):6213-6216). In another embodiment, the serum half-life can also be increased, for example, by attaching other polypeptide sequences. For example, antibodies of this invention or other polypeptide containing the amino acid sequences of this invention can be attached to serum albumin or a portion of serum albumin that binds to the FcRn receptor or a serum albumin binding peptide so that serum albumin binds to the antibody or polypeptide, e.g., such polypeptide sequences are disclosed in WO01/45746. In one embodiment, the serum albumin peptide to be attached comprises an amino acid sequence of DICLPRWGCLW (SEQ ID NO: 4). In another embodiment, the half-life of a Fab according to this invention is increased by these methods. See also, Dennis, M. S., et al., (2002) JBC 277(38):35035-35043 for serum albumin binding peptide sequences.

An “angiogenic factor or agent” is a growth factor which stimulates the development of blood vessels, e.g., which promotes angiogenesis, endothelial cell growth, stability of blood vessels, and/or vasculogenesis, etc. For example, angiogenic factors include, but are not limited to, e.g., VEGF and members of the VEGF family, PIGF, PDGF family, fibroblast growth factor family (FGFs), TIE ligands (Angiopoietins), ephrins, Del-1, fibroblast growth factors: acidic (aFGF) and basic (bFGF), Follistatin, Granulocyte colony-stimulating factor (G-CSF), Hepatocyte growth factor (HGF)/scatter factor (SF), Interleukin-8 (IL-8), Leptin, Midkine, Placental growth factor, Platelet-derived endothelial cell growth factor (PD-ECGF), Platelet-derived growth factor, especially PDGF-BB or PDGFR-beta, Pleiotrophin (PTN), Progranulin, Proliferin, Transforming growth factor-alpha (TGF-alpha), Transforming growth factor-beta (TGF-beta), Tumor necrosis factor-alpha (TNF-alpha), Vascular endothelial growth factor (VEGF)/vascular permeability factor (VPF), etc. The term also includes, but is not limited to, factors that accelerate wound healing, such as growth hormone, insulin-like growth factor-I (IGF-I), VIGF, epidermal growth factor (EGF), CTGF and members of its family, and TGF-alpha and TGF-beta. See, e.g., Klagsbrun and D'Amore, Annu. Rev. Physiol., 53:217-39 (1991); Streit and Detmar, Oncogene, 22:3172-3179 (2003); Ferrara & Alitalo, Nature Medicine 5(12):1359-1364 (1999); Tonini et al., Oncogene, 22:6549-6556 (2003) (e.g., Table 1 listing known angiogenic factors); and, Sato Int. J. Clin. Oncol., 8:200-206 (2003).

An “anti-angiogenesis agent” or “angiogenesis inhibitor” refers to a small molecular weight substance, a polynucleotide, a polypeptide, an isolated protein, a recombinant protein, an antibody, or conjugates or fusion proteins thereof, that inhibits angiogenesis, vasculogenesis, or undesirable vascular permeability, either directly or indirectly. It should be understood that the term anti-angiogenesis agent includes, but is not limited to, those agents that bind and block the angiogenic activity of the angiogenic factor or its receptor. For example, an anti-angiogenesis agent is an antibody or other antagonist to an angiogenic agent as defined above, e.g., antibodies to VEGF-A or to the VEGF-A receptor (e.g., KDR receptor or Flt-1 receptor), and anti-PDGFR inhibitors such as Gleevec™ (Imatinib Mesylate). Anti-angiogenesis agents also include native angiogenesis inhibitors, e.g., angiostatin, endostatin, etc. See, e.g., Klagsbrun and D'Amore, Annu. Rev. Physiol., 53:217-39 (1991); Streit and Detmar, Oncogene, 22:3172-3179 (2003) (e.g., Table 3 listing anti-angiogenic therapy in malignant melanoma); Ferrara & Alitalo, Nature Medicine 5(12): 1359-1364 (1999); Tonini et al., Oncogene, 22:6549-6556 (2003) (e.g., Table 2 listing known antiangiogenic factors); and, Sato Int. J. Clin. Oncol., 8:200-206 (2003) (e.g., Table 1 lists anti-angiogenic agents used in clinical trials).

The “Kd” or “Kd value” is the dissociation constant for the interaction of one molecule with another. In one embodiment, the Kd value is measured by a radiolabeled protein binding assay (RIA). In one embodiment, an RIA for VEGF can be performed with the Fab version of an anti-VEGF antibody and a VEGF molecule as described by the following assay that measures solution binding affinity of Fabs for VEGF by equilibrating a Fab with a minimal concentration of (¹²⁵I)-labeled VEGF in the presence of a titration series of unlabeled VEGF, then capturing bound VEGF with an anti-Fab antibody-coated plate (Chen, et al., (1999) J. Mol Biol 293:865-881). To establish conditions for the assay, microtiter plates (Dynex) are coated overnight with 5 μg/ml of a capturing anti-Fab antibody (Cappel Labs) in 50 mM sodium carbonate (pH 9.6), and subsequently blocked with 2% (w/v) bovine serum albumin in PBS for two to five hours at room temperature (approximately 23° C.). In a non-adsorbant plate (Nunc #269620), 100 pM or 26 pM [¹²⁵I]VEGF are mixed with serial dilutions of a Fab of interest, e.g., Fab-12 (Presta et al., (1997) Cancer Res. 57:4593-4599). The Fab of interest is then incubated overnight; however, the incubation may continue for 65 hours to insure that equilibrium is reached. Thereafter, the mixtures are transferred to the capture plate for incubation at room temperature for one hour. The solution is then removed and the plate washed eight times with 0.1% Tween-20 in PBS. When the plates had dried, 150 μl/well of scintillant (MicroScint-20; Packard) is added, and the plates are counted on a Topcount gamma counter (Packard) for ten minutes. Concentrations of each Fab that give less than or equal to 20% of maximal binding are chosen for use in competitive binding assays. In other embodiments, a similar RIA methodology may be used to determine the Kd of one or more anti-insulin antibodies for insulin, of one or more anti-HER2 antibodies for HER2, of one or more anti-IGF-1 antibodies for IGF-1, and of one or more anti-HGH antibodies for HGH.

According to another embodiment the Kd or Kd value can be measured by using surface plasmon resonance assays using a BIAcore™-2000 or a BIAcore™-3000 instrument (BIAcore, Inc., Piscataway, N.J.). In one embodiment, the Kd value of anti-VEGF antibodies for VEGF is determined using BIAcore™ analysis according to the following protocol. Briefly, carboxymethylated dextran biosensor chips (CM5, BIAcore Inc.) are activated with N-ethyl-N′-(3-dimethylaminopropyl)-carbodiimide hydrochloride (EDC) and N-hydroxysuccinimide (NHS) according to the supplier's instructions. Human VEGF is diluted with 10 mM sodium acetate, pH 4.8, to 5 μg/ml (˜0.2 μM) before injection at a flow rate of 5 μl/minute to achieve approximately 10 response units (RU) of coupled protein. Following the injection of human VEGF, 1M ethanolamine is injected to block unreacted groups. For kinetics measurements, two-fold serial dilutions of Fab (0.78 nM to 500 nM) are injected in PBS with 0.05% Tween 20 (PBST) at 25° C. at a flow rate of approximately 25 μl/min. Association rates (kₒₙ) and dissociation rates (k_{off}) are calculated using a simple one-to-one Langmuir binding model (BIAcore Evaluation Software version 3.2) by simultaneously fitting the association and dissociation sensorgram. The equilibrium dissociation constant (Kd) was calculated as the ratio k_{off}/kₒₙ. See, e.g., Chen, Y., et al., (1999) J. Mol Biol 293:865-881. In other embodiments, a similar BIAcore™ methodology may be used to determine the Kd of one or more anti-insulin antibodies for insulin, of one or more anti-HER2 antibodies for HER2, of one or more anti-IGF-1 antibodies for IGF-1, and of one or more anti-HGH antibodies for HGH.

An “on-rate” or “rate of association” or “association rate” or “kₒₙ” according to this invention is preferably determined with same surface plasmon resonance technique described above using a BIAcore™-2000 or a BIAcore™-3000 (BIAcore, Inc., Piscataway, N.J.) at 25° C. with immobilized hVEGF (8-109) CM5 chips at ˜10 response units (RU). Briefly, carboxymethylated dextran biosensor chips (CM5, BIAcore Inc.) are activated with N-ethyl-N′-(3-dimethylaminopropyl)-carbodiimide hydrochloride (EDC) and N-hydroxysuccinimide (NHS) according to the supplier's instructions. Human VEGF is diluted with 10 mM sodium acetate, pH 4.8, into 5 ug/ml (˜0.2 uM) before injection at a flow rate of 5 ul/minute to achieve approximately 10 response units (RU) of coupled protein. Following the injection of 1M ethanolamine to block unreacted groups. For kinetics measurements, two-fold serial dilutions of Fab (0.78 nM to 500 nM) are injected in PBS with 0.05% Tween 20 (PBST) at 25° C. at a flow rate of approximately 25 ul/min. Association rates (kₒₙ) and dissociation rates (k_{off}) are calculated using a simple one-to-one Langmuir binding model (BIAcore Evaluation Software version 3.2) by simultaneous fitting the association and dissociation sensorgram. The equilibrium dissociation constant (Kd) was calculated as the ratio k_{off}/kₒₙ. See, e.g., Chen, Y., et al., (1999) J. Mol Biol 293:865-881.

In certain embodiments, the on-rate can be determined by fluorescence quenching, for example when the on-rate exceeds 10⁶ M⁻¹ s⁻¹ as determined by surface plasmon resonance analysis. In certain such embodiments, the on-rate can be determined by using a technique that measures the increase or decrease in fluorescence emission intensity (excitation=295 nm; emission=340 min, 16 nm band-pass) at 25° C. of a 20 nM anti-VEGF or anti-insulin antibody (Fab form) in PBS, pH 7.2, in the presence of increasing concentrations of VEGF or insulin, respectively, as measured in a spectrometer, such as a stop-flow equipped spectrophometer (Aviv Instruments) or a 8000-series SLM-Aminco spectrophotometer (ThermoSpectronic) with a stirred cuvette.

The term “VEGF” or “VEGF” as used herein refers to the 165-amino acid human vascular endothelial cell growth factor and related 121-, 189-, and 206-amino acid human vascular endothelial cell growth factors, as described by Leung et al. Science, 246:1306 (1989), and Houck et al. Mol. Endocrin., 5:1806 (1991), together with the naturally occurring allelic and processed forms thereof in native-sequence or in variant form, and from any source, whether natural, synthetic, or recombinant. The term “VEGF” also refers to VEGFs from non-human species such as mouse, rat or primate. Sometimes the VEGF from a specific species is indicated by terms such as hVEGF for human VEGF, mVEGF for murine VEGF, etc. The term “VEGF” is also used to refer to truncated forms of the polypeptide comprising amino acids 8 to 109 or 1 to 109 of the 165-amino acid human vascular endothelial cell growth factor. Reference to any such forms of VEGF may be identified in the present application, e.g., by “VEGF (8-109),” “VEGF (1-109)” or “VEGF₁₆₅.” The amino acid positions for a “truncated” native VEGF are numbered as indicated in the native VEGF sequence. For example, amino acid position 17 (methionine) in truncated native VEGF is also position 17 (methionine) in native VEGF. The truncated native VEGF has binding affinity for the KDR and Flt-1 receptors comparable to native VEGF.

The term “VEGF variant” as used herein refers to a VEGF polypeptide which includes one or more amino acid mutations in the native VEGF sequence. Optionally, the one or more amino acid mutations include amino acid substitution(s). For purposes of shorthand designation of VEGF variants described herein, it is noted that numbers refer to the amino acid residue position along the amino acid sequence of the putative native VEGF (provided in Leung et al., supra and Houck et al., supra.).

The term “IGF-I” refers to insulin-like growth factor-I from any species, including bovine, ovine, porcine, equine, and human, preferably human, and from any source, whether natural, synthetic, or recombinant. This may be prepared, e.g., by the process described in EP 230,869 published Aug. 5, 1987; EP 128,733 published Dec. 19, 1984; or EP 288,451 published Oct. 26, 1988. “Native-sequence human IGF-I” or “wild-type IGF-I” is wild-type human IGF-I.

The term, “growth hormone” or “GH” refers to growth hormone in native-sequence or in variant form, and from any source, whether natural, synthetic, or recombinant. Examples include human growth hormone (hGH), which is natural or recombinant GH with the human native sequence (somatotropin or somatropin), and recombinant growth hormone (rGH), which refers to any GH or variant produced by means of recombinant DNA technology, including somatrem, somatotropin, and somatropin. Preferred herein for human use is recombinant human native-sequence, mature GH with or without a methionine at its N-terminus. More preferred is methionyl human growth hormone (met-hGH) produced in E. coli, e.g., by the process described in U.S. Pat. No. 4,755,465 issued Jul. 5, 1988 and Goeddel et al., Nature, 282: 544 (1979). Met-hGH, which is sold under the trademark Protropin® by Genentech, Inc., is identical to the natural polypeptide, with the exception of the presence of an N-terminal methionine residue. This added amino acid is a result of the bacterial protein synthesis process. Also preferred is recombinant hGH available from Genentech, Inc. under the trademark Nutropin®. This latter hGH lacks this methionine residue and has an amino acid sequence identical to that of the natural hormone. See Gray et al., Biotechnology, 2: 161 (1984). Both methionyl hGH and hGH have equivalent potencies and pharmacokinetic values. Moore et al., Endocrinology, 122: 2920-2926 (1988). Another appropriate hGH candidate is an hGH variant that is a placental form of GH with pure somatogenic and no lactogenic activity as described in U.S. Pat. No. 4,670,393 issued 2 Jun. 1987. Also included are GH variants as described in WO 90/04788 published 3 May 1990 and WO 92/09690 published 11 Jun. 1992.

The term “HER2” refers to human epidermal growth factor receptor 2 (also known as NGL and human c-erbB-2, or ERBB2), the human homolog of the rat proto-oncogene neu, in native-sequence or in variant form, and from any source, whether natural, synthetic, or recombinant.

A “disorder” is any condition that would benefit from treatment with a substance/molecule or method of the invention. This includes chronic and acute disorders or diseases including those pathological conditions which predispose the mammal to the disorder in question. Non-limiting examples of disorders to be treated herein include VEGF-related disorders, insulin-related disorders, IGF-1-related disorders, HER2-related disorders, and HGH-related disorders.

A “VEGF-related disorder” refers to one or more disorders related to VEGF deficiency, misregulation of VEGF, aberrant reactions to VEGF, and/or overproduction of VEGF. VEGF-related disorders include, but are not limited to, malignant and benign tumors, non-leukemias and lymphoid malignancies, neutronal, glial, astrocytal, hypothalamic and other glandular, macrophagal, epithelial, stromal, and blastocoelic disorders; and inflammatory, immunologic and other abnormal angiogenesis or angiogenesis-related disorders (e.g., excessive, inappropriate, or uncontrolled angiogenesis, or aberrant vascular permeability).

The term “abnormal angiogenesis” refers to excessive, insufficient or inappropriate new blood vessel growth (e.g., the location, timing or onset of the angiogenesis being undesired from a medical standpoint) in a disease state or such that it causes a disease state. Excessive, inappropriate or uncontrolled angiogenesis occurs when there is new blood vessel growth that contributes to the worsening of the disease state or causes a disease state, such as in cancer, especially vascularized solid tumors and metastatic tumors (including colon, lung cancer (especially small-cell lung cancer), or prostate cancer), diseases caused by ocular neovascularisation, especially diabetic blindness, retinopathies, primarily diabetic retinopathy or age-induced macular degeneration and rubeosis; psoriasis, psoriatic arthritis, haemangioblastoma, such as haemangioma; inflammatory renal diseases, such as glomerulonephritis, especially mesangioproliferative glomerulonephritis, haemolytic uremic syndrome, diabetic nephropathy or hypertensive nephrosclerosis; various imflammatory diseases, such as arthritis, especially rheumatoid arthritis, inflammatory bowel disease, psorsasis, sarcoidosis, arterial arteriosclerosis and diseases occurring after transplants, endometriosis or chronic asthma, etc.. The new blood vessels can feed the diseased tissues, destroy normal tissues, and in the case of cancer, the new vessels can allow tumor cells to escape into the circulation and lodge in other organs (tumor metastases). Insufficient angiogenesis occurs when inadequate blood vessel growth contributes to the worsening of a disease state, e.g., diseases such as coronary artery disease, stroke, and delayed wound healing. Further, ulcers, strokes, and heart attacks can result from the absence of angiogenesis that is normally required for natural healing. The present invention contemplates treating those patients that are at risk of developing the above-mentioned illnesses.

Other patients that are candidates for receiving the anti-VEGF antibodies or polypeptides of this invention have, or are at risk for developing, abnormal proliferation of fibrovascular tissue, acne rosacea, acquired immune deficiency syndrome, artery occlusion, atopic keratitis, bacterial ulcers, Bechets disease, blood borne tumors, carotid obstructive disease, choroidal neovascularization, chronic inflammation, chronic retinal detachment, chronic uveitis, chronic vitritis, contact lens overwear, corneal graft rejection, corneal neovascularization, corneal graft neovascularization, Crohn's disease, Eales disease, epidemic keratoconjunctivitis, fungal ulcers, Herpes simplex infections, Herpes zoster infections, hyperviscosity syndromes, Kaposi's sarcoma, leukemia, lipid degeneration, Lyme's disease, marginal keratolysis, Mooren ulcer, Mycobacteria infections other than leprosy, myopia, ocular neovascular disease, optic pits, Osler-Weber syndrome (Qsler-Weber-Rendu, osteoarthritis, Pagets disease, pars planitis, pemphigoid, phylectenulosis, polyarteritis, post-laser complications, protozoan infections, pseudoxanthoma elasticum, pterygium keratitis sicca, radial keratotomy, retinal neovascularization, retinopathy of prematurity, retrolental fibroplasias, sarcoid, scleritis, sickle cell anemia, Sogrens syndrome, solid tumors, Stargarts disease, Steven's Johnson disease, superior limbic keratitis, syphilis, systemic lupus, Terrien's marginal degeneration, toxoplasmosis, trauma, tumors of Ewing sarcoma, tumors of neuroblastoma, tumors of osteosarcoma, tumors of retinoblastoma, tumors of rhabdomyosarcoma, ulcerative colitis, vein occlusion, Vitamin A deficiency and Wegeners sarcoidosis, undesired angiogenesis associated with diabetes, parasitic diseases, abnormal wound healing, hypertrophy following surgery, injury or trauma, inhibition of hair growth, inhibition of ovulation and corpus luteum formation, inhibition of implantation and inhibition of embryo development in the uterus.

Anti-angiogenesis therapies are useful in the general treatment of graft rejection, lung inflammation, nephrotic syndrome, preeclampsia, pericardial effusion, such as that associated with pericarditis, and pleural effusion, diseases and disorders characterized by undesirable vascular permeability, e.g., edema associated with brain tumors, ascites associated with malignancies, Meigs' syndrome, lung inflammation, nephrotic syndrome, pericardial effusion, pleural effusion, permeability associated with cardiovascular diseases such as the condition following myocardial infarctions and strokes and the like.

Other angiogenesis-dependent diseases include, but are not limited to, angiofibroma (abnormal blood of vessels which are prone to bleeding), neovascular glaucoma (growth of blood vessels in the eye), arteriovenous malformations (abnormal communication between arteries and veins), nonunion fractures (fractures that will not heal), atherosclerotic plaques (hardening of the arteries), pyogenic granuloma (common skin lesion composed of blood vessels), scleroderma (a form of connective tissue disease), hemangioma (tumor composed of blood vessels), trachoma (leading cause of blindness in the third world), hemophilic joints, vascular adhesions and hypertrophic scars (abnormal scar formation).

An “insulin-related disorder” refers to one or more disorders related to insulin deficiency, misregulation of insulin, aberrant reactions to insulin, and/or overproduction of insulin. Insulin-related disorders include, but are not limited to, diabetes mellitus type I (insulin deficiency), diabetes mellitus type II (insulin resistance), cardiovascular disease (including, but not limited to, hypertension, stroke, hypertriglyceridemia, low HDL-cholesterol, hyperinsulinemia, and hyperglycemia), vision disorders (including, but not limited to, diabetic retinopathy), kidney disorders (including, but not limited to, diabetic nephropathy, diabetic glomerulosclerosis, kidney infection, and renal papillary necrosis), gastrointestinal disease (including, but not limited to, diabetic gastropathy), diabetic foot ulcers, skin disorders (including, but not limited to, diabetic thick skin, yellow skin, macroangiopathy, diabetic demopathy, pigmented purpura, yellow nails, diabetic bullae, granuloma annulare, necrobiosis lipoidica, lichen planus, bullous pemphigoid, fat hypertrophy, candida infections, pseudomonas infections, dermatophytosis, periungual telangiectasia, and erysipelas-like erythema), and diabetic neuropathies (including, but not limited to, autonomic neuropathy, sensory neuropathy, and motor neuropathy).

An “IGF-1-related disorder” refers to one or more disorders related to IGF-1 deficiency, misregulation of IGF-1, aberrant reactions to IGF-1, and/or overproduction of IGF-1. IGF-1-related disorders include, but are not limited to, benign and malignant tumors, leukemias and lymphoid malignancies, neuronal, glial, astrocytal, hypothalamic and other glandular, macrophagal, epithelial, stromal and blastocoelic disorders, and inflammatory, angiogenic and immunologic disorders, diabetic complications such as diabetic retinopathies or neuropathies, age-related macular degeneration, ophthalmic surgery such as cataract extraction, a corneal transplant, glaucoma filtration surgery and keratoplasty, surgery to correct refraction, i.e., a radial keratotomy, also in sclera macular holes and degeneration, retinal tears, vitreoretinopathy, miscellaneous disorders, cataract disorders of the cornea such as the sequelae of radial keratotomy, dry eye, viral conjunctivitis, ulcerative conjunctivitis, wounds such as corneal epithelial wounds, Sjogren's syndrome, retinal disorders such as macular and retinal edema, vision-limited scarring, retinal ischemia, and proliferative vitreous retinopathy, ischemic injury (e.g., strokes, myocardial ischemia, and ischemic injury to the kidneys), diseases associated with undesirable cell proliferation such as cancer, restenosis, and asthma, lung diseases, hyperglycemic disorders, renal disorders, such as acute and chronic renal insufficiency, end-stage chronic renal failure, glomerulonephritis, interstitial nephritis, pyelonephritis, glomerulosclerosis, e.g., Kimmelstiel-Wilson in diabetic patients and kidney failure after kidney transplantation, obesity, GH-insufficiency, Turner's syndrome, Laron's syndrome, short stature, undesirable symptoms associated with aging such as obesity and increased fat mass-to-lean ratios, immunological disorders such as immunodeficiencies including decreased CD4 counts and decreased immune tolerance or chemotherapy-induced tissue damage, bone marrow transplantation, diseases or insufficiencies of cardiac structure or function such as heart dysfunctions and congestive heart failure, neuronal, neurological, or neuromuscular disorders, e.g., peripheral neuropathy, multiple sclerosis, muscular dystrophy, or myotonic dystrophy, and catabolic states associated with wasting caused by any condition, including, e.g., trauma or wounding, or infection such as with a bacterium or human virus such as HIV, wounds, skin disorders, gut structure and function that need restoration, and so forth.

As used herein, the term “hyperglycemic disorders” refers to all forms of diabetes and disorders resulting from insulin resistance, such as Type I and Type II diabetes, as well as severe insulin resistance, hyperinsulinemia, and hyperlipidemia, e.g., obese subjects, and insulin-resistant diabetes, such as Mendenhall's Syndrome, Werner Syndrome, leprechaunism, lipoatrophic diabetes, and other lipoatrophies. The preferred hyperglycemic disorder is diabetes, especially Type 1 and Type II diabetes. “Diabetes” itself refers to a progressive disease of carbohydrate metabolism involving inadequate production or utilization of insulin and is characterized by hyperglycemia and glycosuria.

A “HER2-related disorder” refers to one or more disorders related to HER2 deficiency, misregulation of HER2, aberrant reactions to HER2, and/or overproduction of HER2. A HER2-related disorder includes, but is not limited to, benign and malignant tumors; leukemias and lymphoid malignancies; neuronal, glial, astrocytal, hypothalamic and other glandular, macrophagal, epithelial, stromal and blastocoelic disorders; and inflammatory, angiogenic and immunologic disorders as described herein and generally known in the art.

A “human growth hormone related disorder” or an “HGH-related disorder” refers to one or more disorders related to HGH deficiency, misregulation of HGH, aberrant reactions to HGH, and/or overproduction of HGH. An HGH-related disorder includes, but is not limited to, growth disorders (e.g., Turner's syndrome, idopathic short stature, GH deficiency, and the like), vascular eye disease (e.g., retinopathy of prematurity, retinopathy associated with sickle cell anemia, and age-related macular degeneration), growth- hornone-responsive malignancies (e.g., Wilm's tumor, various sarcomas (e.g., osteogenic sarcoma), and breast, colon, prostate, and thyroid cancer), diabetes and diabetes-related complications (e.g., diabetic retinopathy and diabetic nephropathy), chronic renal insufficiency, and immune disorders as described herein and generally known in the art.

The terms “cell proliferative disorder” and “proliferative disorder” refer to disorders that are associated with some degree of abnormal cell proliferation. In one embodiment, the cell proliferative disorder is cancer.

“Tumor”, as used herein, refers to all neoplastic cell growth and proliferation, whether malignant or benign, and all pre-cancerous and cancerous cells and tissues. The terms “cancer”, “cancerous”, “cell proliferative disorder”, “proliferative disorder” and “tumor” are not mutually exclusive as referred to herein.

The terms “cancer” and “cancerous” refer to or describe the physiological condition in mammals that is typically characterized by unregulated cell growth/proliferation. Examples of cancer include but are not limited to, carcinoma, lymphoma, blastoma, sarcoma, and leukemia. More particular examples of such cancers include squamous cell cancer, small-cell lung cancer, non-small cell lung cancer, adenocarcinoma of the lung, squamous carcinoma of the lung, cancer of the peritoneum, hepatocellular cancer, gastrointestinal cancer, pancreatic cancer, glioblastoma, cervical cancer, ovarian cancer, liver cancer, bladder cancer, hepatoma, breast cancer, colon cancer, colorectal cancer, endometrial or uterine carcinoma, salivary gland carcinoma, kidney cancer, liver cancer, prostate cancer, vulval cancer, thyroid cancer, hepatic carcinoma and various types of head and neck cancer.

Dysregulation of angiogenesis can lead to many disorders that can be treated by compositions and methods of the invention. These disorders include both non-neoplastic and neoplastic conditions. Neoplastics include but are not limited those described above. Non-neoplastic disorders include but are not limited to undesired or aberrant hypertrophy, arthritis, rheumatoid arthritis (RA), psoriasis, psoriatic plaques, sarcoidosis, atherosclerosis, atherosclerotic plaques, diabetic and other proliferative retinopathies including retinopathy of prematurity, retrolental fibroplasia, neovascular glaucoma, age-related macular degeneration, diabetic macular edema, corneal neovascularization, comeal graft neovascularization, corneal graft rejection, retinal/choroidal neovascularization, neovascularization of the angle (rubeosis), ocular neovascular disease, vascular restenosis, arteriovenous malformations (AVM), meningioma, hemangioma, angiofibroma, thyroid hyperplasias (including Grave's disease), corneal and other tissue transplantation, chronic inflammation, lung inflammation, acute lung injury/ARDS, sepsis, primary pulmonary hypertension, malignant pulmonary effusions, cerebral edema (e.g., associated with acute stroke/ closed head injury/ trauma), synovial inflammation, pannus formation in RA, myositis ossificans, hypertropic bone formation, osteoarthritis (OA), refractory ascites, polycystic ovarian disease, endometriosis, 3rd spacing of fluid diseases (pancreatitis, compartment syndrome, burns, bowel disease), uterine fibroids, premature labor, chronic inflammation such as IBD (Crohn's disease and ulcerative colitis), renal allograft rejection, inflammatory bowel disease, nephrotic syndrome, undesired or aberrant tissue mass growth (non-cancer), hemophilic joints, hypertrophic scars, inhibition of hair growth, Osler-Weber syndrome, pyogenic granuloma retrolental fibroplasias, scleroderma, trachoma, vascular adhesions, synovitis, dermatitis, preeclampsia, ascites, pericardial effusion (such as that associated with pericarditis), and pleural effusion.

As used herein, “treatment” refers to clinical intervention in an attempt to alter the natural course of the individual or cell being treated, and can be performed either for prophylaxis or during the course of clinical pathology. Desirable effects of treatment include preventing occurrence or recurrence of disease, alleviation of symptoms, diminishment of any direct or indirect pathological consequences of the disease, preventing metastasis, decreasing the rate of disease progression, amelioration or palliation of the disease state, and remission or improved prognosis. In some embodiments, antibodies of the invention are used to delay development of a disease or disorder.

An “effective amount” refers to an amount effective, at dosages and for periods of time necessary, to achieve the desired therapeutic or prophylactic result.

A “therapeutically effective amount” of a substance/molecule of the invention, agonist or antagonist may vary according to factors such as the disease state, age, sex, and weight of the individual, and the ability of the substance/molecule, agonist or antagonist to elicit a desired response in the individual. A therapeutically effective amount is also one in which any toxic or detrimental effects of the substance/molecule, agonist or antagonist are outweighed by the therapeutically beneficial effects. A “prophylactically effective amount” refers to an amount effective, at dosages and for periods of time necessary, to achieve the desired prophylactic result. Typically but not necessarily, since a prophylactic dose is used in subjects prior to or at an earlier stage of disease, the prophylactically effective amount will be less than the therapeutically effective amount.

“Mammal” for purposes of treatment refers to any animal classified as a mammal, including humans, domestic and farm animals, nonhuman primates, and zoo, sports, or pet animals, such as dogs, horses, cats, cows, etc.

The term “anti-neoplastic composition” refers to a composition useful in treating cancer comprising at least one active therapeutic agent, e.g., “anti-cancer agent.” Examples of therapeutic agents (anti-cancer agents) include, but are not limited to, e.g., chemotherapeutic agents, growth inhibitory agents, cytotoxic agents, agents used in radiation therapy, anti-angiogenesis agents, apoptotic agents, anti-tubulin agents, and other-agents to treat cancer, such as anti-HER-2 antibodies, anti-CD20 antibodies, an epidermal growth factor receptor (EGFR) antagonist (e.g., a tyrosine kinase inhibitor), HER1/EGFR inhibitor (e.g., erlotinib (Tarceva™), platelet derived growth factor inhibitors (e.g., Gleevec™ (Imatinib Mesylate)), a COX-2 inhibitor (e.g., celecoxib), interferons, cytokines, antagonists (e.g., neutralizing antibodies) that bind to one or more of the following targets ErbB2, ErbB3, ErbB4, PDGFR-beta, BlyS, APRIL, BCMA or VEGF receptor(s), TRAIL/Apo2, and other bioactive and organic chemical agents, etc. Combinations thereof are also included in the invention.

The term “epitope tagged” when used herein refers to an antibody mutant fused to an “epitope tag”. The epitope tag polypeptide has enough residues to provide an epitope against which an antibody thereagainst can be made, yet is short enough such that it does not interfere with activity of the antibody mutant. The epitope tag preferably also is fairly unique so that the antibody thereagainst does not substantially cross-react with other epitopes. Suitable tag polypeptides generally have at least 6 amino acid residues and usually between about 8-50 amino acid residues (in certain embodiments between about 9-30 residues). Examples include, but are not limited to, the flu HA tag polypeptide and its antibody 12CA5 (Field et al. Mol. Cell. Biol. 8:2159-2165 (1988)); the c-myc tag and the 8F9, 3C7, 6E10, G4, B7 and 9E10 antibodies thereagainst (Evan et al., Mol. Cell. Biol. 5(12):3610-3616 (1985)); and the Herpes Simplex virus glycoprotein D (gD) tag and its antibody (Paborsky et al, Protein Engineering 3(6):547-553 (1990)). In certain embodiments, the epitope tag is a “salvage receptor binding epitope”.

The term “cytotoxic agent” as used herein refers to a substance that inhibits or prevents the function of cells and/or causes destruction of cells. The term is intended to include radioactive isotopes (e.g., At²¹¹, I¹³¹, I¹²⁵, Y⁹⁰, Re¹⁸⁶, Re¹⁸⁸, Sm¹⁵³, Bi²¹², P³² and radioactive isotopes of Lu), chemotherapeutic agents e.g. methotrexate, adriamicin, vinca alkaloids (vincristine, vinblastine, etoposide), doxorubicin, melphalan, mitomycin C, chlorambucil, daunorubicin or other intercalating agents, enzymes and fragments thereof such as nucleolytic enzymes, antibiotics, and toxins such as small molecule toxins or enzymatically active toxins of bacterial, fungal, plant or animal origin, including fragments and/or variants thereof, and the various antitumor or anticancer agents disclosed below. Other cytotoxic agents are described below. A tumoricidal agent causes destruction of tumor cells.

A “chemotherapeutic agent” is a chemical compound useful in the treatment of cancer. Examples of chemotherapeutic agents include alkylating agents such as thiotepa and CYTOXAN® cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethiylenethiophosphoramide and trimethylolomelamine; acetogenins (especially bullatacin and bullatacinone); delta-9-tetrahydrocannabinol (dronabinol, MARINOL®); beta-lapachone; lapachol; colchicines; betulinic acid; a camptothecin (including the synthetic analogue topotecan (HYCAMTIN®), CPT-11 (irinotecan, CAMPTOSAR®), acetylcamptothecin, scopolectin, and 9-aminocamptothecin); bryostatin; callystatin; CC-1065 (including its adozelesin, carzelesin and bizelesin synthetic analogues); podophyllotoxin; podophyllinic acid; teniposide; cryptophycins (particularly cryptophycin 1 and cryptophycin 8); dolastatin; duocarmycin (including the synthetic analogues, KW-2189 and CB1-TM1); eleutherobin; pancratistatin; a sarcodictyin; spongistatin; nitrogen mustards such as chlorambucil, chlomaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, and ranimnustine; antibiotics such as the enediyne antibiotics (e.g., calicheamicin, especially calicheamicin gamma1I and calicheamicin omegaI1 (see, e.g., Agnew, Chem Intl. Ed. Engl., 33: 183-186 (1994)); dynemicin, including dynemicin A; an esperamicin; as well as neocarzinostatin chromophore and related chromoprotein enediyne antibiotic chromophores), aclacinomysins, actinomycin, authramycin, azaserine, bleomycins, cactinomycin, carabicin, carminomycin, carzinophilin, chromomycinis, dactinomycin, daunorubicin, detorubicin, 6-diazo-5-oxo-L-norleucine, ADRIAMYCIN® doxorubicin (including morpholino-doxorubicin, cyanomorpholino-doxorubicin, 2-pyrrolino-doxorubicin and deoxydoxorubicin), epirubicin, esorubicin, idarubicin, marcellomycin, mitomycins such as mitomycin C, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elfornithine; elliptinium acetate; an epothilone; etoglucid; gallium nitrate; hydroxyurea; lentinan; lonidainine; maytansinoids such as maytansine and ansamitocins; mitoguazone; mitoxantrone; mopidanmol; nitraerine; pentostatin; phenamet; pirarubicin; losoxantrone; 2-ethylhydrazide; procarbazine; PSK® polysaccharide complex (JHS Natural Products, Eugene, Oreg.); razoxane; rhizoxin; sizofiran; spirogermanium; tenuazonic acid; triaziquone; 2,2′,2″-trichlorotriethylamine; trichothecenes (especially T-2 toxin, verracurin A, roridin A and anguidine); urethan; vindesine (ELDISINE®, FILDESIN®); dacarbazine; mannomustine; mitobronitol; mitolactol; pipobroman; gacytosine; arabinoside (“Ara-C”); thiotepa; taxoids, e.g., TAXOL® paclitaxel (Bristol-Myers Squibb Oncology, Princeton, N.J.), ABRAXANE™ Cremophor-free, albumin-engineered nanoparticle formulation of paclitaxel (American Pharmaceutical Partners, Schaumberg, Ill.), and TAXOTERE® doxetaxel (Rhône-Poulenc Rorer, Antony, France); chloranbucil; gemcitabine (GEMZAR®); 6-thioguanine; mercaptopurine; methotrexate; platinum analogs such as cisplatin and carboplatin; vinblastine (VELBAN®); platinum; etoposide (VP-16); ifosfamide; mitoxantrone; vincristine (ONCOVIN®); oxaliplatin; leucovovin; vinorelbine (NAVELBINE®); novantrone; edatrexate; daunomycin; aminopterin; ibandronate; topoisomerase inhibitor RFS 2000; difluorometlhylornithine (DMFO); retinoids such as retinoic acid; capecitabine (XELODA®); pharmaceutically acceptable salts, acids or derivatives of any of the above; as well as combinations of two or more of the above such as CHOP, an abbreviation for a combined therapy of cyclophosphamide, doxorubicin, vincristine, and prednisolone, and FOLFOX, an abbreviation for a treatment regimen with oxaliplatin (ELOXATIN™) combined with 5-FU and leucovovin.

Also included in this definition are anti-hormonal agents that act to regulate, reduce, block, or inhibit the effects of hormones that can promote the growth of cancer, and are often in the form of systemic, or whole-body treatment. They may be hormones themselves. Examples include anti-estrogens and selective estrogen receptor modulators (SERMs), including, for example, tamoxifen (including NOLVADEX® tamoxifen), EVISTA® raloxifene, droloxifene, 4-hydroxytamoxifen, trioxifene, keoxifene, LY117018, onapristone, and FARESTON® toremifene; anti-progesterones; estrogen receptor down-regulators (ERDs); agents that function to suppress or shut down the ovaries, for example, leutinizing hormone-releasing hormone (LHRH) agonists such as LUPRON® and ELIGARD® leuprolide acetate, goserelin acetate, buserelin acetate and tripterelin; other anti-androgens such as flutamide, nilutamide and bicalutamide; and aromatase inhibitors that inhibit the enzyme aromatase, which regulates estrogen production in the adrenal glands, such as, for example, 4(5)-imidazoles, aminoglutethimide, MEGASE® megestrol acetate, AROMASIN® exemestane, formestanie, fadrozole, RIVISOR® vorozole, FEMARA® letrozole, and ARIMIDEX® anastrozole. In addition, such definition of chemotherapeutic agents includes bisphosphonates such as clodronate (for example, BONEFOS® or OSTAC®), DIDROCAL® etidronate, NE-58095, ZOMETA® zoledronic acid/zoledronate, FOSAMAX® alendronate, AREDIA® pamidronate, SKELID® tiludronate, or ACTONEL® risedronate; as well as troxacitabine (a 1,3-dioxolane nucleoside cytosine analog); antisense oligonucleotides, particularly those that inhibit expression of genes in signaling pathways implicated in abberant cell proliferation, such as, for example, PKC-alpha, Raf, H-Ras, and epidermal growth factor receptor (EGF-R); vaccines such as THERATOPE® vaccine and gene therapy vaccines, for example, ALLOVECTIN® vaccine, LEUVECTIN® vaccine, and VAXID® vaccine; LURTOTECAN® topoisomerase 1 inhibitor; ABARELIX® rmRH; lapatinib ditosylate (an ErbB-2 and EGFR dual tyrosine kinase small-molecule inhibitor also known as GW572016); and pharmaceutically acceptable salts, acids or derivatives of any of the above.

A “growth inhibitory agent” when used herein refers to a compound or composition which inhibits growth of a cell whose growth is dependent upon activity of a target molecule of interest either in vitro or in vivo. Thus, the growth inhibitory agent may be one which significantly reduces the percentage of target molecule-dependent cells in S phase. Examples of growth inhibitory agents include agents that block cell cycle progression (at a place other than S phase), such as agents that induce G1 arrest and M-phase arrest. Classical M-phase blockers include the vincas (vincristine and vinblastine), taxanes, and topoisomerase II inhibitors such as doxorubicin, epirubicin, daunorubicin, etoposide, and bleomycin. Those agents that arrest G1 also spill over into S-phase arrest, for example, DNA alkylating agents such as tamoxifen, prednisone, dacarbazine, mechlorethamine, cisplatin, methotrexate, 5-fluorouracil, and ara-C. Further information can be found in The Molecular Basis of Cancer, Mendelsohn and Israel, eds., Chapter 1, entitled “Cell cycle regulation, oncogenes, and antineoplastic drugs” by Murakami et al. (W B Saunders: Philadelphia, 1995), especially p. 13. The taxanes (paclitaxel and docetaxel) are anticancer drugs both derived from the yew tree. Docetaxel (TAXOTERE®, Rhone-Poulenc Rorer), derived from the European yew, is a semisynthetic analogue of paclitaxel (TAXOL®, Bristol-Myers Squibb). Paclitaxel and docetaxel promote the assembly of microtubules from tubulin dimers and stabilize microtubules by preventing depolymerization, which results in the inhibition of mitosis in cells.

“Doxorubicin” is an anthracycline antibiotic. The full chemical name of doxorubicin is (8S-cis)-10-[(3-amino-2,3,6-trideoxy-α-L-lyxo-hexapyranosyl)oxy]-7,8,9,10-tetrahydro-6,8,11-trihydroxy-8-(hydroxyacetyl)-1-methoxy-5,12-naphthacenedione.

The term “prodrug” as used in this application refers to a precursor or derivative form of a pharmaceutically active substance that is less cytotoxic to tumor cells compared to the parent drug and is capable of being enzymatically activated or converted into the more active parent form. See, e.g., Wilman, “Prodrugs in Cancer Chemotherapy” Biochemical Society Transactions, 14, pp. 375-382, 615th Meeting Belfast (1986) and Stella et al., “Prodrugs: A Chemical Approach to Targeted Drug Delivery,” Directed Drug Delivery, Borchardt et al., (ed.), pp. 247-267, Humana Press (1985). The prodrugs of this invention include, but are not limited to, phosphate-containing prodrugs, thiophosphate-containing prodrugs, sulfate-containing prodrugs, peptide-containing prodrugs, D-amino acid-modified prodrugs, glycosylated prodrugs, β-lactam-containing prodrugs, optionally substituted phenoxyacetamide-containing prodrugs or optionally substituted phenylacetamide-containing prodrugs, 5-fluorocytosine and other 5-fluorouridine prodrugs which can be converted into the more active cytotoxic free drug. Examples of cytotoxic drugs that can be derivatized into a prodrug form for use in this invention include, but are not limited to, those chemotherapeutic agents described above.

For the treatment of rheumatoid arthritis (“RA”), the patient can be treated with an antibody of the invention in conjunction with any one or more of the following drugs: DMARDS (disease-modifying anti-rheumatic drugs (e.g., methotrexate), NSAI or NSAID (non-steroidal anti-inflammatory drugs), HUMIRA™ (adalimumab; Abbott Laboratories), ARAVA® (leflunomide), REMICADE® (infliximab; Centocor Inc., of Malvern, Pa.), ENBREL™ (etanercept; Immunex, Wash.), and COX-2 inhibitors. DMARDs commonly used in RA are hydroxycloroquine, sulfasalazine, methotrexate, leflunomide, etanercept, infliximab, azathioprine, D-penicillamine, Gold (oral), Gold (intramuscular), minocycline, cyclosporine, and Staphylococcal protein A immunoadsorption. Adalimumab is a human monoclonal antibody that binds to TNF. Infliximab is a chimeric monoclonal antibody that binds to TNF. Etanercept is an “immunoadhesin” fusion protein consisting of the extracellular ligand binding portion of the human 75 kD (p75) tumor necrosis factor receptor (TNFR) linked to the Fc portion of a human IgG1. For conventional treatment of RA, see, e.g., “Guidelines for the management of rheumatoid arthritis” Arthritis & Rheumatism 46(2): 328-346 (February, 2002). In a specific embodiment, the RA patient is treated with a CD20 antibody of the invention in conjunction with methotrexate (MTX). An exemplary dosage of MTX is about 7.5-25 mg/kg/wk. MTX can be administered orally and subcutaneously.

For the treatment of ankylosing spondylitis, psoriatic arthritis and Crohn's disease, the patient can be treated with an antibody of the invention in conjunction with, for example, Remicade® (infliximab; from Centocor Inc., of Malvern, Pa.), and/or ENBREL (etanercept; Immunex, Wash.).

For treatments for SLE, the patient can be treated with an antibody of the invention in conjunction with, for example, a high-dose corticosteroids and/or cyclophosphamide (HDCC).

For the treatment of psoriasis, patients can be administered an antibody of this invention in conjunction with topical treatments, such as topical steroids, anthralin, calcipotriene, clobetasol, and tazarotene, or with methotrexate, retinoids, cyclosporine, PUVA and UVB therapies. In one embodiment, the psoriasis patient is treated with the antibody sequentially or concurrently with cyclosporine.

An “isolated” nucleic acid molecule is a nucleic acid molecule that is identified and separated from at least one contaminant nucleic acid molecule with which it is ordinarily associated in the natural source of the antibody nucleic acid. An isolated nucleic acid molecule is other than in the form or setting in which it is found in nature. Isolated nucleic acid molecules therefore are distinguished from the nucleic acid molecule as it exists in natural cells. However, an isolated nucleic acid molecule includes a nucleic acid molecule contained in cells that ordinarily express the antibody where, for example, the nucleic acid molecule is in a chromosomal location different from that of natural cells.

The expression “control sequences” refers to DNA sequences necessary for the expression of an operably linked coding sequence in a particular host organism. The control sequences that are suitable for prokaryotes, for example, include a promoter, optionally an operator sequence, and a ribosome binding site. Eukaryotic cells are known to utilize promoters, polyadenylation signals, and enhancers.

Nucleic acid is “operably linked” when it is placed into a functional relationship with another nucleic acid sequence. For example, DNA for a presequence or secretory leader is operably linked to DNA for a polypeptide if it is expressed as a preprotein that participates in the secretion of the polypeptide; a promoter or enhancer is operably linked to a coding sequence if it affects the transcription of the sequence; or a ribosome binding site is operably linked to a coding sequence if it is positioned so as to facilitate translation. Generally, “operably linked” means that the DNA sequences being linked are contiguous, and, in the case of a secretory leader, contiguous and in reading phase. However, enhancers do not have to be contiguous. Linking is accomplished by ligation at convenient restriction sites. If such sites do not exist, the synthetic oligonucleotide adaptors or linkers are used in accordance with conventional practice.

A “variant” or “mutant” of a starting or reference polypeptide (e.g., a source antibody or its variable domain(s)/CDR(s)), such as a fusion protein (polypeptide) or a heterologous polypeptide (heterologous to a phage), is a polypeptide that 1) has an amino acid sequence different from that of the starting or reference polypeptide and 2) was derived from the starting or reference polypeptide through either natural or artificial (manmade) mutagenesis. Such variants include, for example, deletions from, and/or insertions into and/or substitutions of, residues within the amino acid sequence of the polypeptide of interest. For example, a fusion polypeptide of the invention generated using an oligonucleotide comprising a restricted codon set that encodes a sequence with a variant amino acid (with respect to the amino acid found at the corresponding position in a source antibody/antigen binding fragment) would be a variant polypeptide with respect to a source antibody and/or antigen binding fragment and/or CDR. Thus, a variant CDR refers to a CDR comprising a variant sequence with respect to a starting or reference polypeptide sequence (such as that of a source antibody and/or antigen binding fragment and/or CDR). A variant amino acid, in this context, refers to an amino acid different from the amino acid at the corresponding position in a starting or reference polypeptide sequence (such as that of a source antibody and/or antigen binding fragment and/or CDR). Any combination of deletion, insertion, and substitution may be made to arrive at the final variant or mutant construct, provided that the final construct possesses the desired functional characteristics. In some of the examples described herein, binder sequences contain point mutations such as deletions or additions. The amino acid changes also may alter post-translational processes of the polypeptide, such as changing the number or position of glycosylation sites. Methods for generating amino acid sequence variants of polypeptides are described in U.S. Pat. No. 5,534,615, expressly incorporated herein by reference.

A “wild type” or “reference” sequence or the sequence of a “wild type” or “reference” protein/polypeptide, such as a coat protein, or a CDR or variable domain of a source antibody, maybe the reference sequence from which variant polypeptides are derived through the introduction of mutations. In general, the “wild type” sequence for a given protein is the sequence that is most common in nature. Similarly, a “wild type” gene sequence is the sequence for that gene which is most commonly found in nature. Mutations may be introduced into a “wild type” gene (and thus the protein it encodes) either through natural processes or through man induced means. The products of such processes are “variant” or “mutant” forms of the original “wild type” protein or gene.

A “plurality” of a substance, such as a polypeptide or polynucleotide of the invention, as used herein, generally refers to a collection of two or more types or kinds of the substance. There are two or more types or kinds of a substance if two or more of the substances differ from each other with respect to a particular characteristic, such as the variant amino acid found at a particular amino acid position. For example, there is a plurality of polypeptides of the invention if there are two or more polypeptides of the invention that are substantially the same, or are identical in sequence except for the sequence of a variant CDR or except for the variant amino acid at a particular solvent accessible and highly diverse amino acid position. In another example, there is a plurality of polynucleotides of the invention if there are two or more polynucleotides of the invention that are substantially the same or identical in sequence except for the sequence that encodes a variant CDR or except for the sequence that encodes a variant amino acid for a particular solvent accessible and highly diverse amino acid position.

The invention provides methods for generating and isolating novel target antigen binding polypeptides, such as antibodies or antigen binding fragments that can have a high affinity for a selected antigen. A plurality of different binder polypeptides are prepared by mutating (diversifying) one or more selected amino acid positions in a source antibody light chain variable domain and/or heavy chain variable domain with restricted codon sets to generate a library of binder polypeptides with variant amino acids in at least one CDR sequence, wherein the number of types of variant amino acids is kept to a minimum (i.e., 19 or fewer, 15 or fewer, 10 or fewer, 8 or fewer, 6 or fewer, 4 or fewer, or only 2, but generally at least 2). The amino acid positions include those that are solvent accessible, for example as determined by analyzing the structure of a source antibody, and/or that are highly diverse among known and/or natural occurring immunoglobulin polypeptides. A further advantage afforded by the limited nature of diversification of the invention is that additional amino acid positions other than those that are highly diverse and/or solvent accessible can also be diversified in accordance with the need or desire of the practitioner; examples of these embodiments are described herein.

The amino acid positions that are solvent accessible and highly diverse are in certain embodiments those in the CDR regions of the antibody variable domains selected from the group consisting of CDRL1, CDRL2, CDRL3, CDRH1, CDRH2, CDRH3, and mixtures thereof. Amino acid positions are each mutated using a restricted codon set encoding a limited number of amino acids, the choice of amino acids generally being independent of the commonly occurring amino acids at each position. In some embodiments, when a solvent accessible and highly diverse position in a CDR region is to be mutated, a codon set is selected that encodes from 2 to 19, 2 to 15, 2 to 10, from 2 to 8, from 2 to 6, from 2 to 4, and/or only 2 amino acids. In some embodiments, when a solvent accessible and highly diverse position in a CDR region is to be mutated, a codon set is selected that encodes from 2 to 10, from 3 to 9, from 4 to 8, and/or from 5 to 7 amino acids. In some embodiments, a codon set encodes at least 2, but 19 or fewer, 15 or fewer, 10 or fewer, 8 or fewer, 6 or fewer, 4 or fewer amino acids. CDR sequences can also be diversified by varying the length. For example, for CDRH3, variant CDRH3 regions can be generated that have different lengths and/or are randomized at selected positions using restricted codon sets.

The diversity of the library of the polypeptides comprising variant CDRs is designed using codon sets that encode only a limited number of amino acids, such that a minimum but sufficient amount of sequence diversity is introduced into a CDR. The number of positions mutated in the CDR is minimized and the variant amino acids at each position are designed to include a limited number of amino acids, independent of the amino acids that deemed to be commonly occurring at that position in known and/or naturally occurring CDRs. In certain embodiments, a single antibody, including at least one CDR, is used as the source antibody. It is surprising that a library of antibody variable domains having diversity in sequences and size can be generated using a single source antibody as a template and targeting diversity to particular positions using an unconventionally limited number of amino acid substitutions.

Design of Diversity of Antibody Variable Domains

In one aspect of the invention, high quality libraries of antibody variable domains are generated. The libraries have restricted diversity of different sequences of CDR sequences, for example, diversity of the antibody variable domains. The libraries include high affinity binding antibody variable domains for one or more antigens, including, for example, insulin and human VEGF. The diversity in the library is designed by selecting amino acid positions that are solvent accessible and highly diverse in a single source antibody and mutating those positions in at least one CDR using restricted codon sets. The restricted codon set can in certain embodiments encode fewer than 19, 15, 10, 8, 6, or 4 amino acids, or encodes only 2 amino acids.

One source antibody is humanized antibody 4D5, but the methods for diversification can be applied to other source antibodies whose sequence is known. A source antibody can be a naturally occurring antibody, synthetic antibody, recombinant antibody, humanized antibody, germ line derived antibody, chimeric antibody, affinity matured antibody, or antigen binding fragment thereof. The antibodies can be obtained from a variety of mammalian species including humans, mice and rats. In some embodiments, a source antibody is an antibody that is obtained after one or more initial affinity screening rounds, but prior to an affinity maturation step(s). A source antibody may be selected or modified to provide for high yield and stability when produced in cell culture.

Antibody 4D5 is a humanized antibody specific for a cancer-associated antigen known as Her-2 (erbB2). The antibody includes variable domains having consensus framework regions; a few positions were reverted to mouse sequence during the process of increasing affinity of the humanized antibody. The sequence and crystal structure of humanized antibody 4D5 have been described in U.S. Pat. No. 6,054,297, Carter et al, PNAS 89:4285 (1992), the crystal structure is shown in J Mol. Biol. 229:969 (1993) and online at http://www.ncbi.nlm.nih.gov/Structure/mmdb/mmdbsrv.cgi?form=6&db=t&Dopt=s&uid=990, http://www.ncbi.nlm.nih.gov/Structure/mmdb/mmdbsrv.cgi?form=6&db=t&Dopt=s&uid=991, and http://www.ncbi.nlm.nih.gov/Structure/mmdb/mmdbsrv.cgi?fom=6&db=t&Dopt=s&uid=992.

A criterion for generating diversity in antibody variable domains is to mutate residues at positions that are solvent accessible (as defined above). These positions are typically found in the CDRs, and are typically on the exterior of the protein. In certain embodiments, solvent accessible positions are determined using coordinates from a 3-dimensional model of an antibody, using a computer program such as the InsightII program (Accelrys, San Diego, Calif.). Solvent accessible positions can also be determined using algorithms known in the art (e.g., Lee and Richards, J. Mol. Biol. 55, 379 (1971) and Connolly, J. Appl. Cryst. 16, 548 (1983)). Determination of solvent accessible positions can be performed using software suitable for protein modeling and 3-dimensional structural information obtained from an antibody. Software that can be utilized for these purposes includes SYBYL Biopolymer Module software (Tripos Associates). Generally in certain embodiments, where an algorithm (program) requires a user input size parameter, the “size” of a probe which is used in the calculation is set at about 1.4 Angstrom or smaller in radius. In addition, determination of solvent accessible regions and area methods using software for personal computers has been described by Pacios ((1994) “ARVOMOL/CONTOUR: molecular surface areas and volumes on Personal Computers”, Comput. Chem. 18(4): 377-386; and “Variations of Surface Areas and Volumes in Distinct Molecular Surfaces of Biomolecules.” J. Mol. Model. (1995), 1: 46-53).

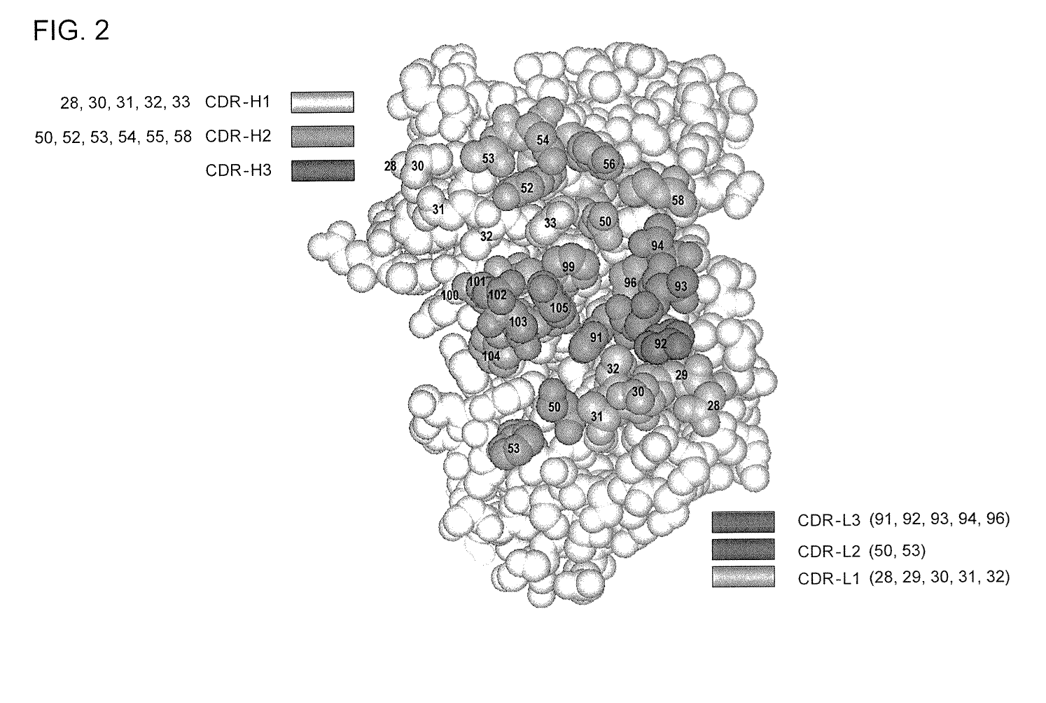

In some instances, selection of solvent accessible residues is further refined by choosing solvent accessible residues that collectively form a minimum contiguous patch, for example when the reference polypeptide or source antibody is in its 3-D folded structure. For example, as shown in FIG. 2, a compact (minimum) contiguous patch is formed by residues selected for CDRH1/H2/H3/L1/L2/L3 of humanized 4D5. A compact (minimum) contiguous patch may comprise only a subset (for example, 2-5 CDRs) of the full range of CDRs, for example, CDRH1/H2/H3/L3. Solvent accessible residues that do not contribute to formation of such a patch may optionally be excluded from diversification. Refinement of selection by this criterion permits the practitioner to minimize, as desired, the number of residues to be diversified. For example, residue 28 in H1 can optionally be excluded in diversification since it is on the edge of the patch. However, this selection criterion can also be used, where desired, to choose residues to be diversified that may not necessarily be deemed solvent accessible. For example, a residue that is not deemed solvent accessible, but forms a contiguous patch in the 3-D folded structure with other residues that are deemed solvent accessible may be selected for diversification. An example of this is CDRL1-29. Selection of such residues would be evident to one skilled in the art, and its appropriateness can also be determined empirically and according to the needs and desires of the skilled practitioner.

The solvent accessible positions identified from the crystal structure of humanized antibody 4D5 for each CDR are as follows (residue position according to Kabat):

CDRL1: 28, 30, 31, 32

CDRL2: 50, 53

CDRL3: 91, 92, 93, 94, 96

CDRH1: 28, 30, 31, 32, 33

CDRH2: 50, 52, 52A, 53, 54, 55, 56, 57, 58.

In addition, in some embodiments, residue 29 of CDRL1 may also be selected based on its inclusion in a contiguous patch comprising other solvent accessible residues. All or a subset of the solvent accessible positions as set forth above may be diversified in methods and compositions of the invention. For example, in some embodiments, only positions 50, 52, 52a, 53-56, and 58 are randomized in CDRH2.

Another criterion for selecting positions to be mutated is those positions which show variability in amino acid sequence when the sequences of known and/or natural antibodies are compared. A highly diverse position refers to a position of an amino acid located in the variable regions of the light or heavy chains that have a number of different amino acids represented at the position when the amino acid sequences of known and/or natural antibodies/antigen binding fragments are compared. The highly diverse positions can be in the CDR regions. The positions of CDRH3 are all considered highly diverse. In certain embodiments, amino acid residues are highly diverse if they have from about 2 to about 19 (although the numbers can range as described herein) different possible amino acid residue variations at that position.

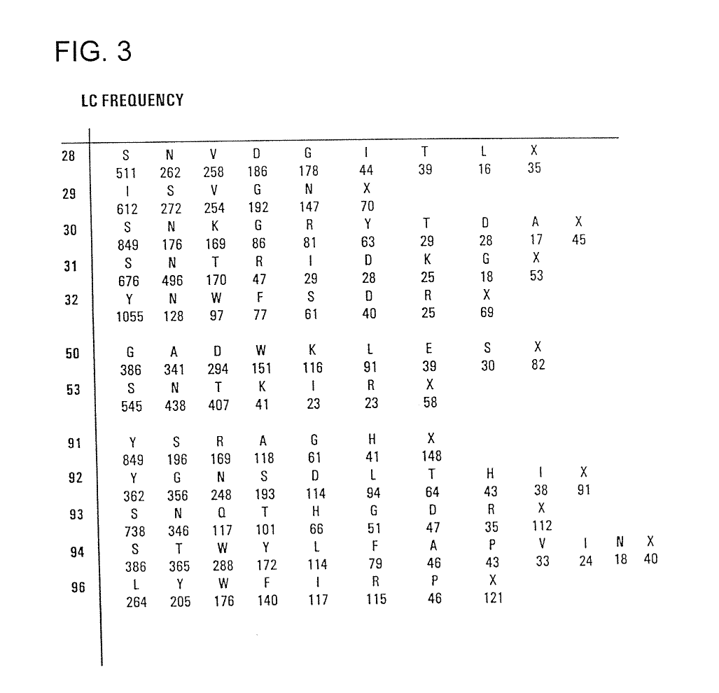

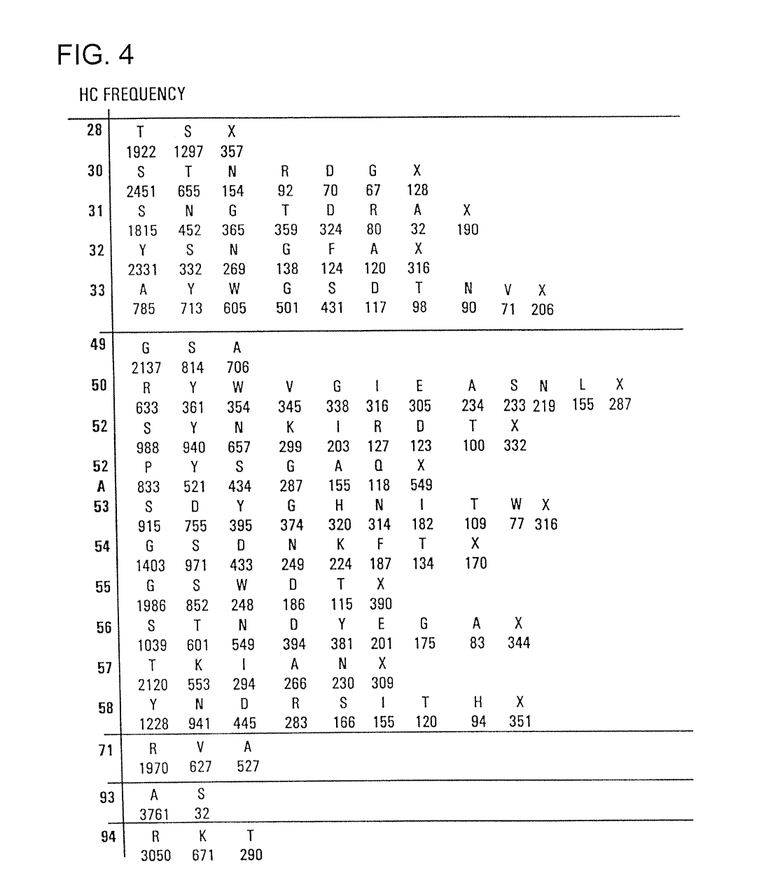

In one aspect, identification of highly diverse positions in known and/or naturally occurring antibodies is facilitated by the data provided by Kabat, Sequences of Proteins of Immunological Interest (National Institutes of Health, Bethesda, Md., 1987 and 1991). An internet-based database located at http://www.bioinf.org.uk/abs/structures.html, provides an extensive collection and alignment of light (http://www.bioinf.org.uk/abs/lc.align) and heavy chain (http://www.bioinf.org.uk/abs/hc.align) sequences and facilitates determination of highly diverse positions in these sequences. The diversity at the solvent accessible positions of humanized antibody 4D5 in known and/or naturally occurring light and heavy chains is shown in FIGS. 3 and 4.

In one aspect of the invention, the highly diverse and solvent accessible residues in at least one, two, three, four, five or all CDRs selected from the group consisting of CDRL1, CDRL2, CDRL3, CDRH1, CDRH2, CDRH3, and mixtures thereof are mutated (i.e., randomized using restricted codon sets as described herein). For example, a population of polypeptides may be generated by diversifying at least one solvent accessible and/or highly diverse residue in CDRL3 and CDRH3 using restricted codons. Accordingly, the invention provides for a large number of novel antibody sequences formed by replacing at least one solvent accessible and highly diverse position of at least one CDR of the source antibody variable domain with variant amino acids encoded by a restricted codon. For example, a variant CDR or antibody variable domain can comprise a variant amino acid in one or more of amino acid positions 28, 29, 30, 31, 32, 33, and/or 34 of CDRH1; and/or in one or more of amino acid positions 50, 52, 52a, 53, 54, 55, 56 and/or 58 of CDRH2; and/or in one or more of amino acid positions 95-100, 100a, 100b, 100c, 101, and/or 102 of CDRH3; and/or in one or more of amino acid positions 28, 29, 30 and/or 31 of CDRL1; and/or in one or more of amino acid positions 50 and/or 53 in CDRL2; and/or in one or more of amino acid positions 91, 92, 93, 94, 95 and/or 96 in CDRL3. In another example, a variant CDR or antibody variable domain can comprise a variant amino acid in one or more of amino acid positions 28, 30, 31, 32, and/or 33 of CDRH1; and/or in one or more of amino acid positions 50, 52, 53, 54, 56 and/or 58 of CDRH2; and/or in one or more of amino acid positions 95-100, 100a, 100b, 100c, 101 and/or 102 of CDRH3; and/or in one or more of amino acid positions 28, 29, 30 and/or 31 of CDRL1; and/or in one or more of amino acid positions 50 and/or 53 in CDRL2; and/or in one or more of amino acid positions 91, 92, 93, 94, and/or 96 in CDRL3. The variant amino acids at these positions are encoded by restricted codon sets, as described herein.

As discussed above, the variant amino acids are encoded by restricted codon sets. A codon set is a set of different nucleotide triplet sequences which can be used to form a set of oligonucleotides used to encode the desired group of amino acids. A set of oligonucleotides can be synthesized, for example, by solid phase synthesis, containing sequences that represent all possible combinations of nucleotide triplets provided by the codon set and that will encode the desired group of amino acids. Synthesis of oligonucleotides with selected nucleotide “degeneracy” at certain positions is well known in that art. Such sets of nucleotides having certain codon sets can be synthesized using commercial nucleic acid synthesizers (available from, for example, Applied Biosystems, Foster City, Calif.), or can be obtained commercially (for example, from Life Technologies, Rockville, Md.). Therefore, a set of oligonucleotides synthesized having a particular codon set will typically include a plurality of oligonucleotides with different sequences, the differences established by the codon set within the overall sequence. Oligonucleotides, as used according to the invention, have sequences that allow for hybridization to a variable domain nucleic acid template and also can include restriction enzyme sites for cloning purposes.

In one aspect, the restricted repertoire of amino acids intended to occupy one or more of the solvent accessible and highly diverse positions in CDRs of humanized antibody 4D5 are determined (based on the desire of the practitioner, which can be based on any of a number of criteria, including specific amino acids desired for particular positions, specific amino acid(s) desired to be absent from a particular position, size of library desired, characteristic of antigen binders sought, etc.).

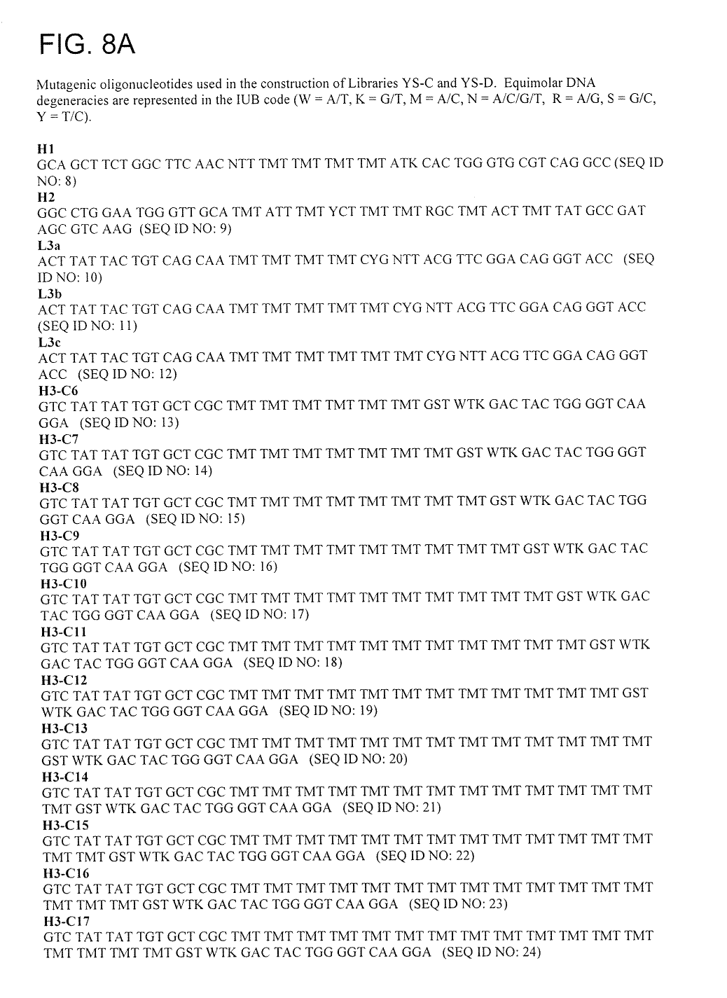

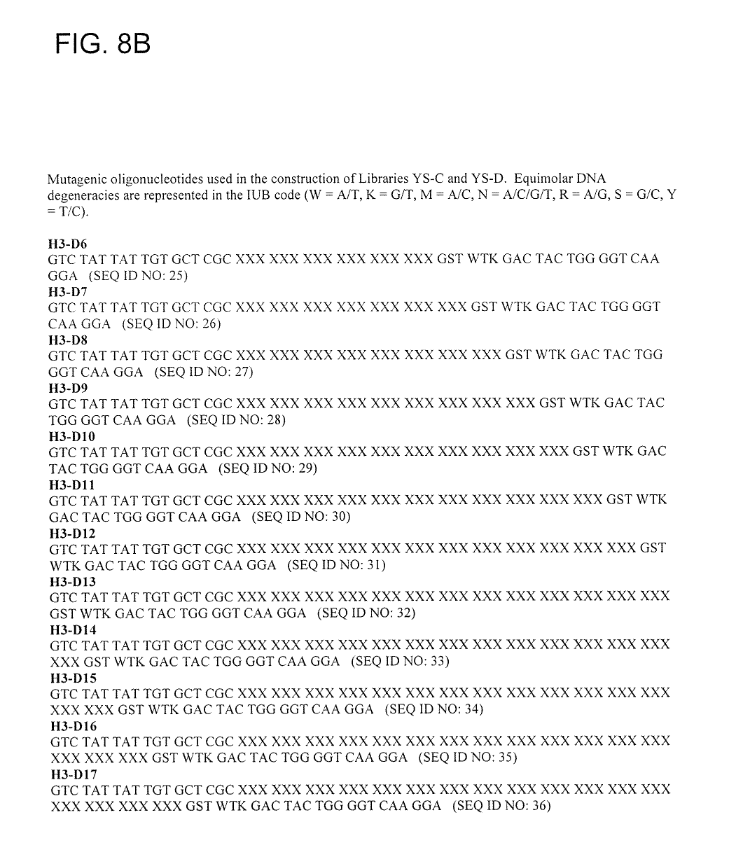

Heavy chain CDR3s (CDRH3s) in known antibodies have diverse sequences, structural conformations, and lengths. CDRH3s are often found in the middle of the antigen binding pocket and often participate in antigen contact. The design of CDRH3 may thus be developed separately from that of the other CDRs because it can be difficult to predict the structural conformation of CDRH3 and the amino acid diversity in this region is especially diverse in known antibodies. In accordance with the present invention, CDRH3 is designed to generate diversity at specific positions within CDRH3, for example, at positions 95, 96, 97, 98, 99, 100, 100a, 100b, 100c, 101, and/or 102 (e.g., according to Kabat numbering in antibody 4D5). In some embodiments, diversity is also generated by varying CDRH3 length using restricted codon sets. Length diversity can be of any range determined empirically to be suitable for generating a population of polypeptides containing substantial proportions of antigen binding proteins. For example, polypeptides comprising variant CDRH3 can be generated having the sequence X1-X2-X3-X4-X5-(X6)ₙ-X7-X8-X9-D-Y (SEQ ID NO: 2658), wherein X1-X9 are amino acids encoded by restricted codon sets, and n is of various lengths, for example, n=3- 11, 5-11, or 7-11. Other examples of possible n values are 5, 6, 7, 8, 9, 10, and 11. Illustrative embodiments of oligonucleotides that can be utilized to provide for variety in CDRH3 sequence length include those shown in FIGS. 8A and 8B, FIGS. 12A-12D, FIGS. 19A-19L, and FIG. 27.

It is contemplated that the sequence diversity of libraries created by introduction of variant amino acids in a particular CDR, for example, CDRH3, can be increased by combining the variant CDR with other CDRs comprising variations in other regions of the antibody, specifically in other CDRs of either the light or heavy chain variable sequences. It is contemplated that the nucleic acid sequences that encode members of this set can be further diversified by introduction of other variant amino acids in the CDRs of either the light or heavy chain sequences, via codon sets. Thus, for example, in one embodiment, CDRH3 sequences from fusion polypeptides that bind a target antigen can be combined with diversified CDRL3, CDRH1, or CDRH2 sequences, or any combination of diversified CDRs.

It should be noted that in some instances framework residues may be varied relative to the sequence of a source antibody or antigen binding fragment, for example, to reflect a consensus sequence or to improve stability or display. For example, framework residues 49, 93, 94 or 71 in the heavy chain may be varied. Heavy chain framework residue 93 may be serine or alanine (which is the human consensus sequence amino acid at that position.) Heavy chain framework residue 94 may be changed to reflect framework consensus sequence from threonine to arginine or lysine. Another example of a framework residue that may be altered is heavy chain framework residue 71, which is R in about 1970 polypeptides, V in about 627 polypeptides and A in about 527 polypeptides, as found in the Kabat database. Heavy chain framework residue 49 may be alanine or glycine. In addition, optionally, the 3 N-terminal amino acids of the heavy chain variable domain can be removed. In the light chain, optionally, the arginine at amino acid position 66 can be changed to glycine. In one embodiment, heavy chain framework residue 93 is alanine and heavy chain framework residue 94 is arginine.

In one aspect, the invention provides vector constructs for generating fusion polypeptides that bind with significant affinity to potential ligands. These constructs comprise a dimerizable domain that when present in a fusion polypeptide provides for increased tendency for heavy chains to dimerize to form dimers of Fab or Fab′ antibody fragments/portions. These dimerization domains may include, e.g., a heavy chain hinge sequence (for example, a sequence comprising TCPPCPAPELLG (SEQ ID NO: 5) that may be present in the fusion polypeptide). Dimerization domains in fusion phage polypeptides bring two sets of fusion polypeptides (LC/HC-phage protein/fragment (such as pIII)) together, thus allowing formation of suitable linkages (such as interheavy chain disulfide bridges) between the two sets of fusion polypeptides. Vector constructs containing such dimerization domains can be used to achieve divalent display of antibody variable domains, for example the diversified fusion proteins described herein, on phage. In certain embodiments, the intrinsic affinity of each monomeric antibody fragment (fusion polypeptide) is not significantly altered by fusion to the dimerization domain. In certain embodiments, dimerization results in divalent phage display which provides increased avidity of pliage binding, with significant decrease in off-rate, which can be determined by methods known in the art and as described herein. Dimerization domain-containing vectors of the invention may or may not also include an amber stop codon after the dimerization domain.

Dimerization can be varied to achieve different display characteristics. Dimerization domains can comprise a sequence comprising a cysteine residue, a hinge region from a full-length antibody, a dimerization sequence such as leucine zipper sequence or GCN4 zipper sequence or mixtures thereof. Dimerization sequences are known in the art, and include, for example, the GCN4 zipper sequence (GRMKQLEDKVEELLSKNYHLENEVARLKKLVGERG) (SEQ ID NO: 3). The dimerization domain is in certain embodiments located at the C-terminal end of the heavy chain variable or constant domain sequence and/or between the heavy chain variable or constant domain sequence and any viral coat protein component sequence. An amber stop codon may also be present at or after the C-terminal end of the dimerization domain. In one embodiment, wherein an amber stop codon is present, the dimerization domain encodes at least one cysteine and a dimerizing sequence such as leucine zipper. In another embodiment, wherein no amber stop codon is present, the dimerization domain may comprise a single cysteine residue.

The polypeptides of the invention can also be fused to other types of polypeptides in order to provide for display of the variant polypeptides or to provide for purification, screening or sorting, and detection of the polypeptide. For embodiment involving phage display, the polypeptides of the invention are fused to all or a portion of a viral coat protein. Examples of viral coat protein include protein PIII, major coat protein, pVIII, Soc, Hoc, gpD, pVI and variants thereof. In addition, the variant polypeptides generated according to the methods of the invention can optionally be fused to a polypeptide marker or tag such as FLAG, polyhistidine, gD, c-myc, B-galactosidase and the like.

Methods of Generating Libraries of Randomized Variable Domains

Methods of substituting an amino acid of choice into a template nucleic acid are well established in the art, some of which are described herein. For example, libraries can be created by targeting solvent accessible and/or highly diverse positions in at least one CDR region for amino acid substitution with variant amino acids using the Kunkel method. See, for example, Kunkel et al., Methods Enzymol. (1987), 154:367-382. Generation of randomized sequences is also described below in the Examples.

The sequence of oligonucleotides includes one or more of the designed restricted codon sets for different lengths of CDRH3 or for the solvent accessible and highly diverse positions in a CDR. A codon set is a set of different nucleotide triplet sequences used to encode desired variant amino acids. Codon sets can be represented using symbols to designate particular nucleotides or equimolar mixtures of nucleotides as shown below according to the IUB code. Typically, a codon set is represented by three capital letters, e.g., KMT, TMT and the like.

IUB CODES

G Guanine

A Adenine

T Thymine

C Cytosine

R (A or G)

Y (C or T)

M (A or C)

K (G or T)

S (C or G)

W (A or T)

H (A or C or T)

B (C or G or T)

V (A or C or G)

D (A or G or T)

N (A or C or G or T)

For example, in the codon set TMT, T is the nucleotide thymine; and M can be A or C. This codon set can present multiple codons and can encode only a limited number of amino acids, namely tyrosine and serine.

Oligonucleotide or primer sets can be synthesized using standard methods. A set of oligonucleotides can be synthesized, for example, by solid phase synthesis, containing sequences that represent all possible combinations of nucleotide triplets provided by the restricted codon set and that will encode the desired restricted group of amino acids. Synthesis of oligonucleotides with selected nucleotide “degeneracy” at certain positions is well known in that art. Such sets of oligonucleotides having certain codon sets can be synthesized using commercial nucleic acid synthesizers (available from, for example, Applied Biosystems, Foster City, Calif.), or can be obtained commercially (for example, from Life Technologies, Rockville, Md.). Therefore, a set of oligonucleotides synthesized having a particular codon set will typically include a plurality of oligonucleotides with different sequences, the differences established by the codon set within the overall sequence. Oligonucleotides, as used according to the invention, have sequences that allow for hybridization to a CDR (for example, as contained within a variable domain) nucleic acid template and also can include restriction enzyme sites for cloning purposes.

In one method, nucleic acid sequences encoding variant amino acids can be created by oligonucleotide-mediated mutagenesis of a nucleic acid sequence encoding a source or template polypeptide such as the antibody variable domain of 4D5. This teclmique is well known in the art as described by Zoller et al. Nucleic Acids Res. 10:6487-6504(1987). Briefly, nucleic acid sequences encoding variant amino acids are created by hybridizing an oligonucleotide set encoding the desired restricted codon sets to a DNA template, where the template is the single-stranded form of the plasmid containing a variable region nucleic acid template sequence. After hybridization, DNA polymerase is used to synthesize an entire second complementary strand of the template that will thus incorporate the oligonucleotide primer, and will contain the restricted codon sets as provided by the oligomicleotide set. Nucleic acids encoding other source or template molecules are known or can be readily determined.

Generally, oligonucleotides of at least 25 nucleotides in length are used. An optimal oligonucleotide will have at least 12 to 15 nucleotides that are completely complementary to the template on either side of the nucleotide(s) coding for the mutation(s). This ensures that the oligonucleotide will hybridize properly to the single-stranded DNA template molecule. The oligonucleotides are readily synthesized using techniques known in the art such as that described by Crea et al., Proc. Natl. Acad. Sci. USA, 75:5765 (1978).

The DNA template is generated by those vectors that are either derived from bacteriophage M13 vectors (the commercially available M13mp18 and M13mp19 vectors are suitable), or those vectors that contain a single-stranded phage origin of replication as described by Viera et al., Meth. Enzymol., 153:3 (1987). Thus, the DNA that is to be mutated can be inserted into one of these vectors in order to generate single-stranded template. Production of the single-stranded template is described in sections 4.21-4.41 of Sambrook et al., above.

To alter the native DNA sequence, the oligonucleotide is hybridized to the single stranded template under suitable hybridization conditions. A DNA polymerizing enzyme, usually T7 DNA polymerase or the Klenow fragment of DNA polymerase I, is then added to synthesize the complementary strand of the template using the oligonucleotide as a primer for synthesis. A heteroduplex molecule is thus formed such that one strand of DNA encodes the mutated form of gene 1, and the other strand (the original template) encodes the native, unaltered sequence of gene 1. This heteroduplex molecule is then transformed into a suitable host cell, usually a prokaryote such as E. coli JM101. After growing the cells, they are plated onto agarose plates and screened using the oligonucleotide primer radiolabelled with a 32-Phosphate to identify the bacterial colonies that contain the mutated DNA.

The method described immediately above may be modified such that a homoduplex molecule is created wherein both strands of the plasmid contain the mutation(s). The modifications are as follows: The single stranded oligonucleotide is annealed to the single-stranded template as described above. A mixture of three deoxyribonucleotides, deoxyriboadenosine (dATP), deoxyriboguanosine (dGTP), and deoxyribothymidine (dTT), is combined with a modified thiodeoxyribocytosine called dCTP-(aS) (which can be obtained from Amersham). This mixture is added to the template-oligonucleotide complex. Upon addition of DNA polymerase to this mixture, a strand of DNA identical to the template except for the mutated bases is generated. In addition, this new strand of DNA will contain dCTP-(aS) instead of dCTP, which serves to protect it from restriction endonuclease digestion. After the template strand of the double-stranded heteroduplex is nicked with an appropriate restriction enzyme, the template strand can be digested with ExoIII nuclease or another appropriate nuclease past the region that contains the site(s) to be mutagenized. The reaction is then stopped to leave a molecule that is only partially single-stranded. A complete double-stranded DNA homoduplex is then formed using DNA polymerase in the presence of all four deoxyribonucleotide triphosphates, ATP, and DNA ligase. This homoduplex molecule can then be transformed into a suitable host cell.

As indicated previously the sequence of the oligonucleotide set is of sufficient length to hybridize to the template nucleic acid and may also, but does not necessarily, contain restriction sites. The DNA template can be generated by those vectors that are either derived from bacteriophage M13 vectors or vectors that contain a single-stranded phage origin of replication as described by Viera et al. ((1987) Meth. Enzymol., 153:3). Thus, the DNA that is to be mutated must be inserted into one of these vectors in order to generate single-stranded template. Production of the single-stranded template is described in sections 4.21-4.41 of Sambrook et al., supra.

According to another method, a library can be generated by providing upstream and downstream oligonucleotide sets, each set having a plurality of oligonucleotides with different sequences, the different sequences established by the codon sets provided within the sequence of the oligonucleotides. The upstream and downstream oligonucleotide sets, along with a variable domain template nucleic acid sequence, can be used in a polymerase chain reaction to generate a “library” of PCR products. The PCR products can be referred to as “nucleic acid cassettes”, as they can be fused with other related or unrelated nucleic acid sequences, for example, viral coat protein components and dimerization domains, using established molecular biology techniques.

The sequence of the PCR primers includes one or more of the designed codon sets for the solvent accessible and highly diverse positions in a CDR region. As described above, a codon set is a set of different nucleotide triplet sequences used to encode desired variant amino acids.

Oligonucleotide sets can be used in a polymerase chain reaction using a variable region nucleic acid template sequence as the template to create nucleic acid cassettes. The variable region nucleic acid template sequence can be any portion of the light or heavy immunoglobulin chains containing the target nucleic acid sequences (i.e., nucleic acid sequences encoding amino acids targeted for substitution). The variable region nucleic acid template sequence is a portion of a double stranded DNA molecule having a first nucleic acid strand and complementary second nucleic acid strand. The variable region nucleic acid template sequence contains at least a portion of a variable domain and has at least one CDR. In some cases, the variable region nucleic acid template sequence contains more than one CDR. An upstream portion and a downstream portion of the variable region nucleic acid template sequence can be targeted for hybridization with members of an upstream oligonucleotide set and a downstream oligonucleotide set.

A first oligonucleotide of the upstream primer set can hybridize to the first nucleic acid strand and a second oligonucleotide of the downstream primer set can hybridize to the second nucleic acid strand. The oligonucleotide primers can include one or more codon sets and be designed to hybridize to a portion of the variable region nucleic acid template sequence. Use of these oligonucleotides can introduce two or more codon sets into the PCR product (i.e., the nucleic acid cassette) following PCR. The oligonucleotide primer that hybridizes to regions of the nucleic acid sequence encoding the antibody variable domain includes portions that encode CDR residues that are targeted for amino acid substitution.

The upstream and downstream oligonucleotide sets can also be synthesized to include restriction sites within the oligonucleotide sequence. These restriction sites can facilitate the insertion of the nucleic acid cassettes [i.e., PCR reaction products] into an expression vector having additional antibody sequences. In certain embodiments, the restriction sites are designed to facilitate the cloning of the nucleic acid cassettes without introducing extraneous nucleic acid sequences or removing original CDR or framework nucleic acid sequences.

Nucleic acid cassettes can be cloned into any suitable vector for expression of a portion or the entire light or heavy chain sequence containing the targeted amino acid substitutions generated. According to methods detailed in the invention, the nucleic acid cassette is cloned into a vector allowing production of a portion or the entire light or heavy chain sequence fused to all or a portion of a viral coat protein (i.e., creating a fusion protein) and displayed on the surface of a particle or cell. While several types of vectors are available and may be used to practice this invention, phagemid vectors are convenient, as they may be constructed with relative ease, and can be readily amplified. Phagemid vectors generally contain a variety of components including promoters, signal sequences, phenotypic selection genes, origin of replication sites, and other necessary components as are known to those of ordinary skill in the art.

In another embodiment, wherein a particular variant amino acid combination is to be expressed, the nucleic acid cassette contains a sequence that is able to encode all or a portion of the heavy or light chain variable domain, and is able to encode the variant amino acid combinations. For production of antibodies containing these variant amino acids or combinations of variant amino acids, as in a library, the nucleic acid cassettes can be inserted into an expression vector containing additional antibody sequence, for example all or portions of the variable or constant domains of the light and heavy chain variable regions. These additional antibody sequences can also be fused to other nucleic acid sequences, such as sequences which encode viral coat protein components and therefore allow production of a fusion protein.

Vectors

One aspect of the invention includes a replicable expression vector comprising a nucleic acid sequence encoding a gene fusion, wherein the gene fusion encodes a fusion protein comprising a CDR-containing polypeptide (such as an antibody variable domain), or an antibody variable domain and a constant domain, fused to all or a portion of a viral coat protein. Also included is a library of diverse replicable expression vectors comprising a plurality of gene fusions encoding a plurality of different fusion proteins including a plurality of the fusion polypeptides generated with diverse sequences as described above. The vectors can include a variety of components and may be constructed to allow for movement of antibody variable domain between different vectors and /or to provide for display of the fusion proteins in different formats.

Examples of vectors include phage vectors and phagemid vectors (which illustrated herein, and described in greater detail above). A phage vector generally has a phage origin of replication allowing phage replication and phage particle formation. The phage is generally a filamentous bacteriophage, such as an M13, f1, fd, Pf3 phage or a derivative thereof, or a lambdoid phage, such as lambda, 21, phi80, phi81, 82, 424, 434, etc., or a derivative thereof.

Examples of viral coat proteins include infectivity protein PIII (sometimes also designated p3), major coat protein PVIII, Soc (T4), Hoc (T4), gpD (of bacteriophage lambda), minor bacteriophage coat protein 6 (pVI) (filamentous phage; J Immunol Methods. Dec. 10, 1999;231(1-2):39-51), variants of the M13 bacteriophage major coat protein (P8) (Protein Sci 2000 April;9(4):647-54). The fusion protein can be displayed on the surface of a phage and suitable phage systems include M13KO7 helper phage, M13R408, M13-VCS, and Phi X 174, pJuFo phage system (J Virol. 2001 August;75(15):7107-13.v), hyperphage (Nat Biotechnol. 2001 January; 19(1):75-8). In certain embodiments, the helper phage is M13KO7, and the coat protein is the M13 Phage gene III coat protein. In certain embodiments, the host is E. coli, and protease deficient strains of E. coli. Vectors, such as the fth1 vector (Nucleic Acids Res. 2001 May 15;29(10):E50-0) can be useful for the expression of the fusion protein.

The expression vector also can have a secretory signal sequence fused to the DNA encoding a CDR-containing fusion polypeptide (e.g., each subunit of an antibody, or fragment thereof). This sequence is typically located immediately 5′ to the gene encoding the fusion protein, and will thus be transcribed at the amino terminus of the fusion protein. However, in certain cases, the signal sequence has been demonstrated to be located at positions other than 5′ to the gene encoding the protein to be secreted. This sequence targets the protein to which it is attached across the inner membrane of the bacterial cell. The DNA encoding the signal sequence may be obtained as a restriction endonuclease fragment from any gene encoding a protein that has a signal sequence. Suitable prokaryotic signal sequences may be obtained from genes encoding, for example, LamB or OmpF (Wong et al., Gene, 68:1931 (1983), MalE, PhoA and other genes. In one embodiment, a prokaryotic signal sequence for practicing this invention is the E. coli heat-stable enterotoxin II (STII) signal sequence as described by Chang et al., Gene 55:189 (1987), and/or malE.

As indicated above, a vector also typically includes a promoter to drive expression of the fusion polypeptide. Promoters most commonly used in prokaryotic vectors include the lac Z promoter system, the alkaline phosphatase pho A promoter (Ap), the bacteriophage l_{PL} promoter (a temperature sensitive promoter), the tac promoter (a hybrid trp-lac promoter that is regulated by the lac repressor), the tryptophan promoter, and the bacteriophage T7 promoter. For general descriptions of promoters, see section 17 of Sambrook et al. supra. While these are the most commonly used promoters, other suitable promoters may be used as well.

The vector can also include other nucleic acid sequences, for example, sequences encoding gD tags, c-Myc epitopes, poly-histidine tags, fluorescence proteins (e.g., GFP), or beta-galactosidase protein which can be useful for detection or purification of the fusion protein expressed on the surface of the phage or cell. Nucleic acid sequences encoding, for example, a gD tag, also provide for positive or negative selection of cells or virus expressing the fusion protein. In some embodiments, the gD tag is fused to an antibody variable domain which is not fused to the viral coat protein component. Nucleic acid sequences encoding, for example, a polyhistidine tag, are useful for identifying fusion proteins including antibody variable domains that bind to a specific antigen using immunohistochemistry. Tags useful for detection of antigen binding can be fused to either an antibody variable domain not fused to a viral coat protein component or an antibody variable domain fused to a viral coat protein component.

Another useful component of the vectors used to practice this invention is phenotypic selection genes. Typical phenotypic selection genes are those encoding proteins that confer antibiotic resistance upon the host cell. By way of illustration, the ampicillin resistance gene (ampr), and the tetracycline resistance gene (tetr) are readily employed for this purpose.

The vector can also include nucleic acid sequences containing unique restriction sites and suppressible stop codons. The unique restriction sites are useful for moving antibody variable domains between different vectors and expression systems, especially useful for production of full-length antibodies or antigen binding fragments in cell cultures. The suppressible stop codons are useful to control the level of expression of the fusion protein and to facilitate purification of soluble antibody fragments. For example, an amber stop codon can be read as Gln in a supE host to enable phage display, while in a non-supE host it is read as a stop codon to produce soluble antibody fragments without fusion to phage coat proteins. These synthetic sequences can be fused to one or more antibody variable domains in the vector.

It is sometimes beneficial to use vector systems that allow the nucleic acid encoding an antibody sequence of interest, for example a CDR having variant amino acids, to be easily removed from the vector system and placed into another vector system. For example, appropriate restriction sites can be engineered in a vector system to facilitate the removal of the nucleic acid sequence encoding an antibody or antibody variable domain having variant amino acids. The restriction sequences are usually chosen to be unique in the vectors to facilitate efficient excision and ligation into new vectors. Antibodies or antibody variable domains can then be expressed from vectors without extraneous fusion sequences, such as viral coat proteins or other sequence tags.

Between nucleic acid encoding antibody variable or constant domain (gene 1) and the viral coat protein component (gene 2), DNA encoding a termination or stop codon may be inserted, such termination codons including UAG (amber), UAA (ocher) and UGA (opel). (Microbiology, Davis et al., Harper & Row, New York, 1980, pp. 237, 245-47 and 374). The termination or stop codon expressed in a wild type host cell results in the synthesis of the gene 1 protein product without the gene 2 protein attached. However, growth in a suppressor host cell results in the synthesis of detectable quantities of fused protein. Such suppressor host cells are well known and described, such as E. coli suppressor strain (Bullock et al., BioTechniques 5:376-379 (1987)). Any acceptable method may be used to place such a termination codon into the mRNA encoding the fusion polypeptide.

The suppressible codon may be inserted between the first gene encoding an antibody variable or constant domain, and a second gene encoding at least a portion of a phage coat protein. Alternatively, the suppressible termination codon may be inserted adjacent to the fusion site by replacing the last amino acid triplet in the antibody variable domain or the first amino acid in the phage coat protein. The suppressible termination codon may be located at or after the C-terminal end of a dimerization domain. When the plasmid containing the suppressible codon is grown in a suppressor host cell, it results in the detectable production of a fusion polypeptide containing the polypeptide and the coat protein. When the plasmid is grown in a non-suppressor host cell, the antibody variable domain is synthesized substantially without fusion to the phage coat protein due to termination at the inserted suppressible triplet UAG, UAA, or UGA. In the non-suppressor cell the antibody variable domain is synthesized and secreted from the host cell due to the absence of the fused phage coat protein which otherwise anchored it to the host membrane.

In some embodiments, the CDR being diversified (randomized) may have a stop codon engineered in the template sequence (referred to herein as a “stop template”). This feature provides for detection and selection of successfully diversified sequences based on successful repair of the stop codon(s) in the template sequence due to incorporation of the oligonucleotide(s) comprising the sequence(s) for the variant amino acids of interest. This feature is further illustrated in the Examples below.

The light and/or heavy chain antibody variable or constant domains can also be fused to an additional peptide sequence, the additional peptide sequence providing for the interaction of one or more fusion polypeptides on the surface of the viral particle or cell. These peptide sequences are herein referred to as “dimerization domains”. Dimerization domains may comprise at least one or more of a dimerization sequence, or at least one sequence comprising a cysteine residue or both. Suitable dimerization sequences include those of proteins having amphipathic alpha helices in which hydrophobic residues are regularly spaced and allow the formation of a dimer by interaction of the hydrophobic residues of each protein; such proteins and portions of proteins include, for example, leucine zipper regions. Dimerization domains can also comprise one or more cysteine residues (e.g. as provided by inclusion of an antibody hinge sequence within the dimerization domain). The cysteine residues can provide for dimerization by formation of one or more disulfide bonds. In one embodiment, wherein a stop codon is present after the dimerization domain, the dimerization domain comprises at least one cysteine residue. In some embodiments, the dimerization domains are located between the antibody variable or constant domain and the viral coat protein component.

In some cases the vector encodes a single antibody-phage polypeptide in a single chain form containing, for example, both the heavy and light chain variable regions fused to a coat protein. In these cases the vector is considered to be “monocistronic”, expressing one transcript under the control of a certain promoter. For example, a vector may utilize a promoter (such as the alkaline phosphatase (AP) or Tac promoter) to drive expression of a monocistronic sequence encoding VL and VH domains, with a linker peptide between the VL and VH domains. This cistronic sequence may be connected at the 5′ end to a signal sequence (such as an E. coli malE or heat-stable enterotoxin II (STII) signal sequence) and at its 3′ end to all or a portion of a viral coat protein (such as the bacteriophage pIII protein). The fusion polypeptide encoded by a vector of this embodiment is referred to herein as “ScFv-pIII”. In some embodiments, a vector may further comprise a sequence encoding a dimerization domain (such as a leucine zipper) at its 3′ end, between the second variable domain sequence (e.g., VH) and the viral coat protein sequence. Fusion polypeptides comprising the dimerization domain are capable of dimerizing to form a complex of two scFv polypeptides (referred to herein as “(ScFv)2-pIII)”).

In other cases, the variable regions of the heavy and light chains can be expressed as separate polypeptides, the vector thus being “bicistronic”, allowing the expression of separate transcripts. In these vectors, a suitable promoter, such as the Ptac or PhoA promoter, is used to drive expression of a bicistronic message. A first cistron encoding, for example, a light chain variable and constant domain, may be connected at the 5′ end to a signal sequence, such as E. coli malE or heat-stable enterotoxin II (STII) signal sequence, and at the 3′ end to a nucleic acid sequence encoding a tag sequence, such as gD tag. A second cistron, encoding, for example, a heavy chain variable domain and constant domain CH1, is connected at its 5′ end to a signal sequence, such as E. coli malE or heat-stable enterotoxin II (STII) signal sequence, and at the 3′ end to all or a portion of a viral coat protein.

In one embodiment of a vector which provides a bicistronic message and for display of F(ab′)₂-pIII, a suitable promoter, such as Ptac or PhoA (AP) promoter, drives expression of a first cistron encoding a light chain variable and constant domain operably linked at 5′ end to a signal sequence such as the E. coli malE or heat stable enteroxtoxin II (STII) signal sequence, and at the 3′ end to a nucleic acid sequence encoding a tag sequence such as gD tag. The second cistron encodes, for example, a heavy chain variable and constant domain operatively linked at 5′ end to a signal sequence such as E. coli malE or heat stable enterotoxin II (STII) signal sequence, and at 3′ end has a dimerization domain comprising IgG hinge sequence and a leucine zipper sequence followed by at least a portion of viral coat protein.

Display of Fusion Polypeptides

Fusion polypeptides of a CDR-containing polypeptide (for example, an antibody variable domain) can be displayed on the surface of a cell, virus, or phagemid particle in a variety of formats. These formats include single chain Fv fragment (scFv), F(ab) fragment and multivalent forms of these fragments. For example, multivalent forms include a dimer of ScFv, Fab, or F(ab′), herein referred to as (ScFv)₂, F(ab)₂ and F(ab′)₂, respectively. The multivalent forms of display are advantageous in some contexts in part because they have more than one antigen binding site which generally results in the identification of lower affinity clones and also allows for more efficient sorting of rare clones during the selection process.

Methods for displaying fusion polypeptides comprising antibody fragments, on the surface of bacteriophage, are well known in the art, for example as described in patent publication number WO 92/01047 and herein. Other patent publications WO 92/20791; WO 93/06213; WO 93/11236 and WO 93/19172, describe related methods and are all herein incorporated by reference. Other publications have shown the identification of antibodies with artificially rearranged V gene repertoires against a variety of antigens displayed on the surface of phage (for example, H. R. Hoogenboom & G. Winter, J. Mol. Biol. 227 381-388 (1992); and as disclosed in WO 93/06213 and WO 93/11236).

When a vector is constructed for display in a scFv format, it includes nucleic acid sequences encoding an antibody variable light chain domain and an antibody variable heavy chain variable domain. Typically, the nucleic acid sequence encoding an antibody variable heavy chain domain is fused to a viral coat protein component. One or both of the antibody variable domains can have variant amino acids in at least one CDR region. The nucleic acid sequence encoding the antibody variable light chain is connected to the antibody variable heavy chain domain by a nucleic acid sequence encoding a peptide linker. The peptide linker typically contains about 5 to 15 amino acids. Optionally, other sequences encoding, for example, tags useful for purification or detection can be fused at the 3′ end of either the nucleic acid sequence encoding the antibody variable light chain or antibody variable heavy chain domain or both.

When a vector is constructed for F(ab) display, it includes nucleic acid sequences encoding antibody variable domains and antibody constant domains. A nucleic acid encoding a variable light chain domain is fused to a nucleic acid sequence encoding a light chain constant domain. A nucleic acid sequence encoding an antibody heavy chain variable domain is fused to a nucleic acid sequence encoding a heavy chain constant CH1 domain. Typically, the nucleic acid sequence encoding the heavy chain variable and constant domains are fused to a nucleic acid sequence encoding all or part of a viral coat protein. One or both of the antibody variable light or heavy chain domains can have variant amino acids in at least one CDR. In some embodiments, the heavy chain variable and constant domains are expressed as a fusion with at least a portion of a viral coat protein, and the light chain variable and constant domains are expressed separately from the heavy chain viral coat fusion protein. The heavy and light chains associate with one another, which may be by covalent or non-covalent bonds. Optionally, other sequences encoding, for example, polypeptide tags useful for purification or detection, can be fused at the 3′ end of either the nucleic acid sequence encoding the antibody light chain constant domain or antibody heavy chain constant domain or both.

In some embodiments, a bivalent moiety, for example, a F(ab)₂ dimer or F(ab′)₂ dimer, is used for displaying antibody fragments with the variant amino acid substitutions on the surface of a particle. It has been found that F(ab′)₂ dimers generally have the same affinity as F(ab) dimers in a solution phase antigen binding assay but the off rate for F(ab′)₂ are reduced because of a higher avidity. Therefore, the bivalent format (for example, F(ab′)₂) is a particularly useful format since it can allow for the identification of lower affinity clones and also allows more efficient sorting of rare clones during the selection process.

Introduction of Vectors into Host Cells

Vectors constructed as described in accordance with the invention are introduced into a host cell for amplification and/or expression. Vectors can be introduced into host cells using standard transformation methods including electroporation, calcium phosphate precipitation and the like. If the vector is an infectious particle such as a virus, the vector itself provides for entry into the host cell. Transfection of host cells containing a replicable expression vector which encodes the gene fusion and production of phage particles according to standard procedures provides phage particles in which the fusion protein is displayed on the surface of the phage particle.

Replicable expression vectors are introduced into host cells using a variety of methods. In one embodiment, vectors can be introduced into cells using electroporation as described in WO/00106717. Cells are grown in culture in standard culture broth, optionally for about 6-48 hours (or to OD₆₀₀=0.6-0.8) at about 37° C., and then the broth is centrifuged and the supernatant removed (e.g. decanted). In some embodiments, initial purification includes resuspending the cell pellet in a buffer solution (e.g. 1.0 mM HEPES pH 7.4) followed by recentrifugation and removal of supernatant. The resulting cell pellet is resuspended in dilute glycerol (e.g. 5-20% v/v) and again recentrifuged to form a cell pellet and the supernatant removed. The final cell concentration is obtained by resuspending the cell pellet in water or dilute glycerol to the desired concentration.

In certain embodiments, the recipient cell is the electroporation competent E. coli strain of the present invention, which is E. coli strain SS320 (Sidhu et al., Methods Enzymol. (2000), 328:333-363). Strain SS320 was prepared by mating MC1061 cells with XL1-BLUE cells under conditions sufficient to transfer the fertility episome (F′ plasmid) or XL1-BLUE into the MC1061 cells. Strain SS320 has been deposited with the American Type Culture Collection (ATCC), 10801 University Boulevard, Manassas, Va. USA, on Jun. 18, 1998 and assigned Deposit Accession No. 98795. Any F′ episome which enables phage replication in the strain may be used in the invention. Suitable episomes are available from strains deposited with ATCC or are commercially available (CJ236, CSH18, DHF′, JM101, JM103, JM105, JM107, JM109, JM110), KS1000, XL1-BLUE, 71-18 and others).

The use of higher DNA concentrations during electroporation (about 10×) increases the transformation efficiency and increases the amount of DNA transformed into the host cells. The use of high cell concentrations also increases the efficiency (about 10×). The larger amount of transferred DNA produces larger libraries having greater diversity and representing a greater number of unique members of a combinatorial library. Transformed cells are generally selected by growth on antibiotic containing medium.

Selection (Sorting) and Screening for Binders to Targets of Choice

Use of phage display for identifying target antigen binders, with its various permutations and variations in methodology, are well established in the art. One approach involves constructing a family of variant replicable vectors containing a transcription regulatory element operably linked to a gene fusion encoding a fusion polypeptide, transforming suitable host cells, culturing the transformed cells to form phage particles which display the fusion polypeptide on the surface of the phage particle, followed by a process that entails selection or sorting by contacting the recombinant phage particles with a target antigen so that at least a portion of the population of particles bind to the target with the objective to increase and enrich the subsets of the particles which bind from particles relative to particles that do not bind in the process of selection. The selected pool can be amplified by infecting host cells, such as fresh XL1-Blue cells, for another round of sorting on the same target with different or same stringency. The resulting pool of variants are then screened against the target antigens to identify novel high affinity binding proteins. These novel high affinity binding proteins can be useful as therapeutic agents as antagonists or agonists, and/or as diagnostic and research reagents.

Fusion polypeptides such as antibody variable domains comprising the variant amino acids can be expressed on the surface of a phage, phagemid particle or a cell and then selected and/or screened for the ability of members of the group of fusion polypeptides to bind a target antigen which is typically an antigen of interest. The processes of selection for binders to target can also be include sorting on a generic protein having affinity for antibody variable domains such as protein L or a tag specific antibody which binds to antibody or antibody fragments displayed on phage, which can be used to enrich for library members that display correctly folded antibody fragments (fusion polypeptides).

Target proteins, such as receptors, may be isolated from natural sources or prepared by recombinant methods by procedures known in the art. Target antigens can include a number of molecules of therapeutic interest.

A variety of strategies of selection (sorting) for affinity can be used. One example is a solid-support method or plate sorting or immobilized target sorting. Another example is a solution-binding method.

For the solid support method, the target protein may be attached to a suitable solid or semi solid matrix. Such matrices are known in the art, such as agarose beads, acrylamide beads, glass beads, cellulose, various acrylic copolymers, hydroxyalkyl methacrylate gels, polyacrylic and polymethacrylic copolymers, nylon, neutral and ionic carriers, and the like. Attachment of the target protein to the matrix may be accomplished by methods described, e.g., in Methods in Enzymology, 44 (1976), or by other means known in the art.

After attachment of the target antigen to the matrix, the immobilized target is contacted with the library expressing the fusion polypeptides under conditions suitable for binding of at least a subset of the phage particle population with the immobilized target antigen. Normally, the conditions, including pH, ionic strength, temperature and the like will mimic physiological conditions. Bound particles (“binders”) to the immobilized target are separated from those particles that do not bind to the target by washing. Wash conditions can be adjusted to result in removal of all but the high affinity binders. Binders may be dissociated from the immobilized target by a variety of methods. These methods include competitive dissociation using the wild-type ligand (e.g. excess target antigen), altering pH and/or ionic strength, and methods known in the art. Selection of binders typically involves elution from an affinity matrix with a suitable elution material such as acid like 0.1M HCl or ligand. Elution with increasing concentrations of ligand could elute displayed binding molecules of increasing affinity.

The binders can be isolated and then re-amplified in suitable host cells by infecting the cells with the viral particles that are binders (and helper phage if necessary, e.g., when the viral particle is a phagemid particle) and the host cells are cultured under conditions suitable for amplification of the particles that display the desired fusion polypeptide. The phage particles are then collected and the selection process is repeated one or more times until binders of the target antigen are enriched. Any number of rounds of selection or sorting can be utilized. One of the selection or sorting procedures can involve isolating binders that bind to a generic affinity protein such as protein L or an antibody to a polypeptide tag present in a displayed polypeptide such as antibody to the gD protein or polyhistidine tag. Counterselection may be included in one or more rounds of selection or sorting to isolate binders that also exhibit undesired binding to one or more non-target antigens.

One aspect of the invention involves selection against libraries of the invention using a novel selection method which is termed “solution-binding method”. The invention allows solution phase sorting with much improved efficiency over conventional solution sorting methods. The solution binding method may be used for finding original binders from a random library or finding improved binders from a library that was designated to improve affinity of a particular binding clone or group of clones. The method comprises contacting a plurality of polypeptides, such as those displayed on phage or phagemid particles (library), with a target antigen labeled or fused with a tag molecule. The tag could be biotin or other moieties for which specific binders are available. The stringency of the solution phase can be varied by using decreasing concentrations of labeled target antigen in the first solution binding phase. To further increase the stringency, the first solution binding phase can be followed by a second solution phase having high concentration of unlabelled target antigen after the initial binding with the labeled target in the first solution phase. Usually, 100 to 1000 fold of unlabelled target over labeled target is used in the second phase (if included). The length of time of incubation of the first solution phase can vary from a few minutes to one to two hours or longer to reach equilibrium. Using a shorter time for binding in this first phase may bias or select for binders that have fast on-rate. The length of time and temperature of incubation in second phase can be varied to increase the stringency. This provides for a selection bias for binders that have slow rate of coming off the target (off-rate). After contacting the plurality of polypeptides (displayed on the phage/phagemid particles) with a target antigen, the phage or phagemid particles that are bound to labeled targets are separated from phage that do not bind. The particle-target mixture from solution phase of binding is isolated by contacting it with the labeled target moiety and allowing for its binding to, a molecule that binds the labeled target moiety for a short period of time (e.g., 2-5 minutes). The initial concentration of the labeled target antigen can range from about 0.1 nM to about 1000 nM. The bound particles are eluted and can be propagated for next round of sorting. In certain embodiments, multiple rounds of sorting are performed using a lower concentration of labeled target antigen with each round of sorting.

For example, an initial sort or selection using about 100 to 250 nM labeled target antigen should be sufficient to capture a wide range of affinities, although this factor can be determined empirically and/or to suit the desire of the practitioner. In the second round of selection, about 25 to 100 nM of labeled target antigen may be used. In the third round of selection, about 0.1 to 25 nM of labeled target antigen may be used. For example, to improve the affinity of a 100 nM binder, it may be desirable to start with 20 nM and then progress to 5 and 1 nM labeled target, then, followed by even lower concentrations such as about 0.1 nM labeled target antigen.

The conventional solution sorting involves use of beads like streptavidin-coated beads, which is very cumbersome to use and often results in very low efficiency of phage binder recovery. The conventional solution sorting with beads takes much longer than 2-5 minutes and is less feasible to adapt to high throughput automation than the invention described above.

As described herein, combinations of solid support and solution sorting methods can be advantageously used to isolate binders having desired characteristics. After selection/sorting on target antigen for a few rounds, screening of individual clones from the selected pool generally is performed to identify specific binders with the desired properties/ characteristics. In some embodiments, the process of screening is carried out by automated systems to allow for high-throughput screening of library candidates.

Two major screening methods are described below. However, other methods known in the art may also be used in the methods of the invention. The first screening method comprises a phage ELISA assay with immobilized target antigen, which provides for identification of a specific binding clone from a non-binding clone. Specificity can be determined by simultaneous assay of the clone on target coated well and BSA or other non-target protein coated wells. This assay is automatable for high throughput screening.

One embodiment provides a method of selecting for an antibody variable domain that binds to a specific target antigen from a library of antibody variable domain by generating a library of replicable expression vectors comprising a plurality of polypeptides; contacting the library with a target antigen and at least one nontarget antigen under conditions suitable for binding; separating the polypeptide binders in the library from the nonbinders; identifying the binders that bind to the target antigen and do not bind to the nontarget antigen; eluting the binders from the target antigen; and amplifying the replicable expression vectors comprising the polypeptide binder that bind to a specific antigen.

The second screening assay is an affinity screening assay that provides for screening for clones that have high affinity from clones that have low affinity in a high throughput manner. In the assay, each clone is assayed with and without first incubating with target antigen of certain concentration for a period of time (e.g., 30-60 minutes) before application to target coated wells briefly (e.g., 5-15 minutes). Then bound phage is measured by usual phage ELISA method, e.g. using anti-M13 HRP conjugates. The ratio of binding signal of the two wells, one well having been preincubated with target and the other well not preincubated with target antigen is an indication of affinity. The selection of the concentration of target for first incubation depends on the affinity range of interest. For example, if binders with affinity higher than 10 nM are desired, 100 nM of target in the first incubation is often used. Once binders are found from a particular round of sorting (selection), these clones can be screened with an affinity screening assay to identify binders with higher affinity.

Combinations of any of the sorting/selection methods described above may be combined with the screening methods. For example, in one embodiment, polypeptide binders are first selected for binding to immobilized target antigen. Polypeptide binders that bind to the immobilized target antigen can then be amplified and screened for binding to the target antigen and for lack of binding to nontarget antigens. Polypeptide binders that bind specifically to the target antigen are amplified. These polypeptide binders can then selected for higher affinity by contact with a concentration of a labeled target antigen to form a complex, wherein the concentration ranges of labeled target antigen from about 0.1 nM to about 1000 nM, the complexes are isolated by contact with an agent that binds to the label on the target antigen. The polypeptide binders are then elited from the labeled target antigen and optionally, the rounds of selection are repeated, each time a lower concentration of labeled target antigen is used. The high affinity polypeptide binders isolated using this selection method can then be screened for high affinity using a variety of methods known in the art, some of which are described herein.

These methods can provide for finding clones with high affinity without having to perform long and complex competition affinity assays on a large number of clones. The intensive aspect of doing complex assays of many clones often is a significant obstacle to finding best clones from a selection. This method is especially useful in affinity improvement efforts where multiple binders with similar affinity can be recovered from the selection process. Different clones may have very different efficiency of expression/display on phage or phagemid particles. Those clones more highly expressed have better chances being recovered. That is, the selection can be biased by the display or expression level of the variants. The solution-binding sorting method of the invention can improve the selection process for finding binders with high affinity. This method is an affinity screening assay that provides a significant advantage in screening for the best binders quickly and easily.

After binders are identified by binding to the target antigen, the nucleic acid can be extracted. Extracted DNA can then be used directly to transform E. coli host cells or alternatively, the encoding sequences can be amplified, for example using PCR with suitable primers, and sequenced by any typical sequencing method. Variable domain DNA of the binders can be restriction enzyme digested and then inserted into a vector for protein expression.

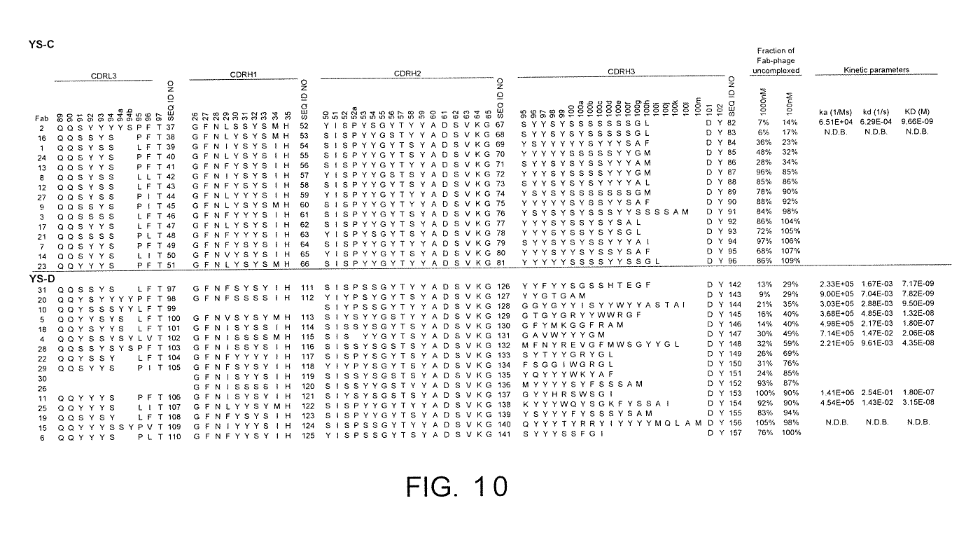

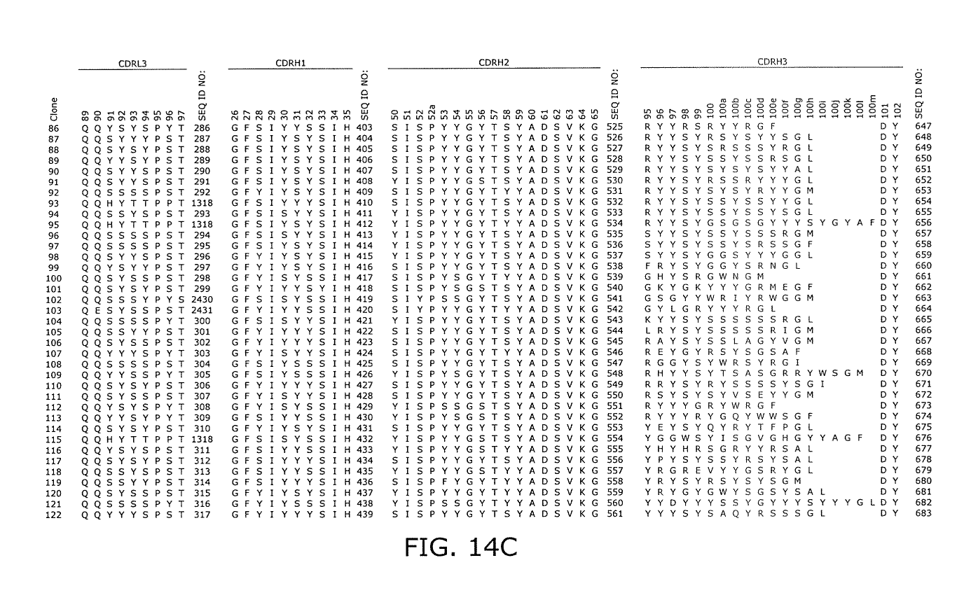

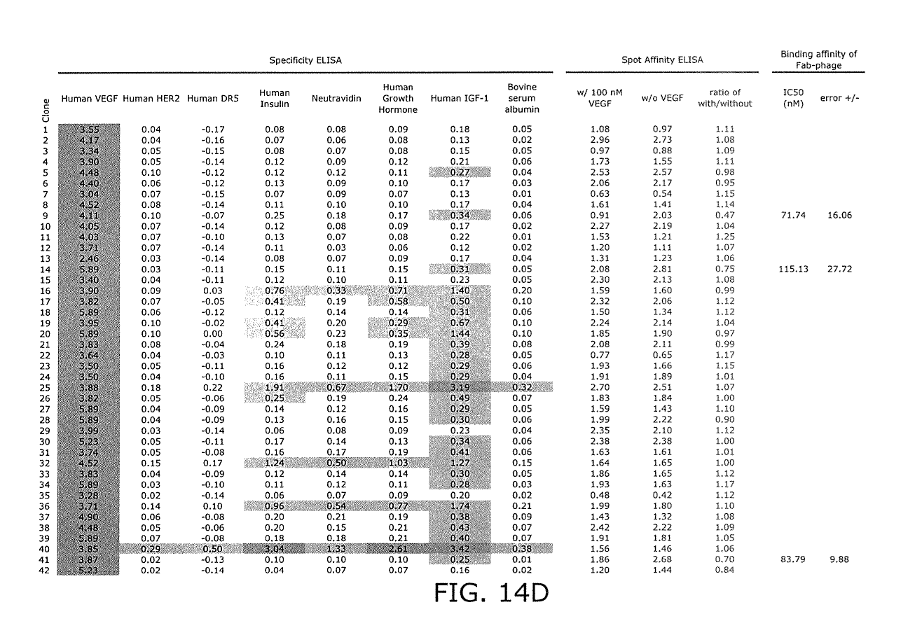

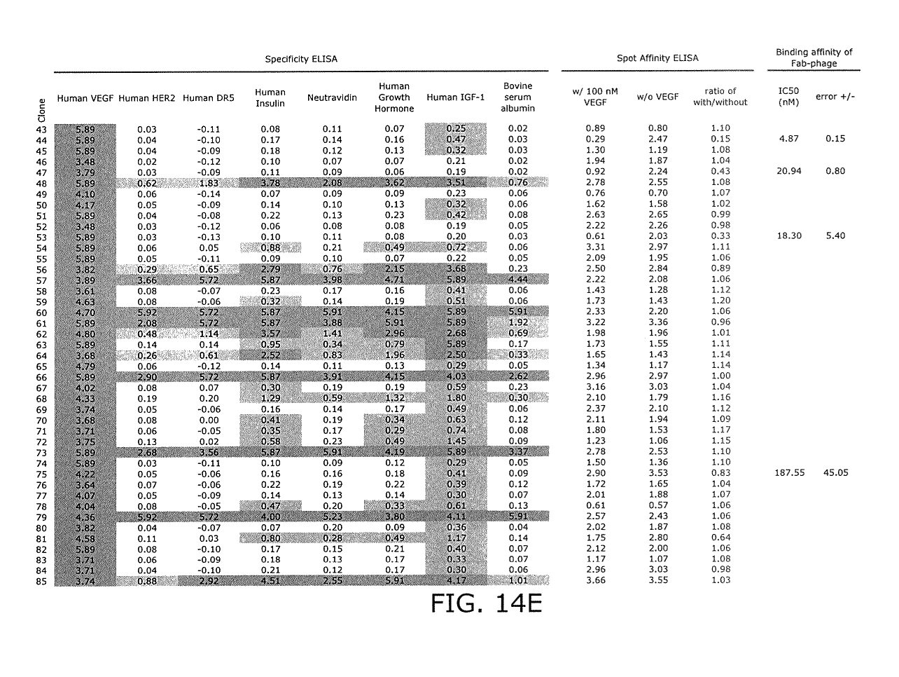

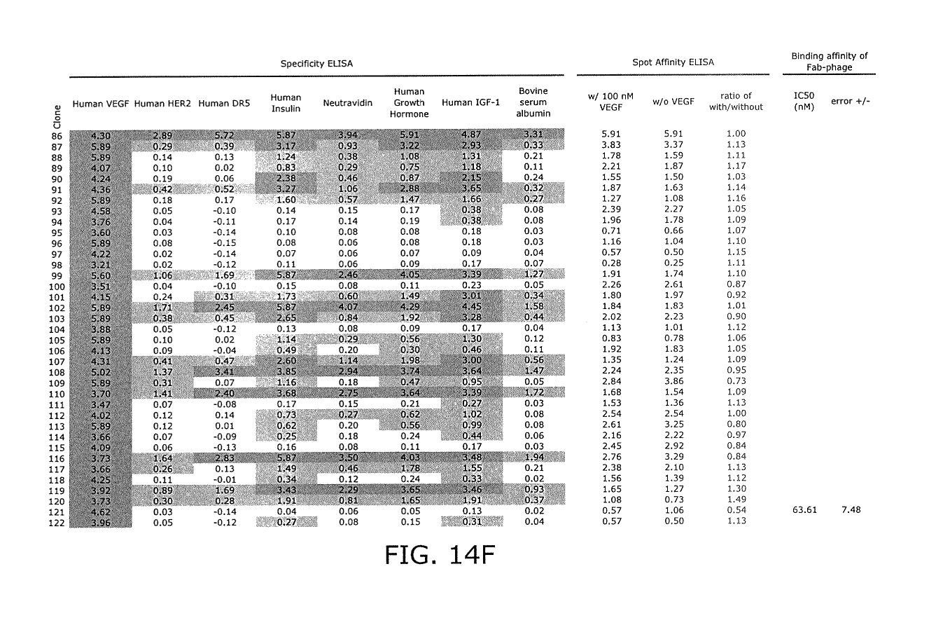

Populations comprising polypeptides having CDR(s) with restricted sequence diversity generated according to methods of the invention can be used to isolate binders against a variety of targets, including those listed in FIGS. 10, 14A-C, 15A-B, 21-25A, and 28-32A. These binders may comprise one or more variant CDRs comprising diverse sequences generated using restricted codons. In some embodiments, a variant CDR is CDRH3 comprising sequence diversity generated by amino acid substitution with restricted codon sets and/or amino acid insertions resulting from varying CDRH3 lengths. Illustrative oligonucleotides useful for generating fusion polypeptides of the invention include those listed in FIGS. 8A and 8B, 12A-12D, 19A-L, and 27. One or more variant CDRs may be combined. In some embodiments, only CDRH3 is diversified. In other embodiments, two or more heavy chain CDRs, including CDRH3, are variant. In other embodiments, one or more heavy chain CDRs, excluding CDRH3, are variant. In some embodiments, at least one heavy chain and at least one light chain CDR are variant. In some embodiments, at least one, two, three, four, five or all of CDRs H1, H2, H3, L1, L2 and L3 are variant.

In some cases, it can be beneficial to combine one or more diversified light chain CDRs with novel binders isolated from a population of polypeptides comprising one or more diversified heavy chain CDRs. This process may be referred to as a 2-step process. An example of a 2-step process comprises first determining binders (generally lower affinity binders) within one or more libraries generated by randomizing one or more CDRs, wherein the CDRs randomized in each library are different or, where the same CDR is randomized, it is randomized to generate different sequences. Binders from a heavy chain library can then be randomized with CDR diversity in a light chain CDRs by, for example, a mutagenesis technique such as that of Kunkel, or by cloning (cut-and-paste (e.g. by ligating different CDR sequences together)) the new light chain library into the existing heavy chain binders that has only a fixed light chain. The pool can then be further sorted against one or more targets to identify binders possessing increased affinity. For example, binders (for example, low affinity binders) obtained from sorting an H1/H2/H3 may be fused with library of an L1/L2/L3 diversity to replace its original fixed L1/L2/L3, wherein the new libraries are then further sorted against a target of interest to obtain another set of binders (for example, high affinity binders). Novel antibody sequences can be identified that display higher binding affinity to any of a variety of target antigens.

In some embodiments, libraries comprising polypeptides of the invention are subjected to a plurality of sorting rounds, wherein each sorting round comprises contacting the binders obtained from the previous round with a target antigen distinct from the target antigen(s) of the previous round(s). Preferably, but not necessarily, the target antigens are homologous in sequence, for example members of a family of related but distinct polypeptides, such as, but not limited to, cytokines (for example, alpha interferon subtypes).

Generation of Libraries Comprising Variant CDR-Containing Polypeptides Libraries of variant CDR polypeptides can be generated by mutating the solvent accessible and/or highly diverse positions in at least one CDR of an antibody variable domain. Some or all of the CDRs can be mutated using the methods of the invention. In some embodiments, it may be preferable to generate diverse antibody libraries by mutating positions in CDRH1, CDRH2 and CDRH3 to form a single library or by mutating positions in CDRL3 and CDRH3 to form a single library or by mutating positions in CDRL3 and CDRH1, CDRH2 and CDRH3 to form a single library.

A library of antibody variable domains can be generated, for example, having mutations in the solvent accessible and/or highly diverse positions of CDRH1, CDRH2 and CDRH3. Another library can be generated having mutations in CDRL1, CDRL2 and CDRL3. These libraries can also be used in conjunction with each other to generate binders of desired affinities. For example, after one or more rounds of selection of heavy chain libraries for binding to a target antigen, a light chain library can be replaced into the population of heavy chain binders for further rounds of selection to increase the affinity of the binders.

In one embodiment, a library is created by substitution of original amino acids with a limited set of variant amino acids in the CDRH1, CDRH2, and/or CDRH3 region of the variable region of the heavy chain sequence and/or the CDRL3 region of the variable region of the light chain sequence. According to the invention, this library can contain a plurality of antibody sequences, wherein the sequence diversity is primarily in the CDRH3 region of the heavy chain sequence.

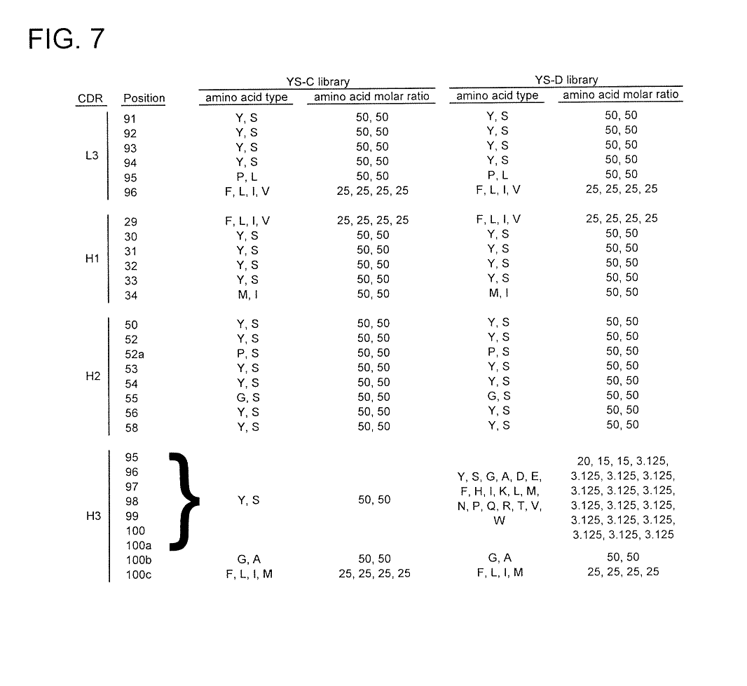

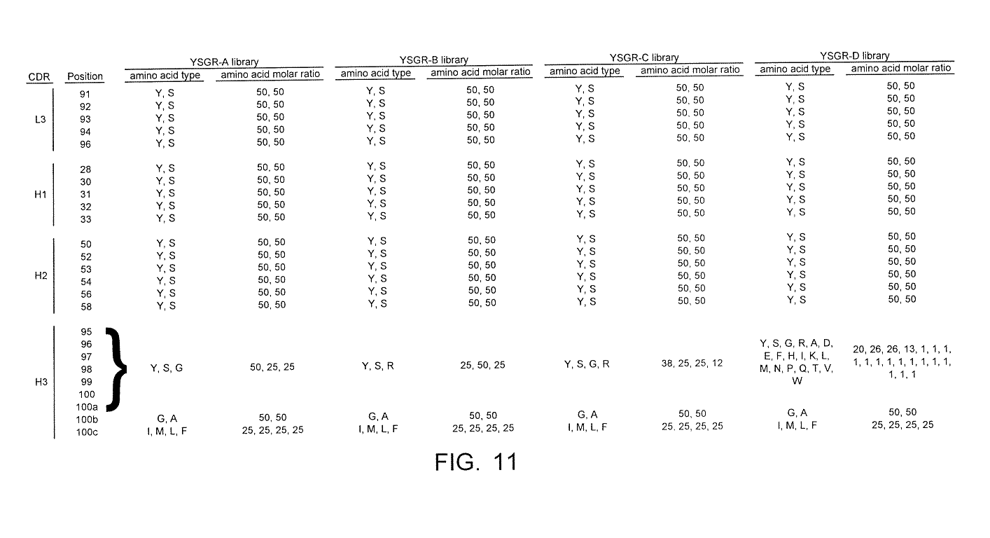

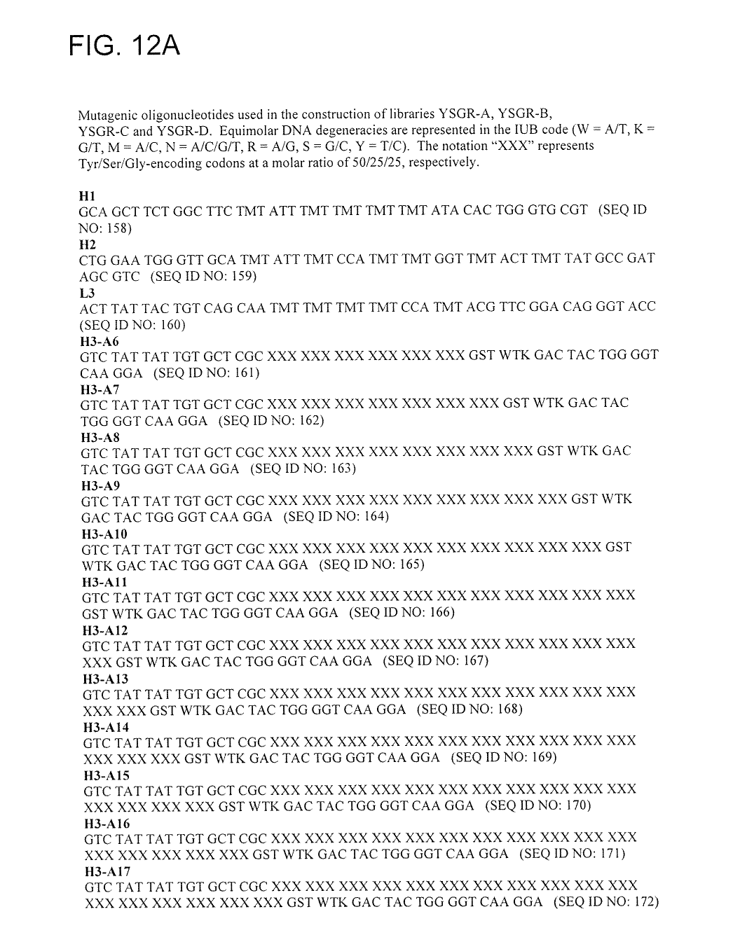

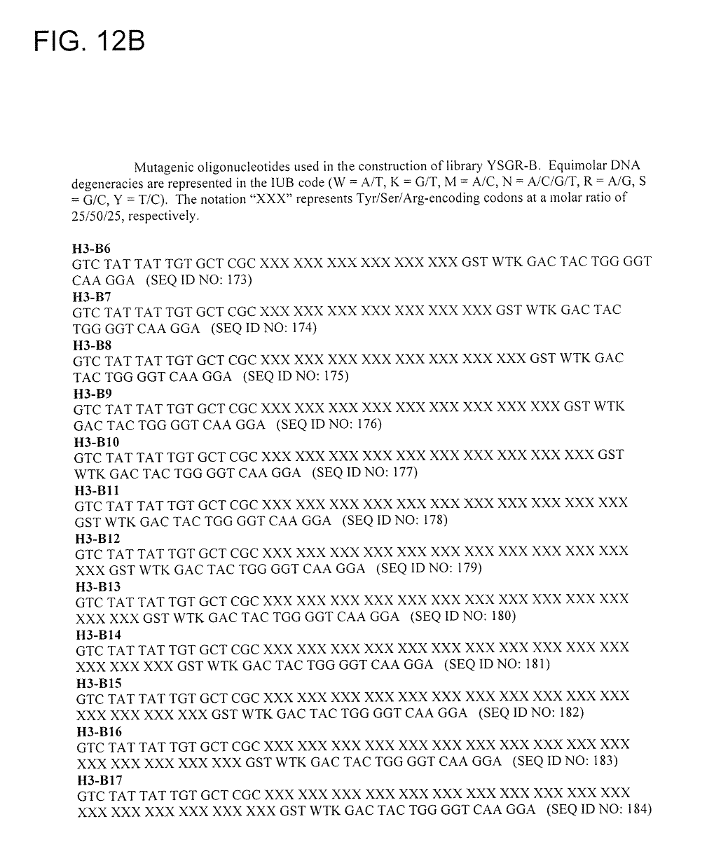

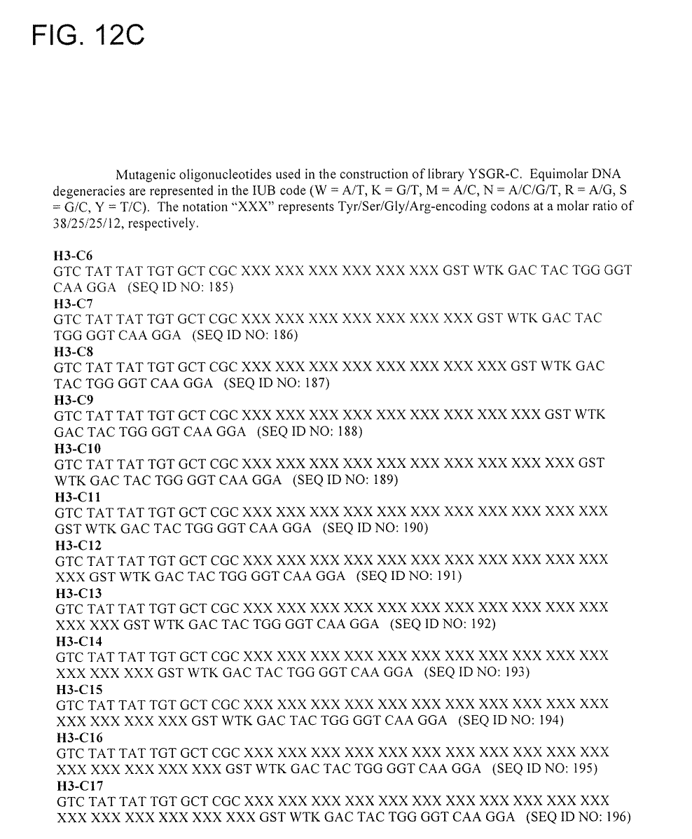

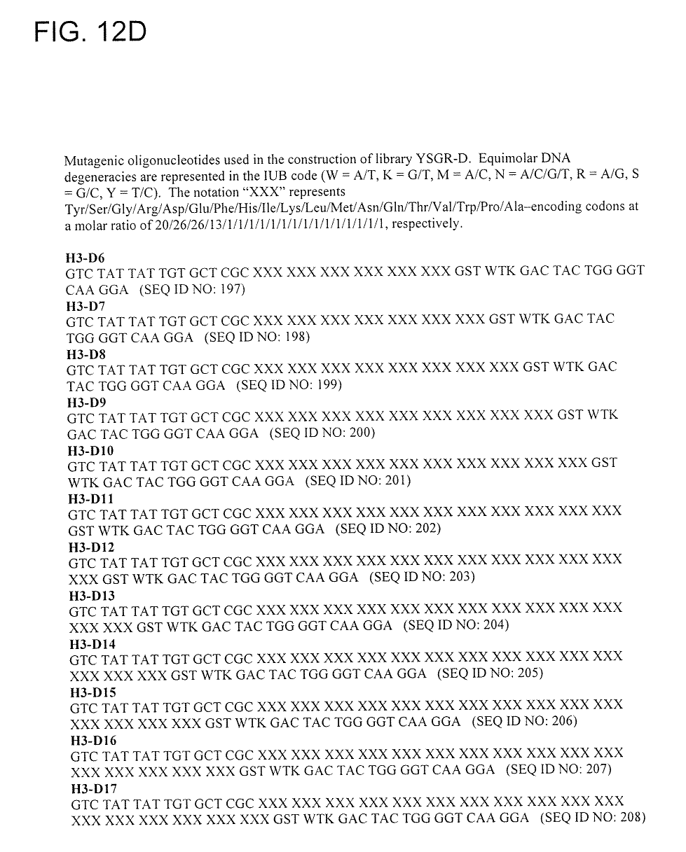

In one aspect, the library is created in the context of the humanized antibody 4D5 sequence, or the sequence of the framework amino acids of the humanized antibody 4D5 sequence. In certain embodiments, the library is created by substitution of at least residues 29-34 of CDRH1, residues 50, 52, 52a, 53-56, and 58 of CDRH2, residues 95-100, 100a, 100b, and 1000c of CDRH3, and residues 91-96 of CDRL3 with the amino acids set forth as shown in FIG. 7 for the “YS-C” library. In certain embodiments, the library is created by substitution of at least residues 29-34 of CDRH1, residues 50, 52, 52a, 53-56, and 58 of CDRH2, residues 95-100, 100a, 100b, and 100c of CDRH3, and residues 91-96 of CDRL3 with the amino acids set forth as shown in FIG. 7 for the “YS-D” library. In certain embodiments, the library is created by substitution of at least residues 28 and 30-33 of CDRH1, residues 50, 52-54, 56, and 58 of CDRH2, residues 95, 96, 97, 98, 99, 100, 100a, 100b, and 100c of CDRH3, and residues 91-94 and 96 of CDRL3 with the amino acids set forth as shown in FIG. 11 for the “YSGR-A” library. In certain embodiments, the library is created by substitution of at least residues 28 and 30-33 of CDRH1, residues 50, 52-54, 56, and 58 of CDRH2, residues 95, 96, 97, 98, 99, 100, 100a, 100b, and 100c of CDRH3, and residues 91-94 and 96 of CDRL3 with the amino acids set forth as shown in FIG. 11 for the “YSGR-B” library. In certain embodiments, the library is created by substitution of at least residues 28 and 30-33 of CDRH1, residues 50, 52-54, 56, and 58 of CDRH2, residues 95, 96, 97, 98, 99, 100, 100a, 100b, and 100c of CDRH3, and residues 91-94 and 96 of CDRL3 with the amino acids set forth as shown in FIG. 11 for the “YSGR-C” library. In certain embodiments, the library is created by substitution of at least residues 28 and 30-33 of CDRH1, residues 50, 52-54, 56, and 58 of CDRH2, residues 95, 96, 97, 98, 99, 100, 100a, 100b, and 100c of CDRH3, and residues 91-94 and 96 of CDRL3 with the amino acids set forth as shown in FIG. 11 for the “YSGR-D” library. Positions 100b or 100c may have a different alphabetical label depending on the length of CDRH3, but correspond to the last two amino acid positions before position 101. Examples of suitable oligonucleotide sequences include, but are not limited to, those listed in FIGS. 8A and 8B and FIGS. 12A-12D, and can be determined by one skilled in the art according to the criteria described herein.

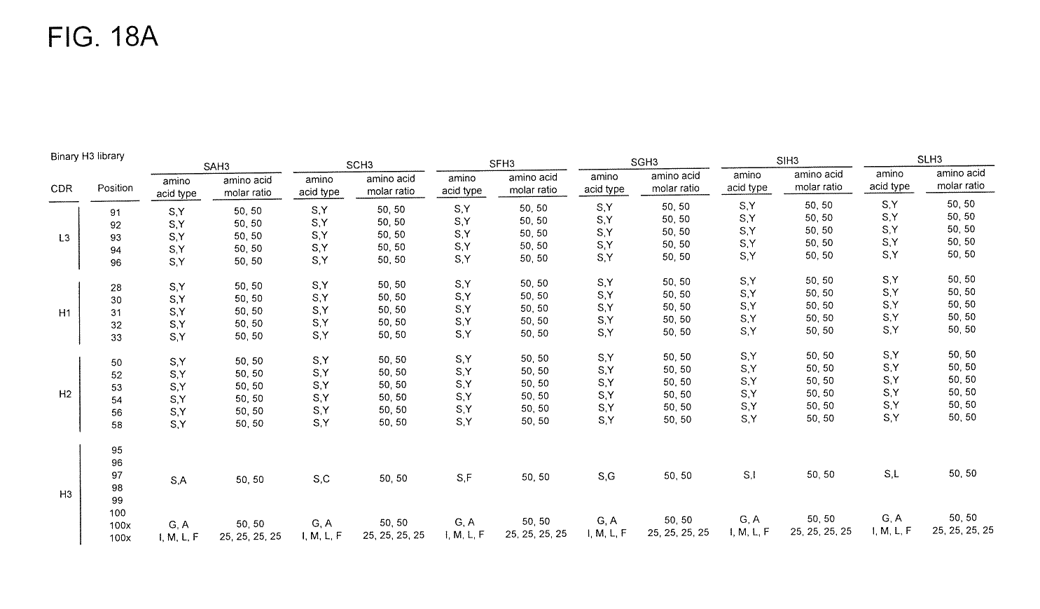

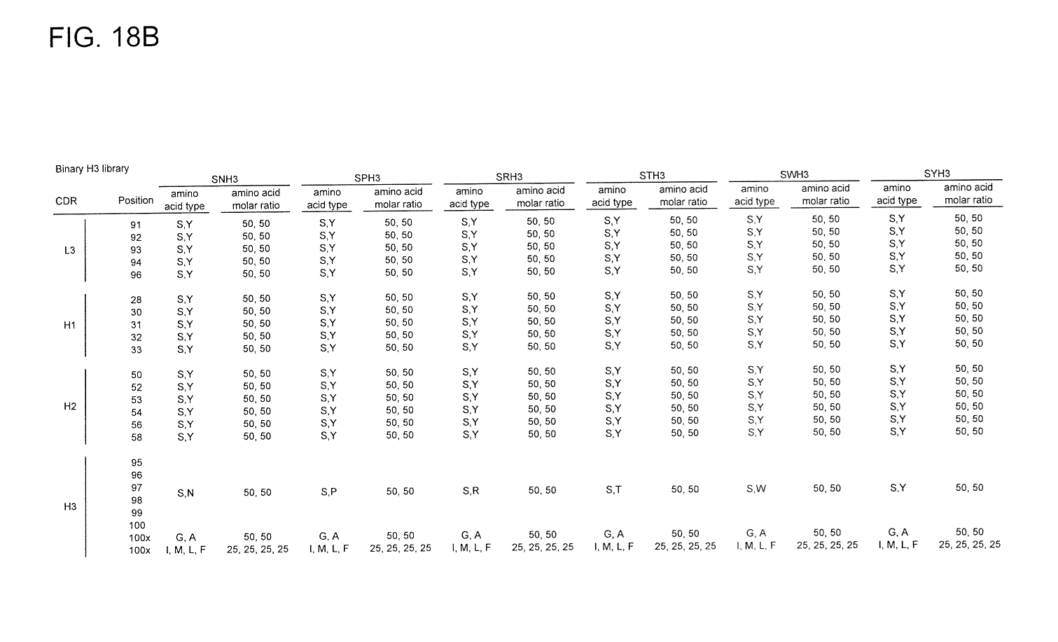

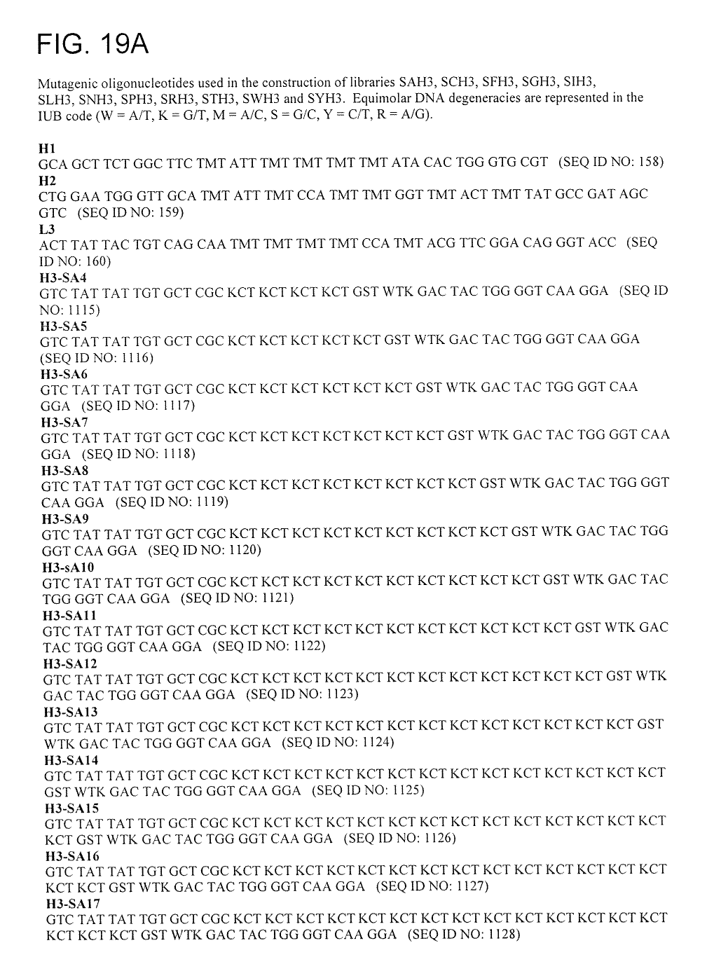

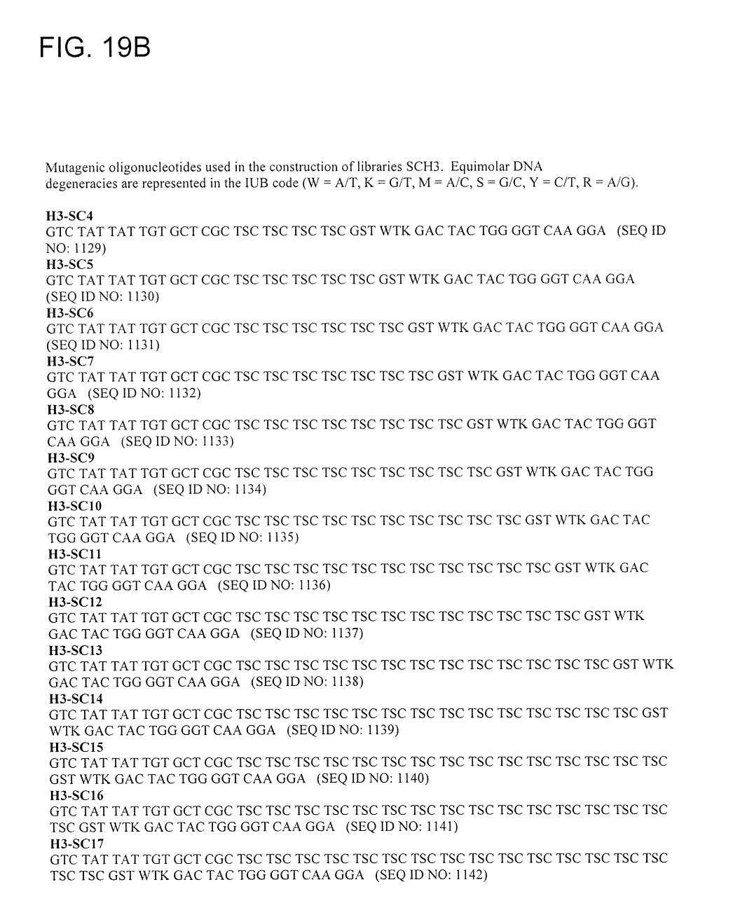

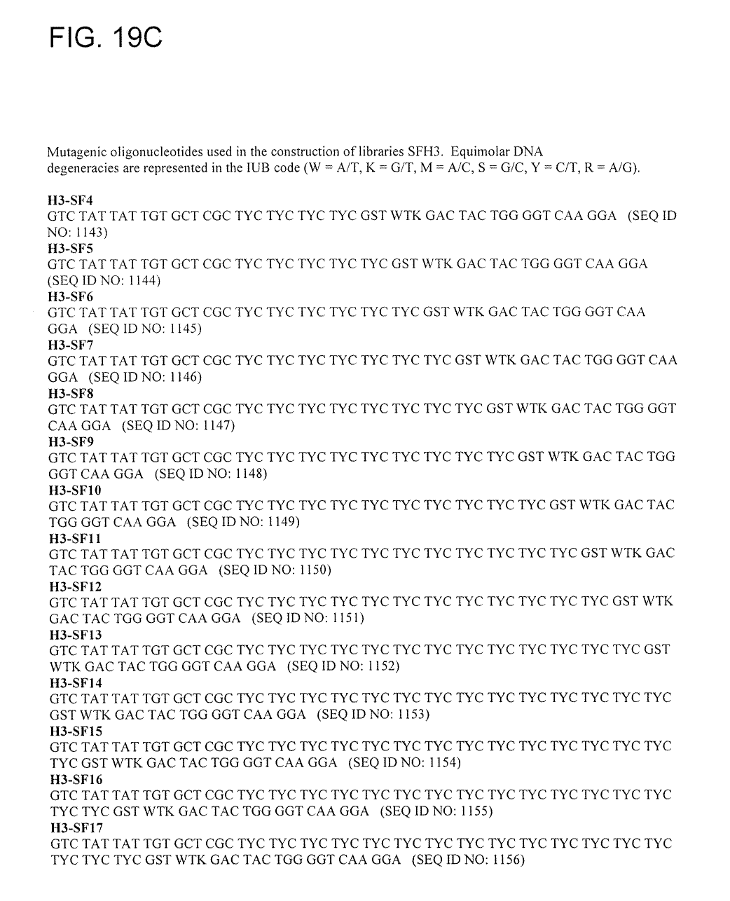

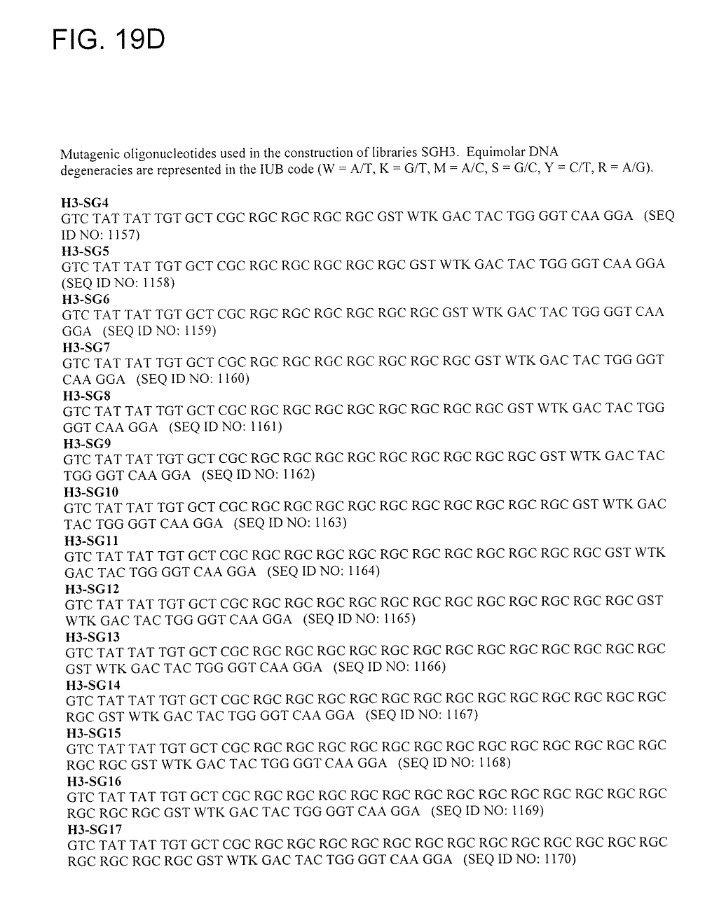

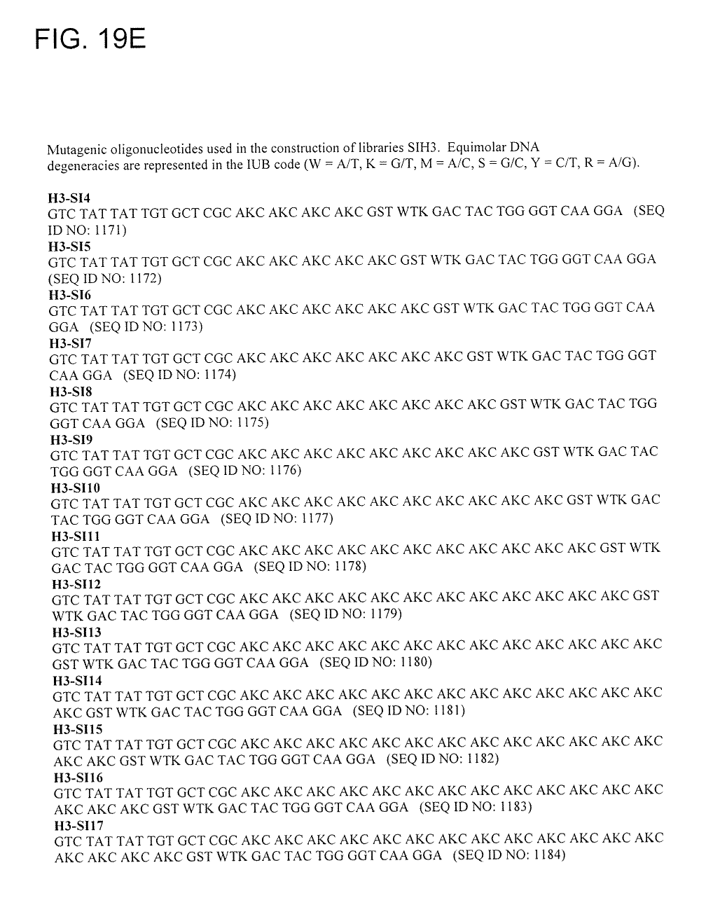

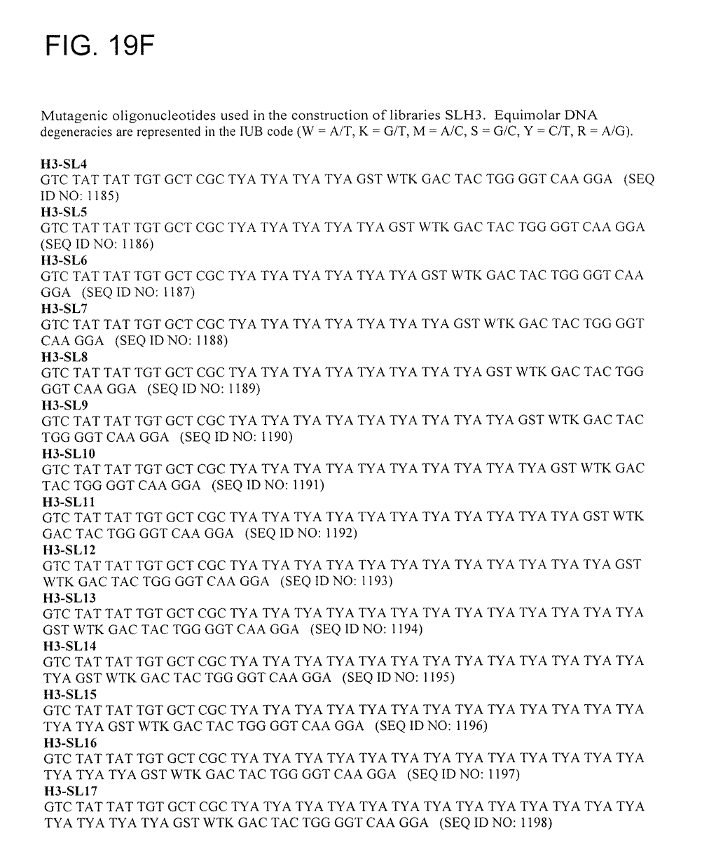

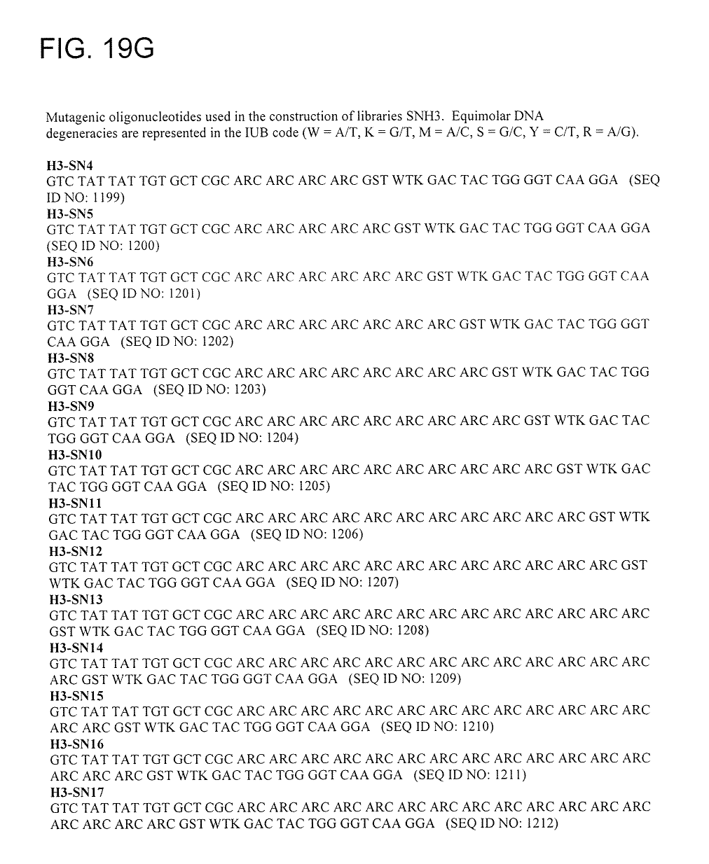

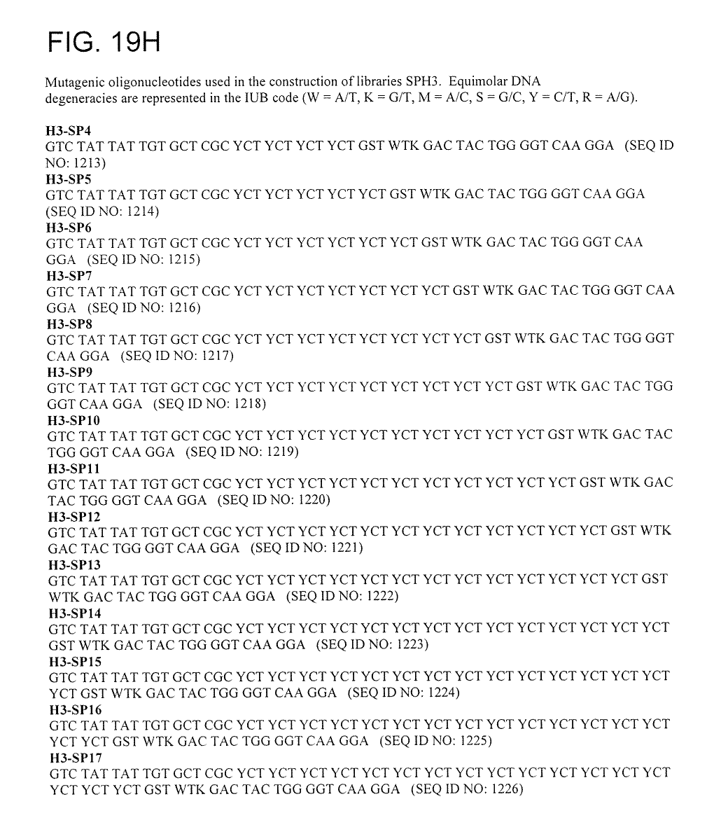

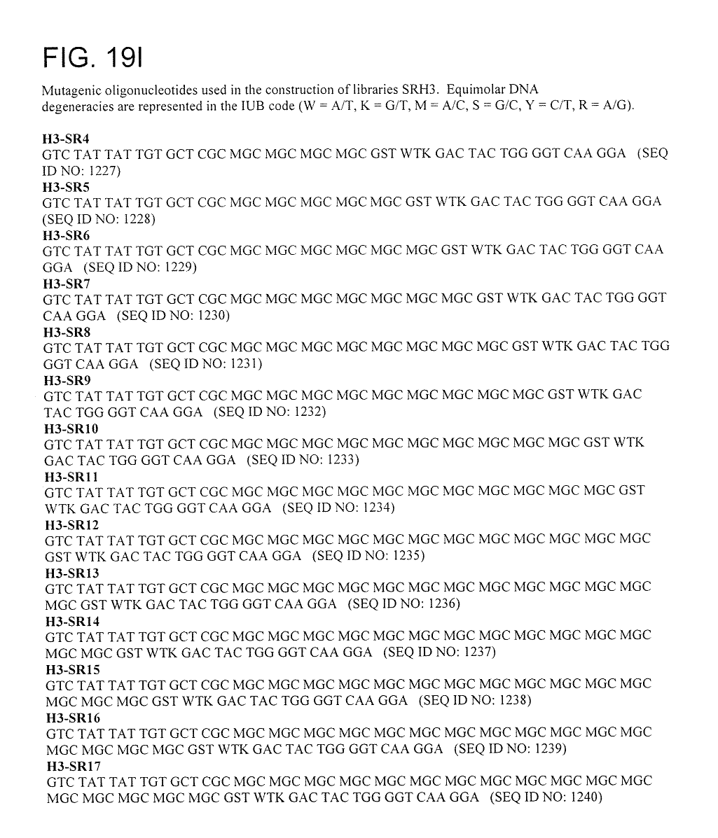

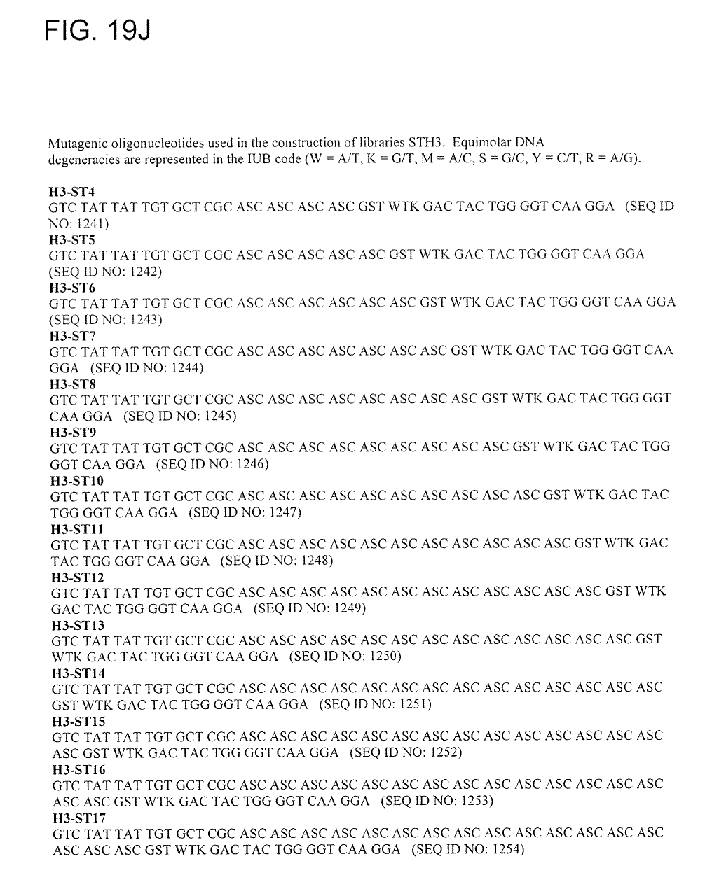

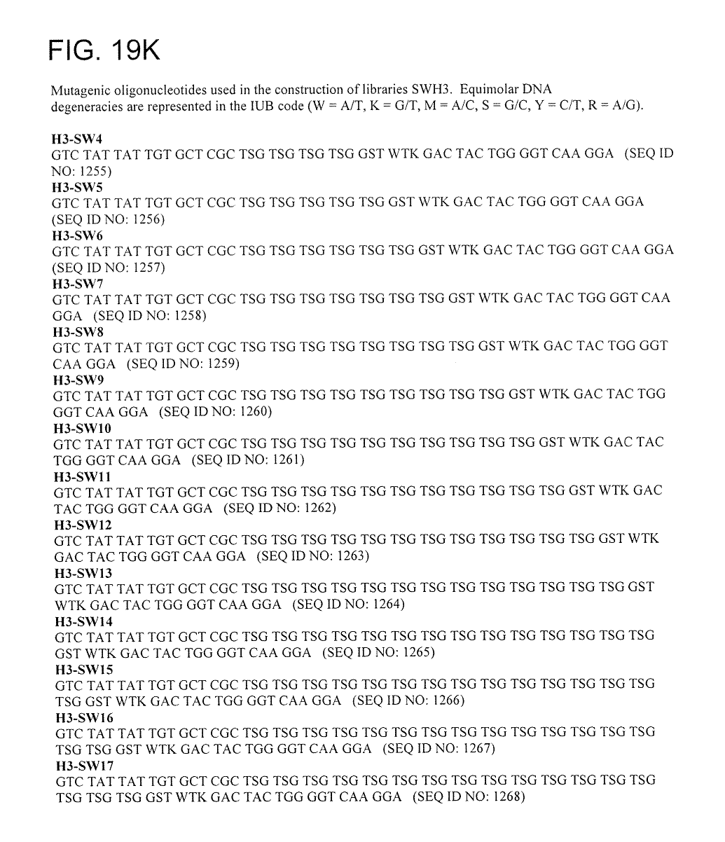

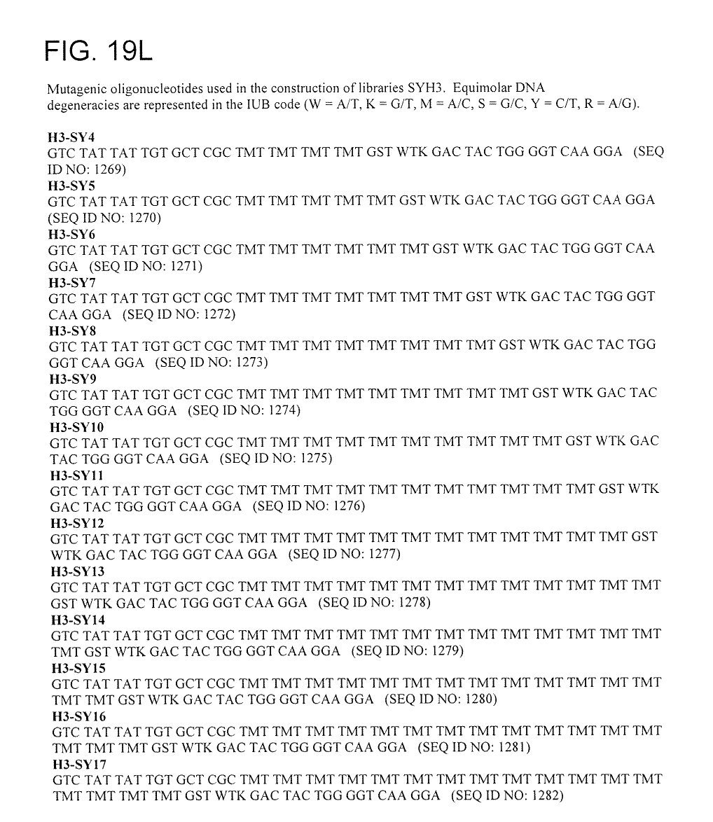

In certain embodiments, the library is created by substitution of at least residues 28 and 30-33 of CDRH1, residues 50, 52, 53, 54, 56, and 58 of CDRH2, residues 95-100m of CDRH3, and residues 91-94 and 96 of CDRL3 with the amino acids set forth as shown in FIG. 18A for the “SAH3” library. In certain embodiments, the library is created by substitution of at least residues 28 and 30-33 of CDRH1, residues 50, 52, 53, 54, 56, and 58 of CDRH2, residues 95-100m of CDRH3, and residues 91-94 and 96 of CDRL3 with the amino acids set forth as shown in FIG. 18A for the “SCH3” library. In certain embodiments, the library is created by substitution of at least residues 28 and 30-33 of CDRH1, residues 50, 52, 53, 54, 56, and 58 of CDRH2, residues 95-100m of CDRH3, and residues 91-94 and 96 of CDRL3 with the amino acids set forth as shown in FIG. 18A for the “SFH3” library. In certain embodiments, the library is created by substitution of at least residues 28 and 30-33 of CDRH1, residues 50, 52, 53, 54, 56, and 58 of CDRH2, residues 95-100m of CDRH3, and residues 91-94 and 96 of CDRL3 with the amino acids set forth as shown in FIG. 18A for the “SGH3” library. In certain embodiments, the library is created by substitution of at least residues 28 and 30-33 of CDRH1, residues 50, 52, 53, 54, 56, and 58 of CDRH2, residues 95-100m of CDRH3, and residues 91-94 and 96 of CDRL3 with the amino acids set forth as shown in FIG. 18A for the “SIH3” library. In certain embodiments, the library is created by substitution of at least residues 28 and 30-33 of CDRH1, residues 50, 52, 53, 54, 56, and 58 of CDRH2, residues 95-100m of CDRH3, and residues 91-94 and 96 of CDRL3 with the amino acids set forth as shown in FIG. 18A for the “SLH3” library. Positions 100l or 100m may have a different alphabetical label depending on the length of CDRH3, but correspond to the last two amino acid positions before position 101. Examples of suitable oligonucleotide sequences include, but are not limited to, those listed in FIGS. 19A-19L, and can be determined by one skilled in the art according to the criteria described herein.

In certain embodiments, the library is created by substitution of at least residues 28 and 30-33 of CDRH1, residues 50, 52, 53, 54, 56, and 58 of CDRH2, residues 95-100m of CDRH3, and residues 91-94 and 96 of CDRL3 with the amino acids set forth as shown in FIG. 18B for the “SNH3” library. In certain embodiments, the library is created by substitution of at least residues 28 and 30-33 of CDRH1, residues 50, 52, 53, 54, 56, and 58 of CDRH2, residues 95-100m of CDRH3, and residues 91-94 and 96 of CDRL3 with the amino acids set forth as shown in FIG. 18B for the “SPH3” library. In certain embodiments, the library is created by substitution of at least residues 28 and 30-33 of CDRH1, residues 50, 52, 53, 54, 56, and 58 of CDRH2, residues 95-100m of CDRH3, and residues 91-94 and 96 of CDRL3 with the amino acids set forth as shown in FIG. 18B for the “SRH3” library. In certain embodiments, the library is created by substitution of at least residues 28 and 30-33 of CDRH1, residues 50, 52, 53, 54, 56, and 58 of CDRH2, residues 95-100m of CDRH3, and residues 91-94 and 96 of CDRL3 with the amino acids set forth as shown in FIG. 18B for the “STH3” library. In certain embodiments, the library is created by substitution of at least residues 28 and 30-33 of CDRH1, residues 50, 52, 53, 54, 56, and 58 of CDRH2, residues 95-100m of CDRH3, and residues 91-94 and 96 of CDRL3 with the amino acids set forth as shown in FIG. 18B for the “SWH3” library. In certain embodiments, the library is created by substitution of at least residues 28 and 30-33 of CDRH1, residues 50, 52, 53, 54, 56, and 58 of CDRH2, residues 95-100m of CDRH3, and residues 91-94 and 96 of CDRL3 with the amino acids set forth as shown in FIG. 18B for the “SYH3” library. Positions 100l or 100m may have a different alphabetical label depending on the length of CDRH3, but correspond to the last two amino acid positions before position 101. Examples of suitable oligonucleotide sequences include, but are not limited to, those listed in FIGS. 19A-19L, and can be determined by one skilled in the art according to the criteria described herein.

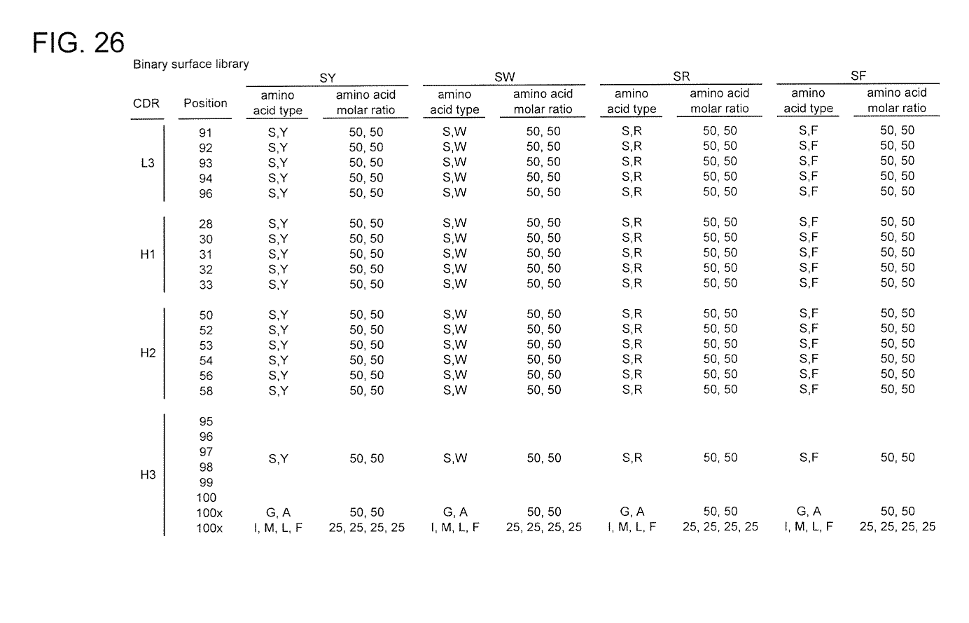

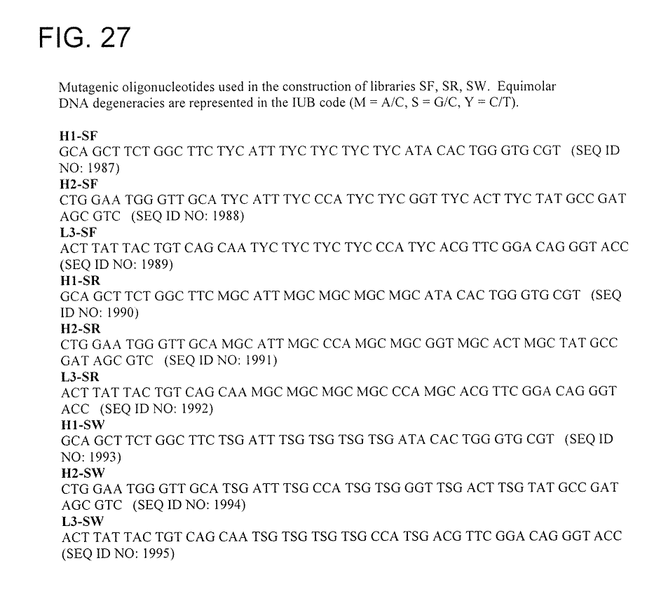

In certain embodiments, the library is created by substitution of at least residues 28 and 30-33 of CDRH1, residues 50, 52, 53, 54, 56, and 58 of CDRH2, residues 95-100m of CDRH3, and residues 91-94 and 96 of CDRL3 with the amino acids set forth as shown in FIG. 26 for the “SY” library. In certain embodiments, the library is created by substitution of at least residues 28 and 30-33 of CDRH1, residues 50, 52, 53, 54, 56, and 58 of CDRH2, residues 95-100m of CDRH3, and residues 91-94 and 96 of CDRL3 with the amino acids set forth as shown in FIG. 26 for the “SW” library. In certain embodiments, the library is created by substitution of at least residues 28 and 30-33 of CDRH1, residues 50, 52, 53, 54, 56, and 58 of CDRH2, residues 95-100m of CDRH3, and residues 91-94 and 96 of CDRL3 with the amino acids set forth as shown in FIG. 26 for the “SR” library. In certain embodiments, the library is created by substitution of at least residues 28 and 30-33 of CDRH1, residues 50, 52, 53, 54, 56, and 58 of CDRH2, residues 95-100m of CDRH3, and residues 91-94 and 96 of CDRL3 with the amino acids set forth as shown in FIG. 26 for the “SF” library. Positions 100l or 100m may have a different alphabetical label depending on the length of CDRH3, but correspond to the last two amino acid positions before position 101. Examples of suitable oligonucleotide sequences include, but are not limited to, those listed in FIG. 27, and can be determined by one skilled in the art according to the criteria described herein.

In certain embodiments, a library is created by pooling other libraries. In one embodiment, the “SXH3” library as used herein comprises the SAH3, SCH3, SFH3, SGH3, SIH3, SLH3, SNH3, SPH3, SRH3, STH3, SWH3, and SYH3 libraries. In another embodiment, the “SX-surface” library comprises the “SY”, “SW”, “SR”, and “SF” libraries.

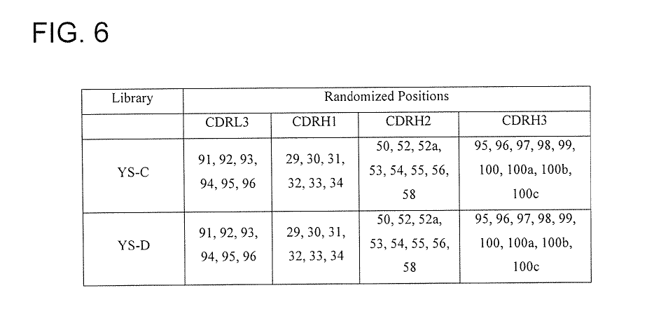

In another embodiment, different CDRH3 designs are utilized to isolate high affinity binders and to isolate binders for a variety of epitopes. For diversity in CDRH3, multiple libraries can be constructed separately with different lengths of H3 and then combined to select for binders to target antigens. The range of lengths of CDRH3 generated in this library can be 10-21, 11-21, 12-21, 13-21, 14-21, 15-21, 16-21, 17-21, 18-21, 19-21, 20-21, amino acids, although lengths different from this can also be generated. Diversity can also be generated in CDRH1 and CDRH2, as indicated above. In one embodiment of a library, diversity in H1 and H2 is generated utilizing the oligonucleotides illustrated in FIGS. 8A and 8B, 12A-12D, 19A-L, and 27. Other oligonucleotides with varying sequences can also be used. Oligonucleotides can be used singly or pooled in any of a variety of combinations depending on practical needs and desires of the practitioner. In some embodiments, randomized positions in heavy chain CDRs include those listed in FIGS. 6, 7, 11, 18A, 18B, and 26.

Multiple libraries can be pooled and sorted using solid support selection and solution sorting methods as described herein. Multiple sorting strategies may be employed. For example, one variation involves sorting on target bound to a solid, followed by sorting for a tag that may be present on the fusion polypeptide (e.g. anti-gD tag) and followed by another sort on target bound to solid. Alternatively, the libraries can be sorted first on target bound to a solid surface, the eluted binders are then sorted using solution phase binding with decreasing concentrations of target antigen. Utilizing combinations of different sorting methods provides for minimization of selection of only highly expressed sequences and provides for selection of a number of different high affinity clones.

Of the binders isolated from the pooled libraries as described above, it has been discovered that in some instances affinity may be further improved by providing limited diversity in the light chain. Light chain diversity may be, but is not necessarily, generated by diversifying amino acid positions 91-96 in CDRL3, or a subset thereof. In one embodiment, the randomized positions are those listed in FIGS. 6, 7, 11, 18A, 18B, and 26.

High affinity binders isolated from the libraries of these embodiments are readily produced in bacterial and eukaryotic cell culture in high yield. The vectors can be designed to readily remove sequences such as gD tags, viral coat protein component sequence, and/or to add in constant region sequences to provide for production of full length antibodies or antigen binding fragments in high yield.

Any combination of codon sets and CDRs can be diversified according to methods of the invention.

Vectors, Host Cells and Recombinant Methods

For recombinant production of an antibody polypeptide of the invention, the nucleic acid encoding it is isolated and inserted into a replicable vector for further cloning (amplification of the DNA) or for expression. DNA encoding the antibody is readily isolated and sequenced using conventional procedures (e.g., by using oligonucleotide probes that are capable of binding specifically to genes encoding the heavy and light chains of the antibody). Many vectors are available. The choice of vector depends in part on the host cell to be used. Generally, host cells are of either prokaryotic or eukaryotic (generally mammalian) origin.

Generating Antibodies Using Prokaryotic Host Cells:

Vector Construction

Polynucleotide sequences encoding polypeptide components of the antibody of the invention can be obtained using standard recombinant techniques. Desired polynucleotide sequences may be isolated and sequenced from antibody producing cells such as hybridoma cells. Alternatively, polynucleotides can be synthesized using nucleotide synthesizer or PCR techniques. Once obtained, sequences encoding the polypeptides are inserted into a recombinant vector capable of replicating and expressing heterologous polynucleotides in prokaryotic hosts. Many vectors that are available and known in the art can be used for the purpose of the present invention. Selection of an appropriate vector will depend mainly on the size of the nucleic acids to be inserted into the vector and the particular host cell to be transformed with the vector. Each vector contains various components, depending on its function (amplification or expression of heterologous polynucleotide, or both) and its compatibility with the particular host cell in which it resides. The vector components generally include, but are not limited to: an origin of replication, a selection marker gene, a promoter, a ribosome binding site (RBS), a signal sequence, the heterologous nucleic acid insert and a transcription termination sequence.

In general, plasmid vectors containing replicon and control sequences which are derived from species compatible with the host cell are used in connection with these hosts. The vector ordinarily carries a replication site, as well as marking sequences which are capable of providing phenotypic selection in transformed cells. For example, E. coli is typically transformed using pBR322, a plasmid derived from an E. coli species. pBR322 contains genes encoding ampicillin (Amp) and tetracycline (Tet) resistance and thus provides easy means for identifying transformed cells. pBR322, its derivatives, or other microbial plasmids or bacteriophage may also contain, or be modified to contain, promoters which can be used by the microbial organism for expression of endogenous proteins. Examples of pBR322 derivatives used for expression of particular antibodies are described in detail in Carter et al., U.S. Pat. No. 5,648,237.

In addition, phage vectors containing replicon and control sequences that are compatible with the host microorganism can be used as transforming vectors in connection with these hosts. For example, bacteriophage such as λGEM.TM.-11 may be utilized in making a recombinant vector which can be used to transform susceptible host cells such as E. coli LE392.

The expression vector of the invention may comprise two or more promoter-cistron pairs, encoding each of the polypeptide components. A promoter is an untranslated regulatory sequence located upstream (5′) to a cistron that modulates its expression. Prokaryotic promoters typically fall into two classes, inducible and constitutive. Inducible promoter is a promoter that initiates increased levels of transcription of the cistron under its control in response to changes in the culture condition, e.g. the presence or absence of a nutrient or a change in temperature.

A large number of promoters recognized by a variety of potential host cells are well known. The selected promoter can be operably linked to cistron DNA encoding the light or heavy chain by removing the promoter from the source DNA via restriction enzyme digestion and inserting the isolated promoter sequence into the vector of the invention. Both the native promoter sequence and many heterologous promoters may be used to direct amplification and/or expression of the target genes. In some embodiments, heterologous promoters are utilized, as they generally permit greater transcription and higher yields of expressed target gene as compared to the native target polypeptide promoter.

Promoters suitable for use with prokaryotic hosts include the PhoA promoter, the β-galactamase and lactose promoter systems, a tryptophan (trp) promoter system and hybrid promoters such as the tac or the trc promoter. However, other promoters that are functional in bacteria (such as other known bacterial or phage promoters) are suitable as well. Their nucleotide sequences have been published, thereby enabling a skilled worker operably to ligate them to cistrons encoding the target light and heavy chains (Siebenlist et al. (1980) Cell 20: 269) using linkers or adaptors to supply any required restriction sites.

In one aspect of the invention, each cistron within the recombinant vector comprises a secretion signal sequence component that directs translocation of the expressed polypeptides across a membrane. In general, the signal sequence may be a component of the vector, or it may be a part of the target polypeptide DNA that is inserted into the vector. The signal sequence selected for the purpose of this invention should be one that is recognized and processed (i.e. cleaved by a signal peptidase) by the host cell. For prokaryotic host cells that do not recognize and process the signal sequences native to the heterologous polypeptides, the signal sequence is substituted by a prokaryotic signal sequence selected, for example, from the group consisting of the alkaline phosphatase, penicillinase, Ipp, or heat-stable enterotoxin II (STII) leaders, LamB, PhoE, PelB, OmpA and MBP. In one embodiment of the invention, the signal sequences used in both cistrons of the expression system are STII signal sequences or variants thereof.

In another aspect, the production of the immunoglobulins according to the invention can occur in the cytoplasm of the host cell, and therefore does not require the presence of secretion signal sequences within each cistron. In that regard, immunoglobulin light and heavy chains are expressed, folded and assembled to form functional immunoglobulins within the cytoplasm. Certain host strains (e.g., the E. coli trxB⁻ strains) provide cytoplasm conditions that are favorable for disulfide bond formation, thereby permitting proper folding and assembly of expressed protein subunits. Proba and Pluckthun Gene, 159:203 (1995).

The present invention provides an expression system in which the quantitative ratio of expressed polypeptide components can be modulated in order to maximize the yield of secreted and properly assembled antibodies of the invention. Such modulation is accomplished at least in part by simultaneously modulating translational strengths for the polypeptide components.

One technique for modulating translational strength is disclosed in Simmons et al., U.S. Pat. No. 5,840,523. It utilizes variants of the translational initiation region (TIR) within a cistron. For a given TIR, a series of amino acid or nucleic acid sequence variants can be created with a range of translational strengths, thereby providing a convenient means by which to adjust this factor for the desired expression level of the specific chain. TIR variants can be generated by conventional mutagenesis techniques that result in codon changes which can alter the amino acid sequence, although silent changes in the nucleotide sequence are preferred. Alterations in the TIR can include, for example, alterations in the number or spacing of Shine-Dalgarno sequences, along with alterations in the signal sequence. One method for generating mutant signal sequences is the generation of a “codon bank” at the beginning of a coding sequence that does not change the amino acid sequence of the signal sequence (i.e., the changes are silent). This can be accomplished by changing the third nucleotide position of each codon; additionally, some amino acids, such as leucine, serine, and arginine, have multiple first and second positions that can add complexity in making the bank. This method of mutagenesis is described in detail in Yansura et al. (1992) METHODS: A Companion to Methods in Enzymol. 4:151-158.

In certain embodiments, a set of vectors is generated with a range of TIR strengths for each cistron therein. This limited set provides a comparison of expression levels of each chain as well as the yield of the desired antibody products under various TIR strength combinations. TIR strengths can be determined by quantifying the expression level of a reporter gene as described in detail in Simmons et al. U.S. Pat. No. 5, 840,523. Based on the translational strength comparison, the desired individual TIRs are selected to be combined in the expression vector constructs of the invention.

Prokaryotic host cells suitable for expressing antibodies of the invention include Archaebacteria and Eubacteria, such as Gram-negative or Gram-positive organisms. Examples of useful bacteria include Escherichia (e.g., E. coli), Bacilli (e.g., B. subtilis), Enterobacteria, Pseudomonas species (e.g., P. aeruginosa), Salmonella typhimurium, Serratia marcescans, Klebsiella, Proteus, Shigella, Rhizobia, Vitreoscilla, or Paracoccus. In one embodiment, gram-negative cells are used. In one embodiment, E. coli cells are used as hosts for the invention. Examples of E. coli strains include strain W3110 (Bachmann, Cellular and Molecular Biology, vol. 2 (Washington, D.C.: American Society for Microbiology, 1987), pp. 1190-1219; ATCC Deposit No. 27,325) and derivatives thereof, including strain 33D3 having genotype W3110 ΔfhuA (ΔtonA) ptr3 lac Iq lacL8 ΔompTΔ(nmpc-fepE) degP41 kan^{R} (U.S. Pat. No. 5,639,635). Other strains and derivatives thereof, such as E. coli 294 (ATCC 31,446), E. coli B, E. coli_{ο}1776 (ATCC 31,537) and E. coli RV308 (ATCC 31,608) are also suitable. These examples are illustrative rather than limiting. Methods for constructing derivatives of any of the above-mentioned bacteria having defined genotypes are known in the art and described in, for example, Bass et al., Proteins, 8:309-314 (1990). It is generally necessary to select the appropriate bacteria taking into consideration replicability of the replicon in the cells of a bacterium. For example, E. coli, Serratia, or Salmonella species can be suitably used as the host when well known plasmids such as pBR322, pBR325, pACYC177, or pKN410 are used to supply the replicon. Typically the host cell should secrete minimal amounts of proteolytic enzymes, and additional protease inhibitors may desirably be incorporated in the cell culture.

Antibody Production

Host cells are transformed with the above-described expression vectors and cultured in conventional nutrient media modified as appropriate for inducing promoters, selecting transformants, or amplifying the genes encoding the desired sequences.

Transformation means introducing DNA into the prokaryotic host so that the DNA is replicable, either as an extrachromosomal element or by chromosomal integrant. Depending on the host cell used, transformation is done using standard techniques appropriate to such cells. The calcium treatment employing calcium chloride is generally used for bacterial cells that contain substantial cell-wall barriers. Another method for transformation employs polyethylene glycol/DMSO. Yet another technique used is electroporation.

Prokaryotic cells used to produce the polypeptides of the invention are grown in media known in the art and suitable for culture of the selected host cells. Examples of suitable media include luria broth (LB) plus necessary nutrient supplements. In some embodiments, the media also contains a selection agent, chosen based on the construction of the expression vector, to selectively permit growth of prokaryotic cells containing the expression vector. For example, ampicillin is added to media for growth of cells expressing ampicillin resistant gene.

Any necessary supplements besides carbon, nitrogen, and inorganic phosphate sources may also be included at appropriate concentrations introduced alone or as a mixture with another supplement or medium such as a complex nitrogen source. Optionally the culture medium may contain one or more reducing agents selected from the group consisting of glutathione, cysteine, cystamine, thioglycollate, dithioerythritol and dithiothreitol.

The prokaryotic host cells are cultured at suitable temperatures. For E. coli growth, for example, the temperature ranges from about 20° C. to about 39° C., from about 25° C. to about 37° C., and/or about 30° C. may be used. The pH of the medium may be any pH ranging from about 5 to about 9, depending mainly on the host organism. For E. coli, the pH can be about 6.8 to about 7.4, and can be about 7.0.

If an inducible promoter is used in the expression vector of the invention, protein expression is induced under conditions suitable for the activation of the promoter. In one aspect of the invention, PhoA promoters are used for controlling transcription of the polypeptides. Accordingly, the transformed host cells are cultured in a phosphate-limiting medium for induction. The phosphate-limiting medium can be C.R.A.P medium (see, e.g., Simmons et al., J. Immunol. Methods (2002), 263:133-147). A variety of other inducers may be used, according to the vector construct employed, as is known in the art.

In one embodiment, the expressed polypeptides of the present invention are secreted into and recovered from the periplasm of the host cells. Protein recovery typically involves disrupting the microorganism, generally by such means as osmotic shock, sonication or lysis. Once cells are disrupted, cell debris or whole cells may be removed by centrifugation or filtration. The proteins may be further purified, for example, by affinity resin chromatography. Alternatively, proteins can be transported into the culture media and isolated therein. Cells may be removed from the culture and the culture supernatant can be filtered and concentrated for further purification of the produced proteins. The expressed polypeptides can be further isolated and identified using commonly known methods such as polyacrylamide gel electrophoresis (PAGE) and Western blot assay.

In one aspect of the invention, antibody production is conducted in large quantity by a fermentation process. Various large-scale fed-batch fermentation procedures are available for production of recombinant proteins. Large-scale fermentations have at least 1000 liters of capacity; in certain embodiments, the large-scale fermentors have about 1,000 to 100,000 liters of capacity. These fermentors use agitator impellers to distribute oxygen and nutrients, especially glucose (a common carbon/energy source). Small scale fermentation refers generally to fermentation in a fermentor that is no more than approximately 100 liters in volumetric capacity, and can range from about 1 liter to about 100 liters.

In a fermentation process, induction of protein expression is typically initiated after the cells have been grown under suitable conditions to a desired density, e.g., an OD₅₅₀ of about 180-220, at which stage the cells are in the early stationary phase. A variety of inducers may be used, according to the vector construct employed, as is known in the art and described above. Cells may be grown for shorter periods prior to induction. Cells are usually induced for about 12-50 hours, although longer or shorter induction times may be used.

To improve the production yield and quality of the polypeptides of the invention, various fermentation conditions can be modified. For example, to improve the proper assembly and folding of the secreted antibody polypeptides, additional vectors overexpressing chaperone proteins, such as Dsb proteins (DsbA, DsbB, DsbC, DsbD and or DsbG) or FkpA (a peptidylprolyl cis,trans-isomerase with chaperone activity) can be used to co-transform the host prokaryotic cells. The chaperone proteins have been demonstrated to facilitate the proper folding and solubility of heterologous proteins produced in bacterial host cells. Chen et al. (1999) J Bio Chem 274:19601-19605; Georgiou et al., U.S. Pat. No. 6,083,715; Georgiou et al., U.S. Pat. No. 6,027,888; Bothmann and Pluckthun (2000) J. Biol. Chem. 275:17100-17105; Ramm and Pluckthun (2000) J. Biol. Chem. 275:17106-17113; Arie et al. (2001) Mol. Microbiol. 39:199-210.

To minimize proteolysis of expressed heterologous proteins (especially those that are proteolytically sensitive), certain host strains deficient for proteolytic enzymes can be used for the present invention. For example, host cell strains may be modified to effect genetic mutation(s) in the genes encoding known bacterial proteases such as Protease III, OmpT, DegP, Tsp, Protease I, Protease Mi, Protease V, Protease VI and combinations thereof. Some E. coli protease-deficient strains are available and described in, for example, Joly et al. (1998), supra; Georgiou et al., U.S. Pat. No. 5,264,365; Georgiou et al., U.S. Pat. No. 5,508,192; Hara et al., Microbial Drug Resistance, 2:63-72 (1996).

In one embodiment, E. coli strains deficient for proteolytic enzymes and transformed with plasmids overexpressing one or more chaperone proteins are used as host cells in the expression system of the invention.

Antibody Purification

In one embodiment, the antibody protein produced herein is further purified to obtain preparations that are substantially homogeneous for further assays and uses. Standard protein purification methods known in the art can be employed. The following procedures are exemplary of suitable purification procedures: fractionation on immunoaffinity or ion-exchange columns, ethanol precipitation, reverse phase HPLC, chromatography on silica or on a cation-exchange resin such as DEAE, chromatofocusing, SDS-PAGE, ammonium sulfate precipitation, and gel filtration using, for example, Sephadex G-75.

In one aspect, Protein A immobilized on a solid phase is used for immunoaffinity purification of the antibody products of the invention. Protein A is a 41 kD cell wall protein from Staphylococcus aureas which binds with a high affinity to the Fc region of antibodies. Lindmark et al (1983) J. Immunol. Meth. 62:1-13. In certain embodiments, the solid phase to which Protein A is immobilized is a column comprising a glass or silica surface. In certain embodiments, the solid phase to which Protein A is immobilized is a controlled pore glass column or a silicic acid column. In some applications, the column has been coated with a reagent, such as glycerol, in an attempt to prevent nonspecific adherence of contaminants.

As the first step of purification, the preparation derived from the cell culture as described above is applied onto the Protein A immobilized solid phase to allow specific binding of the antibody of interest to Protein A. The solid phase is then washed to remove contaminants non-specifically bound to the solid phase. Finally the antibody of interest is recovered from the solid phase by elution.

Generating Antibodies Using Eukaryotic Host Cells:

The vector components generally include, but are not limited to, one or more of the following: a signal sequence, an origin of replication, one or more marker genes, an enhancer element, a promoter, and a transcription termination sequence.

(i) Signal Sequence Component

A vector for use in a eukaryotic host cell may also contain a signal sequence or other polypeptide having a specific cleavage site at the N-terminus of the mature protein or polypeptide of interest. In certain embodiments, the heterologous signal sequence selected is one that is recognized and processed (i.e., cleaved by a signal peptidase) by the host cell. In mammalian cell expression, mammalian signal sequences as well as viral secretory leaders, for example, the herpes simplex gD signal, are available.

The DNA for such precursor region is ligated in reading frame to DNA encoding the antibody.

(ii) Origin of Replication

Generally, an origin of replication component is not needed for mammalian expression vectors. For example, the SV40 origin may typically be used only because it contains the early promoter.

(iii) Selection Gene Component

Expression and cloning vectors may contain a selection gene, also termed a selectable marker. Typical selection genes encode proteins that (a) confer resistance to antibiotics or other toxins, e.g., ampicillin, neomycin, methotrexate, or tetracycline, (b) complement auxotrophic deficiencies, where relevant, or (c) supply critical nutrients not available from complex media.

One example of a selection scheme utilizes a drug to arrest growth of a host cell. Those cells that are successfully transformed with a heterologous gene produce a protein conferring drug resistance and thus survive the selection regimen. Examples of such dominant selection use the drugs neomycin, mycophenolic acid and hygromycin.

Another example of suitable selectable markers for mammalian cells are those that enable the identification of cells competent to take up the antibody nucleic acid, such as DHFR, thymidine kinase, metallothionein-I and -II (e.g., primate metallothionein genes), adenosine deaminase, omithine decarboxylase, etc.

For example, cells transformed with the DHFR selection gene are first identified by culturing all of the transformants in a culture medium that contains methotrexate (Mtx), a competitive antagonist of DHFR. An appropriate host cell when wild-type DHFR is employed is the Chinese hamster ovary (CHO) cell line deficient in DHFR activity (e.g., ATCC CRL-9096).

Alternatively, host cells (particularly wild-type hosts that contain endogenous DHFR) transformed or co-transformed with DNA sequences encoding an antibody, wild-type DHFR protein, and another selectable marker such as aminoglycoside 3′-phosphotransferase (APH) can be selected by cell growth in medium containing a selection agent for the selectable marker such as an aminoglycosidic antibiotic, e.g., kanamycin, neomycin, or G418. See U.S. Pat. No. 4,965,199.

(iv) Promoter Component

Expression and cloning vectors usually contain a promoter that is recognized by the host organism and is operably linked to the antibody polypeptide nucleic acid. Promoter sequences are known for eukaryotes. Virtually all eukaryotic genes have an AT-rich region located approximately 25 to 30 bases upstream from the site where transcription is initiated. Another sequence found 70 to 80 bases upstream from the start of transcription of many genes is a CNCAAT region where N may be any nucleotide. At the 3′ end of most eukaryotic genes is an AATAAA sequence that may be the signal for addition of the poly A tail to the 3′ end of the coding sequence. All of these sequences are suitably inserted into eukaryotic expression vectors.

Antibody polypeptide transcription from vectors in mammalian host cells is controlled, for example, by promoters obtained from the genomes of viruses such as polyoma virus, fowlpox virus, adenovirus (such as Adenovirus 2), bovine papilloma virus, avian sarcoma virus, cytomegalovirus, a retrovirus, hepatitis-B virus and Simian Virus 40 (SV40), from heterologous mammalian promoters, e.g., the actin promoter or an immunoglobulin promoter, from heat-shock promoters, provided such promoters are compatible with the host cell systems.

The early and late promoters of the SV40 virus are conveniently obtained as an SV40 restriction fragment that also contains the SV40 viral origin of replication. The immediate early promoter of the human cytomegalovirus is conveniently obtained as a HindIII E restriction fragment. A system for expressing DNA in mammalian hosts using the bovine papilloma virus as a vector is disclosed in U.S. Pat. No. 4,419,446. A modification of this system is described in U.S. Pat. No. 4,601,978. See also Reyes et al., Nature 297:598-601 (1982) on expression of human β-interferon cDNA in mouse cells under the control of a thymidine kinase promoter from herpes simplex virus. Alternatively, the Rous Sarcoma Virus long terminal repeat can be used as the promoter.

(v) Enhancer Element Component

Transcription of DNA encoding the antibody polypeptide of this invention by higher eukaryotes is often increased by inserting an enhancer sequence into the vector. Many enhancer sequences are now known from mammalian genes (globin, elastase, albumin, α-fetoprotein, and insulin). Typically, however, one will use an enhancer from a eukaryotic cell virus. Examples include the SV40 enhancer on the late side of the replication origin (bp 100-270), the cytomegalovirus early promoter enhancer, the polyoma enhancer on the late side of the replication origin, and adenovirus enhancers. See also Yaniv, Nature 297:17-18 (1982) on enhancing elements for activation of eukaryotic promoters. The enhancer may be spliced into the vector at a position 5′ or 3′ to the antibody polypeptide-encoding sequence. In certain embodiments, the enhancer is located at a site 5′ from the promoter.

(vi) Transcription Termination Component

Expression vectors used in eukaryotic host cells will typically also contain sequences necessary for the termination of transcription and for stabilizing the mRNA. Such sequences are commonly available from the 5′ and, occasionally 3′, untranslated regions of eukaryotic or viral DNAs or cDNAs. These regions contain nucleotide segments transcribed as polyadenylated fragments in the untranslated portion of the mRNA encoding an antibody. One useful transcription termination component is the bovine growth hormone polyadenylation region. See WO94/11026 and the expression vector disclosed therein.

(vii) Selection and Transformation of Host Cells

Suitable host cells for cloning or expressing the DNA in the vectors herein include higher eukaryote cells described herein, including vertebrate host cells. Propagation of vertebrate cells in culture (tissue culture) has become a routine procedure. Examples of useful mammalian host cell lines are monkey kidney CV1 line transformed by SV40 (COS-7, ATCC CRL 1651); human embryonic kidney line (293 or 293 cells subcloned for growth in suspension culture, Graham et al., J. Gen Virol. 36:59 (1977)); baby hamster kidney cells (BHK, ATCC CCL 10); Chinese hamster ovary cells/-DHFR (CHO, Urlaub et al., Proc. Natl. Acad. Sci. USA 77:4216 (1980)); mouse sertoli cells (TM4, Mather, Biol. Reprod. 23:243-251 (1980)); monkey kidney cells (CV1 ATCC CCL 70); African green monkey kidney cells (VERO-76, ATCC CRL-1587); human cervical carcinoma cells (HELA, ATCC CCL 2); canine kidney cells (MDCK, ATCC CCL 34); buffalo rat liver cells (BRL 3A, ATCC CRL 1442); human lung cells (W138, ATCC CCL 75); human liver cells (Hep G2, HB 8065); mouse mammary tumor (MMT 060562, ATCC CCL51); TRI cells (Mather et al., Annals N.Y. Acad. Sci. 383:44-68 (1982)); MRC 5 cells; FS4 cells; and a human hepatoma line (Hep G2).

Host cells are transformed with the above-described expression or cloning vectors for antibody production and cultured in conventional nutrient media modified as appropriate for inducing promoters, selecting transformants, or amplifying the genes encoding the desired sequences.

(viii) Culturing the Host Cells

The host cells used to produce an antibody of this invention may be cultured in a variety of media. Commercially available media such as Ham's F10 (Sigma), Minimal Essential Medium ((MEM), (Sigma), RPMI-1640 (Sigma), and Dulbecco's Modified Eagle's Medium ((DMEM), Sigma) are suitable for culturing the host cells. In addition, any of the media described in Ham et al., Meth. Enz. 58:44 (1979), Barnes et al., Anal. Biochem.102:255 (1980), U.S. Pat. Nos. 4,767,704; 4,657,866; 4,927,762; 4,560,655; or 5,122,469; WO 90/03430; WO 87/00195; or U.S. Pat. Re. 30,985 may be used as culture media for the host cells. Any of these media may be supplemented as necessary with hormones and/or other growth factors (such as insulin, transferrin, or epidermal growth factor), salts (such as sodium chloride, calcium, magnesium, and phosphate), buffers (such as HEPES), nucleotides (such as adenosine and thymidine), antibiotics (such as GENTAMYCIN™ drug), trace elements (defined as inorganic compounds usually present at final concentrations in the micromolar range), and glucose or an equivalent energy source. Any other necessary supplements may also be included at appropriate concentrations that would be known to those skilled in the art. The culture conditions, such as temperature, pH, and the like, are those previously used with the host cell selected for expression, and will be apparent to the ordinarily skilled artisan.

(ix) Purification of Antibody

When using recombinant techniques, the antibody can be produced intracellularly, or directly secreted into the medium. If the antibody is produced intracellularly, as a first step, the particulate debris, either host cells or lysed fragments, are removed, for example, by centrifugation or ultrafiltration. Where the antibody is secreted into the medium, supernatants from such expression systems are generally first concentrated using a commercially available protein concentration filter, for example, an Amicon or Millipore Pellicon ultrafiltration unit. A protease inhibitor such as PMSF may be included in any of the foregoing steps to inhibit proteolysis and antibiotics may be included to prevent the growth of adventitious contaminants.

The antibody composition prepared from the cells can be purified using, for example, hydroxylapatite chromatography, gel electrophoresis, dialysis, and affinity chromatography. The suitability of protein A as an affinity ligand depends on the species and isotype of any immunoglobulin Fc domain that is present in the antibody. Protein A can be used to purify antibodies that are based on human γ1, γ2, or γ4 heavy chains (Lindmark et al., J. Immunol. Meth. 62:1-13 (1983)). Protein G is recommended for all mouse isotypes and for human γ3 (Guss et al., EMBO J. 5:15671575 (1986)). The matrix to which the affinity ligand is attached is most often agarose, but other matrices are available. Mechanically stable matrices such as controlled pore glass or poly(styrenedivinyl)benzene allow for faster flow rates and shorter processing times than can be achieved with agarose. Where the antibody comprises a C_{H}3 domain, the Bakerbond ABX™resin (J. T. Baker, Phillipsburg, N.J.) is useful for purification. Other techniques for protein purification such as fractionation on an ion-exchange column, ethanol precipitation, Reverse Phase HPLC, chromatography on silica, chromatography on heparin SEPHAROSE™ chromatography on an anion or cation exchange resin (such as a polyaspartic acid column), chromatofocusing, SDS-PAGE, and ammonium sulfate precipitation are also available depending on the antibody to be recovered.

Following any preliminary purification step(s), the mixture comprising the antibody of interest and contaminants may be subjected to low pH hydrophobic interaction chromatography using an elution buffer at a pH between about 2.5-4.5. In certain embodiments, the low pH hydrophobic interaction chromatography is performed at low salt concentrations (e.g., from about 0-0.25M salt).

Activity Assays

The antibodies of the present invention can be characterized for their physical/chemical properties and biological functions by various assays known in the art.

The purified immunoglobulins can be further characterized by a series of assays including, but not limited to, N-terminal sequencing, amino acid analysis, non-denaturing size exclusion high pressure liquid chromatography (HPLC), mass spectrometry, ion exchange chromatography and papain digestion.

In certain embodiments, the immunoglobulins produced herein are analyzed for their biological activity. In some embodiments, the immunoglobulins of the present invention are tested for their antigen binding activity. The antigen binding assays that are known in the art and can be used herein include without limitation any direct or competitive binding assays using techniques such as western blots, radioimmunoassays, ELISA (enzyme linked immunosorbent assay), “sandwich” immunoassays, immunoprecipitation assays, fluorescent immunoassays, and protein A immunoassays.

In one embodiment, the present invention contemplates an altered antibody that possesses some but not all effector functions, which make it a desired candidate for many applications in which the half life of the antibody in vivo is important yet certain effector functions (such as complement and ADCC) are unnecessary or deleterious. In certain embodiments, the Fc activities of the produced immunoglobulin are measured to ensure that only the desired properties are maintained. In vitro and/or in vivo cytotoxicity assays can be conducted to confirm the reduction/depletion of CDC and/or ADCC activities. For example, Fc receptor (FcR) binding assays can be conducted to ensure that the antibody lacks FcγR binding (hence likely lacking ADCC activity), but retains FcRn binding ability. The primary cells for mediating ADCC, NK cells, express FcγRIII only, whereas monocytes express FcγRI, FcγRII and FcγRIII. FcR expression on hematopoietic cells is summarized in Table 3 on page 464 of Ravetch and Kinet, Annu. Rev. Immunol 9:457-92 (1991). An example of an in vitro assay to assess ADCC activity of a molecule of interest is described in U.S. Pat. Nos. 5,500,362 or 5,821,337. Useful effector cells for such assays include peripheral blood mononuclear cells (PBMC) and Natural Killer (NK) cells. Alternatively, or additionally, ADCC activity of the molecule of interest may be assessed in vivo, e.g., in a animal model such as that disclosed in Clynes et al. PNAS (USA) 95:652-656 (1998). C1q binding assays may also be carried out to confirm that the antibody is unable to bind C1q and hence lacks CDC activity. To assess complement activation, a CDC assay, for example as described in Gazzano-Santoro et al., J. Immunol. Methods 202:163 (1996), may be performed. FcRn binding and in vivo clearance/half life determinations can also be performed using methods known in the art, e.g., those described in the Examples section.

Humanized Antibodies

The present invention encompasses humanized antibodies. Various methods for humanizing non-human antibodies are known in the art. For example, a humanized antibody can have one or more amino acid residues introduced into it from a source which is non-human. These non-human amino acid residues are often referred to as “import” residues, which are typically taken from an “import” variable domain. Humanization can be essentially performed following the method of Winter and co-workers (Jones et al. (1986) Nature 321:522-525; Riechmann et al. (1988) Nature 332:323-327; Verhoeyen et al. (1988) Science 239:1534-1536), by substituting hypervariable region sequences for the corresponding sequences of a human antibody. Accordingly, such “humanized” antibodies are chimeric antibodies (U.S. Pat. No. 4,816,567) wherein substantially less than an intact human variable domain has been substituted by the corresponding sequence from a non-human species. In practice, humanized antibodies are typically human antibodies in which some hypervariable region residues and possibly some FR residues are substituted by residues from analogous sites in rodent antibodies.

The choice of human variable domains, both light and heavy, to be used in making the humanized antibodies is very important to reduce antigenicity. According to the so-called “best-fit” method, the sequence of the variable domain of a rodent antibody is screened against the entire library of known human variable-domain sequences. The human sequence which is closest to that of the rodent is then accepted as the human framework for the humanized antibody (Sims et al. (1993) J. Immunol. 151:2296; Chothia et al. (1987) J. Mol. Biol. 196:901). Another method uses a particular framework derived from the consensus sequence of all human antibodies of a particular subgroup of light or heavy chains. The same framework may be used for several different humanized antibodies (Carter et al. (1992) Proc. Natl. Acad. Sci. USA, 89:4285; Presta et al. (1 993) J. Immunol., 151:2623).

It is further important that antibodies be humanized with retention of high affinity for the antigen and other favorable biological properties. To achieve this goal, according to one method, humanized antibodies are prepared by a process of analysis of the parental sequences and various conceptual humanized products using three-dimensional models of the parental and humanized sequences. Three-dimensional immunoglobulin models are commonly available and are familiar to those skilled in the art. Computer programs are available which illustrate and display probable three-dimensional conformational structures of selected candidate immunoglobulin sequences. Inspection of these displays permits analysis of the likely role of the residues in the functioning of the candidate immunoglobulin sequence, i.e., the analysis of residues that influence the ability of the candidate immunoglobulin to bind its antigen. In this way, FR residues can be selected and combined from the recipient and import sequences so that the desired antibody characteristic, such as increased affinity for the target antigen(s), is achieved. In general, the hypervariable region residues are directly and most substantially involved in influencing antigen binding.

Antibody Variants

In one aspect, the invention provides antibody fragments comprising modifications in the interface of Fc polypeptides comprising the Fc region, wherein the modifications facilitate and/or promote heterodimerization. These modifications comprise introduction of a protuberance into a first Fc polypeptide and a cavity into a second Fc polypeptide, wherein the protuberance is positionable in the cavity so as to promote complexing of the first and second Fc polypeptides. Methods of generating antibodies with these modifications are known in the art, e.g., as described in U.S. Pat. No. 5,731,168.

In some embodiments, amino acid sequence modification(s) of the antibodies described herein are contemplated. For example, it may be desirable to improve the binding affinity and/or other biological properties of the antibody. Amino acid sequence variants of the antibody are prepared by introducing appropriate nucleotide changes into the antibody nucleic acid, or by peptide synthesis. Such modifications include, for example, deletions from, and/or insertions into and/or substitutions of, residues within the amino acid sequences of the antibody. Any combination of deletion, insertion, and substitution is made to arrive at the final construct, provided that the final construct possesses the desired characteristics. The amino acid alterations may be introduced in the subject antibody amino acid sequence at the time that sequence is made.

A useful method for identification of certain residues or regions of the antibody that are preferred locations for mutagenesis is called “alanine scanning mutagenesis” as described by Cunningham and Wells (1989) Science, 244:1081-1085. Here, a residue or group of target residues are identified (e.g., charged residues such as arg, asp, his, lys, and glu) and replaced by a neutral or negatively charged amino acid (e.g., alanine or polyalanine) to affect the interaction of the amino acids with antigen. Those amino acid locations demonstrating functional sensitivity to the substitutions then are refined by introducing further or other variants at, or for, the sites of substitution. Thus, while the site for introducing an amino acid sequence variation is predetermined, the nature of the mutation per se need not be predetermined. For example, to analyze the performance of a mutation at a given site, ala scanning or random mutagenesis is conducted at the target codon or region and the expressed immunoglobulins are screened for the desired activity.

Amino acid sequence insertions include amino- and/or carboxyl-terminal fusions ranging in length from one residue to polypeptides containing a hundred or more residues, as well as intrasequence insertions of single or multiple amino acid residues. Examples of terminal insertions include an antibody with an N-terminal methionyl residue or the antibody fused to a cytotoxic polypeptide. Other insertional variants of the antibody molecule include the fusion to the N- or C-terminus of the antibody to an enzyme (e.g. for ADEPT) or a polypeptide which increases the serum half-life of the antibody.

Another type of variant is an amino acid substitution variant. These variants have at least one amino acid residue in the antibody molecule replaced by a different residue. The sites of greatest interest for substitutional mutagenesis include the hypervariable regions, but FR alterations are also contemplated. Conservative substitutions are shown in Table 2 under the heading of “preferred substitutions”. If such substitutions result in a change in biological activity, then more substantial changes, denominated “exemplary substitutions” in the table below, or as further described below in reference to amino acid classes, may be introduced and the products screened.

| Original | Exemplary | Preferred |
|---|---|---|
| Residue | Substitutions | Substitutions |
| Ala (A) | Val; Leu; Ile | Val |
| Arg (R) | Lys; Gln; Asn | Lys |
| Asn (N) | Gln; His; Asp, Lys; Arg | Gln |
| Asp (D) | Glu; Asn | Glu |
| Cys (C) | Ser; Ala | Ser |
| Gln (Q) | Asn; Glu | Asn |
| Glu (E) | Asp; Gln | Asp |
| Gly (G) | Ala | Ala |
| His (H) | Asn; Gln; Lys; Arg | Arg |
| Ile (I) | Leu; Val; Met; Ala; Phe; | Leu |
| | Norleucine | |
| Leu (L) | Norleucine; Ile; Val; Met; | Ile |
| | Ala; Phe | |
| Lys (K) | Arg; Gln; Asn | Arg |
| Met (M) | Leu; Phe; Ile | Leu |
| Phe (F) | Trp; Leu; Val; Ile; Ala; Tyr | Tyr |
| Pro (P) | Ala | Ala |
| Ser (S) | Thr | Thr |
| Thr (T) | Val; Ser | Ser |
| Trp (W) | Tyr; Phe | Tyr |
| Tyr (Y) | Trp; Phe; Thr; Ser | Phe |
| Val (V) | Ile; Leu; Met; Phe; Ala; | Leu |
| | Norleucine | |

Substantial modifications in the biological properties of the antibody are accomplished by selecting substitutions that differ significantly in their effect on maintaining (a) the structure of the polypeptide backbone in the area of the substitution, for example, as a sheet or helical conformation, (b) the charge or hydrophobicity of the molecule at the target site, or (c) the bulk of the side chain. Amino acids may be grouped according to similarities in the properties of their side chains (in A. L. Lehninger, in Biochemistry, second ed., pp. 73-75, Worth Publishers, New York (1975)):
(1) non-polar: Ala (A), Val (V), Leu (L), Ile (I), Pro (P), Phe (F), Trp (W), Met (M)
(2) uncharged polar: Gly (G), Ser (S), Thr (T), Cys (C), Tyr (Y), Asn (N), Gln (Q)
(3) acidic: Asp (D), Glu (E)
(4) basic: Lys (K), Arg (R), His(H)

Alternatively, naturally occurring residues may be divided into groups based on common side-chain properties:

(1) hydrophobic: Norleucine, Met, Ala, Val, Leu, Ile;

(2) neutral hydrophilic: Cys, Ser, Thr. Asn, Gln;

(3) acidic: Asp, Glu;

(4) basic: His, Lys, Arg;

(5) residues that influence chain orientation: Gly, Pro;

(6) aromatic: Trp, Tyr, Phe.

Non-conservative substitutions will entail exchanging a member of one of these classes for another class. Such substituted residues also may be introduced into the conservative substitution sites or, into the remaining (non-conserved) sites.

One type of substitutional variant involves substituting one or more hypervariable region residues of a parent antibody (e.g. a humanized or human antibody). Generally, the resulting variant(s) selected for further development will have improved biological properties relative to the parent antibody from which they are generated. A convenient way for generating such substitutional variants involves affinity maturation using phage display. Briefly, several hypervariable region sites (e.g. 6-7 sites) are mutated to generate all possible amino acid substitutions at each site. The antibodies thus generated are displayed from filamentous phage particles as fusions to the gene III product of M13 packaged within each particle. The phage-displayed variants are then screened for their biological activity (e.g. binding affinity) as herein disclosed. In order to identify candidate hypervariable region sites for modification, alanine scanning mutagenesis can be performed to identify hypervariable region residues contributing significantly to antigen binding. Alternatively, or additionally, it may be beneficial to analyze a crystal structure of the antigen-antibody complex to identify contact points between the antibody and antigen. Such contact residues and neighboring residues are candidates for substitution according to the techniques elaborated herein. Once such variants are generated, the panel of variants is subjected to screening as described herein and antibodies with superior properties in one or more relevant assays may be selected for further development.

Nucleic acid molecules encoding amino acid sequence variants of the antibody are prepared by a variety of methods known in the art. These methods include, but are not limited to, isolation from a natural source (in the case of naturally occurring amino acid sequence variants) or preparation by oligonucleotide-mediated (or site-directed) mutagenesis, PCR mutagenesis, and cassette mutagenesis of an earlier prepared variant or a non-variant version of the antibody.

It may be desirable to introduce one or more amino acid modifications in an Fc region of the immunoglobulin polypeptides of the invention, thereby generating an Fc region variant. The Fc region variant may comprise a human Fc region sequence (e.g., a human IgG1, IgG2, IgG3 or IgG4 Fc region) comprising an amino acid modification (e.g. a substitution) at one or more amino acid positions including that of a hinge cysteine.

In accordance with this description and the teachings of the art, it is contemplated that in some embodiments, an antibody used in methods of the invention may comprise one or more alterations as compared to the wild type counterpart antibody, for example in the Fc region. These antibodies would nonetheless retain substantially the same characteristics required for therapeutic utility as compared to their wild type counterpart. For example, it is thought that certain alterations can be made in the Fc region that would result in altered (i.e., either improved or diminished) C1q binding and/or Complement Dependent Cytotoxicity (CDC), for example, as described in WO99/51642. See also Duncan & Winter Nature 322:738-40 (1988); U.S. Pat. No. 5,648,260; U.S. Pat. No. 5,624,821; and WO94/29351 concerning other examples of Fc region variants.

Immunoconjugates

The invention also pertains to immunoconjugates, or antibody-drug conjugates (ADC), comprising an antibody conjugated to a cytotoxic agent such as a chemotherapeutic agent, a drug, a growth inhibitory agent, a toxin (e.g., an enzymatically active toxin of bacterial, fungal, plant, or animal origin, or fragments thereof), or a radioactive isotope (i.e., a radioconjugate).

The use of antibody-drug conjugates for the local delivery of cytotoxic or cytostatic agents, i.e. drugs to kill or inhibit tumor cells in the treatment of cancer (Syrigos and Epenetos (1999) Anticancer Research 19:605-614; Niculescu-Duvaz and Springer (1997) Adv. Drg Del. Rev. 26:151-172; U.S. Pat. No. 4,975,278) theoretically allows targeted delivery of the drug moiety to tumors, and intracellular accumulation therein, where systemic administration of these unconjugated drug agents may result in unacceptable levels of toxicity to normal cells as well as the tumor cells sought to be eliminated (Baldwin et al., (1986) Lancet pp. (Mar. 15, 1986):603-05; Thorpe, (1985) “Antibody Carriers Of Cytotoxic Agents In Cancer Therapy: A Review,” in Monoclonal Antibodies '84: Biological And Clinical Applications, A. Pinchera et al. (eds.), pp. 475-506). Maximal efficacy with minimal toxicity is sought thereby. Both polyclonal antibodies and monoclonal antibodies have been reported as useful in these strategies (Rowland et al., (1986) Cancer Immunol. Immunother., 21:183-87). Drugs used in these methods include daunomycin, doxorubicin, methotrexate, and vindesine (Rowland et al., (1986) supra). Toxins used in antibody-toxin conjugates include bacterial toxins such as diphtheria toxin, plant toxins such as ricin, small molecule toxins such as geldanamycin (Mandler et al (2000) Jour. of the Nat. Cancer Inst. 92(19):1573-1581; Mandler et al (2000) Bioorganic & Med. Chem. Letters 10:1025-1028; Mandler et al (2002) Bioconjugate Chem. 13:786-791), maytansinoids (EP 1391213; Liu et al., (1996) Proc. Natl. Acad. Sci. USA 93:8618-8623), and calicheamicin (Lode et al (1998) Cancer Res. 58:2928; Hinman et al (1993) Cancer Res. 53:3336-3342). The toxins may effect their cytotoxic and cytostatic effects by mechanisms including tubulin binding, DNA binding, or topoisomerase inhibition. Some cytotoxic drugs tend to be inactive or less active when conjugated to large antibodies or protein receptor ligands.

ZEVALIN® (ibritumomab tiuxetan, Biogen/Idec) is an antibody-radioisotope conjugate composed of a murine IgG1 kappa monoclonal antibody directed against the CD20 antigen found on the surface of normal and malignant B lymphocytes and ¹¹¹In or ⁹⁰Y radioisotope bound by a thiourea linker-chelator (Wiseman et al (2000) Eur. Jour. Nucl. Med. 27(7):766-77; Wiseman et al (2002) Blood 99(12):4336-42; Witzig et al (2002) J. Clin. Oncol. 20(10):2453-63; Witzig et al (2002) J. Clin. Oncol. 20(15):3262-69). Although ZEVALIN has activity against B-cell non-Hodgkin's Lymphoma (NHL), administration results in severe and prolonged cytopenias in most patients. MYLOTARG™ (gemtuzumab ozogamicin, Wyeth Pharmaceuticals), an antibody drug conjugate composed of a hu CD33 antibody linked to calicheamicin, was approved in 2000 for the treatment of acute myeloid leukemia by injection (Drugs of the Future (2000) 25(7):686; U.S. Pat. Nos. 4,970,198; 5,079,233; 5,585,089; 5,606,040; 5,693,762; 5,739,116; 5,767,285; 5,773,001). Cantuzumab mertansine (Immunogen, Inc.), an antibody drug conjugate composed of the huC242 antibody linked via the disulfide linker SPP to the maytansinoid drug moiety, DM1, is advancing into Phase II trials for the treatment of cancers that express CanAg, such as colon, pancreatic, gastric, and others. MLN-2704 (Millennium Pharm., BZL Biologics, Immunogen Inc.), an antibody drug conjugate composed of the anti-prostate specific membrane antigen (PSMA) monoclonal antibody linked to the maytansinoid drug moiety, DM1, is under development for the potential treatment of prostate tumors. The auristatin peptides, auristatin E (AE) and monomethylauristatin (MMAE), synthetic analogs of dolastatin, were conjugated to chimeric monoclonal antibodies cBR96 (specific to Lewis Y on carcinomas) and cAC10 (specific to CD30 on hematological malignancies) (Doronina et al (2003) Nature Biotechnology 21(7):778-784) and are under therapeutic development.

Chemotherapeutic agents useful in the generation of such immunoconjugates have been described above. Enzymatically active toxins and fragments thereof that can be used include without limitation diphtheria A chain, nonbinding active fragments of diphtheria toxin, exotoxin A chain (from Pseudomonas aeruginosa), ricin A chain, abrin A chain, modeccin A chain, alpha-sarcin, Aleurites fordii proteins, dianthin proteins, Phytolaca americana proteins (PAPI, PAPII, and PAP-S), momordica charantia inhibitor, curcin, crotin, sapaonaria officinalis inhibitor, gelonin, mitogellin, restrictocin, phenomycin, enomycin, and the tricothecenes. A variety of radionuclides are available for the production of radioconjugated antibodies. Examples include ²¹²Bi, ¹³¹I, ¹³¹In, ⁹⁰Y, and ¹⁸⁶Re. Conjugates of the antibody and cytotoxic agent are made using a variety of bifunctional protein-coupling agents such as N-succinimidyl-3-(2-pyridyldithiol)propionate (SPDP), iminothiolane (IT), bifunctional derivatives of imidoesters (such as dimethyl adipimidate HCl), active esters (such as disuccinimidyl suberate), aldehydes (such as glutaraldehyde), bis-azido compounds (such as bis (p-azidobenzoyl)hexanediamine), bis-diazonium derivatives (such as bis-(p-diazoniumbenzoyl)-ethylenediamine), diisocyanates (such as toluene 2,6-diisocyanate), and bis-active fluorine compounds (such as 1,5-difluoro-2,4-dinitrobenzene). For example, a ricin immunotoxin can be prepared as described in Vitetta et al., Science, 238: 1098 (1987). Carbon-14-labeled 1-isothiocyanatobenzyl-3-methyldiethylene triaminepentaacetic acid (MX-DTPA) is an exemplary chelating agent for conjugation of radionucleotide to the antibody. See WO94/11026.

Conjugates of an antibody and one or more small molecule toxins, such as a calicheamicin, maytansinoids, a trichothecene, and CC1065, and the derivatives of these toxins that have toxin activity, are also contemplated herein.

Maytansine and Maytansinoids

In one embodiment, an antibody (full length or fragments) of the invention is conjugated to one or more maytansinoid molecules.

Maytansinoids are mitotic inhibitors which act by inhibiting tubulin polymerization. Maytansine was first isolated from the east African shrub Maytenus serrata (U.S. Pat. No. 3,896,111). Subsequently, it was discovered that certain microbes also produce maytansinoids, such as maytansinol and C-3 maytansinol esters (U.S. Pat. No. 4,151,042). Synthetic maytansinol and derivatives and analogues thereof are disclosed, for example, in U.S. Pat. Nos. 4,137,230; 4,248,870; 4,256,746; 4,260,608; 4,265,814; 4,294,757; 4,307,016; 4,308,268; 4,308,269; 4,309,428; 4,313,946; 4,315,929; 4,317,821; 4,322,348; 4,331,598; 4,361,650; 4,364,866; 4,424,219; 4,450,254; 4,362,663; and 4,371,533, the disclosures of which are hereby expressly incorporated by reference.

Maytansinoid-Antibody Conjugates

In an attempt to improve their therapeutic index, maytansine and maytansinoids have been conjugated to antibodies specifically binding to tumor cell antigens. Immunoconjugates containing maytansinoids and their therapeutic use are disclosed, for example, in U.S. Pat. Nos. 5,208,020, 5,416,064 and European Patent EP 0 425 235 B1, the disclosures of which are hereby expressly incorporated by reference. Liu et al., Proc. Natl. Acad. Sci. USA 93:8618-8623 (1996) described immunoconjugates comprising a maytansinoid designated DM1 linked to the monoclonal antibody C242 directed against human colorectal cancer. The conjugate was found to be highly cytotoxic towards cultured colon cancer cells, and showed antitumor activity in an in vivo tumor growth assay. Chari et al., Cancer Research 52:127-131 (1992) describe immunoconjugates in which a maytansinoid was conjugated via a disulfide linker to the murine antibody A7 binding to an antigen on human colon cancer cell lines, or to another murine monoclonal antibody TA.1 that binds the HER-2/neu oncogene. The cytotoxicity of the TA.1-maytansonoid conjugate was tested in vitro on the human breast cancer cell line SK-BR-3, which expresses 3×10⁵ HER-2 surface antigens per cell. The drug conjugate achieved a degree of cytotoxicity similar to the free maytansinoid drug, which could be increased by increasing the number of maytansinoid molecules per antibody molecule. The A7-maytansinoid conjugate showed low systemic cytotoxicity in mice.

Antibody-Maytansinoid Conjugates (Immunoconjugates)

Antibody-maytansinoid conjugates are prepared by chemically linking an antibody to a maytansinoid molecule without significantly diminishing the biological activity of either the antibody or the maytansinoid molecule. An average of 3-4 maytansinoid molecules conjugated per antibody molecule has shown efficacy in enhancing cytotoxicity of target cells without negatively affecting the function or solubility of the antibody, although even one molecule of toxin/antibody would be expected to enhance cytotoxicity over the use of naked antibody. Maytansinoids are well known in the art and can be synthesized by known techniques or isolated from natural sources. Suitable maytansinoids are disclosed, for example, in U.S. Pat. No. 5,208,020 and in the other patents and nonpatent publications referred to hereinabove. In certain embodiments, maytansinoids are maytansinol and maytansinol analogues modified in the aromatic ring or at other positions of the maytansinol molecule, such as various maytansinol esters.

There are many linking groups known in the art for making antibody-maytansinoid conjugates, including, for example, those disclosed in U.S. Pat. No. 5,208,020 or EP Patent 0 425 235 B1, and Chari et al., Cancer Research 52:127-131 (1992). The linking groups include disulfide groups, thioether groups, acid labile groups, photolabile groups, peptidase labile groups, or esterase labile groups, as disclosed in the above-identified patents.

Conjugates of the antibody and maytansinoid may be made using a variety of bifunctional protein coupling agents such as N-succinimidyl-3-(2-pyridyldithio)propionate (SPDP), succinimidyl-4-(N-maleimidomethyl)cyclohexane-1-carboxylate, iminothiolane (IT), bifunctional derivatives of imidoesters (such as dimethyl adipimidate HCl), active esters (such as disuccinimidyl suberate), aldehydes (such as glutaraldehyde), bis-azido compounds (such as bis (p-azidobenzoyl)hexanediamine), bis-diazonium derivatives (such as bis-(p-diazoniumbenzoyl)-ethylenediamine), diisocyanates (such as toluene 2,6-diisocyanate), and bis-active fluorine compounds (such as 1,5-difluoro-2,4-dinitrobenzene). In certain embodiments, coupling agents include N-succinimidyl-3-(2-pyridyldithio)propionate (SPDP) (Carlsson et al., Biochem. J. 173:723-737 [1978]) and N-succinimidyl-4-(2-pyridylthio)pentanoate (SPP) to provide for a disulfide linkage.

The linker may be attached to the maytansinoid molecule at various positions, depending on the type of the link. For example, an ester linkage may be formed by reaction with a hydroxyl group using conventional coupling techniques. The reaction may occur at the C-3 position having a hydroxyl group, the C-14 position modified with hydroxymethyl, the C-15 position modified with a hydroxyl group, and the C-20 position having a hydroxyl group. In one embodiment, the linkage is formed at the C-3 position of maytansinol or a maytansinol analogue.

Calicheamicin

Another immunoconjugate of interest comprises an antibody conjugated to one or more calicheamicin molecules. The calicheamicin family of antibiotics are capable of producing double-stranded DNA breaks at sub-picomolar concentrations. For the preparation of conjugates of the calicheamicin family, see U.S. Pat. Nos. 5,712,374, 5,714,586, 5,739,116, 5,767,285, 5,770,701, 5,770,710, 5,773,001, 5,877,296 (all to American Cyanamid Company). Structural analogues of calicheamicin which may be used include, but are not limited to, γ₁^{I}, α₂^{I}, α₃^{I}, N-acetyl-γ₁^{I}, PSAG and θ^{I}₁ (Hinman et al., Cancer Research 53:3336-3342 (1993), Lode et al., Cancer Research 58:2925-2928 (1998) and the aforementioned U.S. patents to American Cyanamid). Another anti-tumor drug that the antibody can be conjugated is QFA which is an antifolate. Both calicheamicin and QFA have intracellular sites of action and do not readily cross the plasma membrane. Therefore, cellular uptake of these agents through antibody mediated internalization greatly enhances their cytotoxic effects.

Other Cytotoxic Agents

Other antitumor agents that can be conjugated to the antibodies of the invention include BCNU, streptozoicin, vincristine and 5-fluorouracil, the family of agents known collectively LL-E33288 complex described in U.S. Pat. Nos. 5,053,394, 5,770,710, as well as esperamicins (U.S. Pat. No. 5,877,296).

Enzymatically active toxins and fragments thereof which can be used include diphtheria A chain, nonbinding active fragments of diphtheria toxin, exotoxin A chain (from Pseudomonas aeruginosa), ricin A chain, abrin A chain, modeccin A chain, alpha-sarcin, Aleurites fordii proteins, dianthin proteins, Phytolaca americana proteins (PAPI, PAPII, and PAP-S), momordica charantia inhibitor, curcin, crotin, sapaonaria officinalis inhibitor, gelonin, mitogellin, restrictocin, phenomycin, enomycin and the tricothecenes. See, for example, WO 93/21232 published Oct. 28, 1993.

The present invention further contemplates an immunoconjugate formed between an antibody and a compound with nucleolytic activity (e.g., a ribonuclease or a DNA endonuclease such as a deoxyribonuclease; DNase).

For selective destruction of the tumor, the antibody may comprise a highly radioactive atom. A variety of radioactive isotopes are available for the production of radioconjugated antibodies. Examples include At²¹¹, I¹³¹, I¹²⁵, Y⁹⁰, Re¹⁸⁶, Re¹⁸⁸, Sm¹⁵³, Bi²¹², P³², Pb²¹² and radioactive isotopes of Lu. When the conjugate is used for detection, it may comprise a radioactive atom for scintigraphic studies, for example tc^{99m} or I¹²³, or a spin label for nuclear magnetic resonance (NMR) imaging (also known as magnetic resonance imaging, MRI), such as iodine-123 again, iodine-131, indium-111, fluorine-19, carbon-13, nitrogen-15, oxygen-17, gadolinium, manganese or iron.

The radio- or other labels may be incorporated in the conjugate in known ways. For example, the peptide may be biosynthesized or may be synthesized by chemical amino acid synthesis using suitable amino acid precursors involving, for example, fluorine-19 in place of hydrogen. Labels such as tc^{99m} or I¹²³, .Re¹⁸⁶, Re¹⁸⁸ and In¹¹¹ can be attached via a cysteine residue in the peptide. Yttrium-90 can be attached via a lysine residue. The IODOGEN method (Fraker et al (1978) Biochem. Biophys. Res. Commun. 80: 49-57) can be used to incorporate iodine-123. “Monoclonal Antibodies in Immunoscintigraphy” (Chatal, CRC Press 1989) describes other methods in detail.

Conjugates of the antibody and cytotoxic agent may be made using a variety of bifunctional protein coupling agents such as N-succinimidyl-3-(2-pyridyldithio)propionate (SPDP), succinimidyl-4-(N-maleimidomethyl)cyclohexane-1-carboxylate, iminothiolane (IT), bifunctional derivatives of imidoesters (such as dimethyl adipimidate HCl), active esters (such as disuccinimidyl suberate), aldehydes (such as glutaraldehyde), bis-azido compounds (such as bis (p-azidobenzoyl)hexanediamine), bis-diazonium derivatives (such as bis-(p-diazoniumbenzoyl)-ethylenediamine), diisocyanates (such as toluene 2,6-diisocyanate), and bis-active fluorine compounds (such as 1,5-difluoro-2,4-dinitrobenzene). For example, a ricin immunotoxin can be prepared as described in Vitetta et al., Science 238:1098 (1987). Carbon-14-labeled 1-isothiocyanatobenzyl-3-methyldiethylene triaminepentaacetic acid (MX-DTPA) is an exemplary chelating agent for conjugation of radionucleotide to the antibody. See WO94/11026. The linker may be a “cleavable linker” facilitating release of the cytotoxic drug in the cell. For example, an acid-labile linker, peptidase-sensitive linker, photolabile linker, dimethyl linker or disulfide-containing linker (Chari et al., Cancer Research 52:127-131 (1992); U.S. Pat. No. 5,208,020) may be used.

The compounds of the invention expressly contemplate, but are not limited to, ADC prepared with cross-linker reagents: BMPS, EMCS, GMBS, HBVS, LC-SMCC, MBS, MPBH, SBAP, SIA, SIAB, SMCC, SMPB, SMPH, sulfo-EMCS, sulfo-GMBS, sulfo-KMUS, sulfo-MBS, sulfo-SIAB, sulfo-SMCC, and sulfo-SMPB, and SVSB (succinimidyl-(4-vinylsulfone)benzoate) which are commercially available (e.g., from Pierce Biotechnology, Inc., Rockford, Ill., U.S.A). See pages 467-498, 2003-2004 Applications Handbook and Catalog.

Preparation of Antibody Drug Conjugates

In the antibody drug conjugates (ADC) of the invention, an antibody (Ab) is conjugated to one or more drug moieties (D), e.g. about 1 to about 20 drug moieties per antibody, through a linker (L). The ADC of Formula I may be prepared by several routes, employing organic chemistry reactions, conditions, and reagents known to those skilled in the art, including: (1) reaction of a nucleophilic group of an antibody with a bivalent linker reagent, to form Ab-L, via a covalent bond, followed by reaction with a drug moiety D; and (2) reaction of a nucleophilic group of a drug moiety with a bivalent linker reagent, to form D-L, via a covalent bond, followed by reaction with the nucleophilic group of an antibody.
Ab−(L−D)ₚ I

Nucleophilic groups on antibodies include, but are not limited to: (i) N-terminal amine groups, (ii) side chain amine groups, e.g. lysine, (iii) side chain thiol groups, e.g. cysteine, and (iv) sugar hydroxyl or amino groups where the antibody is glycosylated. Amine, thiol, and hydroxyl groups are nucleophilic and capable of reacting to form covalent bonds with electrophilic groups on linker moieties and linker reagents including: (i) active esters such as NHS esters, HOBt esters, haloformates, and acid halides; (ii) alkyl and benzyl halides such as haloacetamides; (iii) aldehydes, ketones, carboxyl, and maleimide groups. Certain antibodies have reducible interchain disulfides, i.e. cysteine bridges. Antibodies may be made reactive for conjugation with linker reagents by treatment with a reducing agent such as DTT (dithiothreitol). Each cysteine bridge will thus form, theoretically, two reactive thiol nucleophiles. Additional nucleophilic groups can be introduced into antibodies through the reaction of lysines with 2-iminothiolane (Traut's reagent) resulting in conversion of an amine into a thiol.

Antibody drug conjugates of the invention may also be produced by modification of the antibody to introduce electrophilic moieties, which can react with nucleophilic substituents on the linker reagent or drug. The sugars of glycosylated antibodies may be oxidized, e.g. with periodate oxidizing reagents, to form aldehyde or ketone groups which may react with the amine group of linker reagents or drug moieties. The resulting imine Schiff base groups may form a stable linkage, or may be reduced, e.g. by borohydride reagents to form stable amine linkages. In one embodiment, reaction of the carbohydrate portion of a glycosylated antibody with either galactose oxidase or sodium meta-periodate may yield carbonyl (aldehyde and ketone) groups in the protein that can react with appropriate groups on the drug (Hermanson, Bioconjugate Techniques). In another embodiment, proteins containing N-terminal serine or threonine residues can react with sodium meta-periodate, resulting in production of an aldehyde in place of the first amino acid (Geoghegan & Stroh, (1992) Bioconjugate Chem. 3:138-146; U.S. Pat. No. 5,362,852). Such aldehyde can be reacted with a drug moiety or linker nucleophile.

Likewise, nucleophilic groups on a drug moiety include, but are not limited to: amine, thiol, hydroxyl, hydrazide, oxime, hydrazine, thiosemicarbazone, hydrazine carboxylate, and arylhydrazide groups capable of reacting to form covalent bonds with electrophilic groups on linker moieties and linker reagents including: (i) active esters such as NHS esters, HOBt esters, haloformates, and acid halides; (ii) alkyl and benzyl halides such as haloacetamides; (iii) aldehydes, ketones, carboxyl, and maleimide groups.

Alternatively, a fusion protein comprising the antibody and cytotoxic agent may be made, e.g., by recombinant techniques or peptide synthesis. The length of DNA may comprise respective regions encoding the two portions of the conjugate either adjacent one another or separated by a region encoding a linker peptide which does not destroy the desired properties of the conjugate.

In yet another embodiment, the antibody may be conjugated to a “receptor” (such streptavidin) for utilization in tumor pre-targeting wherein the antibody-receptor conjugate is administered to the patient, followed by removal of unbound conjugate from the circulation using a clearing agent and then administration of a “ligand” (e.g., avidin) which is conjugated to a cytotoxic agent (e.g., a radionucleotide).

Antibody Derivatives

The antibodies of the present invention can be further modified to contain additional nonproteinaceous moieties that are known in the art and readily available. In certain embodiments, the moieties suitable for derivatization of the antibody are water soluble polymers. Non-limiting examples of water soluble polymers include, but are not limited to, polyethylene glycol (PEG), copolymers of ethylene glycol/propylene glycol, carboxymethylcellulose, dextran, polyvinyl alcohol, polyvinyl pyrrolidone, poly-1,3-dioxolane, poly-1,3,6-trioxane, ethylene/maleic anhydride copolymer, polyaminoacids (either homopolymers or random copolymers), and dextran or poly(n-vinyl pyrrolidone)polyethylene glycol, propropylene glycol homopolymers, prolypropylene oxide/ethylene oxide co-polymers, polyoxyethylated polyols (e.g., glycerol), polyvinyl alcohol, and mixtures thereof. Polyethylene glycol propionaldehyde may have advantages in manufacturing due to its stability in water. The polymer may be of any molecular weight, and may be branched or unbranched. The number of polymers attached to the antibody may vary, and if more than one polymers are attached, they can be the same or different molecules. In general, the number and/or type of polymers used for derivatization can be determined based on considerations including, but not limited to, the particular properties or functions of the antibody to be improved, whether the antibody derivative will be used in a therapy under defined conditions, etc.

Pharmaceutical Formulations

Therapeutic formulations comprising an antibody of the invention are prepared for storage by mixing the antibody having the desired degree of purity with optional physiologically acceptable carriers, excipients or stabilizers (Remington's Pharmaceutical Sciences 16th edition, Osol, A. Ed. (1980)), in the form of aqueous solutions, lyophilized or other dried formulations. Acceptable carriers, excipients, or stabilizers are nontoxic to recipients at the dosages and concentrations employed, and include buffers such as phosphate, citrate, histidine and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, niannose, or dextrins; chelating agents such as EDTA; sugars such as sucrose, mannitol, trehalose or sorbitol; salt-forming counter-ions such as sodium; metal complexes (e.g., Zn-protein complexes); and/or non-ionic surfactants such as TWEEN™, PLURONICS™ or polyethylene glycol (PEG).

The formulation herein may also contain more than one active compound as necessary for the particular indication being treated. In certain such embodiments, the compounds have complementary activities that do not adversely affect each other. Such molecules are suitably present in combination in amounts that are effective for the purpose intended.

The active ingredients may also be entrapped in microcapsule prepared, for example, by coacervation techniques or by interfacial polymerization, for example, hydroxymethylcellulose or gelatin-microcapsule and poly-(methylmethacylate) microcapsule, respectively, in colloidal drug delivery systems (for example, liposomes, albumin microspheres, microemulsions, nano-particles and nanocapsules) or in macroemulsions. Such techniques are disclosed in Remington's Pharmaceutical Sciences 16th edition, Osol, A. Ed. (1980).

The formulations to be used for in vivo administration must be sterile. This is readily accomplished, e.g., by filtration through sterile filtration membranes.

Sustained-release preparations may be prepared. Suitable examples of sustained-release preparations include semipermeable matrices of solid hydrophobic polymers containing the immunoglobulin of the invention, which matrices are in the form of shaped articles, e.g., films, or microcapsule. Examples of sustained-release matrices include polyesters, hydrogels (for example, poly(2-hydroxyethyl-methacrylate), or poly(vinylalcohol)), polylactides (U.S. Pat. No. 3,773,919), copolymers of L-glutamic acid and γ ethyl-L-glutamate, non-degradable ethylene-vinyl acetate, degradable lactic acid-glycolic acid copolymers such as the LUPRON DEPOT™ (injectable microspheres composed of lactic acid-glycolic acid copolymer and leuprolide acetate), and poly-D-(−)-3-hydroxybutyric acid. While polymers such as ethylene-vinyl acetate and lactic acid-glycolic acid enable release of molecules for over 100 days, certain hydrogels release proteins for shorter time periods. When encapsulated immunoglobulins remain in the body for a long time, they may denature or aggregate as a result of exposure to moisture at 37° C., resulting in a loss of biological activity and possible changes in immunogenicity. Rational strategies can be devised for stabilization depending on the mechanism involved. For example, if the aggregation mechanism is discovered to be intermolecular S—S bond formation through thio-disulfide interchange, stabilization may be achieved by modifying sulfhydryl residues, lyophilizing from acidic solutions, controlling moisture content, using appropriate additives, and developing specific polymer matrix compositions.

Uses

An antibody of the present invention may be used in, for example, in vitro, ex vivo and in vivo therapeutic methods. Antibodies of the invention can be used as an antagonist to partially or fully block the specific antigen activity in vitro, ex vivo and/or in vivo. Moreover, at least some of the antibodies of the invention can neutralize antigen activity from other species. Accordingly, the antibodies of the invention can be used to inhibit a specific antigen activity, e.g., in a cell culture containing the antigen, in human subjects or in other mammalian subjects having the antigen with which an antibody of the invention cross-reacts (e.g. chimpanzee, baboon, marmoset, cynomolgus and rhesus, pig or mouse). In one embodiment, the antibody of the invention can be used for inhibiting antigen activities by contacting the antibody with the antigen such that antigen activity is inhibited. In certain embodiments, the antigen is a human protein molecule.

In one embodiment, an antibody of the invention can be used in a method for inhibiting an antigen in a subject suffering from a disorder in which the antigen activity is detrimental, comprising administering to the subject an antibody of the invention such that the antigen activity in the subject is inhibited. In certain embodiments, the antigen is a human protein molecule and the subject is a human subject. Alternatively, the subject can be a mammal expressing the antigen with which an antibody of the invention binds. Still further the subject can be a mammal into which the antigen has been introduced (e.g., by administration of the antigen or by expression of an antigen transgene). An antibody of the invention can be administered to a human subject for therapeutic purposes. Moreover, an antibody of the invention can be administered to a non-human mammal expressing an antigen with which the immunoglobulin cross-reacts (e.g., a primate, pig or mouse) for veterinary purposes or as an animal model of human disease. Regarding the latter, such animal models may be useful for evaluating the therapeutic efficacy of antibodies of the invention (e.g., testing of dosages and time courses of administration). Blocking antibodies of the invention that are therapeutically useful include, for example but are not limited to, anti-VEGF and anti-insulin antibodies. For example, the anti-VEGF antibodies of the invention can be used to treat, inhibit, delay progression of, prevent/delay recurrence of, ameliorate, or prevent diseases, disorders or conditions associated with abnormal expression and/or activity of one or more antigen molecules, including but not limited to malignant and benign tumors; non-leukemias and lymphoid malignancies; neuronal, glial, astrocytal, hypothalamic and other glandular, macrophagal, epithelial, stromal and blastocoelic disorders; and inflammatory, angiogenic and immunologic disorders. As another example, the anti-insulin antibodies of the invention can be used to treat, inhibit, delay progression of, prevent/delay recurrence of, ameliorate, or prevent one or more insulin-related disorders (see, e.g., U.S. Patent Application Publication No. US20020081300, describing treating diabetes by administering anti-insulin antibodies in conjunction with anti-glutamic acid decarboxylase antibodies).

In one aspect, a blocking antibody of the invention is specific to a ligand antigen, and inhibits the antigen activity by blocking or interfering with the ligand-receptor interaction involving the ligand antigen, thereby inhibiting the corresponding signal pathway and other molecular or cellular events. The invention also features receptor-specific antibodies which do not necessarily prevent ligand binding but interfere with receptor activation, thereby inhibiting any responses that would normally be initiated by the ligand binding. In certain embodiments, the invention also encompasses antibodies that either preferably or exclusively bind to ligand-receptor complexes. An antibody of the invention can also act as an agonist of a particular antigen receptor, thereby potentiating, enhancing or activating either all or partial activities of the ligand-mediated receptor activation.

In certain embodiments, an immunoconjugate comprising an antibody conjugated with a cytotoxic agent is administered to the patient. In some embodiments, the immunoconjugate and/or antigen to which it is bound is/are internalized by the cell, resulting in increased therapeutic efficacy of the immunoconjugate in killing the target cell to which it binds. In one embodiment, the cytotoxic agent targets or interferes with nucleic acid in the target cell. Examples of such cytotoxic agents include any of the chemotherapeutic agents noted herein (such as a maytansinoid or a calicheamicin), a radioactive isotope, or a ribonuclease or a DNA endonuclease.

Antibodies of the invention can be used either alone or in combination with other compositions in a therapy. For instance, an antibody of the invention may be co-administered with another antibody, chemotherapeutic agent(s) (including cocktails of chemotherapeutic agents), other cytotoxic agent(s), anti-angiogenic agent(s), cytokines, and/or growth inhibitory agent(s). Where an antibody of the invention inhibits tumor growth, it may be particularly desirable to combine it with one or more other therapeutic agent(s) which also inhibits tumor growth. For instance, an antibody of the invention may be combined with an anti-VEGF antibody (e.g., AVASTIN) and/or anti-ErbB antibodies (e.g. HERCEPTIN™ anti-HER2 antibody) in a treatment scheme, e.g. in treating any of the diseases described herein, including colorectal cancer, metastatic breast cancer and kidney cancer. Alternatively, or additionally, the patient may receive combined radiation therapy (e.g. external beam irradiation or therapy with a radioactive labeled agent, such as an antibody). Such combined therapies noted above include combined administration (where the two or more agents are included in the same or separate formulations), and separate administration, in which case, administration of the antibody of the invention can occur prior to, and/or following, administration of the adjunct therapy or therapies.

The antibody of the invention (and adjunct therapeutic agent) is/are administered by any suitable means, including parenteral, subcutaneous, intraperitoneal, intrapulmonary, and intranasal, and, if desired for local treatment, intralesional administration. Parenteral infusions include intramuscular, intravenous, intraarterial, intraperitoneal, or subcutaneous administration. In addition, the antibody is suitably administered by pulse infusion, particularly with declining doses of the antibody. Dosing can be by any suitable route, for example by injections, such as intravenous or subcutaneous injections, depending in part on whether the administration is brief or chronic.

The antibody composition of the invention will be formulated, dosed, and administered in a fashion consistent with good medical practice. Factors for consideration in this context include the particular disorder being treated, the particular mammal being treated, the clinical condition of the individual patient, the cause of the disorder, the site of delivery of the agent, the method of administration, the scheduling of administration, and other factors known to medical practitioners. The antibody need not be, but is optionally formulated with one or more agents currently used to prevent or treat the disorder in question. The effective amount of such other agents depends on the amount of antibodies of the invention present in the formulation, the type of disorder or treatment, and other factors discussed above. These are generally used in the same dosages and with administration routes as used hereinbefore or about from 1 to 99% of the heretofore employed dosages.

For the prevention or treatment of disease, the appropriate dosage of an antibody of the invention (when used alone or in combination with other agents such as chemotherapeutic agents) will depend on the type of disease to be treated, the type of antibody, the severity and course of the disease, whether the antibody is administered for preventive or therapeutic purposes, previous therapy, the patient's clinical history and response to the antibody, and the discretion of the attending physician. The antibody is suitably administered to the patient at one time or over a series of treatments. Depending on the type and severity of the disease, about 1 μg/kg to 15 mg/kg (e.g. 0.1 mg/kg-10 mg/kg) of antibody is an initial candidate dosage for administration to the patient, whether, for example, by one or more separate administrations, or by continuous infusion. One typical daily dosage might range from about 1 μg/kg to 100 mg/kg or more, depending on the factors mentioned above. For repeated administrations over several days or longer, depending on the condition, the treatment is sustained until a desired suppression of disease symptoms occurs. One exemplary dosage of the antibody would be in the range from about 0.05 mg/kg to about 10 mg/kg. Thus, one or more doses of about 0.5 mg/kg, 2.0 mg/kg, 4.0 mg/kg or 10 mg/kg (or any combination thereof) may be administered to the patient. Such doses may be administered intermittently, e.g. every week or every three weeks (e.g. such that the patient receives from about two to about twenty, e.g. about six doses of the antibody). An initial higher loading dose, followed by one or more lower doses may be administered. An exemplary dosing regimen comprises administering an initial loading dose of about 4 mg/kg, followed by a weekly maintenance dose of about 2 mg/kg of the antibody. However, other dosage regimens may be useful. The progress of this therapy is easily monitored by conventional techniques and assays.

Articles of Manufacture

In another aspect of the invention, an article of manufacture containing materials useful for the treatment, prevention and/or diagnosis of the disorders described above is provided. The article of manufacture comprises a container and a label or package insert on or associated with the container. Suitable containers include, for example, bottles, vials, syringes, etc. The containers may be formed from a variety of materials such as glass or plastic. The container holds a composition which is by itself or when combined with another composition effective for treating, preventing and/or diagnosing the condition and may have a sterile access port (for example the container may be an intravenous solution bag or a vial having a stopper pierceable by a hypodermic injection needle). At least one active agent in the composition is an antibody of the invention. The label or package insert indicates that the composition is used for treating the condition of choice, such as cancer. Moreover, the article of manufacture may comprise (a) a first container with a composition contained therein, wherein the composition comprises an antibody of the invention; and (b) a second container with a composition contained therein, wherein the composition comprises a further cytotoxic agent. The article of manufacture in this embodiment of the invention may further comprise a package insert indicating that the first and second antibody compositions can be used to treat a particular condition, for example cancer. Alternatively, or additionally, the article of manufacture may further comprise a second (or third) container comprising a pharmaceutically-acceptable buffer, such as bacteriostatic water for injection (BWFI), phosphate-buffered saline, Ringer's solution and dextrose solution. It may further include other materials desirable from a commercial and user standpoint, including other buffers, diluents, filters, needles, and syringes.

Having generally described the invention, the same will be more readily understood by reference to the following examples, which are provided by way of illustration and are not intended as limiting.

### EXAMPLE 1

### Construction of Phage-Displayed Fab Libraries with CDR Residues Enriched in Tyr or Ser

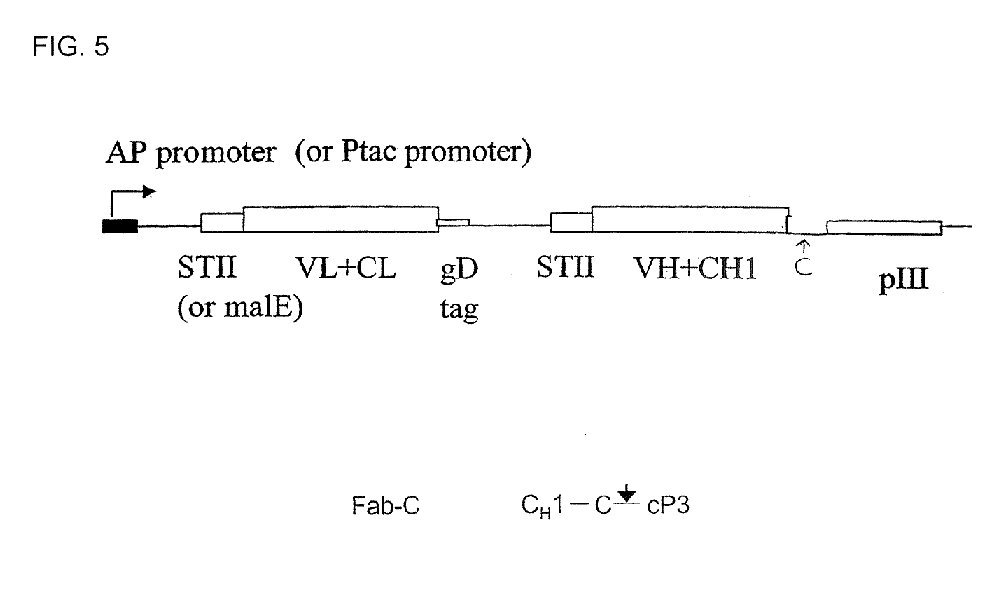

Phage-displayed Fab libraries were constructed using the “Fab-C” phagemid vector that resulted in the display of bivalent Fab moieties dimerized by a free cysteine inserted between the Fab heavy chain and the C-terminal domain of the gene-3 minor coat protein (P3C). This vector was constructed as described in U.S. Patent Application Publication No. US20050119455 and in Lee et al., J. Immunol. Meth. 284: 119-132 (2004). The vector (schematically illustrated in FIG. 5) comprises the humanized antibody 4D5 variable domains under the control of the IPTG-inducible Ptac promoter. The humanized antibody 4D5 has mostly human consensus sequence framework regions in the heavy and light chains, and CDR regions from a mouse monoclonal antibody specific for Her-2. Methods of making the anti-Her-2 antibody and the identity of the variable domain sequences are provided in U.S. Pat. Nos. 5,821,337 and 6,054,297.

Two libraries were constructed, the YS-C library and the YS-D library. Both libraries were constructed with randomized residues in all three heavy chain CDRs and light chain CDR3. The CDR amino acid positions randomized in each library are shown in FIG. 6. The type and ratio of the amino acids allowed at each of the randomized positions is described in FIG. 7.

In addition, the lengths of CDRH3 and CDRL3 were varied. The length of CDRH3 was varied by using oligonucleotides that replaced the seven wild-type codons from positions 95 to 100a with six to seventeen codons. Thus, in certain instances, the codon corresponding to position 100a of the heavy chain was not present (for example, when the mutagenesis was performed with mutagenic oligonucleotides H3-C6 (SEQ ID NO: 13) or H3-D6 (SEQ ID NO: 25), as described below.) The type and ratio of the amino acids allowed at these positions were the same as those described in FIG. 7 for positions 95 to 100a of the heavy chain. The length of CDRL3 was varied by using oligonucleotides that replaced the four wild-type codons from positions 91 to 94 with four to six codons. The type and ratio of the amino acids allowed at these positions were the same as the ones described in FIG. 7 for positions 91 to 94 of the light chain.

Libraries were constructed using the method of Kunkel (Kunkel, T. A., Roberts, J. D. & Zakour, R. A., Methods Enzymol. (1987), 154, 367-382) with previously described methods (Sidhu, S. S., Lowman, H. B., Cunningham, B. C. & Wells, J. A., Methods Enzymol. (2000), 328, 333-363). A unique “stop template” version of the Fab display vector was used to generate both libraries YS-C and YS-D. A template phagemid based on the Fab-C vector further comprising TAA stop codons inserted at positions 30, 33, 52, 54, 56, 57, 60, 102, 103, 104, 107, and 108 of the heavy chain and substitutions of wild-type amino acids by a serine residue at positions 28, 30, 31, 32, 50, and 53 of the light chain was used to perform the mutagenesis. (See U S. Patent Application Publication No. US20050119455 and Lee et al., J. Immunol. Meth. 284: 119-132 (2004) for description of the Fab-C vector). No stop codons were introduced in the light chain CDR3.

Mutagenic oligonucleotides with degenerate codons at the positions to be diversified were used to simultaneously (a) introduce CDR diversity and (b) repair the stop codons. The sequences of those mutagenic oligonucleotides are shown in FIGS. 8A and 8B. For both libraries, diversity was introduced into CDR-H1 and CDR-H2 with oligonucleotides H1 and H2, respectively (SEQ ID NOS: 8 and 9). For both libraries, diversity was introduced into CDR-L3 with an equimolar mixture of oligonucleotides L3a, L3b, and L3c (SEQ ID NOS: 10-12). For library YS-C, diversity was introduced into CDR-H3 with an equimolar mixture of oligonucleotides H3-C6, H3-C7, H3-C8, H3-C9, H3-C10, H3-C11, H3-C12, H3-C13, H3-C14, H3-C15, H3-C16, and H3-C17 (SEQ ID NOS: 13-24). For library YS-D, diversity was introduced into CDR-H3 with an equimolar mixture of oligonucleotides H3-D6, H3-D7, H3-D8, H3-D9, H3-D10, H3-D11, H3-D12, H3-D13, H3-D14, H3-D15, H3-D16, and H3-D17 (SEQ ID NOS: 25-36). Each of mutagenic oligonucleotides H3-C6 to H3-C17 (SEQ ID NOS: 13-24) and H3-D6 to H3-D17 (SEQ ID NOS: 25-36) encoded an alanine at position 93 of the heavy chain. The mutagenic oligonucleotides for all CDRs to be randomized were incorporated simultaneously in a single mutagenesis reaction, so that simultaneous incorporation of all the mutagenic oligonucleotides resulted in the introduction of the designed diversity at each position and simultaneously repaired all the TAA stop codons. Thus, an open reading frame was generated that encoded a Fab library member fused to a homodimerizing cysteine bridge and P3C.

The mutagenesis reactions were electroporated into E. coli SS320 (Sidhu et al., supra). The transformed cells were grown overnight in the presence of M13-K07 helper phage (New England Biolabs, Beverly, Mass.), to produce phage particles that encapsulated the phagemid DNA and displayed Fab fragments on their surfaces. Each library contained greater than 3×10¹⁰ unique members.

### EXAMPLE 2

### Selection of Specific Antibodies from the Naïve Libraries YS-C and YS-D

Phage from library YS-C or YS-D (see Example 1) were cycled through rounds of binding selection to enrich for clones binding to human VEGF. The binding selections were conducted using previously described methods (Sidhu et al., supra).

NUNC 96-well Maxisorp immunoplates were coated overnight at 4° C. with 5 μg/mL human VEGF and blocked for 2 h with a solution of PBT (phosphate buffered saline additionally containing 0.2% BSA and 0.05% Tween-20) (Sigma). After overnight growth at 37° C., phage were concentrated by precipitation with PEG/NaCl and resuspended in PBT, as described previously (Sidhu et al., supra). Phage solutions (about 10¹² phage/mL) were added to the coated immunoplates. Following a two hour incubation to permit phage binding, the plates were washed ten times with PBT. Bound phage were eluted with 0.1 M HCl for 10 minutes and the eluant was neutralized with 1.0 M Tris base. Eluted phage were amplified in E. coli XL1-blue and used for further rounds of selection.

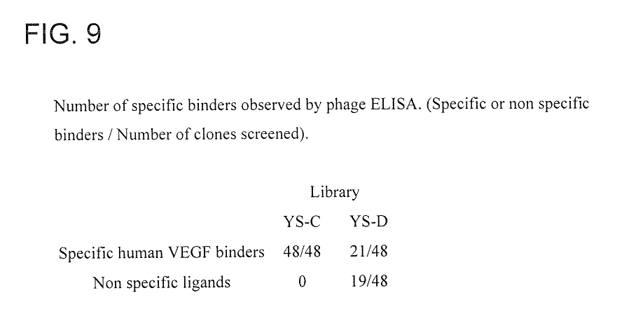

The libraries were subjected to five rounds of selection against each target protein. Individual clones from each round of selection were grown in a 96-well format in 500 μL of 2YT broth supplemented with carbenicillin and M13-K07. The culture supernatants were used directly in phage ELISAs (Sidhu et al., supra) to detect phage-displayed Fabs that bound to plates coated with target protein but not to plates coated with BSA. “Specific binders” were defined as those phage clones that exhibited an ELISA signal at least 15-fold greater on target-coated plates in comparison with their signal on BSA-coated plates. Individual clones were screened after four and five rounds of selection for binding to human VEGF and were subjected to sequence analysis. As shown in FIG. 9, both library YS-C and library YS-D produced specific binders against human VEGF. Library YS-D also produced both non-specific binders (binders that bound to both BSA and VEGF) that were not produced by the YS-C library and nine clones that did not bind to either VEGF or to BSA.

The CDRH1, CDRH2, CDRH3, and CDRL3 sequences for the unique specific binders are shown in FIG. 10. For CDRH3 and CDRL3, where randomization included diversification of length (see Example 1 and FIG. 7), the length of CDRH3 and CDRL3 varied from clone to clone. Thus, originally randomized positions 100b and 100c in CDRH3 appear in FIG. 10 at different Kabat positions depending on the number of amino acid insertions in that particular CDRH3, but always immediately precede invariant positions 101 and 102 (Asp and Tyr, respectively) in any given CDRH3. Similarly, additional length diversity in CDRL3 is shown in FIG. 10 at positions 94a and 94b of CDRL3.

The affinity of each of the unique Fab-expressing phages obtained from the YS-C and YS-D libraries was estimated by a two-spot phage ELISA. A single-point competitive phage ELISA was used to estimate the affinities of phage-displayed Fabs, as follows. Phage were produced in a 96-well format as described, and phage supernatants were diluted fivefold in PBT buffer or PBT buffer including 100 nM or 1000 nM human VEGF. The mixtures were incubated for 1 hour, transferred to plates coated with human VEGF and the plates were incubated for 15 minutes. The plates were washed with PBS including 0.05% Tween 20 and were incubated for 30 minutes with horseradish peroxidase/anti-M13 antibody conjugate (1:5000 dilution in PT buffer) (Pharmacia). The plates were washed, developed with tetramethylbenzidine (TMB) substrate (Kirkegaard and Perry Laboratories) and quenched with 1.0 M H₃PO₄. Absorbance was determined spectrophotometrically at 450 nm. The fraction of Fab-phage uncomplexed with solution-phase human VEGF was calculated by dividing the A450 in the presence of 100 nM or 1000 nM human VEGF by the A450 in the absence of human VEGF. The results are shown in FIG. 10.

Based on this analysis, soluble Fab proteins from the 12 clones that were ranked as the highest affinity binders by the phage ELISA analysis (showing the lowest fraction of uncomplexed Fab-phage after incubation with 1000 nM of hVEGF) were purified and subjected to surface plasmon resonance analysis of binding to human VEGF. BIAcore data was obtained according to Chen et al., J. Mol. Biol. (1999), 293(4): 865-81. Briefly, binding affinities of the purified Fabs for human VEGF were calculated from association and dissociation rate constants measured using a BIAcore™-2000 surface plasmon resonance system (BIACORE, Inc., Piscataway, N.J.). VEGF was covalently coupled to a biosensor chip using N-ethyl-N′-(3-dimethylaminopropyl)-carbodiimide hydrochloride (EDC) and N-hydroxysuccinimide (NHS) according to the supplier's (BIAcore, Inc., Piscataway, N.J.) instructions. Human VEGF was buffer-exchanged into 10 mM sodium acetate, pH 4.8 and diluted to approximately 30 μg/ml. Aliquots of VEGF were injected at a flow rate of 2 μL/min to achieve approximately 200-300 response units (RU) of coupled protein. A solution of 1 M ethanolamine was injected as a blocking agent. For kinetics measurements, twofold serial dilutions of each Fab were injected in PBT at 25° C. at a flow rate of 10 μL/minute. The kₒₙ and k_{off} values were determined from the binding curves using the BIAevaluation software package (BIACORE, Inc., Piscataway, N.J.). The equilibrium dissociation constant, K_{D}, was calculated as K_{off}/kₒₙ. The BIACORE data is summarized in FIG. 10. The language “N.D.B.” denotes that there was no detectable binding for the indicated Fab.

### EXAMPLE 3

### Construction of Phage-Displayed Fab Libraries with CDR Residues Enriched in Tyr, Ser, Gly, and Arg

Phage-displayed Fab libraries were constructed using a phagemid vector, Fab-C, that resulted in the display of bivalent Fab moieties dimerized by a free cysteine inserted between the Fab heavy chain and the C-terminal domain of the gene-3 minor coat protein (P3C), as previously described in Example 1.

Four libraries were constructed: YSGR-A, YSGR-B, YSGR-C, and YSGR-D. The libraries were constructed with randomized residues in all three heavy chain CDRs and light chain CDR3. Each library was randomized at positions 91-94 and 96 of CDRL3, positions 28 and 30-33 of CDRH1, positions 50, 52-54, 56, and 58 of CDRH2, and positions 95-100, 100a, 100b, and 100c of CDRH3. The type and ratio of the amino acids allowed at each of the randomized positions is described in FIG. 11. In addition, the length of CDRH3 was varied by using oligonucleotides that replaced the seven wild-type codons from positions 95 to 100a with six to seventeen codons. Thus, in certain instances, the codon corresponding to position 100a of the heavy chain was not present (for example, when the mutagenesis was performed with mutagenic oligonucleotides H3-A6 (SEQ ID NO: 161), H3-B6 (SEQ ID NO: 173), H3-C6 (SEQ ID NO: 185) or H3-D6 (SEQ ID NO: 197), as described below.) The type and ratio of the amino acids allowed at those positions were the same as the ones described in FIG. 11 for positions 95-100a of CDRH3.

Libraries were constructed using the method of Kunkel (Kunkel, T. A., Roberts, J. D. & Zakour, R. A., Methods Enzymol. (1987), 154, 367-382) with previously described methods (Sidhu, S. S., Lowman, H. B., Cunningham, B. C. & Wells, J. A., Methods Enzymol. (2000), 328, 333-363). A unique “stop template” version of the Fab display vector Fab-C was used to generate all four libraries, as described in Example 1.

Mutagenic oligonucleotides with degenerate codons at the positions to be diversified were used to simultaneously (a) introduce CDR diversity and (b) repair the stop codons. The sequences of those mutagenic oligonucleotides are shown in FIGS. 12A-12D. For all libraries, diversity was introduced into CDR-H1, CDR-H2, and CDR-H3 with oligonucleotides H1, H2 and L3, respectively (SEQ ID NOS: 158, 159, and 160). For library YSGR-A, diversity was introduced into CDR-H3 with an equimolar mixture of oligonucleotides H3-A6, H3-A7, H3-A8, H3-A9, H3-A10, H3-A11, H3-A12, H3-A13, H3-A14, H3-A15, H3-A16, and H3-A17 (SEQ ID NOS: 161-172). For library YSGR-B, diversity was introduced into CDR-H3 with an equimolar mixture of oligonucleotides H3-B6, H3-B7, H3-B8, H3-B9, H3-B10, H3-B11, H3-B12, H3-B13, H3-B14, H3-B15, H3-B16, and H3-B17 (SEQ ID NOS: 173-184). For library YSGR-C, diversity was introduced into CDR-H3 with an equimolar mixture of oligonucleotides H3-C6, H3-C7, H3-C8, H3-C9, H3-C10, H3-C11, H3-C12, H3-C13, H3-C14, H3-C15, H3-C16, and H3-C17 (SEQ ID NOS: 185-196). For library YSGR-D, diversity was introduced into CDR-H3 with an equimolar mixture of oligonucleotides H3-D6, H3-D7, H3-D8, H3-D9, H3-D10, H3-D11, H3-D12, H3-D13, H3-D14, H3-D15, H3-D16, and H3-D17 (SEQ ID NOS: 197-208). Each of mutagenic oligonucleotides H3-A6 to H3-A17 (SEQ ID NOS: 161-172), H3-B6 to H3-B17 (SEQ ID NOS: 173-184), H3-C6 to H3-C17 (SEQ ID NOS: 185-196) and H3-D6 to H3-D17 (SEQ ID NOS: 197-208) encoded an alanine at position 93 of the heavy chain. The mutagenic oligonucleotides for all CDRs to be randomized were incorporated simultaneously in a single mutagenesis reaction, so that simultaneous incorporation of all the mutagenic oligonucleotides resulted in the introduction of the designed diversity at each position and simultaneously repaired all the TAA stop codons. Thus, an open reading frame was generated that encoded a Fab library member fused to a homodimerizing cysteine bridge and P3C. Following mutagenesis, the four libraries were combined to create a single library, called library YSGR-A-D.

The mutagenesis reactions were electroporated into E. coli SS320 (Sidhu et al., supra). The transformed cells were grown overnight in the presence of M13-KO7 helper phage (New England Biolabs, Beverly, Mass.) to produce phage particles that encapsulated the phagemid DNA and displayed Fab fragments on their surfaces. The combined library contained greater than 3×10¹⁰ unique members.

### EXAMPLE 4

### Selection of Specific Antibodies from Naïve Library YSGR-A-D

Phage from library YSGR-A-D (described in Example 3, above) were cycled through rounds of binding selection to enrich for clones binding to human VEGF or human insulin. The binding selections were conducted using previously described methods (Sidhu et al., supra).

NUNC 96-well Maxisorp immunoplates were coated overnight at 4° C. with 5 μg/mL target protein (human VEGF or human insulin) and blocked for 2 hours with a solution of PBT (phosphate buffered saline additionally containing 0.2% BSA and 0.05% Tween 20 (Sigma)). After overnight growth at 37° C., phage were concentrated by precipitation with PEG/NaCl and resuspended in PBT, as described previously (Sidhu et al., supra). Phage solutions (about 10¹² phage/mL) were added to the coated immunoplates. Following a two hour incubation to permit phage binding, the plates were washed ten times with PBT. Bound phage were eluted with 0.1 M HCl for ten minutes and the eluant was neutralized with 1.0 M Tris base. Eluted phage were amplified in E. coli XL1-blue and used for further rounds of selection.

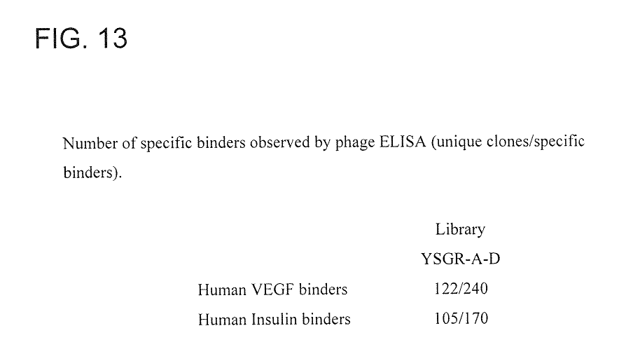

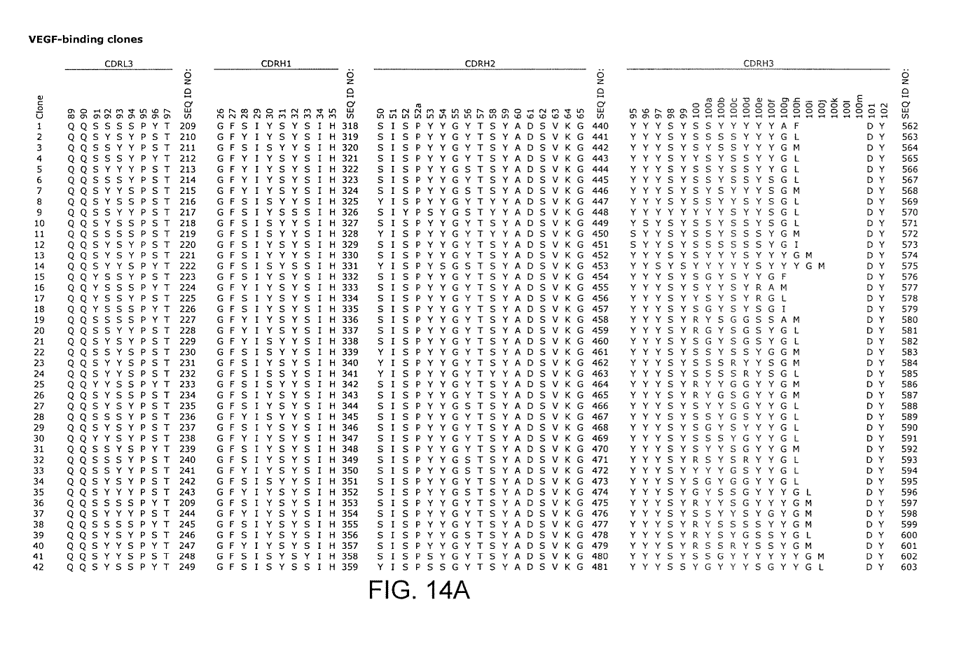

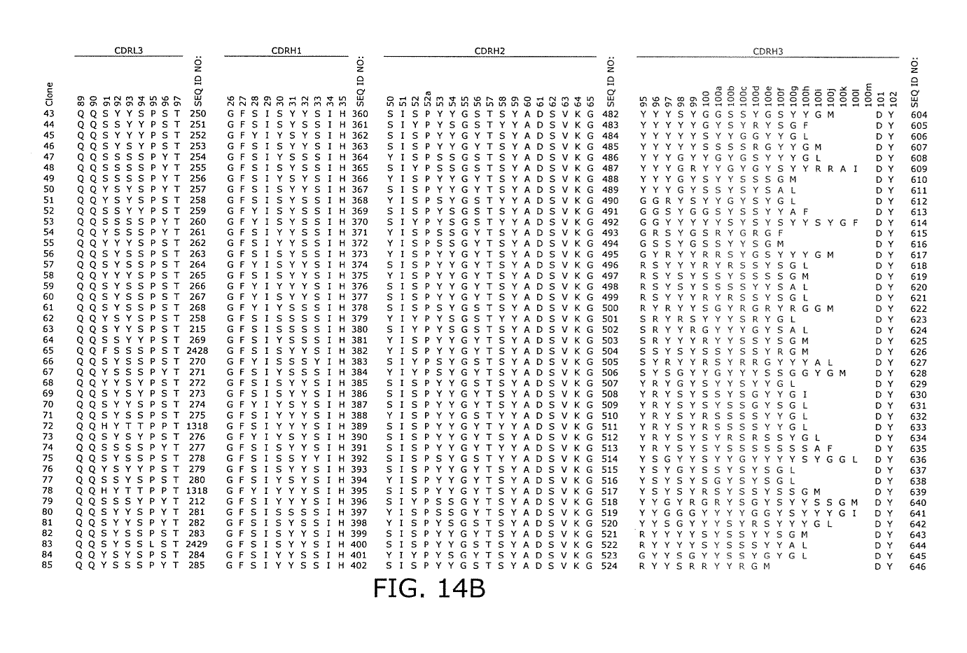

The libraries were subjected to five rounds of selection against each target protein. Individual clones from each round of selection were grown in a 96-well format in 500 μL of 2YT broth supplemented with carbenicillin and M13-K07. The culture supematants were used directly in phage ELISAs (Sidhu et al., supra) to detect phage-displayed Fabs that bound to plates coated with target protein but not to plates coated with BSA. Specific binders were defined as those phage clones that exhibited an ELISA signal at least 10-fold greater on target-coated plates in comparison with BSA-coated plates. Individual clones were screened after 4 and 5 rounds of selection for binding to human VEGF or human insulin. The specific binders were subjected to sequence analysis. As shown in FIG. 13, the YSGR-A-D library produced specific binders against both target proteins.

Of the 240 clones identified that specifically bound to human VEGF, 122 of them had unique CDR sequences (see FIG. 13). Those unique sequences are shown in FIGS. 14A-14C. The unique sequences fell into three categories: (a) CDR sequences with randomized positions limited to binary Tyr/Ser (14 of 122 sequences, clone numbers 1-14); (b) CDR sequences with randomized positions limited to Tyr/Ser/Gly/Arg sequences (84 of 122 sequences, clone numbers 15-98); and (c) CDR sequences with randomized positions having amino acid usages that did not readily fall into either of the other two categories (24 of 122 sequences, clone numbers 99-122). A comparison of the binary Tyr/Ser category sequences (clone numbers 1-14) and the YSGR category sequences (clone numbers 15-45) shows that the preponderance of sequences in both categories comprise Tyr at positions 32 of CDRH1, 53, 54, and 56 of CDRH2, and 95-97 and 99 of CDRH3, and Ser at positions 33 of CDRH1, 50, 52, and 58 of CDRH2, and 98 of CDRH3.

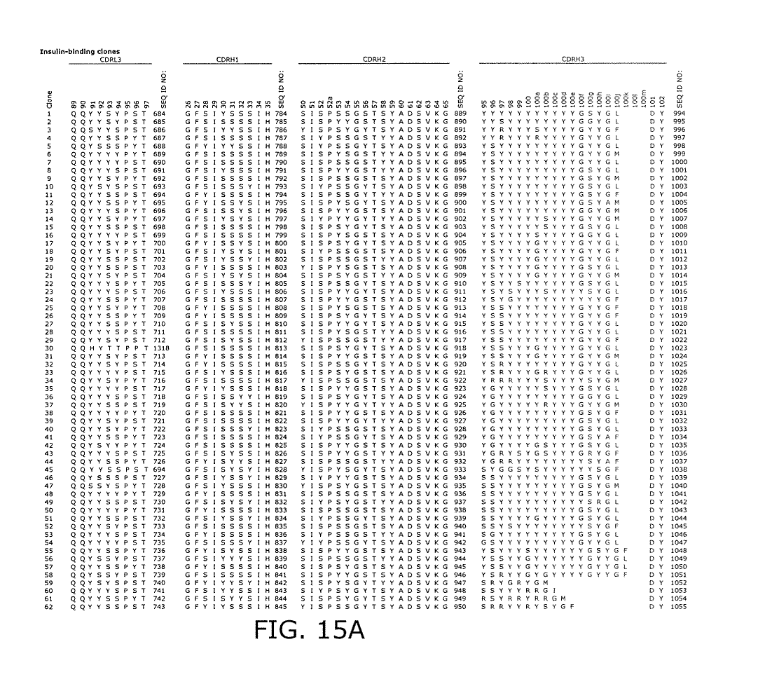

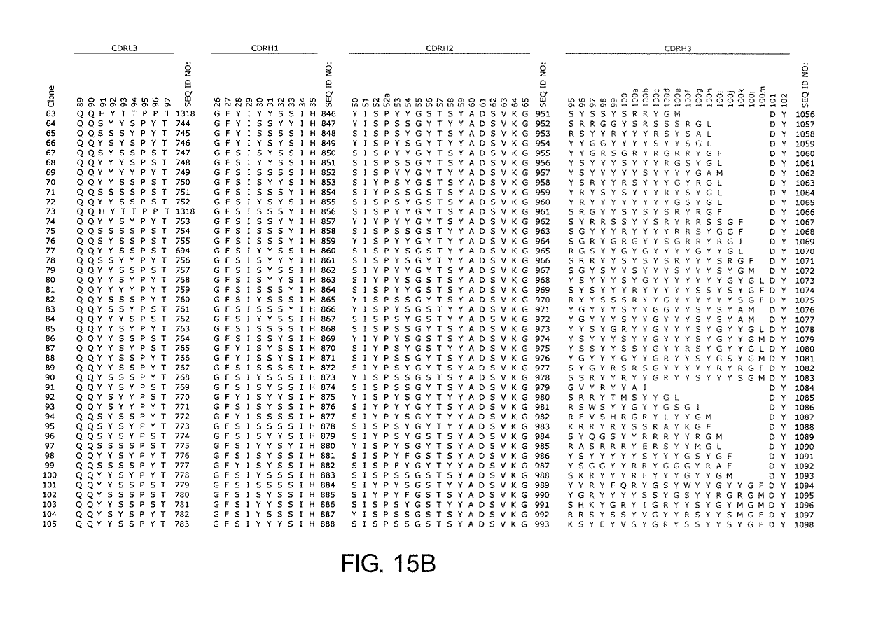

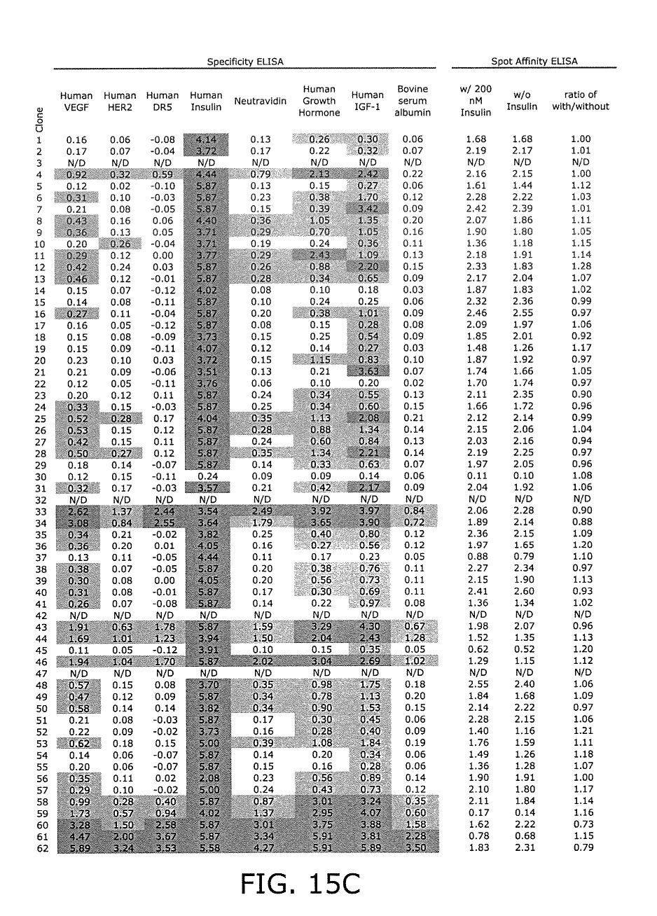

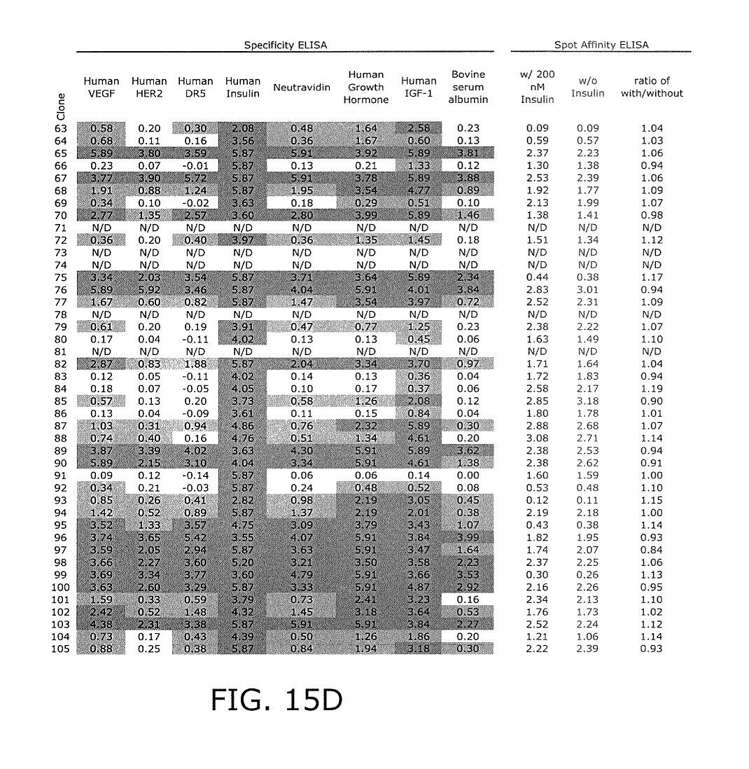

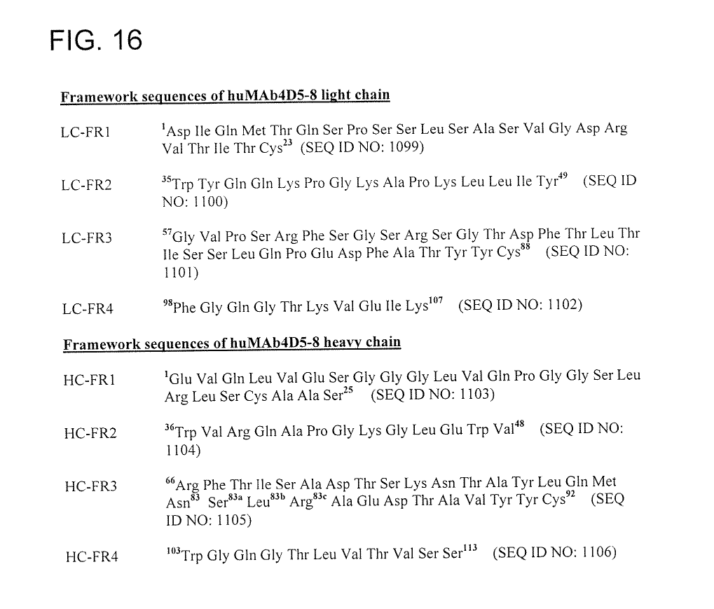

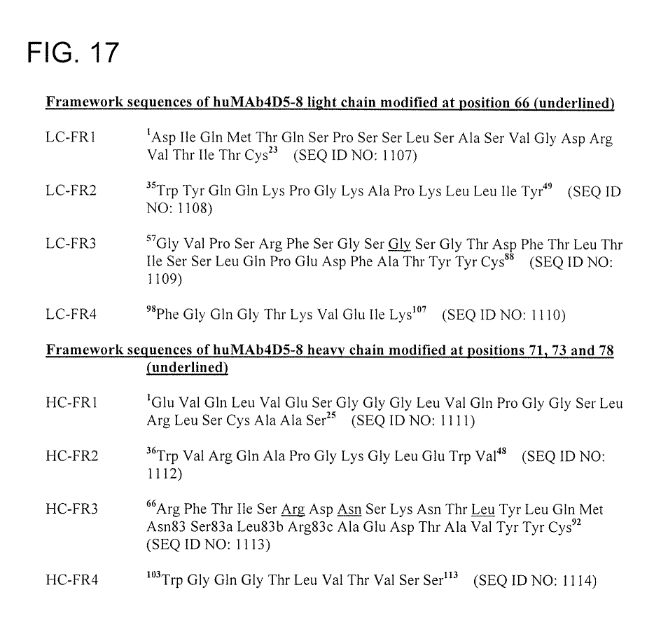

As shown in FIG. 13, 170 clones were identified that expressed Fabs that were specific binders for insulin. Sequence analysis identified 105 unique amino acid sequences from those 170 clones, shown in FIGS. 15A and 15B. The unique sequences fell into three categories: (a) CDRH3 sequences with Tyr-rich randomized positions (58 of 105 sequences, clone nos. 1-58); (b) CDRH3 sequences with randomized positions limited to Tyr/Ser/Gly/Arg sequences (35 of 105 sequences, clones 59-93); and (c) CDRH3 sequences with Tyr/Ser/Arg/Gly/X at the randomized positions (12 of 105 sequences, clones 94-105). A comparison of the TyT-rich category sequences (clone nos. 1-61) and the YSGRX category sequences (clone nos. 62-73) shows that the preponderance of sequences in both categories comprise Ser at position 33 of CDRH1 and Tyr at positions 98 and 100e of CDRH3.

In the CDRH3 sequences shown in both FIGS. 14A-C and FIGS. 15A and 15B, the length of CDRH3 varied from clone to clone due to the length diversification within that CDR (see Example 3 and FIG. 11). Thus, originally randomized positions 100b and 100c in CDRH3 appear in FIGS. 14A-C and 15A-15B at different Kabat positions depending on the number of amino acid insertions in that particular CDRH3, but always immediately precede invariant positions 101 and 102 (Asp and Tyr, respectively) in any given CDRH3.

A phage ELISA was used to test the ability of all clones to cross-react with a panel of six antigens other than the target antigen. Phage were produced in a 96-well format as described and phage supernatants were diluted 3-fold in PBT buffer. The diluted phage supernatant was transferred to plates coated with human VEGF, HER2, human DR5, human insulin, neutravidin, human IGF-1, HGH, or BSA, and incubated for one hour with gentle shaking at room temperature. The plates were washed with PBS including 0.05% Tween 20, and were incubated for 30 minutes with horseradish peroxidase/anti-M13 antibody conjugate (diluted 1:5000 in PT buffer) (Pharmacia). The plates were washed, developed with tetramethylbenzidine (TMB) substrate (Kirkegaard and Perry Laboratories) and quenched with 1.0 M H₃PO₄. Absorbance was determined spectrophotometrically at 450 nm. Weak cross-reactivity was defined as a signal between 0.25-2.0 and strong cross-reactivity was defined as a signal about 2.0. The results for all YSGR-A-D clones are shown in FIGS. 14D-F and FIGS. 15C and 15D. A single-point competitive phage ELISA (“spot affinity ELISA”) was used to roughly estimate the affinities of phage-displayed Fabs. Phage were produced in a 96-well format as described, and phage supematants were diluted five fold in PBT buffer or PBT buffer with 100 nM human VEGF, 100 nM HER2 or 200 nM human insulin. The mixtures were incubated for 1 hour, then transferred to plates coated with human VEGF, HER2, or human insulin and incubated for 15 minutes. The plates were washed with PBS including 0.05% Tween 20, and were incubated for 30 minutes with horseradish peroxidase/anti-M13 antibody conjugate (diluted 1:5000 in PT buffer) (Pharmacia). The plates were washed, developed with tetramethylbenzidine (TMB) substrate (Kirkegaard and Perry Laboratories) and quenched with 1.0 M H₃PO₄. Absorbance was determined spectrophotometrically at 450 nm. The fraction of Fab-phage uncomplexed with solution-phase human VEGF, HER2, or human insulin was calculated by dividing the A450 in the presence of antigen by the A₄₅₀ in the absence of antigen. The results are shown in FIGS. 14D-F and FIGS. 15C-15D.

A competitive phage ELISA was used to estimate the binding affinities of some VEGF-binding phage-displayed Fabs. Phage were produced in a 96-well format as described, and phage supernatants were serially diluted in PBT buffer, then incubated on plates coated with human VEGF for 15 minutes. The plates were washed with PBS including 0.05% Tween 20 and were incubated for 30 minutes with horseradish peroxidase/anti-M13 antibody conjugate (diluted 1:5000 in PT buffer) (Pharmacia). The plates were washed, developed with tetramethylbenzidine (TMB) substrate (Kirkegaard and Perry Laboratories) and quenched with 1.0 M H₃PO₄. Absorbance was measured spectrophotometrically at 450 nm to determine the phage concentration giving about 50% of the signal at saturation. A fixed, sub-saturating concentration of phage was diluted two fold in PBT buffer or PBT buffer containing two-fold serial dilutions of human VEGF protein from 500 nM human VEGF to 0.24 nM human VEGF. The mixtures were incubated for one hour with gentle shaking at room temperature, transferred to plates coated with human VEGF and the plates were incubated for 15 minutes. The plates were washed and treated exactly as above. The binding affinities were estimated as IC₅₀ values (defined as the concentration of antigen that blocked 50% of the phage binding to the immobilized antigen). The results are shown in FIGS. 14D-F.

### EXAMPLE 5

### Construction of Phage-Displayed Fab Libraries with CDRH1, H2, and L3 Residues Enriched in Tyr and Ser and CDRH3 Residues Enriched in Ser and Ala, Cys, Phe, Gly, Ile, Leu, Asn, Pro, Arg, Thr, Trp, or Tyr

Phage-displayed Fab libraries were constructed using a phagemid vector, Fab-C, that resulted in the display of bivalent Fab moieties dimerized by a free cysteine inserted between the Fab heavy chain and the C-terminal domain of the gene-3 minor coat protein (P3C), as previously described in Example 1.

Twelve libraries were constructed: SAH3, SCH3, SFH3, SGH3, SLH3, SNH3, SPH3, SRH3, STH3, SWH3, and SYH3. The libraries were constructed with randomized residues in all three heavy chain CDRs and light chain CDR3. Each library was randomized at positions 91-94 and 96 of CDRL3, positions 28 and 30-33 of CDRH1, positions 50, 52-54, 56, and 58 of CDRH2, and position 95-100, 101, and 102 of CDRH3. The type and ratio of the amino acids allowed at each of the randomized positions is described in FIGS. 18A-18B. In addition, the length of CDRH3 was varied by using oligonucleotides that replaced the six wild-type codons between positions 95 and 100 with 4 to 17 codons. The type and ratio of the amino acids allowed at those positions were the same as the ones described in FIGS. 18A-18B for positions 95-100 of CDRH3.

Libraries were constructed using the method of Kunkel (Kunkel et al., Methods Enzymol. (1987) 154: 367-382) with previously described methods (Sidhu et al., Methods Enzymol. (2000) 328: 333-363). A unique “stop template” version of the Fab display vector Fab-C was used to generate all four libraries, as described in Example 1.

Mutagenic oligonucleotides with degenerate codons at the positions to be diversified were used to simultaneously (a) introduce CDR diversity and (b) repair the stop codons. The sequences of those mutagenic oligonucleotides are shown in FIGS. 19A-19L. For all libraries, diversity was introduced into CDRH1, CDRH2, and CDRL3 with oligonucleotides H1, H2, and L3, respectively (SEQ ID NOS: 158, 159, and 160).

For library SAH3, diversity was introduced into CDRH3 with an equimolar mixture of oligonucleotides H3-SA4, H3-SA5, H3-SA6, H3-SA7, H3-SA8, H3-SA9, H3-SA10, H3-SA11, H3-SA12, H3-SA13, H3-SA14, H3-SA15, H3-SA16, and H3-SA17 (SEQ ID NOS: 1115-1128).

For library SCH3, diversity was introduced into CDRH3 with an equimolar mixture of oligonucleotides H3-SC4, H3-SC5, H3-SC6, H3-SC7, H3-SC8, H3-SC9, H3-SC10, H3-SC11, H3-SC12, H3-SC13, H3-SC14, H3-SC15, H3-SC16, and H3-SC17 (SEQ ID NOS: 1129-1142).

For library SFH3, diversity was introduced into CDR-H3 with an equimolar mixture of oligonucleotides H3-SF4, H3-SF5, H3-SF6, H3-SF7, H3-SF8, H3-SF9, H3-SF10, H3-SF11, H3-SF12, H3-SF13, H3-SF14, H3-SF15, H3-SF16, and H3-SF17 (SEQ ID NOS: 1143-1156).

For library SGH3, diversity was introduced into CDRH3 with an equimolar mixture of oligonucleotides H3-SG4, H3-SG5, H3-SG6, H3-SG7, H3-SG8, H3-SG9, H3-SG10, H3-SG11, H3-SG12, H3-SG13, H3-SG14, H3-SG15, H3-SG16, and H3-SG17 (SEQ ID NOS: 1157-11700.

For library SIH3, diversity was introduced into CDR-H3 with an equimolar mixture of oligonucleotides H3-SI4, H3-SI5, H3-SI6, H3-SI7, H3-SI8, H3-SI9, H3-SI10, H3-SI11, H3-SI12, H3-SI13, H3-SI14, H3-SI15, H3-SI16, and H3-SI17 (SEQ ID NOS: 1171-1184).

For library SLH3, diversity was introduced into CDR-H3 with an equimolar mixture of oligonucleotides H3-SL4, H3-SL5, H3-SL6, H3-SL7, H3-SL8, H3-SL9, H3-SL10, H3-SL11, H3-SL12, H3-SL13, H3-SL14, H3-SL15, H3-SL16, and H3-SL17 (SEQ ID NOS: 1185-1198).

For library SNH3, diversity was introduced into CDR-H3 with an equimolar mixture of oligonucleotides H3-SN4, H3-SN5, H3-SN6, H3-SN7, H3-SN8, H3-SN9, H3-SN10, H3-SN11, H3-SN12, H3-SN13, H3-SN14, H3-SN15, H3-SN16, and H3-SN17 (SEQ ID NOS: 1199-1212).

For library SPH3, diversity was introduced into CDR-H3 with an equimolar mixture of oligonucleotides H3-SP4, H3-SP5, H3-SP6, H3-SP7, H3-SP8, H3-SP9, H3-SP10, H3-SP11, H3-SP12, H3-SP13, H3-SP14, H3-SP15, H3-SP16, and H3-SP17 (SEQ ID NOS: 1213-1226).

For library SRH3, diversity was introduced into CDR-H3 with an equimolar mixture of oligonucleotides H3-SR4, H3-SR5, H3-SR6, H3-SR7, H3-SR8, H3-SR9, H3-SR10, H3-SR11, H3-SR12, H3-SR13, H3-SR14, H3-SR15, H3-SR16, and H3-SR17 (SEQ ID NOS: 1227-1240).

For library STH3, diversity was introduced into CDR-H3 with an equimolar mixture of oligonucleotides H3-ST4, H3-ST5, H3-ST6, H3-ST7, H3-ST8, H3-ST9, H3-ST10, H3-ST11, H3-ST12, H3-ST13, H3-ST14, H3-ST15, H3-ST16, and H3-ST17 (SEQ ID NOS: 1241-1254).

For library SWH3, diversity was introduced into CDR-H3 with an equimolar mixture of oligonucleotides H3-SW4, H3-SW5, H3-SW6, H3-SW7, H3-SW8, H3-SW9, H3-SW10, H3-SW11, H3-SW12, H3-SW13, H3-SW14, H3-SW15, H3-SW16, and H3-SW17 (SEQ ID NOS: 1255-1268).

For library SYH3, diversity was introduced into CDR-H3 with an equimolar mixture of oligonucleotides H3-SY4, H3-SY5, H3-SY6, H3-SY7, H3-SY8, H3-SY9, H3-SY10, H3-SY11, H3-SY12, H3-SY13, H3-SY14, H3-SY15, H3-SY16, and H3-SY17 (SEQ ID NOS: 1269-1282).

The mutagenic oligonucleotides for all CDRs to be randomized were incorporated in a single mutagenesis reaction, so that simultaneous incorporation of all the mutagenic oligonucleotides resulted in the introduction of the designed diversity at each position and repair of all of the TAA stop codons. Thus, an open reading frame was generated that encoded a Fab library member fused to a homodimerizing cysteine bridge and P3C. Following mutagenesis, the twelve libraries were combined to create a single library, called library SXH3.

The mutagenesis reactions were electroporated into E. coli SS320 (Sidhu et al., supra). The transformed cells were grown overnight in the presence of M13-K07 helper phage (New England Biolabs, Beverly, Mass.) to produce phage particles that encapsulated the phagemid DNA and displayed Fab fragments on their surfaces. The combined library contained greater than 3×10¹⁰ unique members.

### EXAMPLE 6

### Selection of Specific Antibodies from Naïve Library SXH3

Phage from library SXH3 (described in Example 5, above) were cycled through rounds of binding selection to enrich for clones binding to human VEGF, HER2, human insulin, human IGF-1, or HGH. The binding selections were conducted using previously described methods (Sidhu et al., supra).

NUNC 96-well Maxisorp immunoplates were coated overnight at 4° C. with 5 μg/mL target protein (human VEGF, HER2, human insulin, human IGF-1, or HGH) and blocked for two hours with a solution of PBT (Sigma). After overnight growth at 37° C., phage were concentrated by precipitation with PEG/NaCl and resuspended in PBT, as described previously (Sidhu et al., supra). Phage solutions (about 10¹² phage/mL) were added to the coated immunoplates. Following a two hour incubation to permit phage binding, the plates were washed ten times with PBT. Bound phage were eluted with 0.1M HCl for ten minutes and the eluant was neutralized with 1.0 M Tris base. Eluted phage were amplified in E. coli XL1-blue and used for further rounds of selection.

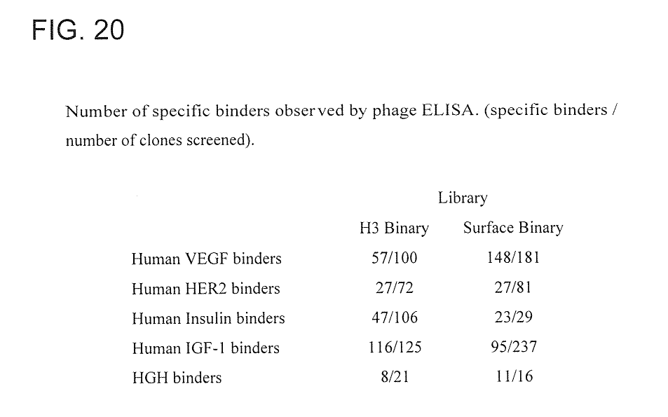

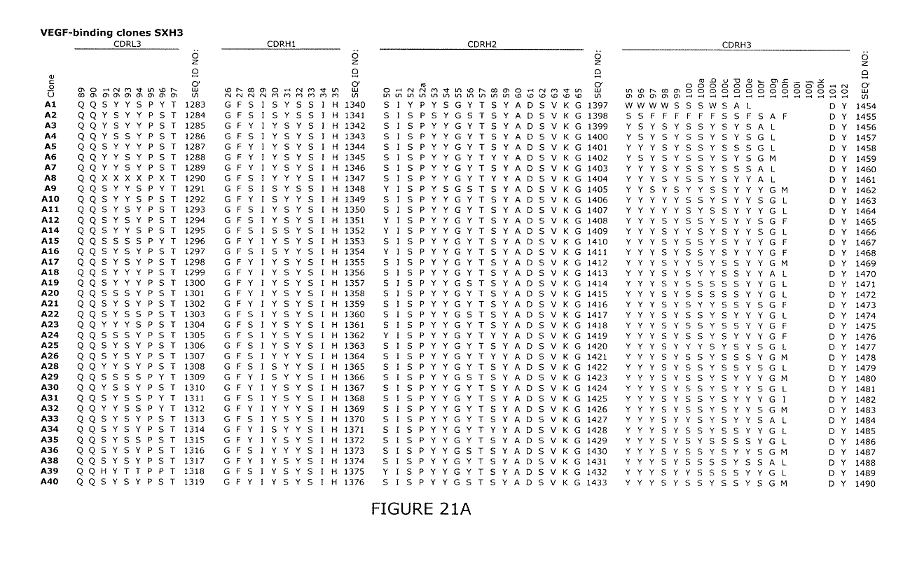

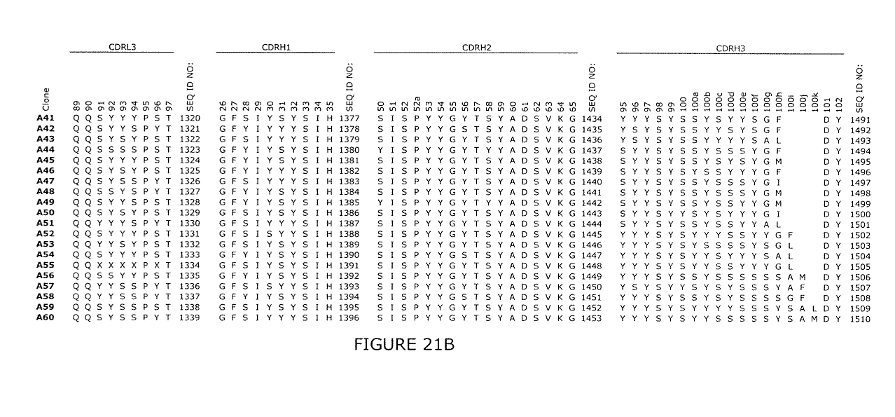

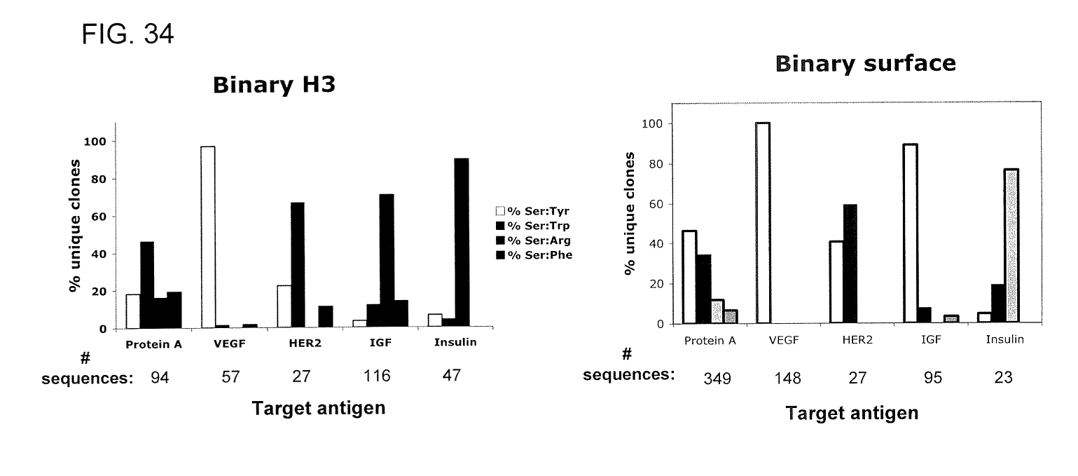

The libraries were subjected to six rounds of selection against each target protein. Individual clones from each round of selection were grown in a 96-well format in 500 μL of 2YT broth supplemented with carbenicillin and M13-K07. The culture supernatants were used directly in phage ELISAs (Sidhu et al., supra) to detect phage-displayed Fabs that bound to plates coated with target protein but not to plates coated with BSA. Specific binders were defined as those phage clones that exhibited an ELISA signal at least 10-fold greater on target-coated plates in comparison with BSA-coated plates. Individual clones were screened after 4, 5, and 6 rounds of selection for binding to human VEGF, HER2, human insulin, human IGF-1, or HGH. The specific binders were subjected to sequence analysis. As shown in FIG. 20, the SXH3 library produced specific binders to all five target proteins. The distribution of target-binding clones from each S:XH3 library is shown in FIG. 34 as well as the distribution of properly folded and displayed S:XH3 antibodies that bound to Protein A.

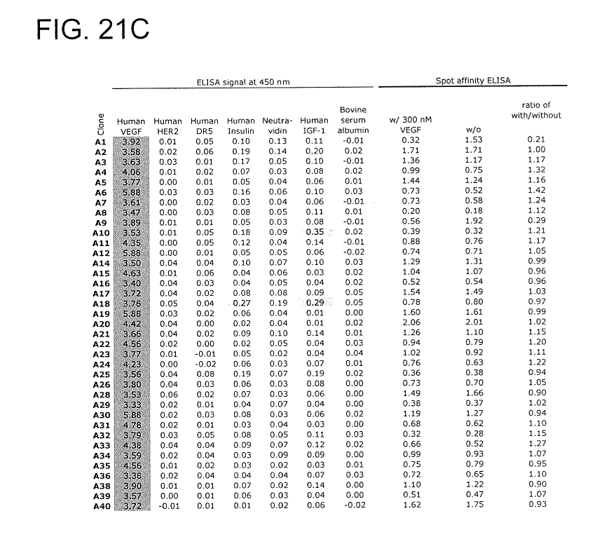

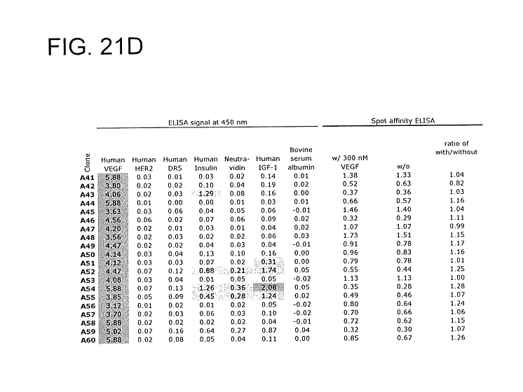

Of the 100 clones identified that specifically bound to human VEGF, 57 of them had unique CDR sequences (see FIGS. 21 A-21B). The unique sequences had randomized positions limited to binary Tyr/Ser (clone nos. A1-A60). The clones were also highly specific for VEGF and did not display significant cross-reactivity to five other control proteins: HER2, human DR5, human insulin, neutravidin, human IGF-1 or HGH (see FIGS. 21C-21D).

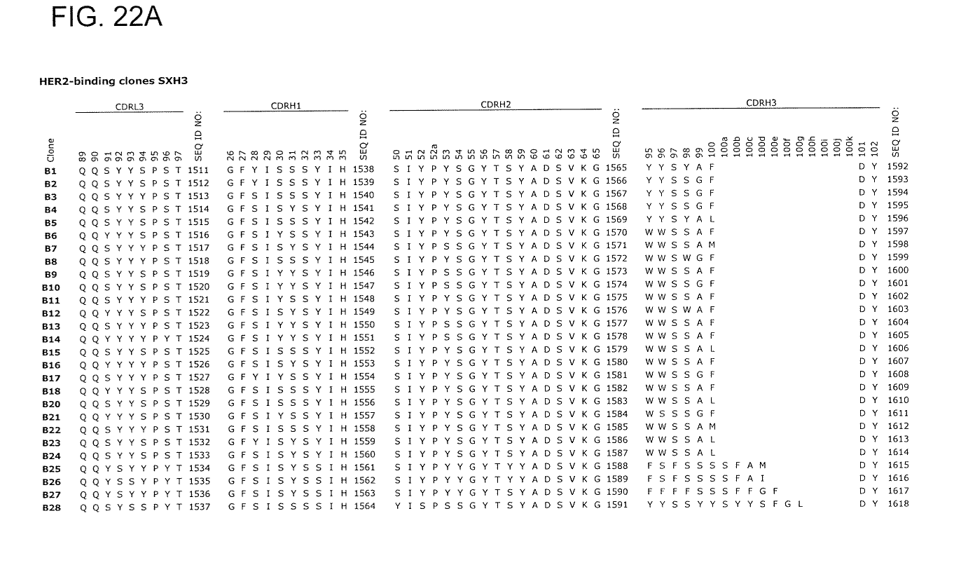

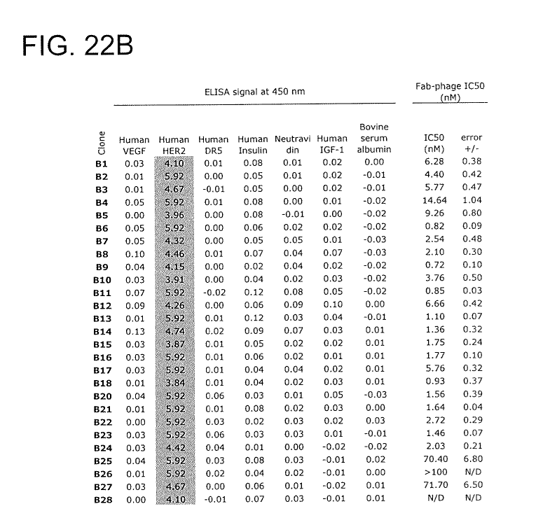

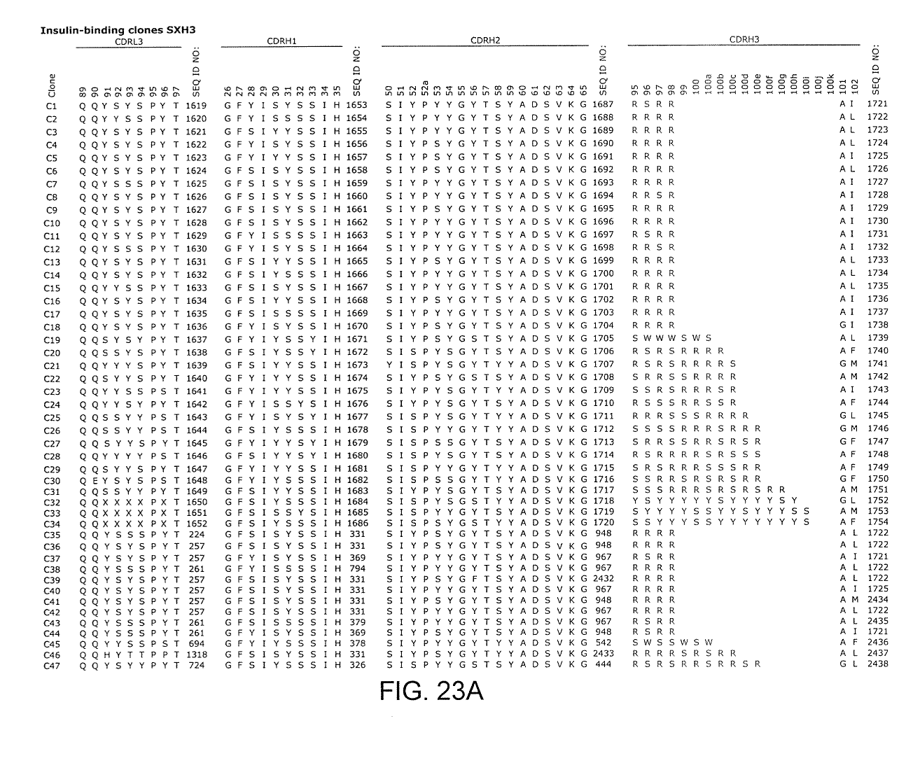

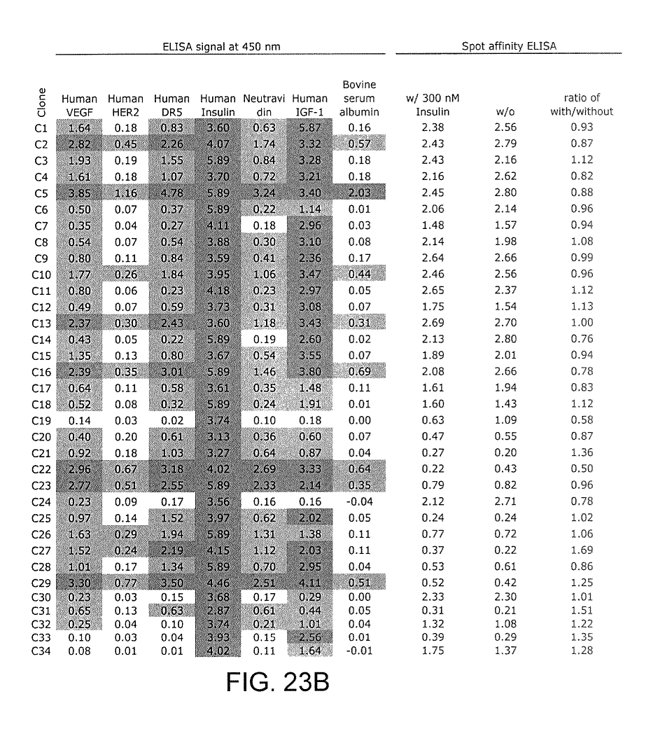

Of the 72 clones identified that specifically bound to HER2, 27 of them had unique CDR sequences (see FIG. 22A). The unique sequences fell into three categories: (1) CDR sequences with randomized positions limited to binary Tyr/Ser (clone nos. B1-5 and B28); (b) CDR sequences with randomized positions limited to binary Trp/Ser (clone nos. B6-24); (c) CDR sequences with randomized positions limited to binary Phe/Ser (clone nos. B25-27). These clones were also highly specific for HER2 and did not display cross-reactivity to five other control proteins, human VEGF, human DR5, human insulin, neutravidin, human IGF-1, or HGH (see FIG. 22B). The inhibitory concentration for each clone is shown in FIG. 22B.

Of the 106 clones identified that specifically bound to human insulin, 47 of them had unique CDR sequences (see FIGS. 23A-B). The unique sequences fell into three categories: (a) CDR sequences with randomized positions limited to binary Tyr/Ser (clone nos. C32-34); (b) CDR sequences with randomized positions limited to binary Trp/Ser (clone no. C19); and (c) CDR sequences with randomized positions limited to binary Arg/Ser (clone nos. C1-18 and C20-31). Additional clones C35 to C47 have been sequenced. The Arg/Ser clones bound with high affinity to human insulin but also displayed cross-reactivity to five other control proteins, human VEGF, HER2, human DR5, neutravidin, human IGF-1, or HGH (see FIG. 23C). The Trp/Ser clone and Tyr/Ser clones has less cross-reactivity than the Arg/Ser clones (FIG. 23C).

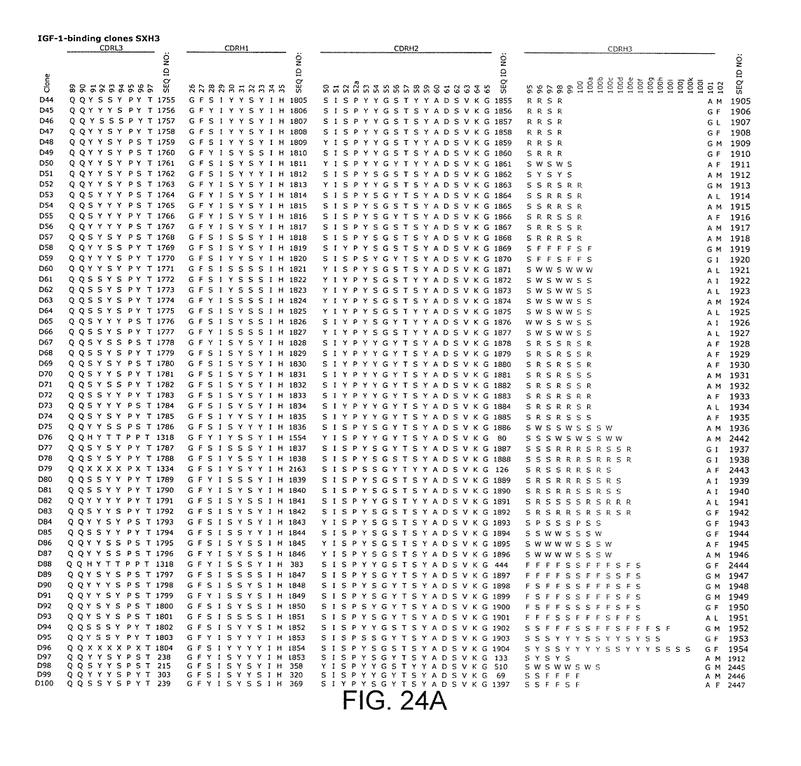

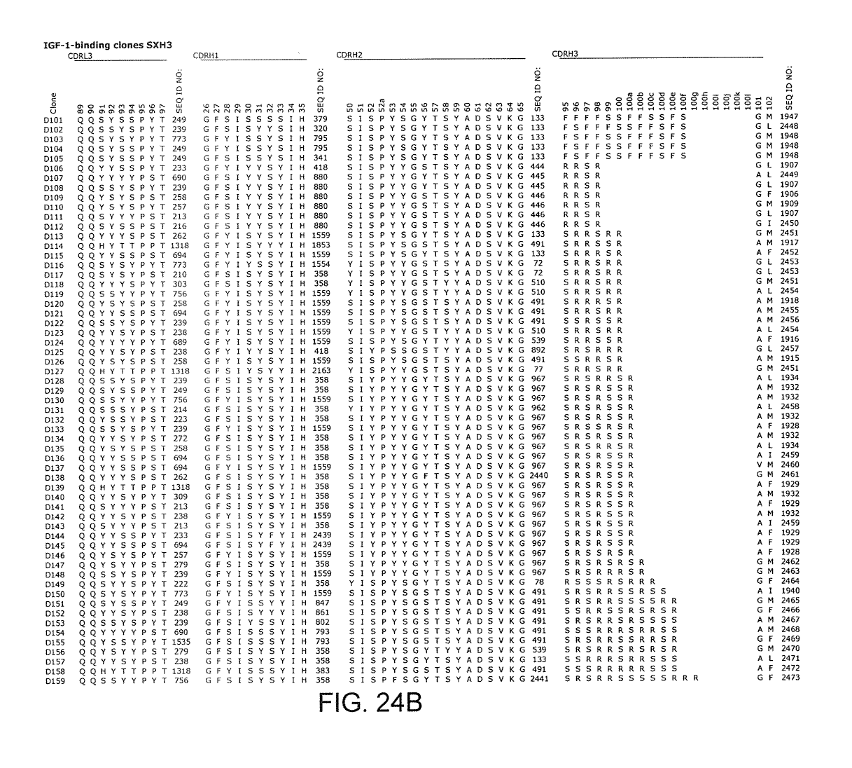

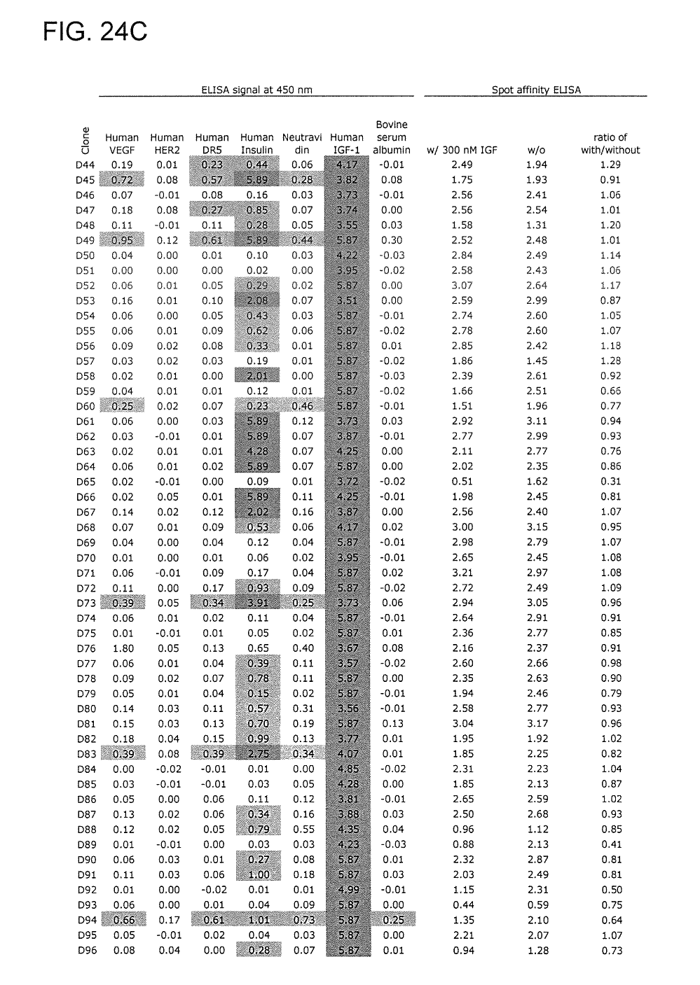

Of the 125 clones identified that specifically bound to human IGF-1, 116 of them had unique CDR sequences (see FIGS. 24A-B). The unique sequences fell into five categories: (a) CDR sequences with randomized positions limited to binary Tyr/Ser (clone nos. D51, D95, D96); (b) CDR sequences with randomized positions limited to binary Trp/Ser (clone nos. D50, D60-66, D75, D85-87); (c) CDR sequences with randomized positions limited to binary Arg/Ser (clone nos. D44-49, D52-57, D67-74, and D77-83); (d) CDR sequences with randomized positions limited to binary Phe/Ser (clone nos. D58, D59, D89-94); (e) CDR sequences with randomized positions limited to binary Pro/Ser (clone no. D84). Additional clones D99-D161 have been sequenced. These clones bound with high affinity to human IGF-1 but some clones did display cross-reactivity to five other control proteins, human VEGF, HER2, human DR5, human insulin, neutravidin, or HGH (see FIG. 24C).

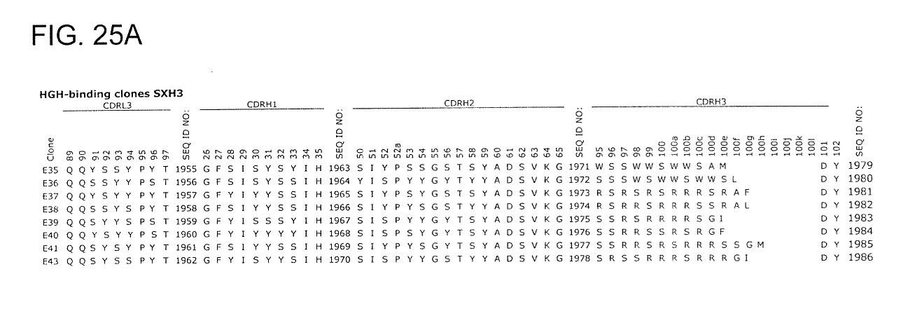

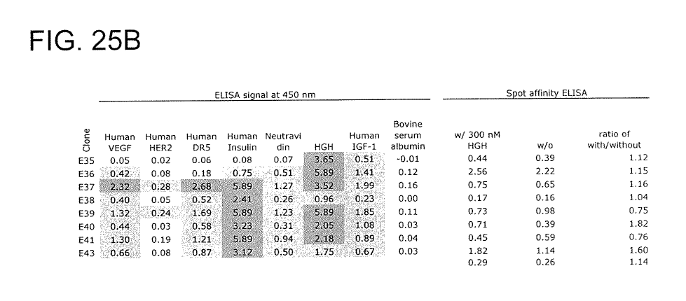

Of the 21 clones identified that specifically bound to HGH, 8 of them had unique CDR sequences (see FIG. 25A). The unique sequences fell into two categories: (a) CDR sequences with randomized positions limited to binary Arg/Ser (clone nos. D37-43); (b) CDR sequences with randomized positions limited to binary Trp/Ser (clone nos. D35, D36). These clones bound to HGH with high affinity but did display cross-reactivity to five other control proteins, human VEGF, HER2, human DR5, human insulin, neutravidin, or human IGF-1 (see FIG. 25B).

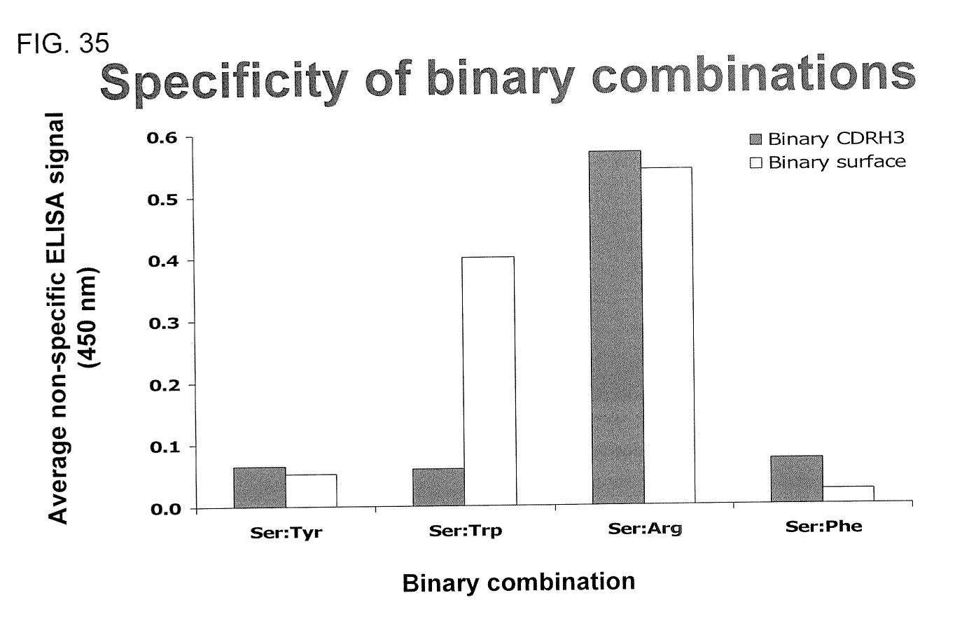

A phage ELISA was used to test the ability of all clones to cross-react with a panel of six antigens other than the target antigen. Phage were produced in a 96-well format as described and phage supematants were diluted 3-fold in PBT buffer. The diluted phage supernatant was transferred to plates coated with human VEGF, HER2, human DR5, human insulin, neutravidin, human IGF-1, HGH, or BSA and incubated for one hour with gentle shaking at room temperature. The plates were washed with PBS including 0.05% Tween 20 and were incubated for 30 minutes with horseradish peroxidase/anti-M13 antibody conjugate (diluted 1:5000 in PT buffer) (Pharmacia). The plates were washed, developed with tetramethylbenzidine (TMB) substrate (Kirkegaard and Perry Laboratories) and quenched with 1.0 M H₃PO₄. Absorbance was determined spectrophotometrically at 450 nm. Weak cross-reactivity was defined as a signal between 0.2-2.0 and strong cross-reactivity was defined as a signal about 2.0. The results for all SXH3 clones are shown in FIGS. 21-25. As shown in FIG. 35, of the SXH3 clones isolated, the S:R clones displayed the greatest average non-specific binding (0.5-0.6 OD at 450 nm by ELISA assay), while the S:W, S:Y, and S:F clones each displayed similar low levels of average non-specific binding (0-0.1 OD at 450 nm by ELISA assay).

A single-point competitive phage ELISA was used to estimate the affinities of the obtained phage-displayed Fabs. Phage were produced in a 96-well format as described, and phage supematants were diluted fifteen-fold in PBT buffer or PBT buffer containing 300 nM human VEGF, human insulin, human IGF-1, or HGH. The mixtures were incubated for 1 hour, then transferred to plates coated with human VEGF, human insulin, human IGF-1 or HGH and incubated for 15 minutes. The plates were washed with PBS including 0.05% Tween 20, and were incubated for 30 minutes with horseradish/peroxidase/anti-M13 antibody conjugate (diluted 1:5000 in PT buffer) (Pharmacia). The plates were washed, developed with tetramethylbenzidine (TMB) substrate (Kirkegaard and Perry Laboratories) and quenched with 1.0 M H₃PO₄. Absorbance was determined spectrophotometrically at 450 nm. The fraction of Fab-phage uncomplexed with solution-phase human VEGF, human insulin, human IGF-1 or HGH was calculated by dividing the A450 in the presence of antigen by the A450 in the absence of antigen. The results are shown in FIGS. 21C-21D, FIG. 23C, FIG. 24C, and FIG. 25B.

A competitive phage ELISA was used to estimate the binding affinities of HER2-binding phage-displayed Fabs. Phage were produced in a 96-well format as described, and phage supernatants were serially diluted in PBT buffer, then incubated on plates coated with HER2 for 15 minutes. The plates were washed with PBS including 0.05% Tween 20 and were incubated for 30 minutes with horseradish peroxidase/anti-M13 antibody conjugate (diluted 1:5000 in PT buffer) (Pharmacia). The plates were washed, developed with tetramethylbenzidine (TMB) substrate (Kirkegaard and Perry Laboratories) and quenched with 1.0 M H₃PO₄. Absorbance was measured spectrophotometrically at 450 nm to determine the phage concentration giving about 50% of the signal at saturation. A fixed, sub-saturating concentration of phage was diluted two fold in PBT buffer or PBT buffer containing two-fold serial dilutions of HER2 protein from 250 nM HER2 to 0.12 nM HER2. The mixtures were incubated for one hour with gentle shaking at room temperature, transferred to plates coated with HER2 and the plates were incubated for 15 minutes. The plates were washed and treated exactly as above. The binding affinities were estimated as IC₅₀ values (defined as the concentration of antigen that blocked 50% of the phage binding to the immobilized antigen). The results are shown in FIG. 22B.

### EXAMPLE 7

### Construction of Phage-Displayed Fab Libraries with CDR Residues Enriched in Ser and Phe, Arg, Trp, or Tyr

Phage-displayed Fab libraries were constructed using a phagemid vector, Fab-C, that resulted in the display of bivalent Fab moieties dimerized by a free cysteine inserted between the Fab heavy chain and the C-terminal domain of the gene-3 minor coat protein (P3C), as previously described in Example 1.

Four libraries were constructed: SFH3, SRH3, SWH3, and SYH3. The libraries were constructed with randomized residues in all three heavy chain CDRs and light chain CDR3. Each library was randomized at positions 91-94 and 96 of CDRL3, positions 28 and 30-33 of CDRH1, positions 50, 52-54, 56, and 58 of CDRH2, and positions 95-100, 101, and 102 of CDRH3. The type and ratio of the amino acids allowed at each of the randomized positions is described in FIG. 26. In addition, the length of CDRH3 was varied by using oligonucleotides that replaced the six wild-type codons between positions 95 and 100 with 4 to 17 codons. The type and ratio of the amino acids allowed at those positions were the same as the ones described in FIG. 26 for positions 95-100 of CDRH3.

Libraries were constructed using the method of Kunkel (Kunkel, T. A., Roberts, J. D. & Zakour, R. A., Methods Enzymol. (1987), 154, 367-382) with previously described methods (Sidhu, S. S., Lowman, H. B., Cunningham, B. C. & Wells, J. A., Methods Enzymol. (2000), 328, 333-363). A unique “stop template” version of the Fab display vector Fab-C was used to generate all four libraries, as described in Example 1.

Mutagenic oligonucleotides with degenerate codons at the positions to be diversified were used to simultaneously (a) introduce CDR diversity and (b) repair the stop codons. The sequences of those mutagenic oligonucleotides are shown in FIGS. 19 and 27. For the library SF-surface, diversity was introduced into CDR-L3, CDR-H1 and CDR-H2 with the oligonucleotides L3-SF, H1-SF and H2-SF respectively (SEQ ID NOS: 1989, 1987, and 1988) (FIG. 27) and diversity was introduced into CDR-H3 with an equimolar mixture of oligonucleotides H3-SF4, H3-SF5, H3-SF6, H3-SF7, H3-SF8, H3-SF9, H3-SF10, H3-SF11, H3-SF12, H3-SF13, H3-SF14, H3-SF15, H3-SF16, and H3-SF17 (SEQ ID NOS: 1143-1156) (FIG. 19C).

For the library SR-surface, diversity was introduced into CDR-L3, CDR-H1 and CDR-H2 with the oligonucleotides L3-SR, H1-SR and H2-SR respectively (SEQ ID NOS: 1992, 1990, and 1991) (FIG. 27) and diversity was introduced into CDR-H3 with an equimolar mixture of oligonucleotides H3-SR4, H3-SR5, H3-SR6, H3-SR7, H3-SR8, H3-SR9, H3-SR10, H3-SR11, H3-SR12, H3-SR13, H3-SR14, H3-SR15, H3-SR16, and H3-SR17 (SEQ ID NOS: 1227-1240) FIG. 19I).

For the library SW-surface, diversity was introduced into CDR-L3, CDR-H1 and CDR-H2 with the oligonucleotides L3-SW, H1-SW and H2-SW respectively (SEQ ID NOS:1995, 1993 and 1994) (FIG. 27) and diversity was introduced into CDR-H3 with an equimolar mixture of oligonucleotides H3-SW4, H3-SW5, H3-SW6, H3-SW7, H3-SW8, H3-SW9, H3-SW10, H3-SW11, H3-SW12, H3-SW13, H3-SW14, H3-SW15, H3-SW16, and H3-SW17 (SEQ ID NOS: 1255-1268) (FIG. 19K).

For the library SY-surface, diversity was introduced into CDR-L3, CDR-H1 and CDR-H2 with the oligonucleotides L3, H1 and H2 respectively (SEQ ID NOS: 160, 158, and 159) (FIG. 19A) and diversity was introduced into CDR-H3 with an equimolar mixture of oligonucleotides H3-SY4, H3-SY5, H3-SY6, H3-SY7, H3-SY8, H3-SY9, H3-SY10, H3-SY11, H3-SY12, H3-SY13, H3-SY14, H3-SY15, H3-SY16, and H3-SY17 (SEQ ID NOS: 1269-1282) (FIG. 19L).

The mutagenic oligonucleotides for all CDRs to be randomized were incorporated in a single mutagenesis reaction, so that simultaneous incorporation of all the mutagenic oligonucleotides resulted in the introduction of the designed diversity at each position and repaired all the TAA stop codons. Thus, an open reading frame was generated that encoded a Fab library member fused to a homodimerizing cysteine bridge and P3C. Following mutagenesis, the four libraries were combined to create a single library, called library SX-surface.

The mutagenesis reactions were electroporated into E. coli SS320 (Sidhu et al., supra). The transformed cells were grown overnight in the presence of M13-KO7 helper phage (New England Biolabs, Beverly, Mass.) to produce phage particles that encapsulated the phagemid DNA and displayed Fab fragments on their surfaces. The combined library contained greater than 3×10¹⁰ unique members.

### EXAMPLE 8

### Selection of Specific Antibodies from Naive Library SX Surface

Phage from library SX-surface (described in Example 7, above) were cycled through rounds of binding selection to enrich for clones binding to human VEGF, HER2, human insulin, human IGF-1, or HGH. The binding selections were conducted using previously described methods (Sidhu et al., supra).

NUNC 96-well Maxisorp immunoplates were coated overnight at 4° C. with 5 μg/mL target protein (human VEGF, HER2, human insulin, human IGF-1, or HGH) and blocked for 2 hours with a solution of PBT (Sigma). After overnight growth at 37° C., phage were concentrated by precipitation with PEG/NaCl and resuspended in PBT, as described previously (Sidhu et al., supra). Phage solutions (about 10¹² phage/mL) were added to the coated immunoplates. Following a two hour incubation to permit phage binding, the plates were washed ten times with PBT. Bound phage were eluted with 0.1 M HCl for ten minutes and the eluant was neutralized with 1.0 M Tris base. Eluted phage were amplified in E. coli XL1-blue and used for further rounds of selection. The libraries were subjected to six rounds of selection against each target protein. Individual clones from each round of selection were grown in a 96-well format in 500 μL of 2YT broth supplemented with carbenicillin and M13-K07. The culture supematants were used directly in phage ELISAs (Sidhu et al., supra) to detect phage-displayed Fabs that bound to plates coated with target protein but not to plates coated with BSA. Specific binders were defined as those phage clones that exhibited an ELISA signal at least 10-fold greater on target-coated plates in comparison with BSA-coated plates. Individual clones were screened after 4, 5 and 6 rounds of selection for binding to human VEGF, HER2, human insulin, human IGF-1, or HGH. The specific binders were subjected to sequence analysis. As shown in FIG. 20, the SX-surface library produced specific binders against all five target proteins. The distribution of target-binding clones from each S:X-surface library is shown in FIG. 34 as well as the distribution of properly folded and displayed S:X-surface antibodies that bound to Protein A.

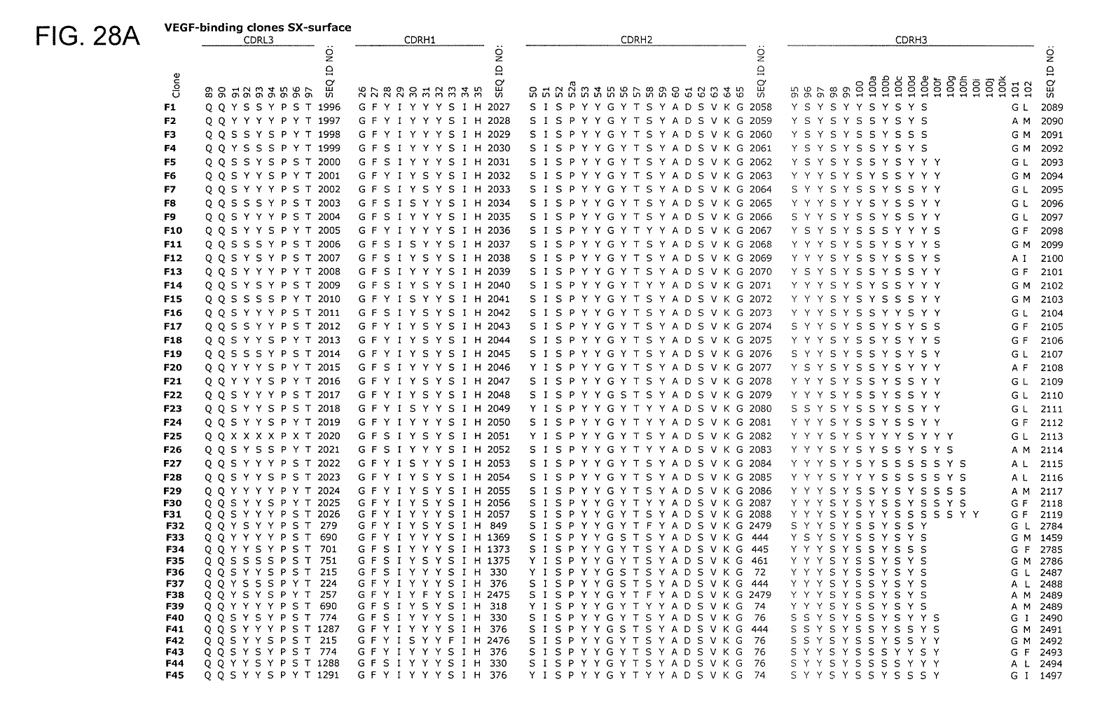

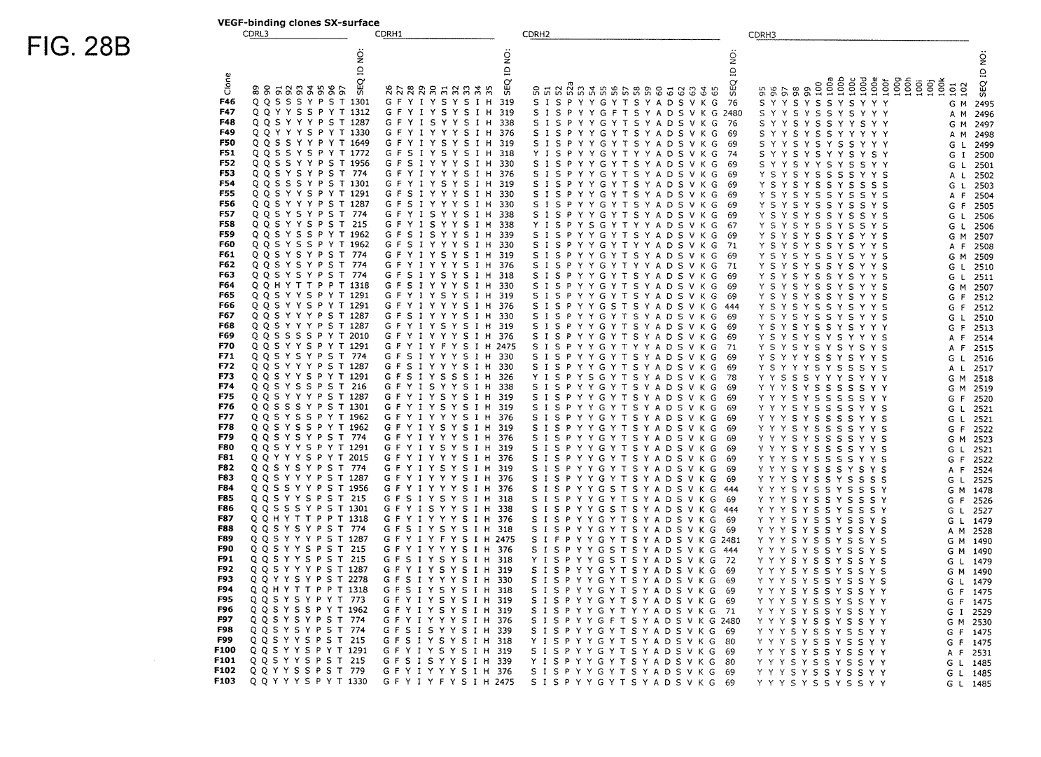

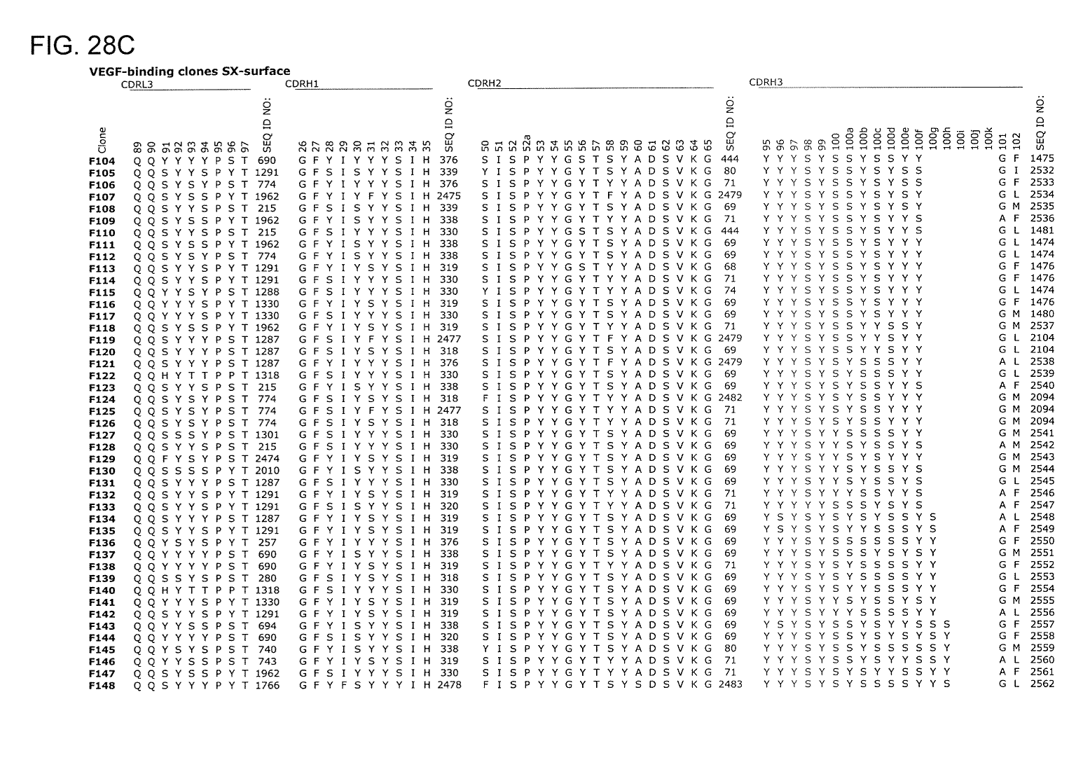

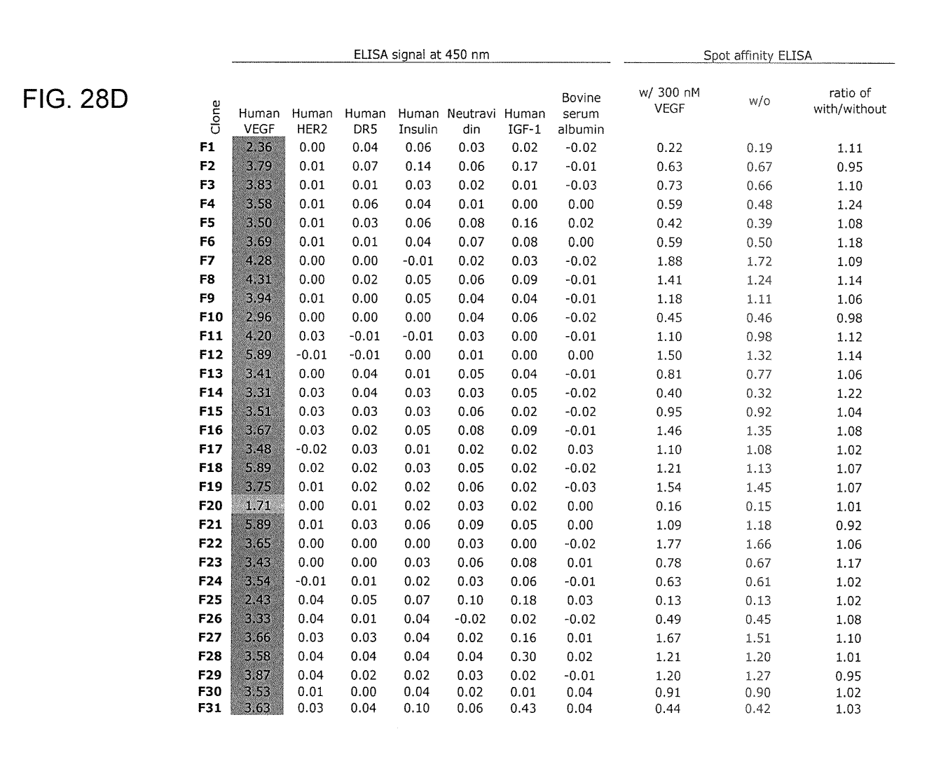

Of the 181 clones identified that specifically bound to human VEGF, 148 of them had unique CDR sequences (see FIGS. 28A-C). Some of the unique sequences had randomized positions limited to binary Tyr/Ser (clone nos. F1-31). Clones F32-148 were additionally sequenced. Clones F1-31 were highly specific for VEGF and did not display cross-reactivity to five other control proteins, HER2, human DR5, human insulin, neutravidin, human IGF-1 or HGH (see FIG. 28D).

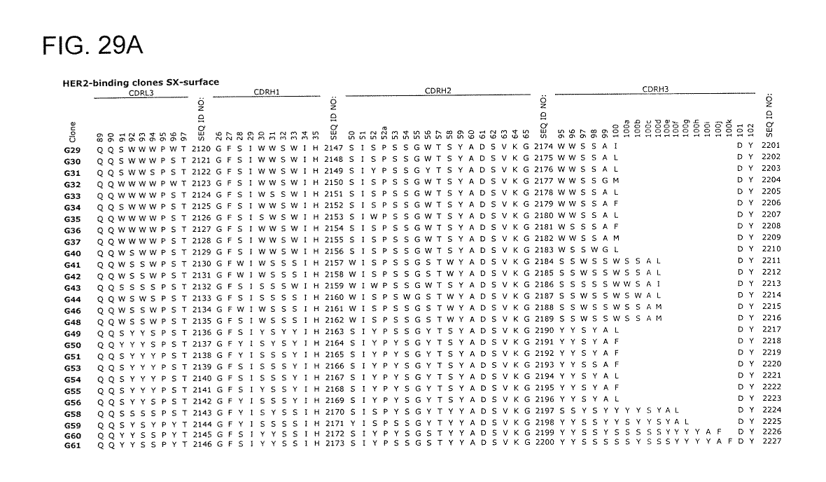

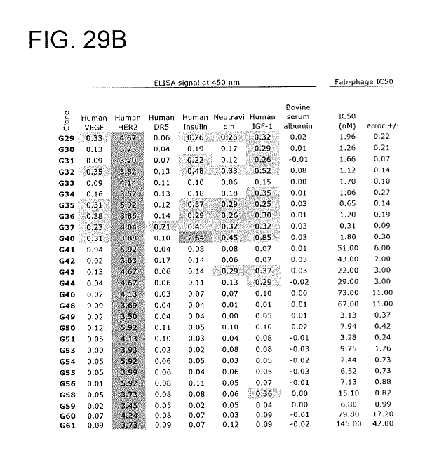

Of the 81 clones identified that specifically bound to HER2, 27 of them had unique CDR sequences (see FIG. 29A). The unique sequences fell into two categories: (a) CDR sequences with randomized positions limited to binary Tyr/Ser (clone nos. G49-61); (b) CDR sequences with randomized positions limited to binary Trp/Ser (clone nos. G29-48). The Tyr/Ser clones were highly specific for HER2 and did not display cross-reactivity to five other control proteins, human VEGF, human DR5, human insulin, neutravidin, human IGF-1 or HGH (see FIG. 29B). However, some of the Trp/Ser clones were cross-reactive (see FIG. 29B). The inhibitory concentration for each clone is shown in FIG. 29B.

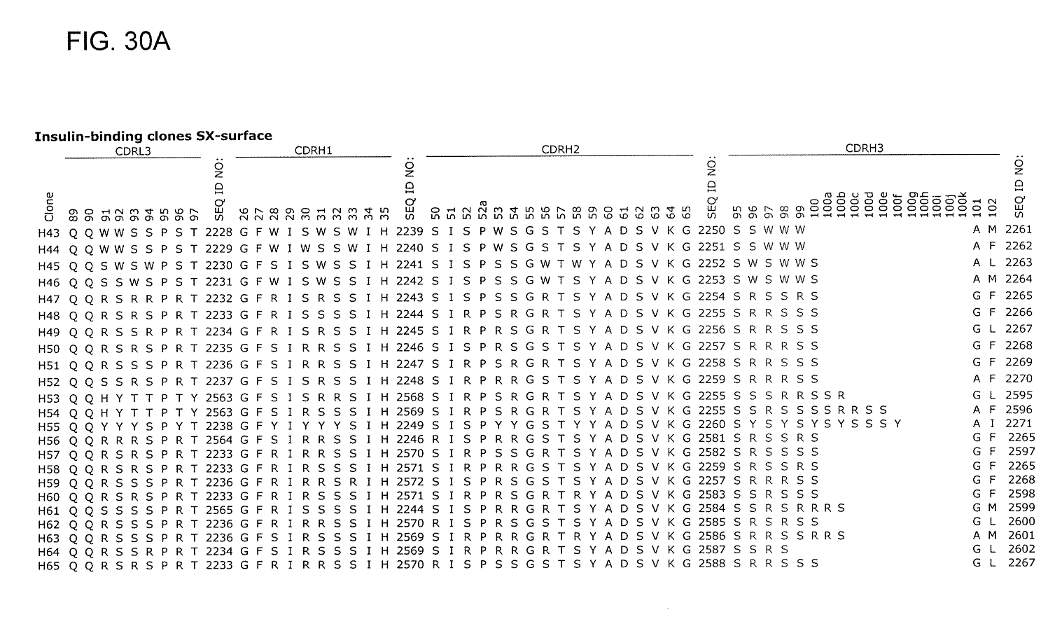

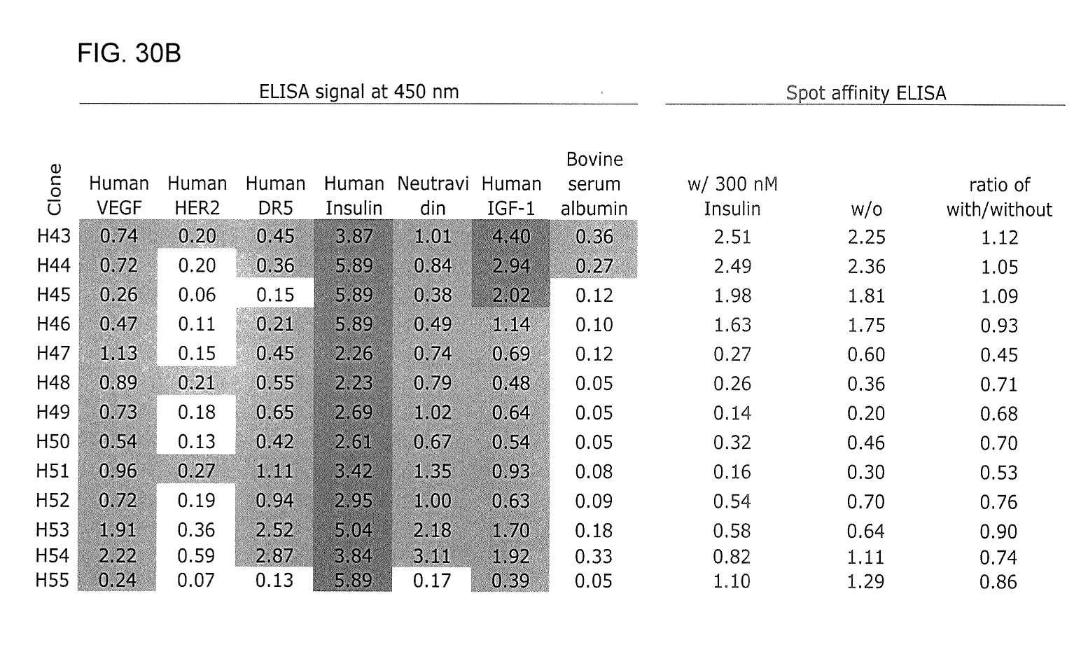

Of the 29 clones identified that specifically bound to human insulin, 23 had unique CDR sequences (see FIG. 30A). The unique sequences fell into three categories: (a) CDR sequences with randomized positions limited to binary Tyr/Ser (clone no. H55); (b) CDR sequences with randomized positions limited to binary Trp/Ser (clone nos. H43-46); (c) CDR sequences with randomized positions limited to binary Arg/Ser (clone nos. H47-52). Clones H56-65 were additionally sequenced. Clones H43-H55 bound with high affinity to human insulin but also displayed cross-reactivity to five other control proteins, human VEGF, HER2, human DR5, neutravidin, human IGF-1 or HGH (see FIG. 30B).

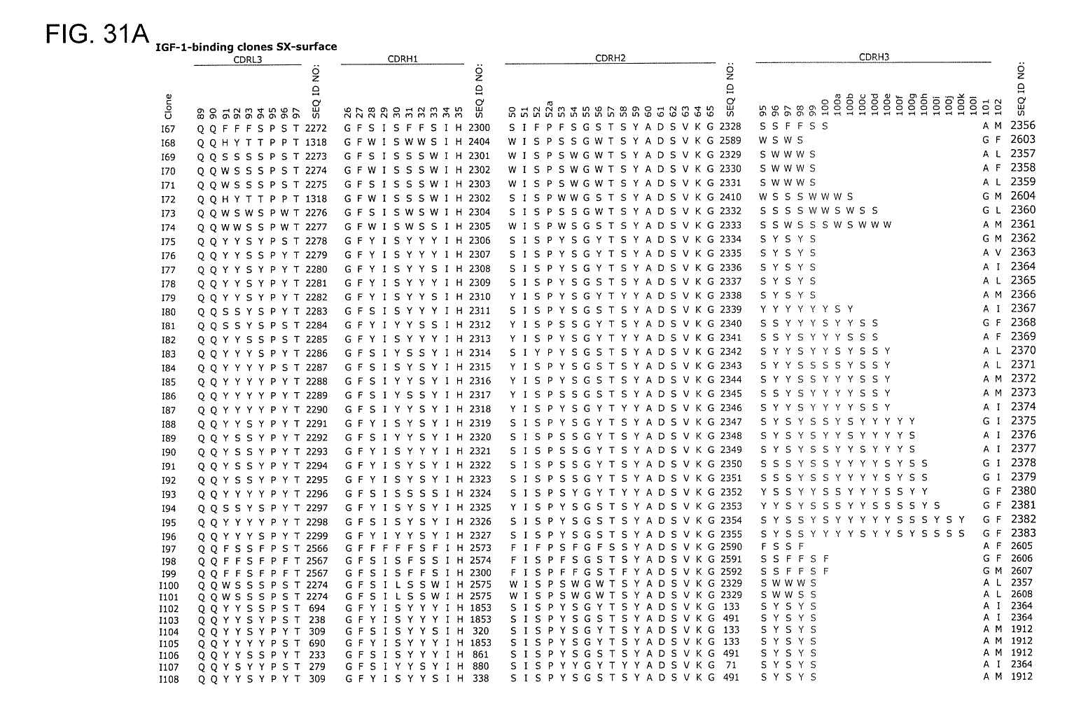

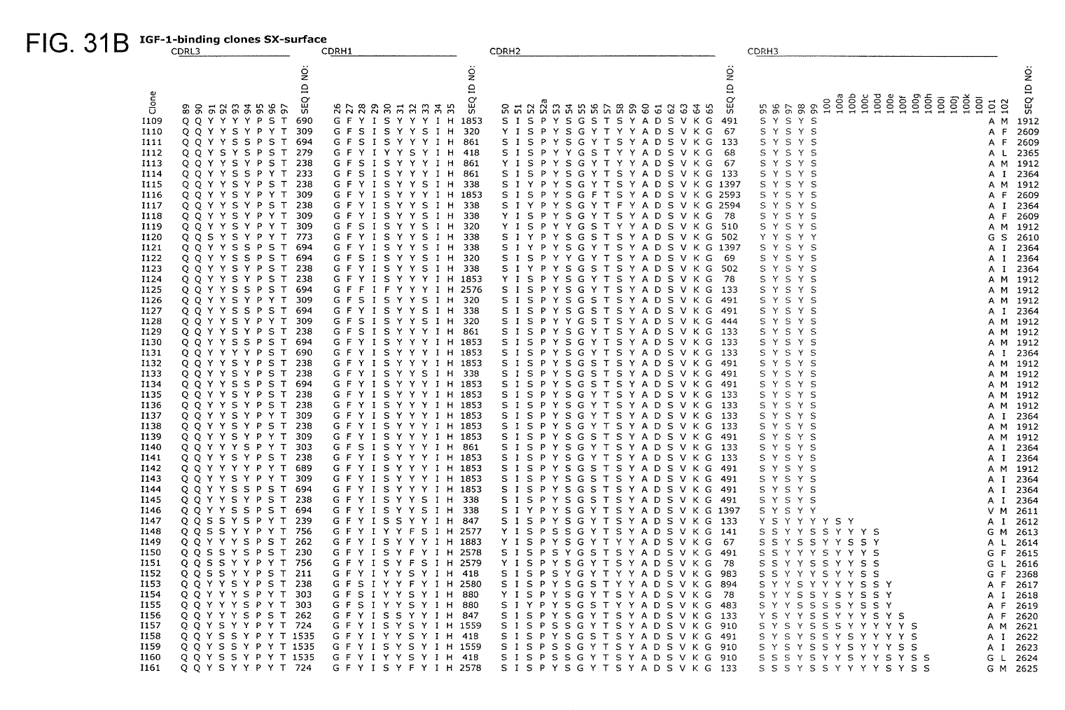

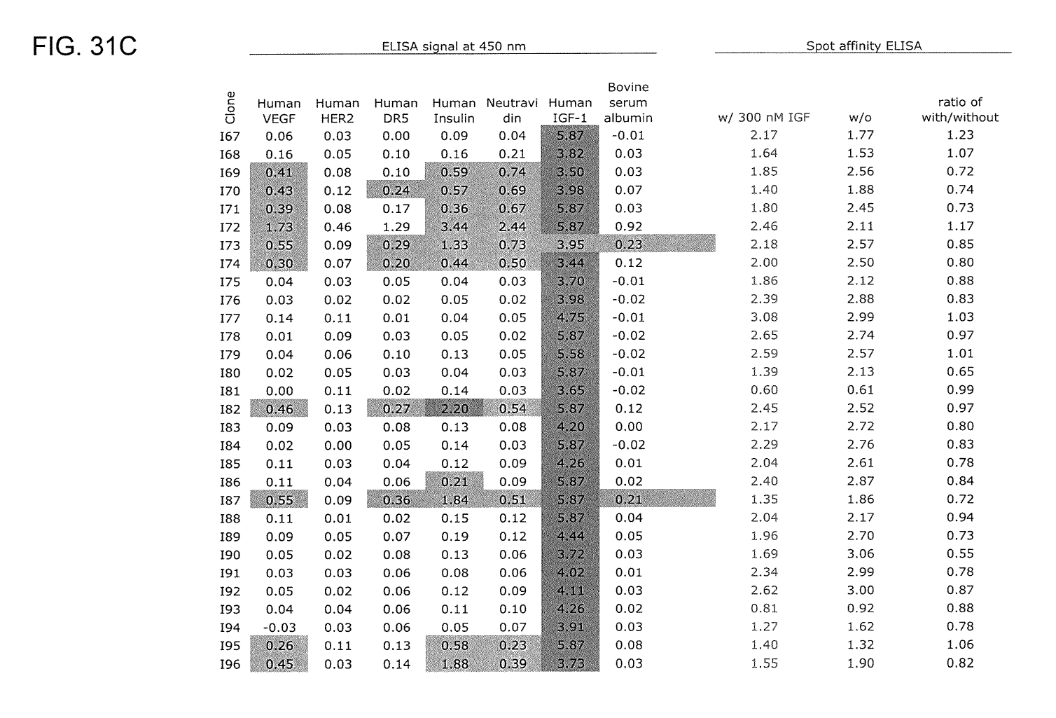

Of the 237 clones identified that specifically bound to human IGF-1, 95 of them had unique CDR sequences (see FIGS. 31A-B). Some of the unique sequences fell into three categories: (a) CDR sequences with randomized positions limited to binary Tyr/Ser (clone nos. I75-96); (b) CDR sequences with randomized positions limited to binary Trp/Ser (clone nos. I69-74); (c) CDR sequences with randomized positions limited to binary Phe/Ser (clone no. I67). Clones I97-161 were additionally sequenced. Clones I167 to I96 bound with high affinity to human IGF-1 but some Trp/Ser and Tyr/Ser clones did display cross-reactivity to five other control proteins, human VEGF, HER2, human DR5, human insulin, neutravidin or HGH (see FIG. 31C).

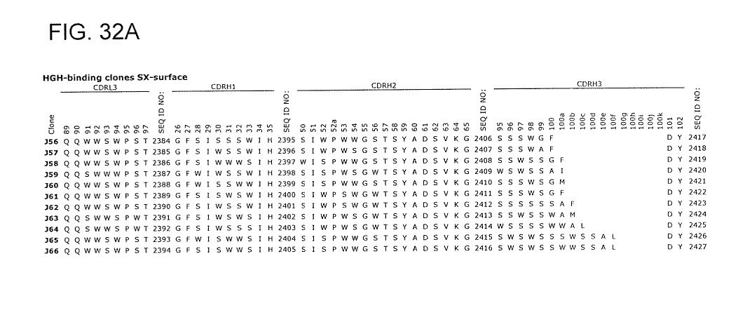

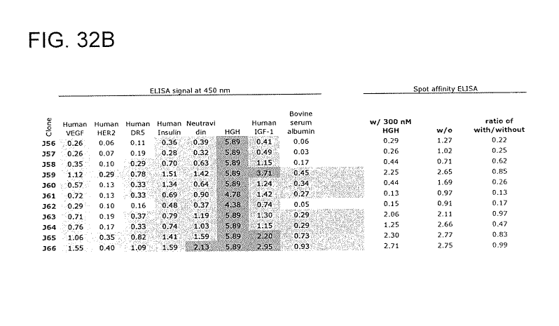

Of the 16 clones identified that specifically bound to HGH, 11 of them had unique CDR sequences (see FIG. 32A). The unique CDR sequences were all Trp/Ser containing and were highly cross-reactive (see FIG. 32B).

A phage ELISA was used to test the ability of all clones to cross-react with a panel of six antigens other than the target antigen. Phage were produced in a 96-well format as described above and phage supernatants were diluted 3-fold in PBT buffer. The diluted phage supernatant was transferred to plates coated with human VEGF, HER2, human DR5, human insulin, neutravidin, human IGF-1, HGH, or BSA and incubated for one hour with gentle shaking at room temperature. The plates were washed with PBS including 0.05% Tween 20 and were incubated for 30 minutes with horseradish peroxidase/anti-M13 antibody conjugate (diluted 1:5000 in PT buffer) (Pharmacia). The plates were washed, developed with tetramethylbenzidine (TMB) substrate (Kirkegaard and Perry Laboratories) and quenched with 1.0 M H₃PO₄. Absorbance was determined spectrophotometrically at 450 nm. Weak cross-reactivity was defined as a signal between 0.2-2.0 and strong cross-reactivity was defined as a signal above 2.0. The results for all SX-surface clones are shown in FIGS. 28, 29, 30, 31 and 32. As shown in FIG. 35, of the SX-surface clones isolated, the S:R and S:W clones displayed the greatest average non-specific binding (0.5-0.6 OD and approximately 4.0 OD, respectively, at 450 mn by ELISA assay), while the S:Y and S:F clones each displayed similar low levels of average non-specific binding (0-0.1 OD at 450 mn by ELISA assay).

A single-point competitive ELISA was used to estimate the affinities of phage-displayed Fabs. Phage were produced in a 96-well format as described above, and phage supernatants were diluted fifteen-fold in PBT buffer or PBT buffer with 300 nM human VEGF, human insulin, human IGF-1, or HGH. The mixtures were incubated for 1 hour, and then transferred to plates coated with human VEGF, human insulin, human IGF-1, or HGH and incubated for 15 minutes. The plates were washed with PBS including 0.05% Tween 20 and were incubated for 30 minutes with horseradish peroxidase/anti-M13 antibody conjugate (diluted 1:5000 in PT buffer) (Pharmacia). The plates were washed, developed with tetramethylbenzidine (TMB) substrate (Kirkegaard and Perry Laboratories) and quenched with 1.0 M H₃PO₄. Absorbance was determined spectrophotometrically at 450 nm. The fraction of Fab-phage uncomplexed with solution-phase human VEGF, human insulin, human IGF-1 or HGH was calculated by dividing the A₄₅₀ in the presence of antigen by the A₄₅₀ in the absence of antigen. The results are shown in FIGS. 28D, 30B, 31C, and 32B.

A competitive phage ELISA was also used to estimate the binding affinities of HER2-binding phage-displayed Fabs. Phage were produced in a 96-well format as described above, and phage supernatants were serially diluted in PBT buffer, then incubated on plates coated with HER2 for 15 minutes. The plates were washed with PBS including 0.05% Tween 20 and were incubated for 30 minutes with horseradish peroxidase/anti-M13 antibody conjugate (diluted 1:5000 in PT buffer) (Pharmacia). The plates were washed, developed with tetramethylbenzidine (TMB) substrate (Kirkegaard and Perry Laboratories) and quenched with 1.0 M H₃PO₄. Absorbance was measured spectrophotometrically at 450 nm to determine the phage concentration giving ˜50% of the signal at saturation. A fixed, sub-saturating concentration of phage was diluted two fold in PBT buffer or PBT buffer containing two-fold serial dilutions of HER2 protein from 250 nM HER2 to 0.12 nM HER2. The mixtures were incubated for one hour with gentle shaking at room temperature, transferred to plates coated with HER2 and the plates were incubated for 15 minutes. The plates were washed and treated exactly as above. The binding affinities were estimated as IC₅₀ values (defined as the concentration of antigen that blocked 50% of the phage binding to the immobilized antigen). The results are shown in FIG. 29B.

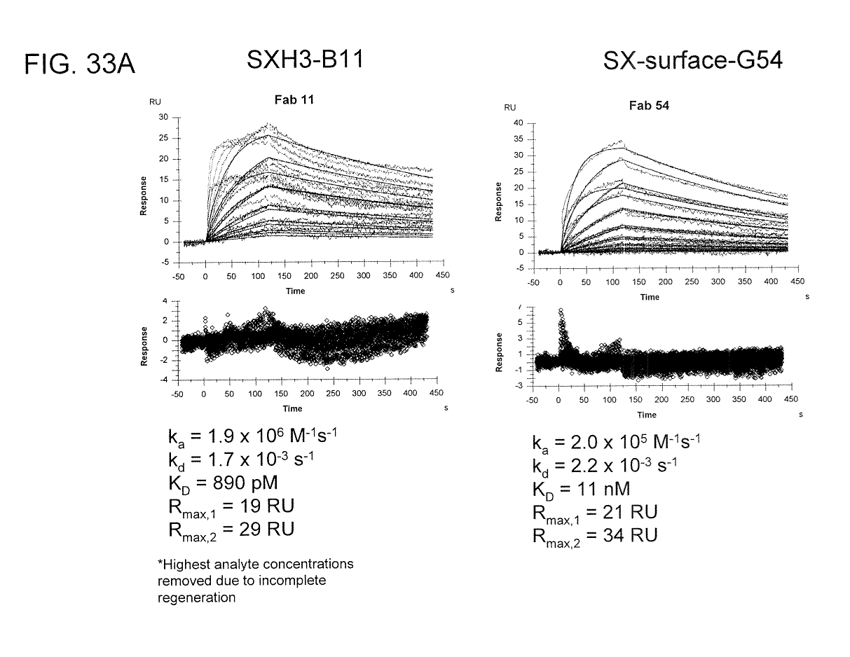

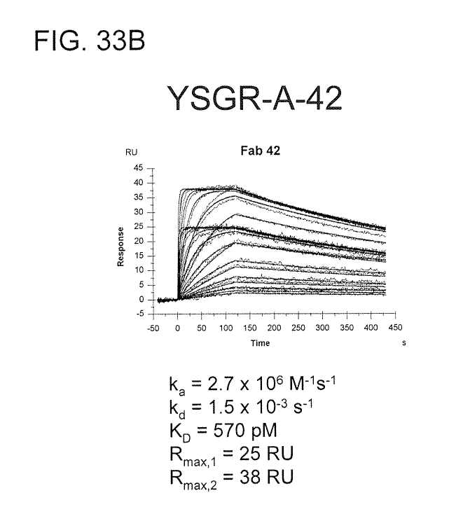

Based on this analysis, the analysis of HER2-binding clones from the SXH3 library (Example 6), and the YSGR-A-D library (Example 4), soluble Fab proteins from three clones (clone nos. 42 (YSGR-A) and B11 (SXH3) and G54 (SX-surface)) were purified and subjected to surface plasmon resonance analysis of binding to human HER2. BIAcore® data was obtained according to Chen et al., J. Mol. Biol. (1999), 293(4): 865-81. Briefly, binding affinities of the purified Fabs for human HER2 were calculated from association and dissociation rate constants measured using a BIAcore®-A100 surface plasmon resonance system (BIACORE, Inc., Piscataway, N.J.). HER2 was covalently coupled to a biosensor chip at two different concentrations using N-ethyl-N′-(3-dimethylaminopropyl)-carbodiimide hydrochloride (EDC) and N-hydroxysuccinimide (NHS) according to the supplier's (BIAcore, Inc., Piscataway, N.J.) instructions. HER2 was buffer-exchanged into 10 mM sodium acetate, pH 5.0 and diluted to approximately 2.5 or 5.0 μg/ml. Aliquots of HER2 were injected at a flow rate of 5 μL/min to achieve approximately 50-170 response units (RU) of coupled protein. A solution of 1 M ethanolamine was injected as a blocking agent. For kinetics measurements, twofold serial dilutions of each Fab were injected in HBT at 25° C. at a flow rate of 10 μL/minute over each flow cell. The kₒₙ and k_{off} values were determined from the binding curves using the BIAevaluation software package (BIACORE, Inc., Piscataway, N.J.) using two-spot global fitting and combining the data from both flow cells. The equilibrium dissociation constant, K_{D}, was calculated as K_{off}/kₒₙ. The BIAcore® data is summarized in FIGS. 33A and B. Clone B11 had a kₐ of 1.9×10⁶ M⁻¹s⁻¹, a k_{d} of 1.7×10⁻³ s⁻¹, and a K_{D} of 890 pM. Rmax1 for the clone B11 experiments was 19 RU, and Rmax2 for the clone B11 experiments was 29 RU. Clone G54 had a kₐ of 2.0×10⁵ M⁻¹s⁻¹, a k_{d} of 2.2×10⁻³ s⁻¹, and a K_{D} of 11 nM. Rₘₐₓ₁ for the clone G54 experiments was 21 RU and Rₘₐₓ₂ for the clone G54 experiments was 34 RU. Clone YSGR-A-42 had a kₐ of 2.7×10⁶ M⁻¹s⁻¹, a k_{d} of 1.5×10⁻³ s⁻¹, and a K_{D} of 570 pM. Rmax1 for the clone 42 experiments was 25 RU, and Rmax2 for the clone 42 experiments was 38 RU. The tryptophan-containing clone (B11) had a faster kₒₙ and correspondingly smaller K_{D} than the tyrosine-containing clone (G54).

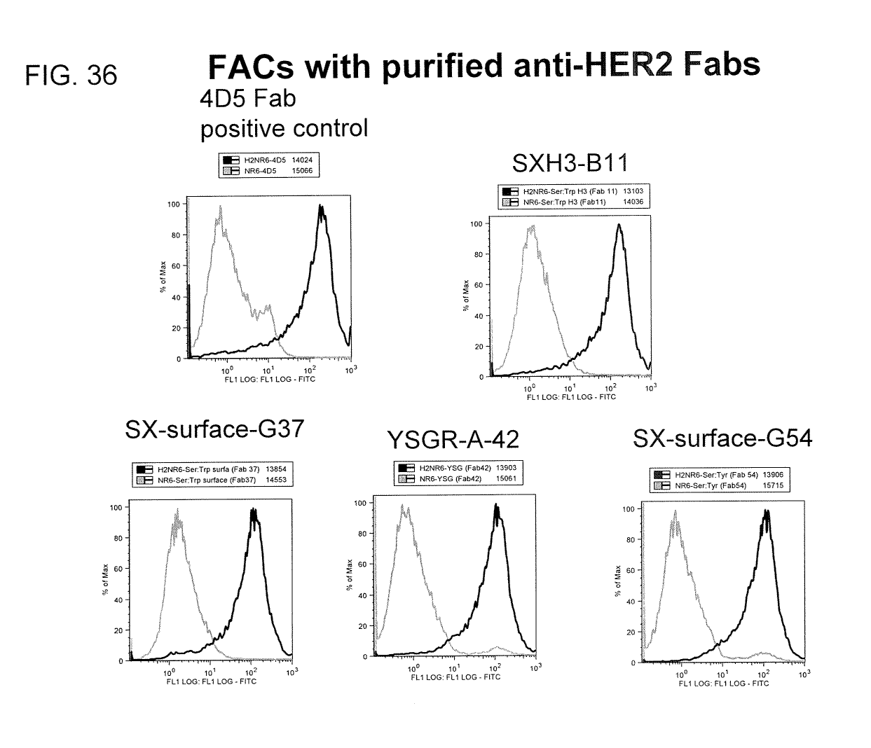

To study binding of anti-HER2 antibodies to HER2 expressed on mammalian cells, the binding of purified Fab protein of clones 42 (YSGR-A), B11 (SXH3), G54 (SX-surface), and G37 (SX-surface) to NR6 fibroblast cells over-expressing HER2 (NR6-HER2) was studied by flow cytometry. One million NR6-HER2 cells were incubated with 10 μg/ml Fab for 1 hour, followed by incubation with an Alexa488-conjugated murine anti-human IgG antibody for 1 hour. As a negative control, Fab binding to non-expressing NR6 cells was studied. As a positive control, 4D5 Fab was used. As demonstrated in FIG. 36, clones 42, B11, G54, and G37 bind specifically to Her2 on NR6 cells.

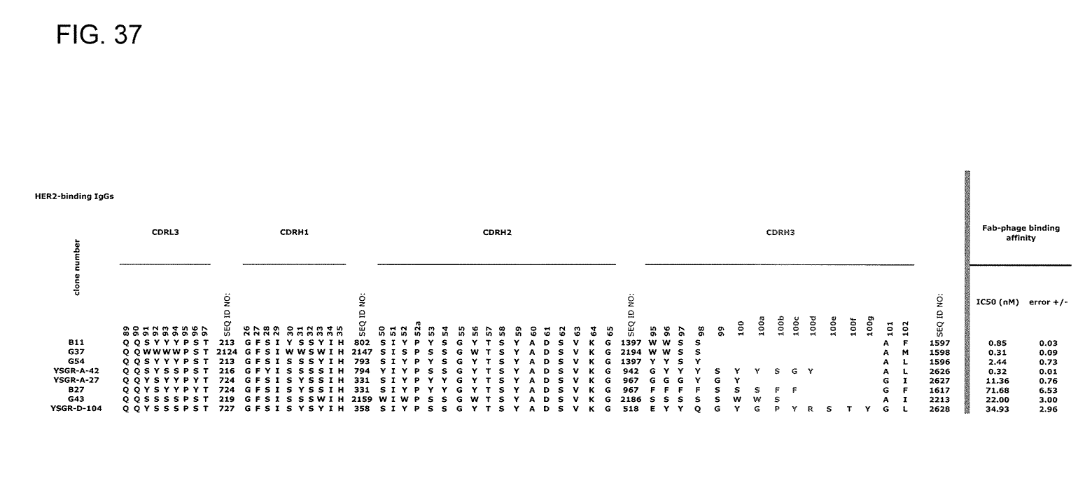

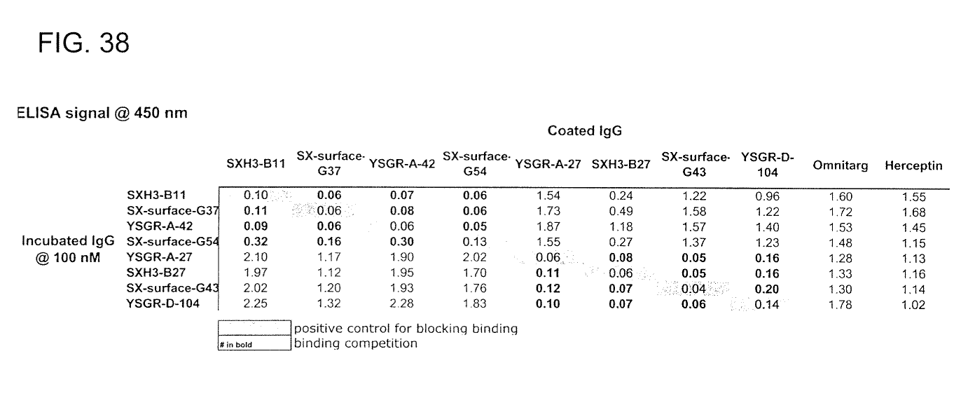

A competitive ELISA was used to test binding competition with Herceptin and Omnitarg and between several HER2-binding clones in IgG format (see FIG. 37 for the CDR sequences of the relevant clones). Biotinylated HER2 protein was serially diluted from 200 nM to 0.39 nM in PBT buffer, then incubated on plates coated with purified IgG proteins for 15 minutes. The plates were washed with PBS containing 0.05% Tween 20, and were incubated for 30 minutes with horseradish peroxidase/anti-M13 antibody conjugate (diluted 1:5000 in PT buffer) (Pharmacia). The plates were washed, developed with tetramethylbenzidine (TMB) substrate (Kirkegaard and Perry Laboratories) and quenched with 1.0 M H₃PO₄. Absorbance was measured spectrophotometrically at 450 nm to determine the biotinylated HER2 concentration giving around 50% of the signal at saturation. A fixed, sub-saturating concentration of biotinylated HER2 was diluted two-fold in PBT buffer or PBT buffer containing 100 nM purified IgG proteins. The mixtures were incubated for one hour with gentle shaking at room temperature, transferred to plates coated with IgG proteins, and the plates were incubated for 15 minutes. The plates were washed and treated as above. As shown in FIG. 38, none of the HER2-binding IgGs blocked binding of biotinylated HER2 to either Omnitarg or Herceptin. The IgGs did block binding between each other in two groups. One group made up of clones B11, G37, G54, and YSGR-A-42 compete for the same epitope and blocked binding to biotinylated HER2 that had been previously incubated with any of those clones. A second group made up of clones YSGR-A-27, B27, G43, and YSGR-D-104 compete for the same epitope on HER2 and blocked binding to biotinylated HER2. Group one clones are all higher-affinity binders than the group two clones.

All publications (including patents and patent applications) cited herein are hereby incorporated in their entirety by reference.

1. A polypeptide comprising an immunoglobulin heavy chain variable domain, wherein:
(i) CDRH1 comprises an amino acid sequence G-F-N-X1-X2-X3-X4-X5-X6-H (SEQ ID NO: 2629), wherein G is position 26 and X1 is position 29 according to the Kabat numbering system; wherein X1 is selected from F, L, I, and V; wherein X2 is selected from Y and S; wherein X3 is selected from Y and S; wherein X4 is selected from Y and S; wherein X5 is selected from Y and S, and wherein X6 is selected from M and I;
(ii) CDRH2 comprises an amino acid sequence: X1-I-X2-X3-X4-X5-X6-X7-T-X8-Y-A-D-S-V-K-G (SEQ ID NO: 2630), wherein X1 is position 50 according to the Kabat numbering system; wherein X1 is selected from Y and S; wherein X2 is selected from Y and S; wherein X3 is selected from P and S; wherein X4 is selected from Y and S; wherein X5 is selected from Y and S; wherein X6 is selected from G and S; wherein X7 is selected from Y and S; and wherein X8 is selected from Y and S; and
(iii) CDRH3 comprises an amino acid sequence: X1-X2-X3-X4-X5-X6-X7-X8-X9-X10-X11-X12-X13-X14-X15-X16-X17-X18-X19-D-Y (SEQ ID NO: 2631), wherein X1 is position 95 according to the Kabat numbering system, and wherein X1 is selected from Y and S; wherein X2 is selected from Y and S; wherein X3 is selected from Y and S, wherein X4 is selected from Y and S; wherein X5 is selected from Y and S; wherein X6 is selected from Y and S; wherein X7 is selected from Y and S or is not present; wherein X8 is selected from Y and S or is not present; wherein X9 is selected from Y and S or is not present; wherein X10 is selected from Y and S or is not present; wherein X11 is selected from Y and S or is not present; wherein X12 is selected from Y and S or is not present; wherein X13 is selected from Y and S or is not present; wherein X14 is selected from Y and S or is not present; wherein X15 is selected from Y and S or is not present; wherein X16 is selected from Y and S or is not present; wherein X17 is selected from Y and S or is not present; wherein X18 is selected from G and A; and wherein X19 is selected from F, L, I, and M.

2. The polypeptide of claim 1, wherein CDRH1 comprises an amino acid sequence selected from SEQ ID NOs: 52-66.

3. The polypeptide of claim 1, wherein CDRH2 comprises an amino acid sequence selected from SEQ ID NOs: 67-81.

4. The polypeptide of claim 1, wherein CDRH3 comprises an amino acid sequence selected from SEQ ID NOs: 82-96.

5. A polypeptide comprising an immunoglobulin heavy chain variable domain, wherein:
(i) CDRH1 comprises an amino acid sequence G-F-N-X1-X2-X3-X4-X5-X6-H (SEQ ID NO: 2629), wherein G is position 26 and X1 is position 29 according to the Kabat numbering system; wherein X1 is selected from F, L, I, and V; wherein X2 is selected from Y and S; wherein X3 is selected from Y and S; wherein X4 is selected from Y and S; wherein X5 is selected from Y and S, and wherein X6 is selected from M and I;
(ii) CDRH2 comprises an amino acid sequence: X1-I-X2-X3-X4-X5-X6-X7-T-X8-Y-A-D-S-V-K-G (SEQ ID NO: 2630), wherein X1 is position 50 according to the Kabat numbering system; wherein X1 is selected from Y and S; wherein X2 is selected from Y and S; wherein X3 is selected from P and S; wherein X4 is selected from Y and S; wherein X5 is selected from Y and S; wherein X6 is selected from G and S; wherein X7 is selected from Y and S; and wherein X8 is selected from Y and S; and
(iii) CDRH3 comprises an amino acid sequence: X1-X2-X3-X4-X5-X6-X7-X8-X9-X10-X11-X12-X13-X14-X15-X16-X17-X18-X19-D-Y (SEQ ID NO: 2632), where X1 is position 95 according to the Kabat numbering system, and wherein the amino acids at each of positions X1-X6 are selected from a pool of amino acids in a molar ratio of 20% Y, 15% S, 15% G, 3.125% A, 3.125% D, 3.125% E, 3.125% F, 3.125% H, 3.125% I, 3.125% K, 3.125% L, 3.125% M, 3.125% N, 3.125% P, 3.125% Q, 3.125% R, 3.125% T. 3.125% V, and 3.125% W; wherein the amino acids at each of positions X7-X17 are selected from a pool of amino acids in a molar ratio of 20% Y, 15% S. 15% G., 3.125% A, 3.125% D, 3.125% E, 3.125% F, 3.125% H, 3.125% I, 3.125% K, 3.125% L, 3.125% M, 3.125% N, 3.125% P, 3.125% Q, 3.125% R, 3.125% T, 3.125% V, and 3.125% W, or are not present; wherein X18 is selected from G and A; and wherein X19 is selected from F, L, I and M.

6. The polypeptide of claim 5, wherein CDRH1 comprises an amino acid sequence selected from SEQ ID NOs: 111-125.

7. The polypeptide of claim 5, wherein CDRH2 comprises an amino acid sequence selected from SEQ ID NOs: 126-141.

8. The polypeptide of claim 5, wherein CDRH3 comprises an amino acid sequence selected from SEQ ID NOs: 142 and 144-157.

9. A polypeptide comprising an immunoglobulin heavy chain variable domain, wherein:
(i) CDRH1 comprises an amino acid sequence G-F-N-X1-X2-X3-X4-X5-X6-H (SEQ ID NO: 2629), wherein G is position 26 and X1 is position 29 according to the Kabat numbering system; wherein X1 is selected from F, L, I, and V; wherein X2 is selected from Y and S; wherein X3 is selected from Y and S; wherein X4 is selected from Y and S; wherein X5 is selected from Y and S, and wherein X6 is selected from M and I;
(ii) CDRH2 comprises an amino acid sequence: X1-I-X2-X3-X4-X5-X6-X7-T-X8-Y-A-D-S-V-K-G (SEQ ID NO: 2630), wherein X1 is position 50 according to the Kabat numbering system; wherein X1 is selected from Y and S; wherein X2 is selected from Y and S; wherein X3 is selected from P and S; wherein X4 is selected from Y and S; wherein X5 is selected from Y and S; wherein X6 is selected from G and S; wherein X7 is selected from Y and S; and wherein X8 is selected from Y and S; and
(iii) CDRH3 comprises an amino acid sequence: X1-X2-X3-X4-X5-X6-X7-D-Y (SEQ ID NO: 2633), wherein X1 is position 95 according to the Kabat numbering system, and wherein the amino acids at each of positions X1-X5 are selected from a pool of amino acids in amolarratio of 20% Y, 15% S, 15% G, 3.125% A, 3.125% D, 3.125% E, 3.125% F, 3.125% H, 3.125% I, 3.125% K, 3.125% L, 3.125% M, 3.125% N, 3.125% P, 3.125% Q, 3.125% R, 3.125% T, 3.125% V, and 3.125% W; wherein X6 is selected from G and A; and wherein X7 is selected from F, L, I and M.

10. The polypeptide of claim 9 wherein CDRH3 comprises the amino acid sequence of SEQ ID NO: 143.

11. A polypeptide comprising an immunoglobulin heavy chain variable domain, wherein:
(i) CDRH1 comprises an amino acid sequence G-F-X1-I-X2-X3-X4-X5-I-H (SEQ ID NO: 2634), wherein G is position 26 and X1 is position 28 according to the Kabat numbering system; wherein X1 is selected from Y and S; wherein X2 is selected from Y and S; wherein X3 is selected from Y and S; wherein X4 is selected from Y and S; and wherein X5 is selected from Y and S;
(ii) CDRH2 comprises an amino acid sequence: X1-I-X2-P-X3-X4-G-X5-T-X6-Y-A-D-S-V-K-G (SEQ ID NO: 2635), wherein X1 is position 50 according to the Kabat numbering system; wherein X1 is selected from Y and S; wherein X2 is selected from Y and S; wherein X3 is selected from Y and S; wherein X4 is selected from Y and S; wherein X5 is selected from Y and S; and wherein X6 is selected from Y and S; and
(iii) CDRH3 comprises an amino acid sequence: X1-X2-X3-X4-X5-X6-X7-X8-X9-X10-X11-X12-X13-X14-X15-X16-X17-X18-X19-D-Y (SEQ ID NO: 2636), wherein X1 is position 95 according to the Kabat numbering system, and wherein the amino acids at each of positions X1-X6 are selected from a pool of amino acids in a molar ratio of 50% Y, 25% S, and 25% G; wherein the amino acids at each of positions X7-X17 are selected from a pool of amino acids in a molar ratio of 50% Y, 25% S, and 25% G, or are not present; wherein X18 is selected from G and A; and wherein X29 is selected from I, M, L, and F.

12. A polypeptide comprising an immunoglobulin heavy chain variable domain, wherein:
(i) CDRH1 comprises an amino acid sequence G-F-X1-I-X2-X3-X4-X5-I-H (SEQ ID NO: 2634), wherein G is position 26 and X1 is position 28 according to the Kabat numbering system; wherein X1 is selected from Y and S; wherein X2 is selected from Y and S; wherein X3 is selected from Y and S; wherein X4 is selected from Y and S; and wherein X5 is selected from Y and S;
(ii) CDRH2 comprises an amino acid sequence: X1-I-X2-P-X3-X4-G-X5-T-X6-Y-A-D-S-V-K-G (SEQ ID NO: 2635), wherein X1 is position 50 according to the Kabat numbering system; wherein X1 is selected from Y and S; wherein X2 is selected from Y and S; wherein X3 is selected from Y and S; wherein X4 is selected from Y and S; wherein X5 is selected from Y and S; and wherein X6 is selected from Y and S; and
(iii) CDRH3 comprises an amino acid sequence: X1-X2-X3-X4-X5-X6-X7-X8-X9-X10-X11-X12-X13-X14-X15-X16-X17-X18-X19-D-Y (SEQ ID NO: 2637), wherein X1 is position 95 according to the Kabat numbering system, and wherein the amino acids at each of positions X1-X6 are selected from a pool of amino acids in a molar ratio of 25% Y, 50% S, and 25% R; wherein the amino acids at each of positions X7-X17 are selected from a pool of amino acids in a molar ratio of 25% Y, 50% S, and 25% R, or are not present; wherein X18 is selected from G and A; and wherein X19 is selected from I, M, L, and F.

13. A polypeptide comprising an immunoglobulin heavy chain variable domain, wherein:
(i) CDRH1 comprises an amino acid sequence G-F-X1-I-X2-X3-X4-X5-I-H (SEQ ID NO: 2634), wherein G is position 26 and X1 is position 28 according to the Kabat numbering system; wherein X1 is selected from Y and S; wherein X2 is selected from Y and S; wherein X3 is selected from Y and S; wherein X4 is selected from Y and S; and wherein X5 is selected from Y and S;
(ii) CDRH2 comprises an amino acid sequence: X1-I-X2-P-X3-X4-G-X5-T-X6-Y-A-D-S-V-K-G (SEQ ID NO: 2635), wherein X1 is position 50 according to the Kabat numbering system; wherein X1 is selected from Y and S; wherein X2 is selected from Y and S; wherein X3 is selected from Y and S; wherein X4 is selected from Y and S; wherein X5 is selected from Y and S; and wherein X6 is selected from Y and S; and
(iii) CDRH3 comprises an amino acid sequence: X1-X2-X3-X4-X5-X6-X7-X8-X9-X10-X11-X12-X13-X14-X15-X16-X17-X18-X19-D-Y (SEQ ID NO: 2638), wherein X1 is position 95 according to the Kabat numbering system, and wherein the amino acids at each of positions X1-X6 are selected from a pool of amino acids in a molar ratio of 38% Y, 25% S, 25% G, and 12% R; wherein the amino acids at each of positions X7-X17 are selected from a pool of amino acids in a molar ratio of 38% Y, 25% S, 25% G, and 12% R, or are not present; wherein X18 is selected from G and A; and wherein X19 is selected from I, M, L, and F.

14. A polypeptide comprising an immunoglobulin heavy chain variable domain, wherein:
(i) CDRH1 comprises an amino acid sequence G-F-X1-I-X2-X3-X4-X5-I-H (SEQ ID NO: 2634), wherein G is position 26 and X1 is position 28 according to the Kabat numbering system; wherein X1 is selected from Y and S; wherein X2 is selected from Y and S; wherein X3 is selected from Y and S; wherein X4 is selected from Y and S; and wherein X5 is selected from Y and S;
(ii) CDRH2 comprises an amino acid sequence: X1-I-X2-P-X3-X4-G-X5-T-X6-Y-A-D-S-V-K-G (SEQ ID NO: 2635), wherein X1 is position 50 according to the Kabat numbering system; wherein X1 is selected from Y and S; wherein X2 is selected from Y and S; wherein X3 is selected from Y and S; wherein X4 is selected from Y and S; wherein X5 is selected from Y and S; and wherein X6 is selected from Y and S; and
(iii) CDRH3 comprises an amino acid sequence: X1-X2-X3-X4-X5-X6-X7-X8-X9-X10-X11-X12-X13-X14-X15-X16-X17-X18-X19-D-Y (SEQ ID NO: 2639), wherein X1 is position 95 according to the Kabat numbering system, and wherein the amino acids at each of positions X1-X6 are selected from a pool of amino acids in a molar ratio of 20% Y, 26% S, 26% G, 13% R, 1% A, 1% D, 1% E, 1% F, 1% H, 1% I, 1% K, 1% L, 1% M, 1% N, 1% P, 1% Q, 1% T, 1% V, and 1% W; wherein the amino acids at each of positions X7-X17 are selected from a pool of amino acids in a molar ratio of 20% Y, 26% S, 26% G, 13% R, 1% A, 1% D, 1% E, 1% F, 1% H, 1% I, 1% K, 1% L, 1% M, 1% N, 1% P, 1% Q, 1% T, 1% V, and 1% W, or are not present; wherein X18 is selected from G and A; and wherein X19 is selected from I, M, L, and F.

15. The polypeptide of claim 14, wherein CDRH1 comprises an amino acid sequence selected from SEQ ID NOs: 318-439.

16. The polypeptide of claim 14, wherein CDRH2 comprises an amino acid sequence selected from SEQ ID NOs: 440-561.

17. The polypeptide of claim 14, wherein CDRH3 comprises an amino acid sequence selected from SEQ ID NOs: 562-683.

18. A polypeptide comprising an immunoglobulin heavy chain variable domain, wherein:
(i) CDRH1 comprises an amino acid sequence G-F-X1-I-X2-X3-X4-X5-I-H (SEQ ID NO: 2634), wherein G is position 26 and X1 is position 28 according to the Kabat numbering system; wherein X1 is selected from Y and S; wherein X2 is selected from Y and S; wherein X3 is selected from Y and S; wherein X4 is selected from Y and S; and wherein X5 is selected from Y and S;
(ii) CDRH2 comprises an amino acid sequence: X1-I-X2-P-X3-X4-S-X5-T-X6-Y-A-D-S-V-K-G (SEQ ID NO: 2635), wherein X1 is position 50 according to the Kabat numbering system; wherein X1 is selected from Y and S; wherein X2 is selected from Y and S; wherein X3 is selected from Y and S; wherein X4 is selected from Y and S; wherein X5 is selected from Y and S; and wherein X6 is selected from Y and S; and
(iii) CDRH3 comprises an amino acid sequence: X1-X2-X3-X4-X5-X6-X7-X8-X9-X10-X11-X12-X13-X14-X15-X16-X17-X18-X19 (SEQ ID NO: 2640), wherein X1 is position 95 according to the Kabat numbering system, and wherein the amino acids at each of positions X1-X19 are selected from S and one of A, C, F, G, I, L, N, P, R, T, W, or Y, or are not present; wherein X18 is selected from G and A; and wherein X19 is selected from F, L, I, and M.

19. The polypeptide of claim 18, wherein CDRHI comprises an amino acid sequence selected from SEQ ID NOs: 1340-1396, 1538-1564, 1653-1686, 1805-1854, and 1963-1970.

20. The polypeptide of claim 18, wherein CDRH2 comprises an amino acid sequence selected from SEQ ID NOs: 1397-1453, 1565-1591, 1687-1720, 1855-1904, and 1971-1978.

21. The polypeptide of claim 18, wherein CDRH3 comprises an amino acid sequence selected from SEQ ID NOs: 1454-1510, 1592-1618, 1721-1754, 1905-1954, and 1979-1986.

22. A polypeptide comprising an immunoglobulin heavy chain variable domain, wherein:
(i) CDRH1 comprises an amino acid sequence G-F-X1-I-X2-X3-X4-X5-I-H (SEQ ID NO: 2641), wherein G is position 26 and X1 is position 28 according to the Kabat numbering system; wherein the amino acid at each of positions X1-X5 is selected from S and one of Y, W, R, or F;
(ii) CDRH2 comprises an amino acid sequence: X1-I-X2-P-X3-X4-S-X5-T-X6-Y-A-D-S-V-K-G (SEQ ID NO: 2642), wherein X1 is position 50 according to the Kabat numbering system; wherein the amino acid at each of positions X1-X6 is selected from S and one of Y, W, R, or F; and
(iii) CDRH3 comprises an amino acid sequence: X1-X2-X3-X4-X5-X6-X7-X8-X9-X10-X11-X12-X13-X14-X15-X16-X17-X18-X19 (SEQ ID NO: 2643), wherein X1 is position 95 according to the Kabat numbering system, and wherein the amino acids at each of positions X1-X19 are selected from S and one of Y, W, R, or F, or are not present; wherein X18 is selected from G and A; and wherein X19 is selected from F, L, I, and M.

23. The polypeptide of claim 22, wherein CDRH1 comprises an amino acid sequence selected from SEQ ID NOs: 2027-2057, 2147-2173, 2239-2249, 2300-2327, and 2395-2405.

24. The polypeptide of claim 22, wherein CDRH2 comprises an amino acid sequence selected from SEQ ID NOs: 2058-2088, 2174-2200, 2250-2260, 2328-2355, and 2406-2416.

25. The polypeptide of claim 22, wherein CDRH3 comprises an amino acid sequence selected from SEQ ID NOs: 2089-2119, 2201-2227, 2261-2271, 2356-2383, and 2417-2427.

26. A polypeptide comprising an immunoglobulin heavy chain variable domain, wherein:
(i) CDRH1 comprises an amino acid sequence G-F-N-X1-X2-X3-X4-X5-X6-H (SEQ ID NO: 2644), wherein G is position 26 and X1 is position 29 according to the Kabat numbering system; and wherein X1-X6 are naturally occurring amino acids other than cysteine;
(ii) CDRH2 comprises an amino acid sequence: X6-I-X7-X8-X9-X10-X11-X12-T-X13-Y-A-D-S-V-K-G (SEQ ID NO: 2645), wherein X6 is position 50 according to the Kabat numbering system, and wherein X6-X13 are naturally occurring amino acids other than cysteine; and
(iii) CDRH3 comprises an amino acid sequence: X14-X15-X16-X17-X18-(X19)ₙ-X20-X21-D-Y (SEQ ID NO: 2646), wherein X14 is position 95 according to the Kabat numbering system, and wherein n is a suitable number that would retain the functional activity of the heavy chain variable domain, and wherein X14-X21 are naturally occurring amino acids other than cysteine.

27. The polypeptide of claim 26, wherein n is 1 to 12.

28. The polypeptide of claim 26, wherein X1 is selected from F, L, I, and V; wherein X2 is selected from Y and S; wherein X3 is selected from Y and S; wherein X4 is selected from Y and S; wherein X5 is selected from Y and S, and wherein X6 is selected from M and I.

29. The polypeptide of claim 26, wherein X6 is selected from Y and S; wherein X7 is selected from Y and S; wherein X8 is selected from P and S; wherein X9 is selected from Y and S; wherein X10 is selected from Y and S; wherein X11 is selected from G and S; wherein X12 is selected from Y and S; and wherein X13 is selected from Y and S.

30. The polypeptide of claim 26, wherein X14 is selected from Y and S; wherein X15 is selected from Y and S; wherein X16 is selected from Y and S, wherein X17 is selected from Y and S; wherein X18 is selected from Y and S; wherein X19 is selected from Y and S; wherein X20 is selected from G and A; and wherein X21 is selected from F, L, I, and M.

31. The polypeptide of claim 26, wherein the amino acids at each of positions X14-X19 are selected from a pool of amino acids in a molar ratio of 20% Y, 15% S, 15% G, 3.125% A, 3.125% D, 3.125% E, 3.125% F, 3.125% H, 3.125% I, 3.125% K, 3.125% L, 3.125% M, 3.125% N, 3.125% P, 3.125% Q, 3.125% R, 3.125% T, 3.125% V, and 3.125% W, wherein X20 is selected from G and A; and wherein X21 is selected from F, L, I, and M.

32. The polypeptide of claim 26, wherein CDRHI comprises an amino acid sequence selected from SEQ ID NOs: 52-66 and 111-125.

33. The polypeptide of claim 26, wherein CDRH2 comprises an amino acid sequence selected from SEQ ID NOs: 67-81 and 126-141.

34. The polypeptide of claim 26, wherein CDRH3 comprises an amino acid sequence selected from SEQ ID NOs: 82-96 and 142-157.

35-67. (canceled)

68. A polypeptide comprising an immunoglobulin light chain variable domain, wherein CDRL3 comprises an amino acid sequence: Q-Q-X1-X2-X3-X4-P-X5-T (SEQ ID NO: 2654), wherein X1 is position 91 according to the Kabat numbering system, and wherein the amino acids at each of positions X1-X5 are selected from S and one of Y, W, R, or F.

69. The polypeptide of claim 68, wherein CDRL3 comprises an amino acid sequence selected from SEQ ID NOs: 1996-2026, 2120-2146, 2228-2238, 2272-2299, and 2384-2394.

70. A polypeptide comprising an immunoglobulin light chain variable domain, wherein:
(i) CDRL1 comprises a first consensus hypervariable sequence or variant thereof comprising substitution at one or more positions compared to a corresponding consensus hypervariable sequence;
(ii) CDRL2 comprises a second consensus hypervariable sequence or variant thereof comprising substitution at one or more positions compared to a corresponding consensus hypervariable sequence; and
(iii) CDRL3 comprises an amino acid sequence: Q-Q-X1-X2-X3-(X4)ₙ-X5-X6-T (SEQ ID NO: 2655), wherein X1-X6 are any naturally occurring amino acids other than cysteine, and wherein X1 is position 91 according to the Kabat numbering system.

71. The polypeptide of claim 70, wherein X1 is position 91 according to the Kabat numbering system, wherein X1 is selected from Y and S; wherein X2 is selected from Y and S; wherein X3 is selected from Y and S; wherein X4 is selected from Y and S; wherein X5 is selected from P and L; and wherein X6 is selected from F, L, I, and V.

72. The polypeptide of claim 70, wherein n is 1 to 3.

73. The polypeptide of claim 72, wherein CDRL3 comprises an amino acid sequence selected from SEQ ID NOs: 37-51 and 97-110.

74. The polypeptide of claim 70, wherein the first consensus hypervariable sequence is R-A-S-Q-D-V-N-T-A-V-A (SEQ ID NO: 6).

75. The polypeptide of claim 70, wherein the second consensus hypervariable sequence is S-A-S-S-L-Y-S (SEQ ID NO: 7).

76-81. (canceled)

82. The antibody of claim 1 further comprising a polypeptide comprising an immunoglobulin light chain variable domain according to claim 68.

83. The polypeptide of claim 5 comprising
a light chain antibody variable domain comprising the polypeptide of claim 68.

84. A polypeptide according to claim 1, further comprising a dimerization domain linked to the C-terminal region of a heavy chain antibody variable domain.

85. A polypeptide according to claim 84, wherein the dimerization domain comprises a leucine zipper domain or a sequence comprising at least one cysteine residue.

86. A polypeptide according to claim 85, wherein the dimerization domain comprises a hinge region from an antibody and a leucine zipper.

87. A polypeptide according to claim 84, wherein the dimerization domain is a single cysteine.

88. A fusion polypeptide comprising a polypeptide according to claim 1, wherein an antibody variable domain comprising the polypeptide is fused to at least a portion of a viral coat protein.

89. The fusion polypeptide of claim 88, wherein the viral coat protein is selected from the group consisting of protein pIII, major coat protein pVIII, Soc, Hoc, gpD, pv1, and variants thereof.

90. The fusion polypeptide of claim 88, further comprising a dimerization domain between the variable domain and the viral coat protein.

91. (canceled)

92. The fusion polypeptide of claim 88, further comprising a variable domain fused to a peptide tag.

93. (canceled)

94. The fusion polypeptide of claim 92, wherein the peptide tag is selected from the group consisting of gD, c-myc, poly-his, a fluorescence protein, and B-galactosidase.

95. A polypeptide of claim 1, further comprising framework regions FR1, FR2, FR3, and/or FR4 for an antibody variable domain corresponding to the variant CDRH1, CDRH2, CDRH3, and/or CDRL3, wherein the framework regions are obtained from a single antibody template.

96. The polypeptide of claim 95, wherein each of the framework regions comprises an amino acid sequence corresponding to the framework region amino acid sequences of antibody 4D5 (SEQ ID NOs: 1099-1102 and 1103-1106) or a variant of antibody 4D5 (SEQ ID NOs: 1107-1110 and 1111-1114).

97. A library comprising a plurality of the polypeptide of claim 1, and wherein the library has at least 1×10⁴ distinct antibody variable domain sequences.

98. A method of generating a composition comprising a plurality of polypeptides comprising:
(a) generating a plurality of polypeptides comprising:
(i) CDRH1 comprising an amino acid sequence G-F-N-X1-X2-X3-X4-X5-X6-H (SEQ ID NO: 2629), wherein G is position 26 and X1 is position 29 according to the Kabat numbering system; wherein X1 is selected from F, L, I, and V; wherein X2 is selected from Y and S; wherein X3 is selected from Y and S; wherein X4 is selected from Y and S; wherein X5 is selected from Y and S, and wherein X6 is selected from M and I;
(ii) CDRH2 comprising an amino acid sequence: X1-I-X2-X3-X4-X5-X6-X7-T-X8-Y-A-D-S-V-K-G (SEQ ID NO: 2630), wherein X1 is position 50 according to the Kabat numbering system; wherein X1 is selected from Y and S; wherein X2 is selected from Y and S; wherein X3 is selected from P and S; wherein X4 is selected from Y and S; wherein X5 is selected from Y and S; wherein X6 is selected from G and S; wherein X7 is selected from Y and S; and wherein X8 is selected from Y and S; and
(iii) CDRH3 comprising an amino acid sequence: X1-X2-X3-X4-X5-X6-X7-X8-X9-X10-X11-X12-X13-X14-X15-X16-X17-X18-X19-D-Y (SEQ ID NO: 2631), wherein X1 is position 95 according to the Kabat numbering system, and wherein X1 is selected from Y and S; wherein X2 is selected from Y and S; wherein X3 is selected from Y and S, wherein X4 is selected from Y and S; wherein X5 is selected from Y and S; wherein X6 is selected from Y and S; wherein X7 is selected from Y and S or is not present; wherein X8 is selected from Y and S or is not present; wherein X9 is selected from Y and S or is not present; wherein X10 is selected from Y and S or is not present; wherein X11 is selected from Y and S or is not present; wherein X12 is selected from Y and S or is not present; wherein X13 is selected from Y and S or is not present; wherein X14 is selected from Y and S or is not present; wherein X15 is selected from Y and S or is not present; wherein X16 is selected from Y and S or is not present; wherein X17 is selected from Y and S or is not present; wherein X18 is selected from G and A; and wherein X19 is selected from F, L, I, and M.

99. A method of generating a composition comprising a plurality of polypeptides comprising:
(a) generating a plurality of polypeptides comprising:
(i) CDRH1 comprising an amino acid sequence G-F-N-X1-X2-X3-X4-X5-X6-H (SEQ ID NO: 2629), wherein G is position 26 and X1 is position 29 according to the Kabat numbering system; wherein X1 is selected from F, L, I, and V; wherein X2 is selected from Y and S; wherein X3 is selected from Y and S; wherein X4 is selected from Y and S; wherein X5 is selected from Y and S, and wherein X6 is selected from M and I;
(ii) CDRH2 comprising an amino acid sequence: X1-I-X2-X3-X4-X5-X6-X7-T-X8-Y-A-D-S-V-K-G (SEQ ID NO: 2630), wherein X1 is position 50 according to the Kabat numbering system; wherein X1 is selected from Y and S; wherein X2 is selected from Y and S; wherein X3 is selected from P and S; wherein X4 is selected from Y and S; wherein X5 is selected from Y and S; wherein X6 is selected from G and S; wherein X7 is selected from Y and S; and wherein X8 is selected from Y and S; and
(iii) CDRH3 comprising an amino acid sequence: X1-X2-X3-X4-X5-X6-X7-X8-X9-X10-X11-X12-X13-X14-X15-X16-X17-X18-X19-D-Y (SEQ ID NO: 2632), wherein X1 is position 95 according to the Kabat numbering system, and wherein the amino acids at each of positions X1-X6 are selected from a pool of amino acids in a molar ratio of 20% Y, 15% S, 15% G, 3.125% A, 3.125% D, 3.125% E, 3.125% F, 3.125% H, 3.125% I, 3.125% K, 3.125% L, 3.125% M, 3.125% N, 3.125% P, 3.125% Q, 3.125% R, 3.125% T, 3.125% V, and 3.125% W; wherein the amino acids at each of positions X7-X17 are selected from a pool of amino acids in a molar ratio of 20% Y, 15% S, 15% G, 3.125% A, 3.125% D, 3.125% E, 3.125% F, 3.125% H, 3.125% I, 3.125% K, 3.125% L, 3.125% M, 3.125% N, 3.125% P, 3.125% Q, 3.125% R, 3.125% T, 3.125% V, and 3.125% W, or are not present; wherein X18 is selected from G and A; and wherein X19 is selected from I, M, L, and F.

100. A method of generating a composition comprising a plurality of polypeptides comprising:
(a) generating a plurality of polypeptides comprising:
(i) CDRH1 comprising an amino acid sequence G-F-X1-I-X2-X3-X4-X5-I-H (SEQ ID NO: 2634), wherein G is position 26 and X1 is position 28 according to the Kabat numbering system; wherein X1 is selected from Y and S; wherein X2 is selected from Y and S; wherein X3 is selected from Y and S; wherein X4 is selected from Y and S; and wherein X5 is selected from Y and S;
(ii) CDRH2 comprising an amino acid sequence: X1-I-X2-P-X3-X4-S-X5-T-X6-Y-A-D-S-V-K-G (SEQ ID NO: 2657), wherein X1 is position 50 according to the Kabat numbering system; wherein X1 is selected from Y and S; wherein X2 is selected from Y and S; wherein X3 is selected from Y and S; wherein X4 is selected from Y and S; wherein X5 is selected from Y and S; and wherein X6 is selected from Y and S; and
(iii) CDRH3 comprising an amino acid sequence: X1-X2-X3-X4-X5-X6-X7-X8-X9-X10-X11-X12-X13-X14-X15-X16-X17-X18-X19 (SEQ ID NO: 2640), wherein X1 is position 95 according to the Kabat numbering system, and wherein the amino acids at each of positions X1-X19 are selected from S and one of A, C, F, G, I, L, N, P, R, T, W, or Y, or are not present; wherein X18 is selected from G and A; and wherein X19 is selected from F, L, I, and M.

101. A method of generating a composition comprising a plurality of polypeptides comprising:
(a) generating a plurality of polypeptides comprising:
(i) CDRH1 comprises an amino acid sequence G-F-X1-I-X2-X3-X4-X5-I-H (SEQ ID NO: 2641), wherein G is position 26 and X1 is position 28 according to the Kabat numbering system; wherein the amino acid at each of positions X1-X5 is selected from S and one of Y, W, R, or F;
(ii) CDRH2 comprises an amino acid sequence: X1-I-X2-P-X3-X4-S-X5-T-X6-Y-A-D-S-V-K-G (SEQ ID NO: 2642), wherein X1 is position 50 according to the Kabat numbering system; wherein the amino acid at each of positions X1-X6 is selected from S and one of Y, W, R, or F; and
(iii) CDRH3 comprises an amino acid sequence: X1-X2-X3-X4-X5-X6-X7-X8-X9-X10-X11-X12-X13-X14-X15-X16-X17-X18-X19 (SEQ ID NO: 2643), wherein X1 is position 95 according to the Kabat numbering system, and wherein the amino acids at each of positions X1-X19 are selected from S and one of Y, W, R, or F, or are not present; wherein X18 is selected from G and A; and wherein X19 is selected from F, L, I, and M.

102. The method of claim 98, wherein the method further comprises:
(b) generating a plurality of polypeptides comprising:
(i) CDRL1 comprising a first consensus hypervariable sequence or variant thereof comprising substitution at one or more positions compared to a corresponding consensus hypervariable sequence;
(ii) CDRL2 comprising a second consensus hypervariable sequence or variant thereof comprising substitution at one or more positions compared to a corresponding consensus hypervariable sequence; and
(iii) CDRL3 comprising an amino acid sequence: Q-Q-X1-X2-X3-X4-X5-X6-X7-X8-T (SEQ ID NO: 2652), wherein X1 is position 91 according to the Kabat numbering system; an wherein X1 is position 91 according to the Kabat numbering system, wherein X1 is selected from Y and S; wherein X2 is selected from Y and S; wherein X3 is selected from Y and S; wherein X4 is selected from Y and S; wherein X5 is selected from Y and S, or is not present; wherein X6 is selected from Y and S, or is not present; wherein X7 is selected from P and L; and wherein X8 is selected from F, L, I, and V.

103. The method of claim 100, wherein the method further comprises:
(b) generating a plurality of polypeptides comprising:
(i) CDRL1 comprising a first consensus hypervariable sequence or variant thereof comprising substitution at one or more positions compared to a corresponding consensus hypervariable sequence;
(ii) CDRL2 comprising a second consensus hypeiwariable sequence or variant thereof comprising substitution at one or more positions compared to a corresponding consensus hypervariable sequence; and
(iii) CDRL3 comprising an amino acid sequence: Q-Q-X1-X2-X3-X4-P-X5-T (SEQ ID NO: 2653), wherein X1 is position 91 according to the Kabat numbering system; wherein X1 is selected from Y and S; wherein X2 is selected from Y and S; wherein X3 is selected from Y and S; wherein X4 is selected from Y and S; and wherein X5 is selected from Y and S.

104. The method of claim 101, wherein the method further comprises:
(b) generating a plurality of polypeptides comprising:
(i) CDRL1 comprising a first consensus hypervariable sequence or variant thereof comprising substitution at one or more positions compared to a corresponding consensus hypervariable sequence;
(ii) CDRL2 comprising a second consensus hypervariable sequence or variant thereof comprising substitution at one or more positions compared to a corresponding consensus hypervariable sequence; and
(iii) CDRL3 comprising an amino acid sequence: Q-Q-X1-X2-X3-X4-P-X5-T (SEQ ID NO: 2654), wherein X1 is position 91 according to the Kabat numbering system; and wherein the amino acids at each of positions X1-X5 are selected from S and one of Y, W, R, and F.

105. A method of generating a composition comprising a plurality of polypeptides of claim 1 comprising:
(a) generating a plurality of polypeptides comprising:
(i) CDRH1 comprises an amino acid sequence G-F-X1-I-X2-X3-X4-X5-I-H (SEQ ID NO: 2634), wherein G is position 26 and X1 is position 28 according to the Kabat numbering system; wherein X1 is selected from Y and S; wherein X2 is selected from Y and S; wherein X3 is selected from Y and S; wherein X4 is selected from Y and S; and wherein X5 is selected from Y and S;
(ii) CDRH2 comprises an amino acid sequence: X1-I-X2-P-X3-X4-G-X5-T-X6-Y-A-D-S-V-K-G (SEQ ID NO: 2635), wherein X1 is position 50 according to the Kabat numbering system; wherein X1 is selected from Y and S; wherein X2 is selected from Y and S; wherein X3 is selected from Y and S; wherein X4 is selected from Y and S; wherein X5 is selected from Y and S; and wherein X6 is selected from Y and S; and
(iii) CDRH3 comprises an amino acid sequence: X1-X2-X3-X4-X5-X6-X7-X8-X9-X10-X11-X12-X13-X14-X15-X16-X17-X18-X19-D-Y (SEQ ID NO: 2636), wherein X1 is position 95 according to the Kabat numbering system, and wherein the amino acids at each of positions X1-X6 are selected from a pool of amino acids in a molar ratio of 50% Y, 25% S, and 25% G; wherein the amino acids at each of positions X7-X17 are selected from a pool of amino acids in a molar ratio of 50% Y, 25% S, and 25% G, or are not present; wherein X18 is selected from G and A; and wherein X19 is selected from I, M, L, and F.

106. A method of generating a composition comprising a plurality of polypeptides of claim 12 comprising:
(a) generating a plurality of polypeptides comprising:
(i) CDRH1 comprising an amino acid sequence G-F-X1-I-X2-X3-X4-X5-I-H (SEQ ID NO: 2634), wherein G is position 26 and X1 is position 28 according to the Kabat numbering system; wherein X1 is selected from Y and S; wherein X2 is selected from Y and S; wherein X3 is selected from Y and S; wherein X4 is selected from Y and S; and wherein X5 is selected from Y and S;
(ii) CDRH2 comprising an amino acid sequence: X1-I-X2-P-X3-X4-G-X5-T-X6-Y-A-D-S-V-K-G (SEQ ID NO: 2635), wherein X1 is position 50 according to the Kabat numbering system; wherein X1 is selected from Y and S; wherein X2 is selected from Y and S; wherein X3 is selected from Y and S; wherein X4 is selected from Y and S; wherein X5 is selected from Y and S; and wherein X6 is selected from Y and S; and
(iii) CDRH3 comprising an amino acid sequence: X1-X2-X3-X4-X5-X6-X7-X8-X9-X10-X11-X12-X13-X14-X15-X16-X17-X18-X19-D-Y (SEQ ID NO: 2637), wherein X1 is position 95 according to the Kabat numbering system, and wherein the amino acids at each of positions X1-X6 are selected from a pool of amino acids in a molar ratio of 25% Y, 50% S, and 25% R; wherein the amino acids at each of positions X7-X17 are selected from a pool of amino acids in a molar ratio of 25% Y, 50% S, and 25% R, or are not present; wherein X18 is selected from G and A; and wherein X19 is selected from I, M, L, and F.

107. A method of generating a composition comprising a plurality of polypeptides of claim 13 comprising:
(a) generating a plurality of polypeptides comprising:
(i) CDRH1 comprising an amino acid sequence G-F-X1-I-X2-X3-X4-X5-I-H (SEQ ID NO: 2934), wherein G is position 26 and X1 is position 28 according to the Kabat numbering system; wherein X1 is selected from Y and S; wherein X2 is selected from Y and S; wherein X3 is selected from Y and S; wherein X4 is selected from Y and S; and wherein X5 is selected from Y and S;
(ii) CDRH2 comprising an amino acid sequence: X1-I-X2-P-X3-X4-G-X5-T-X6-Y-A-D-S-V-K-G (SEQ ID NO: 2935), wherein X1 is position 50 according to the Kabat numbering system; wherein X1 is selected from Y and S; wherein X2 is selected from Y and S; wherein X3 is selected from Y and S; wherein X4 is selected from Y and S; wherein X5 is selected from Y and S; and wherein X6 is selected from Y and S; and
(iii) CDRH3 comprising an amino acid sequence: X1-X2-X3-X4-X5-X6-X7-X8-X9-X10-X11-X12-X13-X14-X15-X16-X17-X18-X19-D-Y (SEQ ID NO: 2938), wherein X1 is position 95 according to the Kabat numbering system, and wherein the amino acids at each of positions X1-X6 are selected from a pool of amino acids in a molar ratio of 38% Y, 25% S, 25% G, and 12% R; wherein the amino acids at each of positions X7-X17 are selected from a pool of amino acids in a molar ratio of 38% Y, 25% S, 25% G, and 12% R, or are not present; wherein X18 is selected from G and A; and wherein X19 is selected from I, M, L, and F.

108. A method of generating a composition comprising a plurality of polypeptides of claim 14 comprising:
(a) generating a plurality of polypeptides comprising:
(i) CDRH1 comprising an amino acid sequence G-F-X1-I-X2-X3-X4-X5-I-H (SEQ ID NO: 2934), wherein G is position 26 and X1 is position 28 according to the Kabat numbering system; wherein X1 is selected from Y and S; wherein X2 is selected from Y and S; wherein X3 is selected from Y and S; wherein X4 is selected from Y and S; and wherein X5 is selected from Y and S;
(ii) CDRH2 comprising an amino acid sequence: X1-I-X2-P-X3-X4-G-X5-T-X6-Y-A-D-S-V-K-G (SEQ ID NO: 2935), wherein X1 is position 50 according to the Kabat numbering system; wherein X1 is selected from Y and S; wherein X2 is selected from Y and S; wherein X3 is selected from Y and S; wherein X4 is selected from Y and S; wherein X5 is selected from Y and S; and wherein X6 is selected from Y and S; and
(iii) CDRH3 comprising an amino acid sequence: X1-X2-X3-X4-X5-X6-X7-X8-X9-X10-X11-X12-X13-X14-X15-X16-X17-X18-X19-D-Y (SEQ ID NO: 2939), wherein X1 is position 95 according to the Kabat numbering system, and wherein the amino acids at each of positions X1-X6 are selected from a pool of amino acids in a molar ratio of 20% Y, 26% S, 26% G, 13% R, 1% A, 1% D, 1% E, 1% F, 1% H, 1% I, 1% K, 1% L, 1% M, 1% N, 1% P, 1% Q, 1% T, 1% V, and 1% W; wherein the amino acids at each of positions X7-X17 are selected from a pool of amino acids in a molar ratio of 20% Y, 26% S, 26% G, 13% R, I% A, 1% D, 1% E, 1% F, 1% H, 1% I, 1% K, 1% L, 1% M, 1% N, 1% P, 1% Q, 1% T, 1% V, and 1% W, or are not present; wherein X18 is selected from G and A; and wherein X19 is selected from I, M, L, and F.

109. The method of claim 105, wherein the method further comprises:
(b) generating a plurality of polypeptides comprising:
(i) CDRL1 comprising a first consensus hypervariable sequence or variant thereof comprising substitution at one or more positions compared to a corresponding consensus hypervariable sequence;
(ii) CDRL2 comprising a second consensus hypervariable sequence or variant thereof comprising substitution at one or more positions compared to a corresponding consensus hypervariable sequence; and
(iii) CDRL3 comprising an amino acid sequence: Q-Q-X1-X2-X3-X4-P-X5-T (SEQ ID NO: 2653), wherein X1 is position 91 according to the Kabat numbering system, and wherein X1 is selected from Y and S, wherein X2 is selected from Y and S; wherein X3 is selected from Y and S; wherein X4 is selected from Y and S; and wherein X5 is selected from Y and S.

110. The method of claim 109, wherein the first consensus hypervariable sequence comprises a Kabat consensus CDRL1 sequence.

111. The method of claim 109, wherein the first consensus hypervariable sequence is R-A-S-Q-D-V-N-T-A-V-A (SEQ ID NO: 6).

112. The method of claim 109, wherein the second consensus hypervariable sequence comprises a Kabat consensus CDRL2 sequence.

113. The method of claim 109, wherein the second consensus hypervariable sequence is S-A-S-S-L-Y-S (SEQ ID NO: 7).

114. The method of claim 108, wherein the plurality of polypeptides are encoded by a plurality of polynucleotides.

115-116. (canceled)

117. A method of selecting for an antigen binding variable domain that binds to a target antigen from a library of antibody variable domains comprising:
(a) contacting the library of claim 97 with a target antigen;
(b) separating one or more polypeptides that specifically bind to the target antigen from polypeptides that do not specifically bind to the target antigen, recovering the one or more polypeptides that specifically bind to the target antigen, and incubating the one or more polypeptides that specifically bind to the target antigen in a series of solutions comprising decreasing amounts of the target antigen in a concentration from about 0.1 nM to about 1000 nM; and
(c) selecting the one or more polypeptides that specifically bind to the target antigen and that can bind to the lowest concentration of the target antigen or that have an affinity of about 0.1 nM to about 200 nM.

118. The method according to claim 117, wherein the target antigen is VEGF, insulin, HER2, IGF-1, or growth hormone.

119. The method according to claim 117, wherein the concentration of the target antigen is about 100 to about 250 nM.

120. The method according to claim 117, wherein the concentration of target antigen is about 25 to about 100 nM.

121-130. (canceled)

131. The antibody of claim 82, wherein the antibody specifically binds human VEGF.

132-140. (canceled)

141. The antibody of claim 126 or 127 131, comprising CDRH1, CDRH2, CDRH3, and CDRL3 sequences corresponding to the CDRH1, CDRH2, CDRH3, and CDRL3 sequences set forth in FIG. 10 for any one of Fabs 1-31, set forth in FIGS. 21A-21B for any one of clones A1-A60, or set forth in FIG. 28A for any one of clones F1-F31.

142. The antibody of claim 126 or 127 131, comprising CDRH1, CDRH2, CDRH3, and CDRL3 sequences corresponding to the CDRH1, CDRH2, CDRH3, and CDRL3 sequences set forth in FIGS. 14A-C for any one of clones 1-122.

143. An isolated polynucleotide encoding the antibody of claim 131.

144. A vector comprising the nucleic acid of claim 143.

145. A host cell transformed with the vector of claim 144.

146-148. (canceled)

149. A method of using the antibody of claim 131 for treating a disorder associated with abnormal angiogenesis in a mammal in need of treatment thereof comprising the step of administering the antibody to the mammal.

150-154. (canceled)

155. A method of treating a mammal suffering from or at risk of developing an inflammatory or immune disorder comprising the step of treating the mammal with a Fab of the antibody of claim 131.

156. The method of claim 155, wherein the inflammatory or immune disorder is rheumatoid arthritis.

157. The polypeptide of claim 11, wherein the polypeptide specifically binds insulin.

158-168. (canceled)

169. The antibody of claim 158, comprising CDRH1, CDRH2, CDRH3, and CDRL3 sequences corresponding to the CDRH1, CDRH2, CDRH3, and CDRL3 sequences set forth in FIGS. 15A-15B for any one of clones 1-105, set forth in FIG. 23A for any one of clones C1-C34, or set forth in FIG. 30A for any one of clones H43-H55.

170. An isolated polynucleotide encoding the polypeptide claim 157.

171. A vector comprising the nucleic acid of claim 170.

172. A host cell transformed with the vector of claim 171.

173-175. (canceled)

176. A method of using the polypeptide of claim 158 for treating an insulin-related disorder in a mammal in need of treatment thereof comprising the step of administering the antibody to the mammal.

177. (canceled)

178. The polypeptide of claim 18, wherein the polypeptide specifically binds HER2.

179-189. (canceled)

190. The antibody of claim 179, comprising CDRH1, CDRH2, CDRH3, and CDRL3 sequences corresponding to the CDRH1, CDRH2, CDRH3, and CDRL3 sequences set forth in FIG. 22A for any one of clones B1-B28 or FIG. 29A for any one of clones G29-G61.

191. An isolated polynucleotide encoding the polypeptide of claim 178.

192. A vector comprising the nucleic acid of claim 191.

193. A host cell transformed with the vector of claim 192.

194-196. (canceled)

197. A method of using the polypeptide of claim 179 for treating a HER2-related disorder in a mammal in need of treatment thereof comprising the step of administering the antibody to the mammal.

198. (canceled)

199. The polypeptide of claim 18, wherein the polypeptide specifically binds IGF-1.

200-210. (canceled)

211. The antibody of claim 200, comprising CDRH1, CDRH2, CDRH3, and CDRL3 sequences corresponding to the CDRH1, CDRH2, CDRH3, and CDRL3 sequences set forth in FIG. 24A for any one of clones D44-D96 or in FIG. 31A for any one of clones I67-I96.

212. An isolated polynucleotide encoding the polypeptide of claim 199.

213. A vector comprising the nucleic acid of claim 212.

214. A host cell transformed with the vector of claim 213.

215-217. (canceled)

218. A method of using the polypeptide of claim 199 for treating an IGF-1-related disorder in a mammal in need of treatment thereof comprising the step of administering the antibody to the mammal.

219. (canceled)

220. The polypeptide of claim 18 wherein the polypeptide specifically binds HGH.

221-231. (canceled)

232. The antibody of claim 220, comprising CDRH1, CDRH2, CDRH3, and CDRL3 sequences corresponding to the CDRH1, CDRH2, CDRH3, and CDRL3 sequences set forth in FIG. 25A for any one of clones E35-E43 or FIG. 32A for any one of clones J56-J66.

233. An isolated polynucleotide encoding the polypeptide of claim 220.

234. A vector comprising the nucleic acid of claim 233.

235. A host cell transformed with the vector of claim 234.

236-238. (canceled)

239. A method of using the polypeptide of claim 220 for treating an HGH-related disorder in a mammal in need of treatment thereof comprising the step of administering the antibody to the mammal.

240. (canceled)
